SEQUENCE LISTING (34 sequences)

SEQ ID NO: 1 - protein, 536 aa, synthetic construct
MEFSSPSREECPKPLSRVSIMAGSLTGLLLLQAVSWASGARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWRRQ

SEQ ID NO: 2 - protein, 497 aa, synthetic construct
ARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWRRQ

SEQ ID NO: 3 - DNA, 1611 bp, synthetic construct
  source 1..1611: mol_type = other DNA
atggagttcagctctccgagccgggaagagtgccccaaacccctgtcgagagtgtccatcatggccggcagcctgaccggcctgctgctgctgcaagctgttagctgggccagcggggccagaccttgtatccccaagagcttcggatatagcagcgtggtctgcgtgtgcaacgccacctactgcgatagcttcgacccacctacctttccagctctgggcaccttctccagatacgagtctacaagaagcggcagaagaatggaactgtccatgggccctatccaggccaaccacaccggcacaggcctcctgttgaccctgcagcccgagcagaaatttcagaaagtgaagggattcggcggcgccatgaccgatgccgccgctctgaatatcctggcgctgagccctcctgcccagaacctgctgctgaagagctactttagcgaggaggggatcggctacaacattatcagagtgcccatggccagctgcgactttagcatcagaacctatacatacgccgacaccccagatgacttccagctgcacaacttcagcctgcctgaggaagatacaaagctgaaaatccccctgatccaccgggccctgcaactggctcagcggccagtgtccctgctggcctctccttggaccagccctacctggctgaagaccaatggcgccgtgaacggcaagggctctctgaagggccagcctggcgacatctaccatcagacctgggccagatacttcgtgaagttcctggatgcctacgctgagcacaagctgcagttctgggccgtgaccgccgagaacgagccttccgccggactgctcagcggctatcctttccagtgcctgggcttcacacctgagcaccagcgcgacttcatcgccagggacctgggccctacactggccaatagcacacaccacaacgtgagactgctgatgctggatgatcagagactgctgcttccccactgggctaaggtcgtgctgaccgaccctgaagccgctaagtacgtgcacggcatcgccgtgcactggtacctggacttcctggcccctgccaaggccaccctgggagaaacccaccggctgttccctaacaccatgctgtttgcctcagaggcctgtgtgggctccaaattctgggagcaaagcgtgcggctgggctcttgggacagaggaatgcagtacagccacagcatcatcaccaacctgctgtaccacgtggtgggctggaccgactggaacctggccctgaaccccgagggaggccctaactgggtcagaaacttcgtggacagccccatcattgtggatatcaccaaggacacgttctacaagcagccaatgttctaccacctgggccacttcagcaagttcatccctgaaggcagtcagagagtgggcctggtggcctctcagaagaacgacctggacgccgttgcgctgatgcaccccgacggcagcgccgtggtggtggtgctgaatagatcttctaaggatgtgcctctgacaatcaaggaccctgctgtgggatttctggaaacaatcagccctggatacagcatccatacatacctgtggcggcggcagtga

SEQ ID NO: 4 - DNA, 1548 bp, synthetic construct
  source 1..1548: mol_type = other DNA
atggcctctctgtggctgctgagctgcttcagcctggtgggcgccgctttcggcgccagaccttgtatccccaagagcttcggatatagcagcgtggtctgcgtgtgcaacgccacctactgcgatagcttcgacccacctacctttccagctctgggcaccttctccagatacgagtctacaagaagcggcagaagaatggaactgtccatgggccctatccaggccaaccacaccggcacaggcctcctgttgaccctgcagcccgagcagaaatttcagaaagtgaagggattcggcggcgccatgaccgatgccgccgctctgaatatcctggcgctgagccctcctgcccagaacctgctgctgaagagctactttagcgaggaggggatcggctacaacattatcagagtgcccatggccagctgcgactttagcatcagaacctatacatacgccgacaccccagatgacttccagctgcacaacttcagcctgcctgaggaagatacaaagctgaaaatccccctgatccaccgggccctgcaactggctcagcggccagtgtccctgctggcctctccttggaccagccctacctggctgaagaccaatggcgccgtgaacggcaagggctctctgaagggccagcctggcgacatctaccatcagacctgggccagatacttcgtgaagttcctggatgcctacgctgagcacaagctgcagttctgggccgtgaccgccgagaacgagccttccgccggactgctcagcggctatcctttccagtgcctgggcttcacacctgagcaccagcgcgacttcatcgccagggacctgggccctacactggccaatagcacacaccacaacgtgagactgctgatgctggatgatcagagactgctgcttccccactgggctaaggtcgtgctgaccgaccctgaagccgctaagtacgtgcacggcatcgccgtgcactggtacctggacttcctggcccctgccaaggccaccctgggagaaacccaccggctgttccctaacaccatgctgtttgcctcagaggcctgtgtgggctccaaattctgggagcaaagcgtgcggctgggctcttgggacagaggaatgcagtacagccacagcatcatcaccaacctgctgtaccacgtggtgggctggaccgactggaacctggccctgaaccccgagggaggccctaactgggtcagaaacttcgtggacagccccatcattgtggatatcaccaaggacacgttctacaagcagccaatgttctaccacctgggccacttcagcaagttcatccctgaaggcagtcagagagtgggcctggtggcctctcagaagaacgacctggacgccgttgcgctgatgcaccccgacggcagcgccgtggtggtggtgctgaatagatcttctaaggatgtgcctctgacaatcaaggaccctgctgtgggatttctggaaacaatcagccctggatacagcatccatacatacctgtggcggcggcagtga

SEQ ID NO: 5 - DNA, 1551 bp, synthetic construct
  source 1..1551: mol_type = other DNA
atgttccagaccggcggactgatcgtgttctacggcctgctggcccagacaatggccgccagaccttgtatccccaagagcttcggatatagcagcgtggtctgcgtgtgcaacgccacctactgcgatagcttcgacccacctacctttccagctctgggcaccttctccagatacgagtctacaagaagcggcagaagaatggaactgtccatgggccctatccaggccaaccacaccggcacaggcctcctgttgaccctgcagcccgagcagaaatttcagaaagtgaagggattcggcggcgccatgaccgatgccgccgctctgaatatcctggcgctgagccctcctgcccagaacctgctgctgaagagctactttagcgaggaggggatcggctacaacattatcagagtgcccatggccagctgcgactttagcatcagaacctatacatacgccgacaccccagatgacttccagctgcacaacttcagcctgcctgaggaagatacaaagctgaaaatccccctgatccaccgggccctgcaactggctcagcggccagtgtccctgctggcctctccttggaccagccctacctggctgaagaccaatggcgccgtgaacggcaagggctctctgaagggccagcctggcgacatctaccatcagacctgggccagatacttcgtgaagttcctggatgcctacgctgagcacaagctgcagttctgggccgtgaccgccgagaacgagccttccgccggactgctcagcggctatcctttccagtgcctgggcttcacacctgagcaccagcgcgacttcatcgccagggacctgggccctacactggccaatagcacacaccacaacgtgagactgctgatgctggatgatcagagactgctgcttccccactgggctaaggtcgtgctgaccgaccctgaagccgctaagtacgtgcacggcatcgccgtgcactggtacctggacttcctggcccctgccaaggccaccctgggagaaacccaccggctgttccctaacaccatgctgtttgcctcagaggcctgtgtgggctccaaattctgggagcaaagcgtgcggctgggctcttgggacagaggaatgcagtacagccacagcatcatcaccaacctgctgtaccacgtggtgggctggaccgactggaacctggccctgaaccccgagggaggccctaactgggtcagaaacttcgtggacagccccatcattgtggatatcaccaaggacacgttctacaagcagccaatgttctaccacctgggccacttcagcaagttcatccctgaaggcagtcagagagtgggcctggtggcctctcagaagaacgacctggacgccgttgcgctgatgcaccccgacggcagcgccgtggtggtggtgctgaatagatcttctaaggatgtgcctctgacaatcaaggaccctgctgtgggatttctggaaacaatcagccctggatacagcatccatacatacctgtggcggcggcagtga

SEQ ID NO: 6 - DNA, 1545 bp, synthetic construct
  source 1..1545: mol_type = other DNA
atgctgctgctgctgctgctgctcggcctgagactgcagctgagcctgggcgccagaccttgtatccccaagagcttcggatatagcagcgtggtctgcgtgtgcaacgccacctactgcgatagcttcgacccacctacctttccagctctgggcaccttctccagatacgagtctacaagaagcggcagaagaatggaactgtccatgggccctatccaggccaaccacaccggcacaggcctcctgttgaccctgcagcccgagcagaaatttcagaaagtgaagggattcggcggcgccatgaccgatgccgccgctctgaatatcctggcgctgagccctcctgcccagaacctgctgctgaagagctactttagcgaggaggggatcggctacaacattatcagagtgcccatggccagctgcgactttagcatcagaacctatacatacgccgacaccccagatgacttccagctgcacaacttcagcctgcctgaggaagatacaaagctgaaaatccccctgatccaccgggccctgcaactggctcagcggccagtgtccctgctggcctctccttggaccagccctacctggctgaagaccaatggcgccgtgaacggcaagggctctctgaagggccagcctggcgacatctaccatcagacctgggccagatacttcgtgaagttcctggatgcctacgctgagcacaagctgcagttctgggccgtgaccgccgagaacgagccttccgccggactgctcagcggctatcctttccagtgcctgggcttcacacctgagcaccagcgcgacttcatcgccagggacctgggccctacactggccaatagcacacaccacaacgtgagactgctgatgctggatgatcagagactgctgcttccccactgggctaaggtcgtgctgaccgaccctgaagccgctaagtacgtgcacggcatcgccgtgcactggtacctggacttcctggcccctgccaaggccaccctgggagaaacccaccggctgttccctaacaccatgctgtttgcctcagaggcctgtgtgggctccaaattctgggagcaaagcgtgcggctgggctcttgggacagaggaatgcagtacagccacagcatcatcaccaacctgctgtaccacgtggtgggctggaccgactggaacctggccctgaaccccgagggaggccctaactgggtcagaaacttcgtggacagccccatcattgtggatatcaccaaggacacgttctacaagcagccaatgttctaccacctgggccacttcagcaagttcatccctgaaggcagtcagagagtgggcctggtggcctctcagaagaacgacctggacgccgttgcgctgatgcaccccgacggcagcgccgtggtggtggtgctgaatagatcttctaaggatgtgcctctgacaatcaaggaccctgctgtgggatttctggaaacaatcagccctggatacagcatccatacatacctgtggcggcggcagtga

SEQ ID NO: 7 - DNA, 1548 bp, synthetic construct
  source 1..1548: mol_type = other DNA
atgaccatcctgttcctgacaatggtgatcagctacttcggctgcatgaaggccgccagaccttgtatccccaagagcttcggatatagcagcgtggtctgcgtgtgcaacgccacctactgcgatagcttcgacccacctacctttccagctctgggcaccttctccagatacgagtctacaagaagcggcagaagaatggaactgtccatgggccctatccaggccaaccacaccggcacaggcctcctgttgaccctgcagcccgagcagaaatttcagaaagtgaagggattcggcggcgccatgaccgatgccgccgctctgaatatcctggcgctgagccctcctgcccagaacctgctgctgaagagctactttagcgaggaggggatcggctacaacattatcagagtgcccatggccagctgcgactttagcatcagaacctatacatacgccgacaccccagatgacttccagctgcacaacttcagcctgcctgaggaagatacaaagctgaaaatccccctgatccaccgggccctgcaactggctcagcggccagtgtccctgctggcctctccttggaccagccctacctggctgaagaccaatggcgccgtgaacggcaagggctctctgaagggccagcctggcgacatctaccatcagacctgggccagatacttcgtgaagttcctggatgcctacgctgagcacaagctgcagttctgggccgtgaccgccgagaacgagccttccgccggactgctcagcggctatcctttccagtgcctgggcttcacacctgagcaccagcgcgacttcatcgccagggacctgggccctacactggccaatagcacacaccacaacgtgagactgctgatgctggatgatcagagactgctgcttccccactgggctaaggtcgtgctgaccgaccctgaagccgctaagtacgtgcacggcatcgccgtgcactggtacctggacttcctggcccctgccaaggccaccctgggagaaacccaccggctgttccctaacaccatgctgtttgcctcagaggcctgtgtgggctccaaattctgggagcaaagcgtgcggctgggctcttgggacagaggaatgcagtacagccacagcatcatcaccaacctgctgtaccacgtggtgggctggaccgactggaacctggccctgaaccccgagggaggccctaactgggtcagaaacttcgtggacagccccatcattgtggatatcaccaaggacacgttctacaagcagccaatgttctaccacctgggccacttcagcaagttcatccctgaaggcagtcagagagtgggcctggtggcctctcagaagaacgacctggacgccgttgcgctgatgcaccccgacggcagcgccgtggtggtggtgctgaatagatcttctaaggatgtgcctctgacaatcaaggaccctgctgtgggatttctggaaacaatcagccctggatacagcatccatacatacctgtggcggcggcagtga

SEQ ID NO: 8 - DNA, 145 bp, Adeno-associated virus type 2
  source 1..145: mol_type = unassigned DNA
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 9 - DNA, 145 bp, Adeno-associated virus type 2
  source 1..145: mol_type = unassigned DNA
aggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 10 - DNA, 381 bp, synthetic construct
  source 1..381: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgg

SEQ ID NO: 11 - DNA, 323 bp, synthetic construct
  source 1..323: mol_type = other DNA
tcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgcc

SEQ ID NO: 12 - DNA, 594 bp, synthetic construct
  source 1..594: mol_type = other DNA
ttaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctg

SEQ ID NO: 13 - protein, 736 aa, Adeno-associated virus serotype 9
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 14 - protein, 611 aa, Adeno-associated virus serotype 9
TAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAAAKGGGFHGASAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 15 - protein, 534 aa, Adeno-associated virus serotype 9
MASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 16 - protein, 7 aa, synthetic construct
KGGGFHG

SEQ ID NO: 17 - protein, 12 aa, synthetic construct
AAAKGGGFHGAS

SEQ ID NO: 18 - protein, 748 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAAAKGGGFHGASAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 19 - DNA, 10864 bp, synthetic construct
  source 1..10864: mol_type = other DNA
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttccttacgtacaattgggatcccggaccgtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgccaccatggagttcagctctccgagccgggaagagtgccccaaacccctgtcgagagtgtccatcatggccggcagcctgaccggcctgctgctgctgcaagctgttagctgggccagcggggccagaccttgtatccccaagagcttcggatatagcagcgtggtctgcgtgtgcaacgccacctactgcgatagcttcgacccacctacctttccagctctgggcaccttctccagatacgagtctacaagaagcggcagaagaatggaactgtccatgggccctatccaggccaaccacaccggcacaggcctcctgttgaccctgcagcccgagcagaaatttcagaaagtgaagggattcggcggcgccatgaccgatgccgccgctctgaatatcctggcgctgagccctcctgcccagaacctgctgctgaagagctactttagcgaggaggggatcggctacaacattatcagagtgcccatggccagctgcgactttagcatcagaacctatacatacgccgacaccccagatgacttccagctgcacaacttcagcctgcctgaggaagatacaaagctgaaaatccccctgatccaccgggccctgcaactggctcagcggccagtgtccctgctggcctctccttggaccagccctacctggctgaagaccaatggcgccgtgaacggcaagggctctctgaagggccagcctggcgacatctaccatcagacctgggccagatacttcgtgaagttcctggatgcctacgctgagcacaagctgcagttctgggccgtgaccgccgagaacgagccttccgccggactgctcagcggctatcctttccagtgcctgggcttcacacctgagcaccagcgcgacttcatcgccagggacctgggccctacactggccaatagcacacaccacaacgtgagactgctgatgctggatgatcagagactgctgcttccccactgggctaaggtcgtgctgaccgaccctgaagccgctaagtacgtgcacggcatcgccgtgcactggtacctggacttcctggcccctgccaaggccaccctgggagaaacccaccggctgttccctaacaccatgctgtttgcctcagaggcctgtgtgggctccaaattctgggagcaaagcgtgcggctgggctcttgggacagaggaatgcagtacagccacagcatcatcaccaacctgctgtaccacgtggtgggctggaccgactggaacctggccctgaaccccgagggaggccctaactgggtcagaaacttcgtggacagccccatcattgtggatatcaccaaggacacgttctacaagcagccaatgttctaccacctgggccacttcagcaagttcatccctgaaggcagtcagagagtgggcctggtggcctctcagaagaacgacctggacgccgttgcgctgatgcaccccgacggcagcgccgtggtggtggtgctgaatagatcttctaaggatgtgcctctgacaatcaaggaccctgctgtgggatttctggaaacaatcagccctggatacagcatccatacatacctgtggcggcggcagtgagattaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgatcctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctcgaaattaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaatcgcgacggccgacgtctttgttacaacttactatatatatgcacacatatatatatatttgggtatattgggggggttctaatttaagaaatgcataattggctatagacagacagttgtcagaacttggcaatgggtacgtgcaggttcattataccaagtctacttgtagttgttcaaaatgtatcataatacaaggccgggcgaggtcgtcacgcctgtaatcccagcattttgggaggctaaggcaggaggattgcttgaggtcaggagtttgtgaccagcctgggcaacagagcaagaccctgtctccaaaaagaaaaaaaataattttttacaaaataaaaacaaaatgtatcatcagacgaaattaaataagaggcaattcattgtaatgacaacttttcccagcttgacatttaacaaaaagtctaagtcctcttaattcatatttaatgatcaaatatcaaatactaattttttttttttttttttttttgagacggagtctcgctctgtcgcccaggctggagtgcagtggcgcgatcctggctcactgcaagctccgcctcccgggttcacgccattctcctgcctcagcctcccgagtagctgggattacagacatgcgccaccacgcccggctaattttgtatttttagtagagatggggtttctccatgttggtcaggctggtcttgaatttcccacctcaggtgatctgcctgcctcagcctcacaaagcagtagctgggactacaggcacccaccaccacacttggttaattcttttgtattttttttgtaaagacgggatttcaccatgttagccaggatggtctcgatctcctgatctcatgatccgcccgcctcagcctcccaaagtgctgggattacaggcgtgagccaccccgcccggccatcaaatactaattcttaaatggtaaggacccactattcagaacctgtatccttatcactaatatgcaaatatttattgaatacttactatgtcatgcatactagagagagttagataaatttgatacagctaccctcacagaacttacagtgtaatagatggcatgacatgtacatgagtaactgtgaacagtgttaaattgctatttaaaaaaaaagacggctgggcgctgtggctcatgcctgtaatcccagcactttgggaggccaaggcaagttgatcgctcgaggtcaagagttcgagaccagcctggccaacgtggtaaaaccccgtctctactaaaaatacaaaaaaaaaattagccaggcatggtggcacaggcctgtaatcccagctactagggaggctgagacatggagaactgcttgaatccaggaggcagaggttacagtgagccgagatcataccactacactccagcctgagtgacagagcgagactcctgtctaaaaaaaaaaaaaaaaaaaaagatacaggttaagtgttatggtagttgaagagagaactcaaactctgtctcagaagcctcacttgcatgtggaccactgatatgaaataatataaataggtataattcaataaataggaacttcagttttaatcatcccaaacaccaaaacttcctatcaaacaggtccaataaactcaatctctataagagctagacagaaatctacttggtggcctataatcttattagcccttacttgtcccatctgatattaattaaccccatctaatatggattagttaacaatccagtggctgctttgacaggaacagttggagagagttggggattgcaacatattcaattatacaaaaatgcattcagcatctaccttgattaaggcagtgtgcaacagaatttgcaggagagtaaaagaatgattataaatttacaacccttaaagagctatagctgggcgtggtggctcatgcctgtaaatcccagcactttgggaggctgaggcgggtggatcacctgaggccagaagttcaagaccagcctagccaacatggcgaaaccctgtctctacaaaaaatacaaaaattagccgggtgtggtggcacgtgcctgtagtcccagttacttgggaggccgaggcaggagaatcgcttgaacctaggaggtggaggctgcagtgagccgagattgtgccactgcactccacttcagcctgggcgacaagagcaagactccgtcacaaaaaaaaaaaaaaaaaaaaaggctaaaatctagtgggaaaggcatatatacatacaactaactgtatagcataataaagctcataatctgtaacaaaatctaattcgacaagcccagaaacttgtgatttaccaaaaacagttatatatacacaaaaagtaaacctagaacccaaagttacccagcaccaatgattctctccctaagcagtatcaagtttaaagcagtgattacattctactgcctagattgtaaactgagtaaaggagaccagcacctttctgctactgaactagcacagccgtgtaaaccaacaaggcaatggcagtgcccaactttctgtatgaatataagttacatctgttttattatttgtgacttggtgttgcatgtggttattatcaacaccttctgaaagaacaactacctgctcaggctgccataacaaaataccacagactgagtgacttaacagaaacttatttctcacagttttggaggctgggaagtccaaaattaagctaactgcaaggtaggtttcaatctcaggcctcttctttggcttgaaggtcttctaactgtgtgctcacatgacctcttctaacaagctctctggtgtctctttttttttttttttcttttttgagacagagtctcactctgtcacccaggctggagtacagtggcacaatctgggctcactgcaacctccaactcccgggttcaagtgattctcatgcctcaccctcccgagtagcttggatgacaggagcccgctaccacacccagctaatttttgtatttttagtagagatggtgtttcactacattggccaggctggtctcaaactcctgacctcgtgatccacccaccttggcctcccaaagtgctgggattacaggtgtgagccactgcgcccgtcctggtgtcttttcatataagggcactaatccaatcagacctgggcccaaccctcccgacttcttctaactgtaattaccttccaaaggccctgtctccaaataccatcacactgggggttaggacttcaaaaaaggtatggggggggtgtgggaggacataaatgctcagtccataacaagcacccaacataaaaatggctagaacagatcacaaaaaaaaggtcctgtatggctttggggaagggctcaaccccaaaatatctgaaagctctggaggggcctagaagtggtaaatgaatgaaaacgtggttactctcccgatctgcctttcccaaatatggccattcttggctgaatcagaaatcaaaggacaggttattaattactagctctaagttacttaccatttgctgagacagttcagaaatctgactgcatctcctcagggatctagaacacagttctcaaattctaacttacttgtgatatacttgtgaatgataaaaatcgctacaggtacttttattaatctgaaagagtattgagaaattacctttcattctgacttttgtctggaatgaaaatcaatacttttgctatattccattactgaaataattttactttccagtaaaactggcattataattttttttaatttttaaaacttcataattttttgccagactgacccatgtaaacatacaaattactaataattatgcacgtcacatctgtaataatggccttcatgtaaacatttttgtggtttacacataaaatctctaattacaaagctatattatctaaaattacagtaagcaagaaaattaatccaagctaagacaatacttgcaacatcaattcatcatctgtgacaaggactgcttaagtctctttgtggttgacgtcattaattaactggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggcctaggcgcgccaacgcgt

SEQ ID NO: 20 - DNA, 10801 bp, synthetic construct
  source 1..10801: mol_type = other DNA
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttccttacgtacaattgggatcccggaccgtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgccaccatggcctctctgtggctgctgagctgcttcagcctggtgggcgccgctttcggcgccagaccttgtatccccaagagcttcggatatagcagcgtggtctgcgtgtgcaacgccacctactgcgatagcttcgacccacctacctttccagctctgggcaccttctccagatacgagtctacaagaagcggcagaagaatggaactgtccatgggccctatccaggccaaccacaccggcacaggcctcctgttgaccctgcagcccgagcagaaatttcagaaagtgaagggattcggcggcgccatgaccgatgccgccgctctgaatatcctggcgctgagccctcctgcccagaacctgctgctgaagagctactttagcgaggaggggatcggctacaacattatcagagtgcccatggccagctgcgactttagcatcagaacctatacatacgccgacaccccagatgacttccagctgcacaacttcagcctgcctgaggaagatacaaagctgaaaatccccctgatccaccgggccctgcaactggctcagcggccagtgtccctgctggcctctccttggaccagccctacctggctgaagaccaatggcgccgtgaacggcaagggctctctgaagggccagcctggcgacatctaccatcagacctgggccagatacttcgtgaagttcctggatgcctacgctgagcacaagctgcagttctgggccgtgaccgccgagaacgagccttccgccggactgctcagcggctatcctttccagtgcctgggcttcacacctgagcaccagcgcgacttcatcgccagggacctgggccctacactggccaatagcacacaccacaacgtgagactgctgatgctggatgatcagagactgctgcttccccactgggctaaggtcgtgctgaccgaccctgaagccgctaagtacgtgcacggcatcgccgtgcactggtacctggacttcctggcccctgccaaggccaccctgggagaaacccaccggctgttccctaacaccatgctgtttgcctcagaggcctgtgtgggctccaaattctgggagcaaagcgtgcggctgggctcttgggacagaggaatgcagtacagccacagcatcatcaccaacctgctgtaccacgtggtgggctggaccgactggaacctggccctgaaccccgagggaggccctaactgggtcagaaacttcgtggacagccccatcattgtggatatcaccaaggacacgttctacaagcagccaatgttctaccacctgggccacttcagcaagttcatccctgaaggcagtcagagagtgggcctggtggcctctcagaagaacgacctggacgccgttgcgctgatgcaccccgacggcagcgccgtggtggtggtgctgaatagatcttctaaggatgtgcctctgacaatcaaggaccctgctgtgggatttctggaaacaatcagccctggatacagcatccatacatacctgtggcggcggcagtgagattaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgatcctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctcgaaattaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaatcgcgacggccgacgtctttgttacaacttactatatatatgcacacatatatatatatttgggtatattgggggggttctaatttaagaaatgcataattggctatagacagacagttgtcagaacttggcaatgggtacgtgcaggttcattataccaagtctacttgtagttgttcaaaatgtatcataatacaaggccgggcgaggtcgtcacgcctgtaatcccagcattttgggaggctaaggcaggaggattgcttgaggtcaggagtttgtgaccagcctgggcaacagagcaagaccctgtctccaaaaagaaaaaaaataattttttacaaaataaaaacaaaatgtatcatcagacgaaattaaataagaggcaattcattgtaatgacaacttttcccagcttgacatttaacaaaaagtctaagtcctcttaattcatatttaatgatcaaatatcaaatactaattttttttttttttttttttttgagacggagtctcgctctgtcgcccaggctggagtgcagtggcgcgatcctggctcactgcaagctccgcctcccgggttcacgccattctcctgcctcagcctcccgagtagctgggattacagacatgcgccaccacgcccggctaattttgtatttttagtagagatggggtttctccatgttggtcaggctggtcttgaatttcccacctcaggtgatctgcctgcctcagcctcacaaagcagtagctgggactacaggcacccaccaccacacttggttaattcttttgtattttttttgtaaagacgggatttcaccatgttagccaggatggtctcgatctcctgatctcatgatccgcccgcctcagcctcccaaagtgctgggattacaggcgtgagccaccccgcccggccatcaaatactaattcttaaatggtaaggacccactattcagaacctgtatccttatcactaatatgcaaatatttattgaatacttactatgtcatgcatactagagagagttagataaatttgatacagctaccctcacagaacttacagtgtaatagatggcatgacatgtacatgagtaactgtgaacagtgttaaattgctatttaaaaaaaaagacggctgggcgctgtggctcatgcctgtaatcccagcactttgggaggccaaggcaagttgatcgctcgaggtcaagagttcgagaccagcctggccaacgtggtaaaaccccgtctctactaaaaatacaaaaaaaaaattagccaggcatggtggcacaggcctgtaatcccagctactagggaggctgagacatggagaactgcttgaatccaggaggcagaggttacagtgagccgagatcataccactacactccagcctgagtgacagagcgagactcctgtctaaaaaaaaaaaaaaaaaaaaagatacaggttaagtgttatggtagttgaagagagaactcaaactctgtctcagaagcctcacttgcatgtggaccactgatatgaaataatataaataggtataattcaataaataggaacttcagttttaatcatcccaaacaccaaaacttcctatcaaacaggtccaataaactcaatctctataagagctagacagaaatctacttggtggcctataatcttattagcccttacttgtcccatctgatattaattaaccccatctaatatggattagttaacaatccagtggctgctttgacaggaacagttggagagagttggggattgcaacatattcaattatacaaaaatgcattcagcatctaccttgattaaggcagtgtgcaacagaatttgcaggagagtaaaagaatgattataaatttacaacccttaaagagctatagctgggcgtggtggctcatgcctgtaaatcccagcactttgggaggctgaggcgggtggatcacctgaggccagaagttcaagaccagcctagccaacatggcgaaaccctgtctctacaaaaaatacaaaaattagccgggtgtggtggcacgtgcctgtagtcccagttacttgggaggccgaggcaggagaatcgcttgaacctaggaggtggaggctgcagtgagccgagattgtgccactgcactccacttcagcctgggcgacaagagcaagactccgtcacaaaaaaaaaaaaaaaaaaaaaggctaaaatctagtgggaaaggcatatatacatacaactaactgtatagcataataaagctcataatctgtaacaaaatctaattcgacaagcccagaaacttgtgatttaccaaaaacagttatatatacacaaaaagtaaacctagaacccaaagttacccagcaccaatgattctctccctaagcagtatcaagtttaaagcagtgattacattctactgcctagattgtaaactgagtaaaggagaccagcacctttctgctactgaactagcacagccgtgtaaaccaacaaggcaatggcagtgcccaactttctgtatgaatataagttacatctgttttattatttgtgacttggtgttgcatgtggttattatcaacaccttctgaaagaacaactacctgctcaggctgccataacaaaataccacagactgagtgacttaacagaaacttatttctcacagttttggaggctgggaagtccaaaattaagctaactgcaaggtaggtttcaatctcaggcctcttctttggcttgaaggtcttctaactgtgtgctcacatgacctcttctaacaagctctctggtgtctctttttttttttttttcttttttgagacagagtctcactctgtcacccaggctggagtacagtggcacaatctgggctcactgcaacctccaactcccgggttcaagtgattctcatgcctcaccctcccgagtagcttggatgacaggagcccgctaccacacccagctaatttttgtatttttagtagagatggtgtttcactacattggccaggctggtctcaaactcctgacctcgtgatccacccaccttggcctcccaaagtgctgggattacaggtgtgagccactgcgcccgtcctggtgtcttttcatataagggcactaatccaatcagacctgggcccaaccctcccgacttcttctaactgtaattaccttccaaaggccctgtctccaaataccatcacactgggggttaggacttcaaaaaaggtatggggggggtgtgggaggacataaatgctcagtccataacaagcacccaacataaaaatggctagaacagatcacaaaaaaaaggtcctgtatggctttggggaagggctcaaccccaaaatatctgaaagctctggaggggcctagaagtggtaaatgaatgaaaacgtggttactctcccgatctgcctttcccaaatatggccattcttggctgaatcagaaatcaaaggacaggttattaattactagctctaagttacttaccatttgctgagacagttcagaaatctgactgcatctcctcagggatctagaacacagttctcaaattctaacttacttgtgatatacttgtgaatgataaaaatcgctacaggtacttttattaatctgaaagagtattgagaaattacctttcattctgacttttgtctggaatgaaaatcaatacttttgctatattccattactgaaataattttactttccagtaaaactggcattataattttttttaatttttaaaacttcataattttttgccagactgacccatgtaaacatacaaattactaataattatgcacgtcacatctgtaataatggccttcatgtaaacatttttgtggtttacacataaaatctctaattacaaagctatattatctaaaattacagtaagcaagaaaattaatccaagctaagacaatacttgcaacatcaattcatcatctgtgacaaggactgcttaagtctctttgtggttgacgtcattaattaactggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggcctaggcgcgccaacgcgt

SEQ ID NO: 21 - DNA, 10804 bp, synthetic construct
  source 1..10804: mol_type = other DNA
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttccttacgtacaattgggatcccggaccgtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgccaccatgttccagaccggcggactgatcgtgttctacggcctgctggcccagacaatggccgccagaccttgtatccccaagagcttcggatatagcagcgtggtctgcgtgtgcaacgccacctactgcgatagcttcgacccacctacctttccagctctgggcaccttctccagatacgagtctacaagaagcggcagaagaatggaactgtccatgggccctatccaggccaaccacaccggcacaggcctcctgttgaccctgcagcccgagcagaaatttcagaaagtgaagggattcggcggcgccatgaccgatgccgccgctctgaatatcctggcgctgagccctcctgcccagaacctgctgctgaagagctactttagcgaggaggggatcggctacaacattatcagagtgcccatggccagctgcgactttagcatcagaacctatacatacgccgacaccccagatgacttccagctgcacaacttcagcctgcctgaggaagatacaaagctgaaaatccccctgatccaccgggccctgcaactggctcagcggccagtgtccctgctggcctctccttggaccagccctacctggctgaagaccaatggcgccgtgaacggcaagggctctctgaagggccagcctggcgacatctaccatcagacctgggccagatacttcgtgaagttcctggatgcctacgctgagcacaagctgcagttctgggccgtgaccgccgagaacgagccttccgccggactgctcagcggctatcctttccagtgcctgggcttcacacctgagcaccagcgcgacttcatcgccagggacctgggccctacactggccaatagcacacaccacaacgtgagactgctgatgctggatgatcagagactgctgcttccccactgggctaaggtcgtgctgaccgaccctgaagccgctaagtacgtgcacggcatcgccgtgcactggtacctggacttcctggcccctgccaaggccaccctgggagaaacccaccggctgttccctaacaccatgctgtttgcctcagaggcctgtgtgggctccaaattctgggagcaaagcgtgcggctgggctcttgggacagaggaatgcagtacagccacagcatcatcaccaacctgctgtaccacgtggtgggctggaccgactggaacctggccctgaaccccgagggaggccctaactgggtcagaaacttcgtggacagccccatcattgtggatatcaccaaggacacgttctacaagcagccaatgttctaccacctgggccacttcagcaagttcatccctgaaggcagtcagagagtgggcctggtggcctctcagaagaacgacctggacgccgttgcgctgatgcaccccgacggcagcgccgtggtggtggtgctgaatagatcttctaaggatgtgcctctgacaatcaaggaccctgctgtgggatttctggaaacaatcagccctggatacagcatccatacatacctgtggcggcggcagtgagattaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgatcctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctcgaaattaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaatcgcgacggccgacgtctttgttacaacttactatatatatgcacacatatatatatatttgggtatattgggggggttctaatttaagaaatgcataattggctatagacagacagttgtcagaacttggcaatgggtacgtgcaggttcattataccaagtctacttgtagttgttcaaaatgtatcataatacaaggccgggcgaggtcgtcacgcctgtaatcccagcattttgggaggctaaggcaggaggattgcttgaggtcaggagtttgtgaccagcctgggcaacagagcaagaccctgtctccaaaaagaaaaaaaataattttttacaaaataaaaacaaaatgtatcatcagacgaaattaaataagaggcaattcattgtaatgacaacttttcccagcttgacatttaacaaaaagtctaagtcctcttaattcatatttaatgatcaaatatcaaatactaattttttttttttttttttttttgagacggagtctcgctctgtcgcccaggctggagtgcagtggcgcgatcctggctcactgcaagctccgcctcccgggttcacgccattctcctgcctcagcctcccgagtagctgggattacagacatgcgccaccacgcccggctaattttgtatttttagtagagatggggtttctccatgttggtcaggctggtcttgaatttcccacctcaggtgatctgcctgcctcagcctcacaaagcagtagctgggactacaggcacccaccaccacacttggttaattcttttgtattttttttgtaaagacgggatttcaccatgttagccaggatggtctcgatctcctgatctcatgatccgcccgcctcagcctcccaaagtgctgggattacaggcgtgagccaccccgcccggccatcaaatactaattcttaaatggtaaggacccactattcagaacctgtatccttatcactaatatgcaaatatttattgaatacttactatgtcatgcatactagagagagttagataaatttgatacagctaccctcacagaacttacagtgtaatagatggcatgacatgtacatgagtaactgtgaacagtgttaaattgctatttaaaaaaaaagacggctgggcgctgtggctcatgcctgtaatcccagcactttgggaggccaaggcaagttgatcgctcgaggtcaagagttcgagaccagcctggccaacgtggtaaaaccccgtctctactaaaaatacaaaaaaaaaattagccaggcatggtggcacaggcctgtaatcccagctactagggaggctgagacatggagaactgcttgaatccaggaggcagaggttacagtgagccgagatcataccactacactccagcctgagtgacagagcgagactcctgtctaaaaaaaaaaaaaaaaaaaaagatacaggttaagtgttatggtagttgaagagagaactcaaactctgtctcagaagcctcacttgcatgtggaccactgatatgaaataatataaataggtataattcaataaataggaacttcagttttaatcatcccaaacaccaaaacttcctatcaaacaggtccaataaactcaatctctataagagctagacagaaatctacttggtggcctataatcttattagcccttacttgtcccatctgatattaattaaccccatctaatatggattagttaacaatccagtggctgctttgacaggaacagttggagagagttggggattgcaacatattcaattatacaaaaatgcattcagcatctaccttgattaaggcagtgtgcaacagaatttgcaggagagtaaaagaatgattataaatttacaacccttaaagagctatagctgggcgtggtggctcatgcctgtaaatcccagcactttgggaggctgaggcgggtggatcacctgaggccagaagttcaagaccagcctagccaacatggcgaaaccctgtctctacaaaaaatacaaaaattagccgggtgtggtggcacgtgcctgtagtcccagttacttgggaggccgaggcaggagaatcgcttgaacctaggaggtggaggctgcagtgagccgagattgtgccactgcactccacttcagcctgggcgacaagagcaagactccgtcacaaaaaaaaaaaaaaaaaaaaaggctaaaatctagtgggaaaggcatatatacatacaactaactgtatagcataataaagctcataatctgtaacaaaatctaattcgacaagcccagaaacttgtgatttaccaaaaacagttatatatacacaaaaagtaaacctagaacccaaagttacccagcaccaatgattctctccctaagcagtatcaagtttaaagcagtgattacattctactgcctagattgtaaactgagtaaaggagaccagcacctttctgctactgaactagcacagccgtgtaaaccaacaaggcaatggcagtgcccaactttctgtatgaatataagttacatctgttttattatttgtgacttggtgttgcatgtggttattatcaacaccttctgaaagaacaactacctgctcaggctgccataacaaaataccacagactgagtgacttaacagaaacttatttctcacagttttggaggctgggaagtccaaaattaagctaactgcaaggtaggtttcaatctcaggcctcttctttggcttgaaggtcttctaactgtgtgctcacatgacctcttctaacaagctctctggtgtctctttttttttttttttcttttttgagacagagtctcactctgtcacccaggctggagtacagtggcacaatctgggctcactgcaacctccaactcccgggttcaagtgattctcatgcctcaccctcccgagtagcttggatgacaggagcccgctaccacacccagctaatttttgtatttttagtagagatggtgtttcactacattggccaggctggtctcaaactcctgacctcgtgatccacccaccttggcctcccaaagtgctgggattacaggtgtgagccactgcgcccgtcctggtgtcttttcatataagggcactaatccaatcagacctgggcccaaccctcccgacttcttctaactgtaattaccttccaaaggccctgtctccaaataccatcacactgggggttaggacttcaaaaaaggtatggggggggtgtgggaggacataaatgctcagtccataacaagcacccaacataaaaatggctagaacagatcacaaaaaaaaggtcctgtatggctttggggaagggctcaaccccaaaatatctgaaagctctggaggggcctagaagtggtaaatgaatgaaaacgtggttactctcccgatctgcctttcccaaatatggccattcttggctgaatcagaaatcaaaggacaggttattaattactagctctaagttacttaccatttgctgagacagttcagaaatctgactgcatctcctcagggatctagaacacagttctcaaattctaacttacttgtgatatacttgtgaatgataaaaatcgctacaggtacttttattaatctgaaagagtattgagaaattacctttcattctgacttttgtctggaatgaaaatcaatacttttgctatattccattactgaaataattttactttccagtaaaactggcattataattttttttaatttttaaaacttcataattttttgccagactgacccatgtaaacatacaaattactaataattatgcacgtcacatctgtaataatggccttcatgtaaacatttttgtggtttacacataaaatctctaattacaaagctatattatctaaaattacagtaagcaagaaaattaatccaagctaagacaatacttgcaacatcaattcatcatctgtgacaaggactgcttaagtctctttgtggttgacgtcattaattaactggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggcctaggcgcgccaacgcgt

SEQ ID NO: 22 - DNA, 10798 bp, synthetic construct
  source 1..10798: mol_type = other DNA
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttccttacgtacaattgggatcccggaccgtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgccaccatgctgctgctgctgctgctgctcggcctgagactgcagctgagcctgggcgccagaccttgtatccccaagagcttcggatatagcagcgtggtctgcgtgtgcaacgccacctactgcgatagcttcgacccacctacctttccagctctgggcaccttctccagatacgagtctacaagaagcggcagaagaatggaactgtccatgggccctatccaggccaaccacaccggcacaggcctcctgttgaccctgcagcccgagcagaaatttcagaaagtgaagggattcggcggcgccatgaccgatgccgccgctctgaatatcctggcgctgagccctcctgcccagaacctgctgctgaagagctactttagcgaggaggggatcggctacaacattatcagagtgcccatggccagctgcgactttagcatcagaacctatacatacgccgacaccccagatgacttccagctgcacaacttcagcctgcctgaggaagatacaaagctgaaaatccccctgatccaccgggccctgcaactggctcagcggccagtgtccctgctggcctctccttggaccagccctacctggctgaagaccaatggcgccgtgaacggcaagggctctctgaagggccagcctggcgacatctaccatcagacctgggccagatacttcgtgaagttcctggatgcctacgctgagcacaagctgcagttctgggccgtgaccgccgagaacgagccttccgccggactgctcagcggctatcctttccagtgcctgggcttcacacctgagcaccagcgcgacttcatcgccagggacctgggccctacactggccaatagcacacaccacaacgtgagactgctgatgctggatgatcagagactgctgcttccccactgggctaaggtcgtgctgaccgaccctgaagccgctaagtacgtgcacggcatcgccgtgcactggtacctggacttcctggcccctgccaaggccaccctgggagaaacccaccggctgttccctaacaccatgctgtttgcctcagaggcctgtgtgggctccaaattctgggagcaaagcgtgcggctgggctcttgggacagaggaatgcagtacagccacagcatcatcaccaacctgctgtaccacgtggtgggctggaccgactggaacctggccctgaaccccgagggaggccctaactgggtcagaaacttcgtggacagccccatcattgtggatatcaccaaggacacgttctacaagcagccaatgttctaccacctgggccacttcagcaagttcatccctgaaggcagtcagagagtgggcctggtggcctctcagaagaacgacctggacgccgttgcgctgatgcaccccgacggcagcgccgtggtggtggtgctgaatagatcttctaaggatgtgcctctgacaatcaaggaccctgctgtgggatttctggaaacaatcagccctggatacagcatccatacatacctgtggcggcggcagtgagattaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgatcctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctcgaaattaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaatcgcgacggccgacgtctttgttacaacttactatatatatgcacacatatatatatatttgggtatattgggggggttctaatttaagaaatgcataattggctatagacagacagttgtcagaacttggcaatgggtacgtgcaggttcattataccaagtctacttgtagttgttcaaaatgtatcataatacaaggccgggcgaggtcgtcacgcctgtaatcccagcattttgggaggctaaggcaggaggattgcttgaggtcaggagtttgtgaccagcctgggcaacagagcaagaccctgtctccaaaaagaaaaaaaataattttttacaaaataaaaacaaaatgtatcatcagacgaaattaaataagaggcaattcattgtaatgacaacttttcccagcttgacatttaacaaaaagtctaagtcctcttaattcatatttaatgatcaaatatcaaatactaattttttttttttttttttttttgagacggagtctcgctctgtcgcccaggctggagtgcagtggcgcgatcctggctcactgcaagctccgcctcccgggttcacgccattctcctgcctcagcctcccgagtagctgggattacagacatgcgccaccacgcccggctaattttgtatttttagtagagatggggtttctccatgttggtcaggctggtcttgaatttcccacctcaggtgatctgcctgcctcagcctcacaaagcagtagctgggactacaggcacccaccaccacacttggttaattcttttgtattttttttgtaaagacgggatttcaccatgttagccaggatggtctcgatctcctgatctcatgatccgcccgcctcagcctcccaaagtgctgggattacaggcgtgagccaccccgcccggccatcaaatactaattcttaaatggtaaggacccactattcagaacctgtatccttatcactaatatgcaaatatttattgaatacttactatgtcatgcatactagagagagttagataaatttgatacagctaccctcacagaacttacagtgtaatagatggcatgacatgtacatgagtaactgtgaacagtgttaaattgctatttaaaaaaaaagacggctgggcgctgtggctcatgcctgtaatcccagcactttgggaggccaaggcaagttgatcgctcgaggtcaagagttcgagaccagcctggccaacgtggtaaaaccccgtctctactaaaaatacaaaaaaaaaattagccaggcatggtggcacaggcctgtaatcccagctactagggaggctgagacatggagaactgcttgaatccaggaggcagaggttacagtgagccgagatcataccactacactccagcctgagtgacagagcgagactcctgtctaaaaaaaaaaaaaaaaaaaaagatacaggttaagtgttatggtagttgaagagagaactcaaactctgtctcagaagcctcacttgcatgtggaccactgatatgaaataatataaataggtataattcaataaataggaacttcagttttaatcatcccaaacaccaaaacttcctatcaaacaggtccaataaactcaatctctataagagctagacagaaatctacttggtggcctataatcttattagcccttacttgtcccatctgatattaattaaccccatctaatatggattagttaacaatccagtggctgctttgacaggaacagttggagagagttggggattgcaacatattcaattatacaaaaatgcattcagcatctaccttgattaaggcagtgtgcaacagaatttgcaggagagtaaaagaatgattataaatttacaacccttaaagagctatagctgggcgtggtggctcatgcctgtaaatcccagcactttgggaggctgaggcgggtggatcacctgaggccagaagttcaagaccagcctagccaacatggcgaaaccctgtctctacaaaaaatacaaaaattagccgggtgtggtggcacgtgcctgtagtcccagttacttgggaggccgaggcaggagaatcgcttgaacctaggaggtggaggctgcagtgagccgagattgtgccactgcactccacttcagcctgggcgacaagagcaagactccgtcacaaaaaaaaaaaaaaaaaaaaaggctaaaatctagtgggaaaggcatatatacatacaactaactgtatagcataataaagctcataatctgtaacaaaatctaattcgacaagcccagaaacttgtgatttaccaaaaacagttatatatacacaaaaagtaaacctagaacccaaagttacccagcaccaatgattctctccctaagcagtatcaagtttaaagcagtgattacattctactgcctagattgtaaactgagtaaaggagaccagcacctttctgctactgaactagcacagccgtgtaaaccaacaaggcaatggcagtgcccaactttctgtatgaatataagttacatctgttttattatttgtgacttggtgttgcatgtggttattatcaacaccttctgaaagaacaactacctgctcaggctgccataacaaaataccacagactgagtgacttaacagaaacttatttctcacagttttggaggctgggaagtccaaaattaagctaactgcaaggtaggtttcaatctcaggcctcttctttggcttgaaggtcttctaactgtgtgctcacatgacctcttctaacaagctctctggtgtctctttttttttttttttcttttttgagacagagtctcactctgtcacccaggctggagtacagtggcacaatctgggctcactgcaacctccaactcccgggttcaagtgattctcatgcctcaccctcccgagtagcttggatgacaggagcccgctaccacacccagctaatttttgtatttttagtagagatggtgtttcactacattggccaggctggtctcaaactcctgacctcgtgatccacccaccttggcctcccaaagtgctgggattacaggtgtgagccactgcgcccgtcctggtgtcttttcatataagggcactaatccaatcagacctgggcccaaccctcccgacttcttctaactgtaattaccttccaaaggccctgtctccaaataccatcacactgggggttaggacttcaaaaaaggtatggggggggtgtgggaggacataaatgctcagtccataacaagcacccaacataaaaatggctagaacagatcacaaaaaaaaggtcctgtatggctttggggaagggctcaaccccaaaatatctgaaagctctggaggggcctagaagtggtaaatgaatgaaaacgtggttactctcccgatctgcctttcccaaatatggccattcttggctgaatcagaaatcaaaggacaggttattaattactagctctaagttacttaccatttgctgagacagttcagaaatctgactgcatctcctcagggatctagaacacagttctcaaattctaacttacttgtgatatacttgtgaatgataaaaatcgctacaggtacttttattaatctgaaagagtattgagaaattacctttcattctgacttttgtctggaatgaaaatcaatacttttgctatattccattactgaaataattttactttccagtaaaactggcattataattttttttaatttttaaaacttcataattttttgccagactgacccatgtaaacatacaaattactaataattatgcacgtcacatctgtaataatggccttcatgtaaacatttttgtggtttacacataaaatctctaattacaaagctatattatctaaaattacagtaagcaagaaaattaatccaagctaagacaatacttgcaacatcaattcatcatctgtgacaaggactgcttaagtctctttgtggttgacgtcattaattaactggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggcctaggcgcgccaacgcgt

SEQ ID NO: 23 - DNA, 10801 bp, synthetic construct
  source 1..10801: mol_type = other DNA
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttccttacgtacaattgggatcccggaccgtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgccaccatgaccatcctgttcctgacaatggtgatcagctacttcggctgcatgaaggccgccagaccttgtatccccaagagcttcggatatagcagcgtggtctgcgtgtgcaacgccacctactgcgatagcttcgacccacctacctttccagctctgggcaccttctccagatacgagtctacaagaagcggcagaagaatggaactgtccatgggccctatccaggccaaccacaccggcacaggcctcctgttgaccctgcagcccgagcagaaatttcagaaagtgaagggattcggcggcgccatgaccgatgccgccgctctgaatatcctggcgctgagccctcctgcccagaacctgctgctgaagagctactttagcgaggaggggatcggctacaacattatcagagtgcccatggccagctgcgactttagcatcagaacctatacatacgccgacaccccagatgacttccagctgcacaacttcagcctgcctgaggaagatacaaagctgaaaatccccctgatccaccgggccctgcaactggctcagcggccagtgtccctgctggcctctccttggaccagccctacctggctgaagaccaatggcgccgtgaacggcaagggctctctgaagggccagcctggcgacatctaccatcagacctgggccagatacttcgtgaagttcctggatgcctacgctgagcacaagctgcagttctgggccgtgaccgccgagaacgagccttccgccggactgctcagcggctatcctttccagtgcctgggcttcacacctgagcaccagcgcgacttcatcgccagggacctgggccctacactggccaatagcacacaccacaacgtgagactgctgatgctggatgatcagagactgctgcttccccactgggctaaggtcgtgctgaccgaccctgaagccgctaagtacgtgcacggcatcgccgtgcactggtacctggacttcctggcccctgccaaggccaccctgggagaaacccaccggctgttccctaacaccatgctgtttgcctcagaggcctgtgtgggctccaaattctgggagcaaagcgtgcggctgggctcttgggacagaggaatgcagtacagccacagcatcatcaccaacctgctgtaccacgtggtgggctggaccgactggaacctggccctgaaccccgagggaggccctaactgggtcagaaacttcgtggacagccccatcattgtggatatcaccaaggacacgttctacaagcagccaatgttctaccacctgggccacttcagcaagttcatccctgaaggcagtcagagagtgggcctggtggcctctcagaagaacgacctggacgccgttgcgctgatgcaccccgacggcagcgccgtggtggtggtgctgaatagatcttctaaggatgtgcctctgacaatcaaggaccctgctgtgggatttctggaaacaatcagccctggatacagcatccatacatacctgtggcggcggcagtgagattaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgatcctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctcgaaattaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaatcgcgacggccgacgtctttgttacaacttactatatatatgcacacatatatatatatttgggtatattgggggggttctaatttaagaaatgcataattggctatagacagacagttgtcagaacttggcaatgggtacgtgcaggttcattataccaagtctacttgtagttgttcaaaatgtatcataatacaaggccgggcgaggtcgtcacgcctgtaatcccagcattttgggaggctaaggcaggaggattgcttgaggtcaggagtttgtgaccagcctgggcaacagagcaagaccctgtctccaaaaagaaaaaaaataattttttacaaaataaaaacaaaatgtatcatcagacgaaattaaataagaggcaattcattgtaatgacaacttttcccagcttgacatttaacaaaaagtctaagtcctcttaattcatatttaatgatcaaatatcaaatactaattttttttttttttttttttttgagacggagtctcgctctgtcgcccaggctggagtgcagtggcgcgatcctggctcactgcaagctccgcctcccgggttcacgccattctcctgcctcagcctcccgagtagctgggattacagacatgcgccaccacgcccggctaattttgtatttttagtagagatggggtttctccatgttggtcaggctggtcttgaatttcccacctcaggtgatctgcctgcctcagcctcacaaagcagtagctgggactacaggcacccaccaccacacttggttaattcttttgtattttttttgtaaagacgggatttcaccatgttagccaggatggtctcgatctcctgatctcatgatccgcccgcctcagcctcccaaagtgctgggattacaggcgtgagccaccccgcccggccatcaaatactaattcttaaatggtaaggacccactattcagaacctgtatccttatcactaatatgcaaatatttattgaatacttactatgtcatgcatactagagagagttagataaatttgatacagctaccctcacagaacttacagtgtaatagatggcatgacatgtacatgagtaactgtgaacagtgttaaattgctatttaaaaaaaaagacggctgggcgctgtggctcatgcctgtaatcccagcactttgggaggccaaggcaagttgatcgctcgaggtcaagagttcgagaccagcctggccaacgtggtaaaaccccgtctctactaaaaatacaaaaaaaaaattagccaggcatggtggcacaggcctgtaatcccagctactagggaggctgagacatggagaactgcttgaatccaggaggcagaggttacagtgagccgagatcataccactacactccagcctgagtgacagagcgagactcctgtctaaaaaaaaaaaaaaaaaaaaagatacaggttaagtgttatggtagttgaagagagaactcaaactctgtctcagaagcctcacttgcatgtggaccactgatatgaaataatataaataggtataattcaataaataggaacttcagttttaatcatcccaaacaccaaaacttcctatcaaacaggtccaataaactcaatctctataagagctagacagaaatctacttggtggcctataatcttattagcccttacttgtcccatctgatattaattaaccccatctaatatggattagttaacaatccagtggctgctttgacaggaacagttggagagagttggggattgcaacatattcaattatacaaaaatgcattcagcatctaccttgattaaggcagtgtgcaacagaatttgcaggagagtaaaagaatgattataaatttacaacccttaaagagctatagctgggcgtggtggctcatgcctgtaaatcccagcactttgggaggctgaggcgggtggatcacctgaggccagaagttcaagaccagcctagccaacatggcgaaaccctgtctctacaaaaaatacaaaaattagccgggtgtggtggcacgtgcctgtagtcccagttacttgggaggccgaggcaggagaatcgcttgaacctaggaggtggaggctgcagtgagccgagattgtgccactgcactccacttcagcctgggcgacaagagcaagactccgtcacaaaaaaaaaaaaaaaaaaaaaggctaaaatctagtgggaaaggcatatatacatacaactaactgtatagcataataaagctcataatctgtaacaaaatctaattcgacaagcccagaaacttgtgatttaccaaaaacagttatatatacacaaaaagtaaacctagaacccaaagttacccagcaccaatgattctctccctaagcagtatcaagtttaaagcagtgattacattctactgcctagattgtaaactgagtaaaggagaccagcacctttctgctactgaactagcacagccgtgtaaaccaacaaggcaatggcagtgcccaactttctgtatgaatataagttacatctgttttattatttgtgacttggtgttgcatgtggttattatcaacaccttctgaaagaacaactacctgctcaggctgccataacaaaataccacagactgagtgacttaacagaaacttatttctcacagttttggaggctgggaagtccaaaattaagctaactgcaaggtaggtttcaatctcaggcctcttctttggcttgaaggtcttctaactgtgtgctcacatgacctcttctaacaagctctctggtgtctctttttttttttttttcttttttgagacagagtctcactctgtcacccaggctggagtacagtggcacaatctgggctcactgcaacctccaactcccgggttcaagtgattctcatgcctcaccctcccgagtagcttggatgacaggagcccgctaccacacccagctaatttttgtatttttagtagagatggtgtttcactacattggccaggctggtctcaaactcctgacctcgtgatccacccaccttggcctcccaaagtgctgggattacaggtgtgagccactgcgcccgtcctggtgtcttttcatataagggcactaatccaatcagacctgggcccaaccctcccgacttcttctaactgtaattaccttccaaaggccctgtctccaaataccatcacactgggggttaggacttcaaaaaaggtatggggggggtgtgggaggacataaatgctcagtccataacaagcacccaacataaaaatggctagaacagatcacaaaaaaaaggtcctgtatggctttggggaagggctcaaccccaaaatatctgaaagctctggaggggcctagaagtggtaaatgaatgaaaacgtggttactctcccgatctgcctttcccaaatatggccattcttggctgaatcagaaatcaaaggacaggttattaattactagctctaagttacttaccatttgctgagacagttcagaaatctgactgcatctcctcagggatctagaacacagttctcaaattctaacttacttgtgatatacttgtgaatgataaaaatcgctacaggtacttttattaatctgaaagagtattgagaaattacctttcattctgacttttgtctggaatgaaaatcaatacttttgctatattccattactgaaataattttactttccagtaaaactggcattataattttttttaatttttaaaacttcataattttttgccagactgacccatgtaaacatacaaattactaataattatgcacgtcacatctgtaataatggccttcatgtaaacatttttgtggtttacacataaaatctctaattacaaagctatattatctaaaattacagtaagcaagaaaattaatccaagctaagacaatacttgcaacatcaattcatcatctgtgacaaggactgcttaagtctctttgtggttgacgtcattaattaactggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggcctaggcgcgccaacgcgt

SEQ ID NO: 24 - DNA, 78 bp, synthetic construct
  source 1..78: mol_type = other DNA
cactccctctctgcgcgctcgctcgctcactgaggccgggcgaccaaaggtcgcccacgcccgggctttgcccgggcg

SEQ ID NO: 25 - protein, 536 aa, synthetic construct; identical to SEQ ID NO: 1

SEQ ID NO: 26 - protein, 515 aa, synthetic construct
MASLWLLSCFSLVGAAFGARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWRRQ

SEQ ID NO: 27 - protein, 516 aa, synthetic construct
MFQTGGLIVFYGLLAQTMAARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWRRQ

SEQ ID NO: 28 - protein, 514 aa, synthetic construct
MLLLLLLLGLRLQLSLGARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWRRQ

SEQ ID NO: 29 - protein, 515 aa, synthetic construct
MTILFLTMVISYFGCMKAARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWRRQ

SEQ ID NO: 30 - protein, 39 aa, synthetic construct
MEFSSPSREECPKPLSRVSIMAGSLTGLLLLQAVSWASG

SEQ ID NO: 31 - protein, 18 aa, synthetic construct
MASLWLLSCFSLVGAAFG

SEQ ID NO: 32 - protein, 19 aa, synthetic construct
MFQTGGLIVFYGLLAQTMA

SEQ ID NO: 33 - protein, 17 aa, synthetic construct
MLLLLLLLGLRLQLSLG

SEQ ID NO: 34 - protein, 18 aa, synthetic construct
MTILFLTMVISYFGCMKA

(19) United States
(12) Patent Application Publication
(10) Pub. No.: US 2025/0152747 A1
(43) Pub. Date: May 15, 2025

(54) METHODS OF TREATING GAUCHER DISEASE AND GBA-PARKINSON’S DISEASE

(71) Applicant: Genzyme Corporation, Cambridge, MA (US)

(72) Inventors: Swathi AYLOO, Natick, MA (US); Shyam RAMACHANDRAN, Winchester, MA (US)

(21) Appl. No.: 18/949,549

(22) Filed: Nov. 15, 2024

Related U.S. Application Data
(60) Provisional application No. 63/599,430, filed on Nov. 15, 2023.

(51) Int. Cl. A61K 48/00; A61K 38/47; A61P 25/16; C12N 15/86

(52) U.S. Cl. CPC A61K 48/0066; A61K 38/47; A61K 48/0075; A61P 25/16; C12N 15/86; C12Y 302/01045; C12N 2750/14122; C12N 2750/14143

(57) ABSTRACT

Provided herein are expression cassettes for expressing a transgene in a cell, wherein the transgene encodes a GCase polypeptide. Also provided are methods to treat Gaucher Disease or GBA-PD. Further provided herein are vectors (e.g., rAAV vectors), viral particles, pharmaceutical compositions, and kits for expressing an GCase polypeptide in an individual in need thereof.

### CROSS-REFERENCE TO RELATED APPLICATIONS

This application claims the priority benefit of U.S. Provisional Application No. 63/599,430, filed Nov. 15, 2023, which is incorporated by reference in its entirety.

### REFERENCE TO AN ELECTRONIC SEQUENCE LISTING

The content of the electronic sequence listing (159792018700seqlist.xml; Size: 100,264 bytes; and Date of Creation: Nov. 13, 2024) is herein incorporated by reference in its entirety.

### FIELD OF THE INVENTION

The present invention relates to methods for treating Gaucher Disease or GBA-PD in a patient in need thereof.

### BACKGROUND

Gaucher Disease (GD) is an autosomal recessive lysosomal storage disorder caused by mutations in GBA1, the gene encoding glucocerebrosidase (GCase). Reduction or loss of GCase activity leads to the accumulation of toxic lipid substrates, disrupting cellular homeostasis. GD type 1 (GD1) patients typically present with splenomegaly, hepatomegaly, and anemia or thrombocytopenia, while GD type 3 (GD3) patients present with debilitating neurological symptoms and associated systemic manifestations of GD1. GD1 patients typically rely on enzyme replacement therapy (ERT) for symptom management. ERTs are taken as bi-weekly infusions, however, they fail to alleviate the neurological symptoms in GD3 patients.

Furthermore, heterozygous mutations in GBA1 are a major risk factor for Parkinson's Disease (PD) with GBA-PD patients making up 1 million of PD patients worldwide. Clinically, GBA-PD patients present with disease onset at an earlier age experience faster cognitive decline. About 5-10% of Parkinson's disease (PD) patients, which is approximately 0.5-1 million patients, are carriers of the GBA1 mutation in one of their alleles. GBA1 is an extremely well-credentialed target in GBA-PD. Current treatment options include Levodopa and/or dopamine agonists for symptom management but most patients notice the effects of these drugs wearing off over time. Accordingly, similar to sporadic PD, there are no disease-modifying therapies yet to treat GBA-PD.

Accordingly, there exists an urgent need to develop therapeutic agents to treat and/or alleviate symptoms associated with GD and GBA-PD.

### BRIEF SUMMARY OF THE INVENTION

The disclosure provides methods for treating or improving symptoms associated with Gaucher Disease (GD) or GBA-PD in patients in need thereof, said methods comprising administering to the patient a recombinant adeno-associated virus (rAAV) particle comprising (1) a rAAV vector comprising an expression cassette for expressing a glucocerebrosidase (GCase) enzyme, wherein the expression cassette comprises a gene encoding the GCase enzyme operably linked to a promoter and optionally an enhancer, and (2) a capsid protein as set forth herein. In some embodiments, a recombinant adeno-associated virus (rAAV) viral particle comprising (a) a vector encoding the enzyme glucocerebrosidase (GCase); and (b) a capsid protein as set forth herein. The GCase enzyme will also be referred to herein as the GCase polypeptide.

In some embodiments, the capsid protein of the rAAV particle is an AAV9 capsid protein or a modified AAV capsid protein. In some embodiments, the modified AAV capsid protein is a modified AAV9 capsid protein as set forth herein. In some embodiments, a modified AAV9 capsid protein comprising a targeting peptide that comprises SEQ ID NO: 16.

In some aspects, the disclosure provides vectors encoding a signal peptide and a GCase polypeptide. In some embodiments, the nucleotide sequences encoding signal peptide and GCase polypeptide are transcriptionally regulated under the same promoter. In some embodiments, the nucleic acid sequence encoding the signal peptide and GCase polypeptide is codon-optimized. In some embodiments, the GCase polypeptide and signal peptide is encoded by a nucleic acid sequence of SEQ ID NO: 3. In some embodiments, the GCase polypeptide and signal peptide is encoded by a nucleic acid sequence of SEQ ID NO: 4. In some embodiments, the GCase polypeptide and signal peptide is encoded by a nucleic acid sequence of SEQ ID NO: 5. In some embodiments, the GCase polypeptide and signal peptide is encoded by a nucleic acid sequence of SEQ ID NO: 6. In some embodiments, the GCase polypeptide and signal peptide is encoded by a nucleic acid sequence of SEQ ID NO: 7.

In some embodiments, the GCase polypeptide and signal peptide comprises an amino acid sequence of SEQ ID NO: 25. In some embodiments, the GCase polypeptide and signal peptide is of the amino acid sequence of SEQ ID NO: 25. In some embodiments, the GCase polypeptide and signal peptide comprises an amino acid sequence of SEQ ID NO: 26. In some embodiments, the GCase polypeptide and signal peptide is of the amino acid sequence of SEQ ID NO: 26. In some embodiments, the GCase polypeptide and signal peptide comprises an amino acid sequence of SEQ ID NO: 27. In some embodiments, the GCase polypeptide and signal peptide is of the amino acid sequence of SEQ ID NO: 27. In some embodiments, the GCase polypeptide and signal peptide comprises an amino acid sequence of SEQ ID NO: 28. In some embodiments, the GCase polypeptide and signal peptide is of the amino acid sequence of SEQ ID NO: 28. In some embodiments, the GCase polypeptide and signal peptide comprises an amino acid sequence of SEQ ID NO: 29. In some embodiments, the GCase polypeptide and signal peptide is of the amino acid sequence of SEQ ID NO: 29.

In some embodiments, the vector comprises an expression cassette for expressing a glucocerebrosidase (GCase) enzyme, wherein the expression cassette comprises a gene encoding the GCase enzyme and a signal peptide. In some embodiments, the signal peptide comprises an amino acid sequence of SEQ ID NO: 30. In some embodiments, the signal peptide is of the amino acid sequence of SEQ ID NO: 30. In some embodiments, the signal peptide comprises an amino acid sequence of SEQ ID NO: 31. In some embodiments, the signal peptide is of the amino acid sequence of SEQ ID NO: 31. In some embodiments, the signal peptide comprises an amino acid sequence of SEQ ID NO: 32. In some embodiments, the signal peptide is of the amino acid sequence of SEQ ID NO: 32. In some embodiments, the signal peptide comprises an amino acid sequence of SEQ ID NO: 33. In some embodiments, the signal peptide is of the amino acid sequence of SEQ ID NO: 33. In some embodiments, the signal peptide comprises an amino acid sequence of SEQ ID NO: 34. In some embodiments, the signal peptide is of the amino acid sequence of SEQ ID NO: 34.

In some aspects, the methods provided herein provide an AAV vector encoding a human GCase protein engineered for cross-correction by appending a signal peptide and the polypeptide (e.g., human GBA1) driven from a promoter. In some embodiments, the signal peptide is not the signal peptide endogenous to GBA1. In some embodiments, the signal peptide is not of amino acid sequence of SEQ ID NO: 30.

In some aspects, a polypeptide comprising a signal peptide comprising an amino acid sequence selected from the group consisting of SEQ ID NO: 30, SEQ ID NO: 31, SEQ ID NO: 32, SEQ ID NO:33, and SEQ ID NO: 34 and a GCase polypeptide is provided herein. In some aspects, a GCase polypeptide and a signal peptide comprising an amino acid sequence selected from the group consisting of SEQ ID NOs: 25-29.

In some aspects, the method of treatment and compositions provided herein increase the efficiency of AAV-mediated GCase repletion by enabling cross-correction of the therapeutic transgene product. Current AAV-mediated therapeutics in pre-clinical development or clinical trials are limited by AAV biodistribution and only restore GCase protein and function to individual cells that have taken up and are expressing from the AAV vector genome. By engineering the human GBA1 transgene, as described herein, secretion of GCase from primary cells that are actively expressing the AAV vector genome as well as uptake of GCase by secondary cells that may or may not be expressing the AAV vector genome is enabled. In some embodiments, the AAV-methods provided herein result in GCase observed in non-transduced cells. In some embodiments, the polypeptide (e.g., GCase) is observed in the cerebellum. In some embodiments, the cerebellum is not transduced but the AAV-expressed protein is observed. In some embodiments, cross-corrected tissue areas have reduced total Lyso-GL compared to control treated samples. In some embodiments, the cross-corrected tissue areas are relevant for treatment of Gaucher disease.

In some embodiments, the vector encodes a GCase enzyme (polypeptide) comprising or having a sequence of SEQ ID NO:1. In some embodiments, the vector encodes a GCase enzyme (polypeptide) comprising or having a sequence of at least 85%, at least 90%, at least 95%, at least 98% or at least 99% homology to a sequence of SEQ ID NO:1. SEQ ID NO:1 is the wild-type amino acid sequence of glucocerebrosidase (GCase). The sequence includes an N-terminal 39-amino acid signaling peptide that is cleaved after expression of the protein, hence generating a mature protein having an amino acid sequence of SEQ ID NO: 2. In some embodiments, the vector encodes a GBA1 enzyme (polypeptide) comprising an amino acid sequence of SEQ ID NO:2. In some embodiments, the vector encodes a GBA1 polypeptide comprising or having an amino acid sequence of at least 85%, at least 90%, at least 95%, at least 98% or at least 99% homology to a sequence of SEQ ID NO:2. In some embodiments, the vector encodes a GBA1 enzyme (polypeptide) comprising or having an amino acid sequence of SEQ ID NO:2 following cleavage (removal) of the signaling peptide. In some embodiments, the vector encodes a GBA1 enzyme (polypeptide) comprising or having a sequence of at least 85%, at least 90%, at least 95%, at least 98% or at least 99% homology to SEQ ID NO:2 following cleavage (removal) of the signaling peptide.

As set forth herein, the human GBA1 gene can be engineered to express a GCase enzyme (polypeptide) having signaling peptides that differ from the wild-type (endogenous) sequence in SEQ ID NO:1. Upon cleavage of the signaling peptide, the same mature proteins, for instance, a GCase enzyme having a sequence of SEQ ID NO:2 is produced. It has been found that certain GCase enzymes with particular signaling peptides are more readily secretable than the wild-type GCase enzymes having a sequence of SEQ ID NO: 1.

In some embodiments, the sequences used to express the GCase enzyme are codon-optimized sequences. In some embodiments, the codon-optimized nucleic acid sequence has a sequence of SEQ ID NO:3. In some embodiments, the codon-optimized nucleic acid sequence has a sequence of SEQ ID NO: 4 (also referred to herein as SS1-GBA1). In some embodiments, the codon-optimized nucleic acid sequence has a sequence of SEQ ID NO: 5 (also referred to herein as SS2-GBA1). In some embodiments, the codon-optimized nucleic acid sequence has a sequence of SEQ ID NO: 6 (also referred to herein as SS3-GBA1). In some embodiments, the codon-optimized nucleic acid sequence has a sequence of SEQ ID NO:7 (also referred to herein as SS4-GBA1). The nucleic acid sequence of SEQ ID NO:3 expresses the wild-type human GCase enzyme (e.g., the enzyme having SEQ ID NO: 1), which includes the endogenous signaling peptide. Polynucleotides of SEQ ID NOs:4-7 express variant GCase enzymes that have different signaling peptides.

In some embodiments, the expression cassette comprises a nucleic acid sequence of SEQ ID NO: 19, SEQ ID NO: 20, SEQ ID NO: 21, SEQ ID NO: 22, or SEQ ID NO: 23.

In some embodiments, the AAV viral particles comprise a capsid protein. In some embodiments, the capsid protein comprises an AAV serotype. In some embodiments, the AAV serotype is 1, 2, 5, 8, 9, or recombinant human (rh)10. In some embodiments, the capsid protein is a modified capsid protein. In some embodiments, the modified capsid protein comprises a sequence comprising SEQ ID NO: 16.

In some embodiments, the AAV capsid proteins of the AAV viral particles comprise targeting peptides inserted into the capsid that alter the transduction and/or endosomal release of GBA1 following administration to the patient.

In some embodiments, the modified AAV9 capsid proteins of the AAV viral particles comprise targeting peptides inserted into the AAV9 capsid that alter the transduction and/or endosomal release of GBA1 following administration to the patient. The rAAV particles comprising modified AAV9 capsid proteins, as disclosed herein, comprise three structural capsid proteins, VP1, VP2 and VP3. The three capsid proteins are alternative splice variants. In some embodiments, the targeting peptide is inserted into the VP1, VP2, and VP3 capsid proteins within the rAAV particle.

In particular embodiments, the targeting peptide of the modified AAV9 capsids are inserted after residue 588 of the AAV9 structural protein (numbering based on VP1 numbering of AAV9). In some embodiments, the targeting peptide has SEQ ID NO: 16. In some embodiments, the targeting peptide is flanked by linker sequences on the N-terminal and the C-terminal end of the targeting peptide. In some embodiments, the linker sequence on the N-terminal side has the sequence AAA. In some embodiments, the linker sequence on the C-terminal side is AS. In some embodiments, the full sequence inserted after residue 588 of the AAV9 capsid structural protein has SEQ ID NO: 17. In some embodiments, the full modified AAV9 capsid structural protein has SEQ ID NO: 18. In some embodiments, the full modified AAV9 capsid structural protein that it at least 90% (e.g., at least 92%, at least 95%, at least 98%, at least 98.5%, at least 99%, at least 99.2%, at least 99.5%, or at least 99.8%) identical to SEQ ID NO: 18, wherein the modified AAV9 structural capsid comprises the targeting peptide of SEQ ID NO: 16. The capsid having SEQ ID NO: 18 will also be referred to herein as SAN006.

In some aspects, the rAAV viral particle is used to treat GD type 3 or GD type 2. In some embodiments, the rAAV viral particle can be administered to the cerebrospinal fluid (CSF) of the GD type 3 patient or the GD type 2 patient. In some embodiments, the viral particle is administered directly by intra-CSF administration to the patient with GD type 3 or GD type 2. In some embodiments of the above aspects, the rAAV is administered via direct injection into the spinal cord, via intrathecal injection, or via intracisternal injection to the patient with GD type 3 or GD type 2. In some embodiments, the rAAV is administered to more than one location of the spinal cord or cisterna magna of the patient with GD type 3 or GD type 2. In some embodiments, the rAAV is administered to more than one location of the spinal cord of the patient with GD type 3 or GD type 2. In some embodiments, the rAAV is administered to one or more of a lumbar subarachnoid space, thoracic subarachnoid space, and a cervical subarachnoid space of the spinal cord of the patient with GD type 3 or GD type 2. In some embodiments, the rAAV is administered to the cisterna magna of the patient with GD type 3 or GD type 2. In some embodiments, the method may comprise treating GD type 3 in a patient in need thereof. In other embodiments, the method may comprise treating GD type 2 in a patient in need thereof. In particular embodiments, the expression cassette of the viral particle is able to drive transgene expression in the central and peripheral nervous systems to treat GD type 3 or GD type 2. In some embodiments, administration of the rAAV particles ameliorates symptoms associated with GD2 or GD3. For instance, administration of the viral particle may reduce or impede the progression of brainstem and corticol dysfunction, seizures, and cognitive defects. In some embodiments, administration of the viral particle reduces or clears accumulated toxic lipid substrates (e.g., Lyso-GL1) in the brain of the GD type 3 or GD type 2 patient.

In other aspects, the rAAV viral particle is used to treat GD type 1. In certain embodiments, the rAAV viral particle can be administered parenterally (e.g., intravenously, subcutaneously, or intramuscularly) to the GD type 1 patient. In some embodiments, the rAAV viral particle can be administered intravenously to the GD type 1 patient. In some embodiments, administration of the rAAV viral particle reduces splenomegaly in the GD type 1 patient. In some embodiments, administration of the rAAV viral particle reduces hepatomegaly in the GD type 1 patient. In some embodiments, administration of the rAAV viral particle reduces anemia in the GD type 1 patient. In some embodiments, administration of the rAAV viral particle reduces thrombocytopenia in the GD type 1 patient. In some embodiments, administration of the viral particle reduces or clears accumulated toxic lipid substrates (e.g., Lyso-GL1) in peripheral organs such as the liver, spleen, kidney and/or lungs. In some embodiments, administration of the viral particle reduces or clears accumulated toxic lipid substrates (e.g., Lyso-GL1) in muscle tissues such as the heart, diaphragm, quadriceps, and gastrocnemius.

In other aspects, the rAAV viral particle is used to treat GBA-PD. In some embodiments, the rAAV viral particle can be administered to the cerebrospinal fluid (CSF) of the GBA-PD patient. In some embodiments, the viral particle is administered directly by intra-CSF administration of the patient with GBA-PD. In some embodiments, the rAAV is administered via direct injection into the spinal cord, via intrathecal injection, or via intracisternal injection of the patient with GBA-PD. In some embodiments, the rAAV is administered to more than one location of the spinal cord or cisterna magna of the patient with GBA-PD. In some embodiments, the rAAV is administered to more than one location of the spinal cord of the patient with GBA-PD. In some embodiments, the rAAV is administered to one or more of a lumbar subarachnoid space, thoracic subarachnoid space and a cervical subarachnoid space of the spinal cord of the patient with GBA-PD. In some embodiments, the rAAV is administered to the cisterna magna of the patient with GBA-PD. In some embodiments, the rAAV viral particle is administered to a patient with early stages of PD. In some such embodiments, administration of the viral particle reduces the cognitive decline and disease progression of the PD. In some embodiments, administration of the viral particle reduces or clears accumulated toxic lipid substrates (e.g., Lyso-GL1) in the brain of the GBA-PD patient. In some embodiments, administration of the viral particle reduces α-synuclein in the brain of the GBA-PD patient.

In some embodiments, transgene expression was further enhanced by the addition of the woodchuck hepatitis virus post-transcriptional regulatory element (WPRE). In some embodiments, the vector comprises a Chicken 3-actin (CBA) promoter. In some embodiments, the vector comprises a WPRE element and a CBA promoter.

In some embodiments, the method may comprise the use of SAN006-CBA-SS3-GBA1-WPRE. In some embodiments, the method may comprise the use of SAN006-CBA-SS3-GBA1-WPRE for the treatment of GBA-PD and/or Gaucher Disease Type 3 by intra-CSF administration. In some embodiments, the method may comprise the use of SAN006-CBA-SS3-GBA1-WPRE for the treatment of Gaucher Disease Type 1 by intravenous dosing. In some embodiments, the method may comprise a vector expressing codon-optimized and/or engineered human GBA1 gene for the treatment of GBA-PD and/or Gaucher Disease.

In some embodiments, the rAAV vector particle may comprise pAAV-CBA-GBA-WPRE-bGH. In some embodiments, the rAAV vector particle may comprise SAN006-CBA-GBA-WPRE-bGH.

In some embodiments, the rAAV vector particle may comprise pAAV-CBA-SS1-GBA-WPRE-bGH. In some embodiments, the rAAV vector particle may comprise SAN006-CBA-SS1-GBA1-WPRE. In some embodiments, the rAAV vector particle may comprise SAN006-CBA-SS1-GBA-WPRE.

In some embodiments, the rAAV vector particle may comprise pAAV-CBA-SS2-GBA-WPRE-bGH. In some embodiments, the rAAV vector particle may comprise SAN006-CBA-SS2-GBA1-WPRE. In some embodiments, the rAAV vector particle may comprise SAN006-CBA-SS2-GBA-WPRE.

In some embodiments, the rAAV vector particle may comprise pAAV-CBA-SS3-GBA-WPRE-bGH. In some embodiments, the rAAV vector particle may comprise SAN006-CBA-SS3-GBA1-WPRE. In some embodiments, the rAAV vector particle may comprise SAN006-CBA-SS3-GBA-WPRE.

In some embodiments, the rAAV vector particle may comprise pAAV-CBA-SS4-GBA-WPRE-bGH. In some embodiments, the rAAV vector particle may comprise SAN006-CBA-SS4-GBA1-WPRE. In some embodiments, the rAAV vector particle may comprise SAN006-CBA-SS4-GBA-WPRE.

In some embodiments of the above aspects, the rAAV particle comprises a vector comprising an expression cassette flanked by one or more AAV inverted terminal repeat (ITR) sequences. In some embodiments, the expression cassette is flanked by two AAV ITRs. In some embodiments, the AAV ITRs are AAV1, AAV2, AAV3, AAV4, AAV5, AAV6, AAV7, AAV8, AAVrh8, AAVrh8R, AAV9, AAV10, AAV.rh10, AAV11, AAV12, AAV2R471A, AAV DJ, a goat AAV, bovine AAV, or mouse AAV serotype ITRs. In some embodiments, the AAV ITRs are AAV2 ITRs. In some embodiments, the vector is a self-complimenting vector. In some embodiments, the vector comprises first nucleic acid sequence encoding the GBA1 polypeptide and a second nucleic acid sequence encoding a complement of the GBA1 polypeptide, wherein the first nucleic acid sequence can form intrastrand base pairs with the second nucleic acid sequence along most or all of its length. In some embodiments, the first nucleic acid sequence and the second nucleic acid sequence are linked by a mutated AAV ITR, wherein the mutated AAV ITR comprises a deletion of the D region and comprises a mutation of the terminal resolution sequence.

In some aspects, the disclosure provides a composition comprising any of the rAAV particles described herein. In some embodiments, the composition further comprises a pharmaceutically acceptable carrier.

In some aspects, the disclosure provides a cell comprising any of the rAAV particles described herein. In some aspects, the disclosure provides a method of producing a GCase enzyme, the method comprising culturing a cell as described herein under conditions to produce the GBA1 polypeptide. In some embodiments, the methods further comprise the step of purifying the GBA1 polypeptide.

In some aspects, the disclosure provides methods for treating GBA-PD AND/OR GD in an individual in need thereof, comprising administering to the individual a rAAV particle as described herein. In some aspects, the disclosure provides methods for treating GBA-PD AND/OR GD in an individual in need thereof, comprising administering to the individual a composition as described herein. In some embodiments, the disclosure provides methods for treating GBA-PD AND/OR GD in an individual in need thereof, comprising administering to the individual the cell as described herein. In some embodiments, the individual lacks GCase activity.

In some embodiments, the disclosure provides methods of increasing GCase activity by at least about 5% in an individual in need thereof, comprising administering the individual a rAAV particle as described herein. In other embodiments, the disclosure provides methods of increasing GCase activity by at least about 10% in an individual in need thereof, comprising administering the individual a rAAV particle as described herein. In other embodiments, the disclosure provides methods of increasing GCase activity by at least about 20% in an individual in need thereof, comprising administering the individual a rAAV particle as described herein. In other embodiments, the disclosure provides methods of increasing GCase activity by at least about 30% in an individual in need thereof, comprising administering the individual a rAAV particle as described herein. In other embodiments, the disclosure provides methods of increasing GCase activity by at least about 50% in an individual in need thereof, comprising administering the individual a rAAV particle as described herein.

In some embodiments of the above aspects, the rAAV particle is administered only one time to a patient in need thereof. In some embodiments, the rAAV particle is administered multiple times to a patient in need thereof (e.g., over one or more months or years). In other embodiments, the rAAV particle is administered once every year to a patient in need thereof. In other embodiments, the rAAV particle is administered twice yearly to a patient in need thereof.

In some embodiments, the disclosure provides kits comprising any of the rAAV particles, the compositions, or the cell as described herein. In some embodiments, the kit further comprises instructions for use; buffers and/or pharmaceutically acceptable excipients; and/or bottles, vials and/or syringes.

### BRIEF DESCRIPTION OF THE DRAWINGS

FIGS. 1A-1B show a strategy to generate engineered GBA1 variants and in vitro validation of constructs' activity. FIG. 1A depicts a strategy to generate GBA1 variants with enhanced secretion. Endogenous signal sequence of human GBA1 was swapped with signal sequences from highly secreted proteins. Top 4 signal sequences (SS) were narrowed down using in-silico tools that predicted robust secretion as well as high (>98%) probability of cleavage at the end of signal sequence (e.g., cleavage site). FIG. 1B depicts cells transfected with GBA containing the indicated SS variants were lysed and GCase enzyme activity was determined in cell lysates. Mean±SEM, each color represents data point from a single experiment. Untransfected (“Utx”) versus all GBA1 constructs as well as GFP transfected versus all GBA1 constructs: ***p<0.001; One way-ANOVA with Tukey's multiple comparison test.

FIGS. 2A-2B show robust secretion and parenchymal diffusion of GBA1 protein from AAV.SAN006 GBA1 viruses in vivo. FIG. 2A depicts vector biodistribution with in situ hybridization to WPRE (top panels) and huGBA1 immunohistochemistry (bottom panels) in sagittal sections of WT mice injected with AAV. SAN006-GBA1 variants (Bilateral ICV, 1e11 VG/mouse, 5 μl per hemisphere) and analyzed after 4 weeks of expression. FIG. 2B depicts higher magnification images of WPRE mRNA and GBA1 protein in SS3-GBA1 injected mice. Image corresponds to the purple box in FIG. 2A. Representative cells positive for both WPRE mRNA and GBA1 protein are shown in red arrows (e.g., AAV-transduced cells) while mRNA negative and GBA1 protein positive cells are shown in green (e.g., cross-corrected cells).

FIGS. 3A-3C show efficient substrate clearance by SS3-GBA1 variant in multiple brain regions distal to site of AAV injection. FIG. 3A shows WPRE mRNA in situ hybridization showing AAV distribution around the ventricles. FIGS. 3B-3C show lyso-GL1 (FIG. 3B) and total GL-1 (FIG. 3A) in cerebellum, hindbrain, and midbrain of the injected mice (e.g., 4-month-old WT mice injected with 1e11 VG each of the AAV.SAN006-GBA1 variants were IP injected with 100 mg/kg CBE (conduritol β-epoxide) 24 hours prior to necropsy). “No CBE” group in the graphs shown is control mice that did not receive any virus or CBE injection. N=8 mice per group. ***p<0.001, **p<0.01, *p<0.05; One-way ANOVA with Tukey's multiple comparison test with all groups compared to CBE-treated vehicle injected group.

FIGS. 4A-4E show development of conduritol-β-epoxide (CBE)-induced lipid flux model in NHPs. FIG. 4A depicts an illustration of study design of CBE administration in NHPs. FIG. 4B depicts Lyso-GL1 in plasma samples prior to CBE dosing and 24 hours post CBE (left) and (FIG. 4C) dose-dependent increase in Lyso-GL1 in liver tissue homogenates (right). FIGS. 4D-4E depict dose-dependent increase in Lyso-GL1 (FIG. 4D) and concomitant decrease in GCase enzyme activity (FIG. 4E) in 47 grey mater punches of NHP brains, representing 20 grey mater regions. N=1 NHP per dose. Median with inter-quartile range across 47 punches. ***p<0.001; Two-way ANOVA with Tukey's multiple comparison test with all groups compared to 0 mg/kg CBE group or no CBE group.

FIGS. 5A-5E depict effective reduction of GL1 lipids in SS3-GBA1 treated NHPs. FIG. 5A depicts Lyso-GL1 in plasma spiked across all NHPs post CBE dosing. FIGS. 5B-5C depict vector genomes and huGBA1 transgene expression, respectively, in NHPs treated with vehicle, WT-GBA1, and SS3-GBA1 NHPs. 3 NHPs in vehicle and WT-GBA1 groups and 4 NHPs in SS3-GBA1 group. NHPs were dosed with 1.25e13 VGs via iCM dosing, 6 weeks in-life and injected with 30 mg/kg CBE (IV dosing) 48 hours before necropsy. Median with inter-quartile range, each data point is average of all NHPs in the group across 64 brain punches, representing 17 gray mater regions and 7 white mater regions. FIGS. 5D-5E depict Lyso-GL1 and C18 GL1, respectively, across 47 grey mater brain punches. Each data point is average of all NHPs in the group for that punch. ***p<0.001; Two-way ANOVA with Tukey's multiple comparison test. Purple line indicates lipid levels under physiological conditions.

FIGS. 6A-6B show robust secretion and diffusion of SS3-GBA1 in brain sections of SS3-GBA1 ICM dosed NHPs. FIG. 6A depicts low magnification images of WPRE mRNA in situ hybridization (ISH; top panel) and huGBA1 immunohistochemistry (IHC; bottom panel) in representative NHP brain sections. FIG. 6B depicts higher magnification of insets from FIG. 6A of mRNA (left) and GBA1 protein (right). Cells positive for both WPRE mRNA and GBA1 protein are shown in red arrows (e.g., AAV-transduced cells) while mRNA negative and GBA1 protein positive cells are shown in green (e.g., cross-corrected cells).

FIGS. 7A-7B depict effective lipid clearance in mice injected with SS3-GBA1 intravenously. FIG. 7A depicts 3-month-old WT mice were injected with 4e13 VG/kg of vehicle or SS3-GBA1 intravenously. AAVs were expressed for 4 weeks followed by 100 mg/kg CBE (IP injection) 24 hours prior to necropsy. Lyso-GL1 quantification across major peripheral tissues such as liver, spleen, kidney, and lung. FIG. 7B depicts Lyso-GL1 quantification across multiple muscles surveyed such as heart, diaphragm, quadriceps, and gastrocnemius. “No CBE” group in the graphs shown is control mice that did not receive any virus or CBE injection. N=8 mice per group. ***p<0.001; One-way ANOVA with Tukey's multiple comparison test with all groups compared to Vehicle group.

FIG. 8 shows broad biodistribution of AAV.SAN006-GFP in peripheral tissues of NHPs, 3 weeks post intravenous dosing. Vector genomes were quantified per cell (e.g., VGs/cell) across multiple peripheral tissues. Data are mean±SEM. Each data point is tissue from 1 NHP, total of 4 NHPs in the study.

FIGS. 9A-9B depict broad expression of transgene and protein in peripheral tissues of NHPs injected IV with AAV.SAN006-GFP. FIG. 9A depicts GFP mRNA transcript levels. FIG. 9B corresponding GFP ELISA in peripheral tissues of NHPs. Data are mean±SEM. Each data point is tissue from 1 NHP, total of 4 NHPs in the study.

FIG. 10 depicts a SnapGene Map File image of pAAV-CBA-GBA-WPRE-bGH.

FIG. 11 depicts a SnapGene Map File image of pAAV-CBA-SS1-GBA-WPRE-bGH.

FIG. 12 depicts a SnapGene Map File image of pAAV-CBA-SS2-GBA-WPRE-bGH.

FIG. 13 depicts a SnapGene Map File image of pAAV-CBA-SS3-GBA-WPRE-bGH.

FIG. 14 depicts a SnapGene Map File image of pAAV-CBA-SS4-GBA-WPRE-bGH.

FIGS. 15A-15B show increased efficacy of engineered lysosomal protein in distal regions of CNS. FIG. 15A shows WPRE mRNA in situ hybridization (e.g., WPRE ISH) showing AAV distribution in brain tissue. The cortex, hindbrain, and cerebellum regions are circled. FIG. 15B shows quantification of AAV transduction (e.g., vector genomes per cell) and Lyso-GL1 in the corresponding regions of the injected mice. 4-month-old WT mice were injected with 1e11 VG each of the AAV.SAN006-GBA1 variants (e.g., WT-GBA, SS1-GBA, SS2-GBA, SS3-GBA, and SS4-GBA) and IP injected with 100 mg/kg CBE (conduritol β-epoxide) 24 hours prior to necropsy.

FIGS. 16A-16E show cross-correction promotes lipid clearance in peripheral tissues, muscles, and bone marrow with systemic AAV administration. FIGS. 16A-16C show quantification of vector genomes per cell (e.g., VGs/cell; FIG. 16A), GBA protein levels (FIG. 16B), and lipid clearance (FIG. 16C) in samples from 3-month-old mice at 4 weeks after intravenous AAV encoding an engineered lysosomal protein (4e13 VG/kg) or vehicle (e.g., Ctrl-AAV or AAV expressing SS3-GBA). Quantification of in liver, heart, and spleen samples was performed. FIG. 16D shows Lyso-GL1 quantification across soleuc (e.g., muscle tissue) and bone marrow. “No CBE” group is control mice that did not receive any virus or CBE injection. N=8 mice per group. ***p<0.001; One-way ANOVA with Tukey's multiple comparison test with all groups compared to vehicle group. FIG. 16E shows chromogenic dual in situ hybridization and immunohistochemistry to co-detect WPRE mRNA and huGBA1 protein on FFPE sections of liver tissue.

FIGS. 17A-17E show engineered therapeutic protein (e.g., huGBA1) is secreted and taken up by non-transduced cells in NHP brain. Multiplex-imaging in NHP brain was used to co-detect AAV transduced cells (e.g., WPRE mRNA in green) and huGBA1 protein-positive cells (red). NeuN in magenta is a neuronal cell marker. Low magnification image to show the entire brain slab of NHP treated with SS3-GBA1 via ICM dosing at 1.25e13 VGs. Zoomed images are representative images of the motor cortex. Yellow arrows indicate AAV-transduced cells and red arrows indicate cross-corrected cells.

FIGS. 18A-18D show identification of MED of SS3-GBA1 in NHPs from dose-range finding (DRF) study. FIG. 18A shows vector genomes per cell in DRF study were quantified in NHPs treated with 3 doses (e.g., 2.5e12, 7.5e12, or 2.5e13). Data from 5 NHPs per group across 47 grey matter punches and 19 white matter punches in the brain are included. FIG. 18B shows vector genome data plotted across different brain regions. FIG. 18C shows Lyso-GL1 data in plasma and FIG. 18D shows Lyso-GL1 data from all 47 grey mater punches. All scatter plots are median with inter-quartile range. ***p<0.0001; Two-way ANOVA with Tukey's multiple comparison test

FIGS. 19A-19F show histopathological analyses in NHP DRF study. Histopathology findings reported with severity scores across both central nervous system and peripheral tissues. Scores reported for brain (FIG. 19A), peripheral organs (FIG. 19B), sciatic nerve (FIG. 19C), spinal cord (C), DRGs (FIG. 19D and FIG. 19E) and sciatic nerve (FIG. 19F). Data are Mean SEM. Each dot is score for individual NHP. 5 NHPs per treatment group.

FIGS. 20A-20E show long-term efficacy and durability with AAV.SAN006 SS3-GBA1. Vector genomes in cortex (FIG. 20A) and cerebellum (FIG. 20B) were quantified in the longitudinal pharmacology study performed with in-life duration of 3-months, 6-months, and 9-months post AAV-dosing. Cortex is proximal to site of injection and cerebellum is distal to site of injection. Lyso-GL1 clearance in cortex (FIG. 20C), cerebellum (FIG. 20D) and plasma (FIG. 20E) across all mice in the study is shown.

### DETAILED DESCRIPTION

In some aspects, the disclosure provides pharmaceutical compositions comprising rAAV viral particles encapsulating an rAAV vector comprising a transgene capable of encoding a GCase enzyme (polypeptide). In some embodiments, the transgene further encodes a signal peptide. In some embodiments, the signal peptide is not endogenous to the transgene. In some aspects, the disclosure provides methods of treating GD or GBA-PD by administering the rAAV viral particles of the disclosure. In yet further aspects, the disclosure provides kits for treating GD or GBA-PD in an individual with a viral particles of the present disclosure.

### Definitions

A “vector,” as used herein, refers to a recombinant plasmid or virus that comprises a nucleic acid to be delivered into a host cell, either in vitro or in vivo.

The terms “polypeptide” and “protein” are used interchangeably to refer to a polymer of amino acid residues, and are not limited to a minimum length. Such polymers of amino acid residues may contain natural or non-natural amino acid residues, and include, but are not limited to, peptides, oligopeptides, dimers, trimers, and multimers of amino acid residues. Both full-length proteins and fragments thereof are encompassed by the definition. The terms also include post-expression modifications of the polypeptide, for example, glycosylation, sialylation, acetylation, phosphorylation, and the like. Furthermore, for purposes of the present disclosure, a “polypeptide” refers to a protein which includes modifications, such as deletions, additions, and substitutions (generally conservative in nature), to the native sequence, as long as the protein maintains the desired activity. These modifications may be deliberate, as through site-directed mutagenesis, or may be accidental, such as through mutations of hosts which produce the proteins or errors due to PCR amplification.

A “recombinant viral vector” refers to a recombinant polynucleotide vector comprising one or more heterologous sequences (i.e., nucleic acid sequence not of viral origin). In the case of recombinant AAV vectors, the recombinant nucleic acid is flanked by at least one and in embodiments two, inverted terminal repeat sequences (ITRs).

A “recombinant AAV vector (rAAV vector)” refers to a polynucleotide vector comprising one or more heterologous sequences (i.e., nucleic acid sequence not of AAV origin) that are flanked by at least one, and in embodiments two, AAV inverted terminal repeat sequences (ITRs). Such rAAV vectors can be replicated and packaged into infectious viral particles when present in a host cell that has been infected with a suitable helper virus (or that is expressing suitable helper functions) and that is expressing AAV rep and cap gene products (i.e. AAV Rep and Cap proteins). When a rAAV vector is incorporated into a larger polynucleotide (e.g., in a chromosome or in another vector such as a plasmid used for cloning or transfection), then the rAAV vector may be referred to as a “pro-vector” which can be “rescued” by replication and encapsidation in the presence of AAV packaging functions and suitable helper functions. A rAAV vector can be in any of a number of forms, including, but not limited to, plasmids, linear artificial chromosomes, complexed with lipids, encapsulated within liposomes, and encapsidated in a viral particle, particularly an AAV particle. A rAAV vector can be packaged into an AAV virus capsid to generate a “recombinant adeno-associated viral particle (rAAV particle)”.

“Heterologous” means derived from a genotypically distinct entity from that of the rest of the entity to which it is compared or into which it is introduced or incorporated. For example, a polynucleotide introduced by genetic engineering techniques into a different cell type is a heterologous polynucleotide (and, when expressed, can encode a heterologous polypeptide). Similarly, a cellular sequence (e.g., a gene or portion thereof) that is incorporated into a viral vector is a heterologous nucleotide sequence with respect to the vector.

The term “transgene” refers to a polynucleotide that is introduced into a cell and is capable of being transcribed into RNA and optionally, translated and/or expressed under appropriate conditions. In aspects, it confers a desired property to a cell into which it was introduced, or otherwise leads to a desired therapeutic or diagnostic outcome.

“Chicken β-actin (CBA) promoter” refers to a polynucleotide sequence derived from a chicken β-actin gene (e.g., Gallus beta actin, represented by GenBank Entrez Gene ID 396526). As used herein, “chicken β-actin promoter” may refer to a promoter containing a cytomegalovirus (CMV) early enhancer element, the promoter and first exon and intron of the chicken β-actin gene, and the splice acceptor of the rabbit beta-globin gene, such as the sequences described in Miyazaki, J. et al. (1989) Gene 79(2):269-77. As used herein, the term “CAG promoter” may be used interchangeably. As used herein, the term “CMV early enhancer/chicken beta actin (CAG) promoter” may be used interchangeably.

The terms “genome particles (gp),” “genome equivalents,” or “genome copies” as used in reference to a viral titer, refer to the number of virions containing the recombinant AAV DNA genome, regardless of infectivity or functionality. The number of genome particles in a particular vector preparation can be measured by procedures such as described in the Examples herein, or for example, in Clark et al. (1999) Hum. Gene Ther., 10:1031-1039; Veldwijk et al. (2002) Mol. Ther., 6:272-278.

The term “vector genome (vg)” as used herein may refer to one or more polynucleotides comprising a set of the polynucleotide sequences of a vector, e.g., a viral vector. A vector genome may be encapsidated in a viral particle. Depending on the particular viral vector, a vector genome may comprise single-stranded DNA, double-stranded DNA, or single-stranded RNA, or double-stranded RNA. A vector genome may include endogenous sequences associated with a particular viral vector and/or any heterologous sequences inserted into a particular viral vector through recombinant techniques. For example, a recombinant AAV vector genome may include at least one ITR sequence flanking a promoter, a stuffer, a sequence of interest (e.g., an RNAi), and a polyadenylation sequence. A complete vector genome may include a complete set of the polynucleotide sequences of a vector. In some embodiments, the nucleic acid titer of a viral vector may be measured in terms of vg/mL. Methods suitable for measuring this titer are known in the art (e.g., quantitative PCR).

The terms “infection unit (iu),” “infectious particle,” or “replication unit,” as used in reference to a viral titer, refer to the number of infectious and replication-competent recombinant AAV vector particles as measured by the infectious center assay, also known as replication center assay, as described, for example, in McLaughlin et al. (1988) J. Virol., 62:1963-1973.

The term “transducing unit (tu)” as used in reference to a viral titer, refers to the number of infectious recombinant AAV vector particles that result in the production of a functional transgene product as measured in functional assays such as described in Examples herein, or for example, in Xiao et al. (1997) Exp. Neurobiol., 144:113-124; or in Fisher et al. (1996) J. Virol., 70:520-532 (LFU assay).

An “inverted terminal repeat” or “ITR” sequence is a term well understood in the art and refers to relatively short sequences found at the termini of viral genomes which are in opposite orientation.

An “AAV inverted terminal repeat (ITR)” sequence, a term well-understood in the art, is an approximately 145-nucleotide sequence that is present at both termini of the native single-stranded AAV genome. The outermost 125 nucleotides of the ITR can be present in either of two alternative orientations, leading to heterogeneity between different AAV genomes and between the two ends of a single AAV genome. The outermost 125 nucleotides also contains several shorter regions of self-complementarity (designated A, A′, B, B′, C, C′ and D regions), allowing intrastrand base-pairing to occur within this portion of the ITR.

A “terminal resolution sequence” or “trs” is a sequence in the D region of the AAV ITR that is cleaved by AAV rep proteins during viral DNA replication. A mutant terminal resolution sequence is refractory to cleavage by AAV rep proteins.

“AAV helper functions” refer to functions that allow AAV to be replicated and packaged by a host cell. AAV helper functions can be provided in any of a number of forms, including, but not limited to, helper virus or helper virus genes which aid in AAV replication and packaging. Other AAV helper functions are known in the art such as genotoxic agents.

A “helper virus” for AAV refers to a virus that allows AAV (which is a defective parvovirus) to be replicated and packaged by a host cell. A helper virus provides “helper functions” which allow for the replication of AAV. A number of such helper viruses have been identified, including adenoviruses, herpesviruses and, poxviruses such as vaccinia and baculovirus. The adenoviruses encompass a number of different subgroups, although Adenovirus type 5 of subgroup C (Ad5) is most commonly used. Numerous adenoviruses of human, non-human mammalian and avian origin are known and are available from depositories such as the ATCC. Viruses of the herpes family, which are also available from depositories such as ATCC, include, for example, herpes simplex viruses (HSV), Epstein-Barr viruses (EBV), cytomegaloviruses (CMV) and pseudorabies viruses (PRV). Examples of adenovirus helper functions for the replication of AAV include ElA functions, E1B functions, E2A functions, VA functions and E4orf6 functions. Baculoviruses available from depositories include Autographa californica nuclear polyhedrosis virus.

A preparation of rAAV is said to be “substantially free” of helper virus if the ratio of infectious AAV particles to infectious helper virus particles is at least about 10²:1; at least about 10⁴:1, at least about 10⁶:1; or at least about 10⁸:1 or more. In some embodiments, preparations are also free of equivalent amounts of helper virus proteins (i.e., proteins as would be present as a result of such a level of helper virus if the helper virus particle impurities noted above were present in disrupted form). Viral and/or cellular protein contamination can generally be observed as the presence of Coomassie staining bands on SDS gels (e.g., the appearance of bands other than those corresponding to the AAV capsid proteins VP1, VP2, and VP3).

An “effective amount” is an amount sufficient to effect beneficial or desired results, including clinical results (e.g., amelioration of symptoms, achievement of clinical endpoints, and the like). An effective amount can be administered in one or more administrations. In terms of a disease state, an effective amount is an amount sufficient to ameliorate, stabilize, or delay development of a disease.

An “individual” or “subject” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain embodiments, the individual or subject is a human.

As used herein, “treatment” is an approach for obtaining beneficial or desired clinical results. For purposes of this disclosure, beneficial or desired clinical results include, but are not limited to, alleviation of symptoms, diminishment of extent of disease, stabilized (e.g., not worsening) state of disease, preventing spread (e.g., metastasis) of disease, delay or slowing of disease progression, amelioration or palliation of the disease state, and remission (whether partial or total), whether detectable or undetectable. “Treatment” can also mean prolonging survival as compared to expected survival if not receiving treatment.

As used herein, the term “prophylactic treatment” refers to treatment, wherein an individual is known or suspected to have or be at risk for having a disorder but has displayed no symptoms or minimal symptoms of the disorder. An individual undergoing prophylactic treatment may be treated prior to onset of symptoms.

Reference to “about” a value or parameter herein includes (and describes) embodiments that are directed to that value or parameter per se. For example, description referring to “about X” includes description of “X.”

As used herein, the singular form of the articles “a,” “an,” and “the” includes plural references unless indicated otherwise.

It is understood that aspects and embodiments of the disclosure described herein include “comprising,” “consisting,” and/or “consisting essentially of” aspects and embodiments.

### Expression Cassettes

In some embodiments, the transgene encoding an GCase enzyme is codon-optimized. In some embodiments, the transgene encoding GCase enzyme is codon optimized for expression in a particular cell, such as a eukaryotic cell. Eukaryotic cells may be those of or derived from a particular organism, such as a mammal, including but not limited to human, mouse, rat, rabbit, dog, or non-human primate. In general, codon optimization refers to a process of modifying a nucleic acid sequence for enhanced expression in the host cells of interest by replacing at least one codon of the native sequence with codons that are more frequently or most frequently used in the genes of that host cell while maintaining the native amino acid sequence. Various species exhibit particular bias for certain codons of a particular amino acid. Codon usage tables are readily available, for example, at the “Codon Usage Database”, and these tables can be adapted in a number of ways (see, e.g., Nakamura, Y. et al. (2000) Nucleic Acids Res. 28:292). Computer algorithms for codon optimizing a particular sequence for expression in a particular host cell are also available, such as Gene Forge (Aptagen; Jacobus, Pa.), DNA2.0, GeneArt (GA) or Genscript (GS) and a GS algorithm combined with reduction in CpG content. In some embodiments, a transgene encoding the GCase enzyme is is codon optimized using the GA algorithm.

In some embodiments, the vector encodes a GCase enzyme comprising or having a sequence of SEQ ID NO:1. In some embodiments, the vector encodes a GCase enzyme comprising or having a sequence of at least 85%, at least 90%, at least 95%, at least 98% or at least 99% homology to a sequence of SEQ ID NO:1. SEQ ID NO:1 is the wild-type amino acid sequence of glucocerebrosidase (GCase). The sequence includes an N-terminal 39-amino acid signaling peptide that is cleaved after expression of the protein, hence generating a mature protein having an amino acid sequence of SEQ ID NO: 2. In some embodiments, the vector encodes a GCase enzyme comprising an amino acid sequence of SEQ ID NO:2. In some embodiments, the vector encodes a GCase enzyme comprising or having an amino acid sequence of at least 85%, at least 90%, at least 95%, at least 98% or at least 99% homology to a sequence of SEQ ID NO:2. In some embodiments, the vector encodes a GCase enzyme comprising or having an amino acid sequence of SEQ ID NO:2 following cleavage (removal) of the signaling peptide. In some embodiments, the vector encodes a GCase enzyme comprising or having a sequence of at least 85%, at least 90%, at least 95%, at least 98% or at least 99% homology to SEQ ID NO:2 following cleavage (removal) of the signaling peptide.

As set forth herein, the human GBA1 gene can be engineered to express GCase enzymes (polypeptides) having signaling peptides that differ from the wild-type (endogenous) sequence in SEQ ID NO:1. Upon cleavage of the signaling peptide, the same mature proteins, for instance, a GCase enzyme having a sequence of SEQ ID NO:2 is produced. It has been found that certain GCase enzymes with particular signaling peptides are more readily secretable than the wild-type GCase enzymes having a sequence of SEQ ID NO: 1. In certain embodiments, GCase enzymes with signaling peptides that differ from the wild-type peptide provide greater levels of transgene expression than the wild-type GCase enzyme.

In some embodiments, the sequences used to express the GCase enzyme are codon-optimized sequences. In some embodiments, the codon-optimized nucleic acid sequence has a sequence of SEQ ID NO:3. In some embodiments, the codon-optimized nucleic acid sequence has a sequence of SEQ ID NO:4 (also referred to herein as SS1-GBA1). In some embodiments, the codon-optimized nucleic acid sequence has a sequence of SEQ ID NO:5 (also referred to herein as SS2-GBA1). In some embodiments, the codon-optimized nucleic acid sequence has a sequence of SEQ ID NO:6 (also referred to herein as SS3-GBA1). In some embodiments, the codon-optimized nucleic acid sequence has a sequence of SEQ ID NO:7 (also referred to herein as SS4-GBA1). The nucleic acid sequence of SEQ ID NO:3 expresses the wild-type human GCase enzyme (e.g., the enzyme having SEQ ID NO: 1), which includes the endogenous signaling peptide. In some embodiments, polynucleotides of SEQ ID NOs:4-7 express variant GCase enzymes that have different signaling peptides.

In some embodiments, the expression cassette further comprises an intron. A variety of introns for use in the disclosure are known to those of skill in the art, and include the MVM intron, the F IX truncated intron 1, the β-globin SD/immunoglobin heavy chain SA, the adenovirus SD/immunoglobin SA, the SV40 late SD/SA (19S/16S), and the hybrid adenovirus SD/IgG SA. (Wu et al. 2008, Kurachi et al., 1995, Choi et al. 2014, Wong et al., 1985, Yew et al. 1997, Huang and Gorman (1990). In some embodiments, the intron is a chicken β-actin (CBA)/rabbit β-globin hybrid intron. In some embodiments, intron is a chicken β-actin (CBA)/rabbit β-globin hybrid promoter and intron where all the ATG sites are removed to minimize false translation start sites. In some embodiments the intron is an MVM intron, a F IX truncated intron 1, a β-globin SD/immunoglobin heavy chain SA, an adenovirus SD/immunoglobin SA, a SV40 late SD/SA (19S/16S), or a hybrid adenovirus SD/IgG SA. In some embodiments, the intron is a chicken β-actin (CBA)/rabbit β-globin hybrid intron.

In some embodiments, the expression cassette further comprises a polyadenylation signal. In some embodiments, the polyadenylation signal is a bovine growth hormone polyadenylation signal, an SV40 polyadenylation signal, or a HSV TK pA. In some embodiments, the polyadenylation signal is a synthetic polyadenylation signal as described in Levitt, N et al. (1989), Genes Develop. 3:1019-1025.

In some embodiments, the expression cassette comprises a stuffer nucleic acid. In some embodiments, the stuffer nucleic acid may comprise a sequence that encodes a reporter polypeptide. As will be appreciated by those of skill in the art, the stuffer nucleic acid may be located in a variety of regions within the nucleic, and may be comprised of a continuous sequence (e.g., a single stuffer nucleic acid in a single location) or multiple sequences (e.g., more than one stuffer nucleic acid in more than one location (e.g., 2 locations, 3 locations, etc.) within the nucleic acid. In some embodiments, the stuffer nucleic acid may be located downstream of the transgene encoding the GCase enzyme. In embodiments, the stuffer nucleic acid may be located upstream of the transgene encoding the GCase enzyme (e.g., between the promoter and the transgene). As will also be appreciated by those of skill in the art a variety of nucleic acids may be used as a stuffer nucleic acid. In some embodiments, the stuffer nucleic acid comprises all or a portion of a human alpha-1-antitrypsin (AAT) stuffer sequence or a C16 P1 chromosome 16 P1 clone (human C16) stuffer sequence. In some embodiments, the stuffer sequence comprises all or a portion of a gene. For example, the stuffer sequence comprises a portion of the human AAT sequence. One skilled in the art would recognize that different portions of a gene (e.g., the human AAT sequence) can be used as a stuffer fragment. For example, the stuffer fragment may be from the 5′ end of the gene, the 3′ end of the gene, the middle of a gene, a non-coding portion of the gene (e.g., an intron), a coding region of the gene (e.g. an exon), or a mixture of non-coding and coding portions of a gene. One skilled in the art would also recognize that all or a portion of stuffer sequence may be used as a stuffer sequence. In some embodiments, the stuffer sequence is modified to remove internal ATG codons.

In some embodiments, the expression cassette is incorporated into a vector. In some embodiments, the expression cassette is incorporated into a viral vector. In some embodiments, the viral vector is a rAAV vector as described herein.

### Vectors and Viral Particles

In certain aspects, the expression cassette for expressing a GCase enzyme is (e.g., a wild type human GCase enzyme or a GCase enzyme with a mutated signaling peptide) is contained in a vector. In some embodiments, the present disclosure contemplates the use of a recombinant viral genome for introduction of nucleic acid sequences encoding the GCase enzyme is for packaging into a viral particle, e.g., a viral particle described below. The recombinant viral genome may include any element to establish the expression of the GCase enzyme, for example, a promoter, an ITR, a ribosome binding element, terminator, enhancer, selection marker, intron, polyA signal, and/or origin of replication. Exemplary viral genome elements and delivery methods for viral particles are described in greater detail below.

### Non-Viral Delivery Systems

Conventional non-viral gene transfer methods may also be used to introduce nucleic acids into cells or target tissues. Non-viral vector delivery systems include DNA plasmids, naked nucleic acid, and nucleic acid complexed to a delivery system. For example, the vector may be complexed to a lipid (e.g., a cationic or neutral lipid), a liposome, a polycation, a nanoparticle, or an agent that enhances the cellular uptake of nucleic acid. The vector may be complexed to an agent suitable for any of the delivery methods described herein. In some embodiments, the nucleic acid comprises one or more viral ITRs (e.g., AAV ITRs).

### Viral Particles

In some embodiments, the vector comprising the expression cassette for expressing a GCase enzyme (e.g., a wild type human GCase enzyme or a GCase enzyme with a mutated signaling peptide) is a recombinant viral vector. Some examples of recombinant viral vectors include AAV, lentivirus and adenovirus. In one embodiment, the viral vector is a recombinant adeno-associated virus (rAAV) vector. In some embodiments, the expression cassette for expressing a GCase enzyme (e.g., a wild type human GCase enzyme or a GCase enzyme with a mutated signaling peptide) is flanked by one or more AAV inverted terminal repeat (ITR) sequences. In some embodiments, the viral particle is a recombinant AAV particle comprising an expression cassette for expressing a GCAse enzyme is flanked by one or two ITRs. In some embodiments, the expression cassette for expressing a GCAse enzyme is flanked by two AAV ITRs.

In some embodiments, the expression cassette for expressing a GCase enzyme of the present disclosure operatively linked components in the direction of transcription, control sequences including transcription initiation and termination sequences, thereby forming an expression cassette. The expression cassette is flanked on the 5′ and 3′ end by at least one functional AAV ITR sequence. By “functional AAV ITR sequences” it is meant that the ITR sequences function as intended for the rescue, replication and packaging of the AAV virion. See Davidson et al., PNAS, 2000, 97(7)3428-32; Passini et al., J. Virol., 2003, 77(12):7034-40; and Pechan et al., Gene Ther., 2009, 16:10-16, all of which are incorporated herein in their entirety by reference. For practicing some aspects of the disclosure, the recombinant vectors comprise at least all of the sequences of AAV essential for encapsidation and the physical structures for infection by the rAAV. AAV ITRs for use in the vectors of the disclosure need not have a wild-type nucleotide sequence (e.g., as described in Kotin, Hum. Gene Ther., 1994, 5:793-801), and may be altered by the insertion, deletion or substitution of nucleotides or the AAV ITRs may be derived from any of several AAV serotypes. More than 40 serotypes of AAV are currently known, and new serotypes and variants of existing serotypes continue to be identified. See Gao et al., PNAS, 2002, 99(18): 11854-6; Gao et al., PNAS, 2003, 100(10):6081-6; and Bossis et al., J. Virol., 2003, 77(12):6799-810.

Use of any AAV serotype is considered within the scope of the present disclosure. In some embodiments, a rAAV vector is a vector derived from an AAV serotype, including without limitation, AAV ITRs are AAV1, AAV2, AAV3, AAV4, AAV5, AAV6, AAV7, AAV8, AAVrh8, AAVrh8R, AAV9, AAV10, AAV.rh10, AAV11, AAV12, a goat AAV, bovine AAV, or mouse AAV ITRs or the like. In some embodiments, the nucleic acid in the AAV comprises an ITR of AAV ITRs are AAV1, AAV2, AAV3, AAV4, AAV5, AAV6, AAV7, AAV8, AAVrh8, AAVrh8R, AAV9, AAV10, AAVrh10, AAV11, AAV12, a goat AAV, bovine AAV, or mouse AAV or the like. In certain embodiments, the AAV ITRs are AAV2 ITRs.

In some embodiments, a vector may include a stuffer nucleic acid. In some embodiments, the stuffer nucleic acid may encode a green fluorescent protein (GFP). In some embodiments, the stuffer nucleic acid may be located 3′ to expression cassette for expressing a GCase enzyme of the present disclosure.

In some embodiments, the disclosure provides viral particles comprising a single-stranded genome. In some embodiments, the disclosure provides viral particles comprising a recombinant self-complementing genome. In some embodiments, the vector is a self-complementary vector. AAV viral particles with self-complementing genomes and methods of use of self-complementing AAV genomes are described in U.S. Pat. Nos. 6,596,535; 7,125,717; 7,765,583; 7,785,888; 7,790,154; 7,846,729; 8,093,054; and 8,361,457; and Wang Z., et al., (2003) Gene Ther 10:2105-2111, each of which are incorporated herein by reference in its entirety. A rAAV comprising a self-complementing genome will quickly form a double stranded DNA molecule by virtue of its partially complementing sequences (e.g., complementing coding and non-coding strands of a transgene). In some embodiments, the disclosure provides an AAV viral particle comprising an AAV genome, wherein the rAAV genome comprises a first heterologous polynucleotide sequence (e.g., the coding strand of the GBA1 polypeptide of the disclosure) and a second heterologous polynucleotide sequence (e.g., the noncoding or antisense strand of the GBA1 polypeptide of the present disclosure) wherein the first heterologous polynucleotide sequence can form intrastrand base pairs with the second polynucleotide sequence along most or all of its length.

In some embodiments, the first heterologous polynucleotide sequence and a second heterologous polynucleotide sequence are linked by a sequence that facilitates intrastrand basepairing; e.g., a hairpin DNA structure. Hairpin structures are known in the art, for example in siRNA molecules. In some embodiments, the first heterologous polynucleotide sequence and a second heterologous polynucleotide sequence are linked by a mutated ITR (e.g., the right ITR). The mutated ITR comprises a deletion of the D region comprising the terminal resolution sequence. As a result, on replicating an AAV viral genome, the rep proteins will not cleave the viral genome at the mutated ITR and as such, a recombinant viral genome comprising the following in 5′ to 3′ order will be packaged in a viral capsid: an AAV ITR, the first heterologous polynucleotide sequence including regulatory sequences, the mutated AAV ITR, the second heterologous polynucleotide in reverse orientation to the first heterologous polynucleotide and a third AAV ITR.

In some embodiments, the first heterologous nucleic acid sequence and a second heterologous nucleic acid sequence are linked by a mutated ITR (e.g., the right ITR). In some embodiments, the ITR comprises the polynucleotide sequence 5′-CACTCCCTCTCTGCGCGCT CGCTCGCTCACTGAGGCCGGGCGACCAAAGGTCGCCCACGCCCGGGCTTTGCCCGG GCG-3′ (SEQ ID NO:24). The mutated ITR comprises a deletion of the D region comprising the terminal resolution sequence. As a result, on replicating an AAV viral genome, the rep proteins will not cleave the viral genome at the mutated ITR and as such, a recombinant viral genome comprising the following in 5′ to 3′ order will be packaged in a viral capsid: an AAV ITR, the first heterologous polynucleotide sequence including regulatory sequences, the mutated AAV ITR, the second heterologous polynucleotide in reverse orientation to the first heterologous polynucleotide and a third AAV ITR.

In some embodiments, the vector is encapsidated in a viral particle. In some embodiments, the viral particle is a recombinant AAV viral particle comprising a recombinant AAV vector. Different AAV serotypes are used to optimize transduction of particular target cells or to target specific cell types within a particular target tissue (e.g., brain or spinal). A rAAV particle can comprise viral proteins and viral nucleic acids of the same serotype or a mixed serotype. For example, in some embodiments a rAAV particle can comprise SAN006 capsid proteins and at least one AAV2 ITR or it can comprise SAN006 capsid proteins and at least one AAV1 ITR. Any combination of AAV serotypes for production of a rAAV particle is provided herein as if each combination had been expressly stated herein.

The capsids encapsulating the vectors encoding the GBA1 transgene are modified AAV9 vectors. The capsid of AAV9 is known to include three capsid proteins: VP1 (SEQ ID NO: 13), VP2 (SEQ ID NO: 14), and VP3 (SEQ ID NO: 15). These proteins contain significant amounts of overlapping amino acid sequence and unique N-terminal sequences. An AAV9 capsid includes 60 subunits arranged by icosahedral symmetry. AAV9 includes VP1, VP2, and VP3 capsid proteins in a ratio of about 5:5:50. The VP proteins of AAV9 are products of the structural protein-encoding open reading frame of the genome, designated cap, VP1 (˜82 kDa) and VP2 (˜73 kDa), which are the minor capsid proteins, and VP3 (˜61 kDa), the major capsid protein. Due to the utilization of both alternative splicing and leaky scanning, when expressed, the individual VPs share a C terminus that encompasses the entire VP3, while VP1 and VP2 are N-terminal VP3 extensions. VP1 and VP2 share a region of ˜73 amino acids amino acids which is extended by an additional 137 amino acids in VP1, designated the VP1 unique region (VP1u). See Penzes et al., (2021), Journal of Virology 95(19)e0084321.

In particular embodiments, the targeting peptide of the modified AAV9 capsids are inserted after residue 588 of the AAV9 structural protein (numbering based on VP1 numbering of AAV9). In some embodiments, the targeting peptide has SEQ ID NO: 16. In some embodiments, the targeting peptide is flanked by linker sequences on the N-terminal and the C-terminal end of the targeting peptide. In some embodiments, the linker sequence on the N-terminal side has the sequence AAA. In some embodiments, the linker sequence on the C-terminal side is AS. In some embodiments, the full sequence inserted after residue 588 of the AAV9 capsid structural protein has SEQ ID NO: 17. In some embodiments, the full modified AAV9 capsid structural protein has SEQ ID NO: 18. In some embodiments, the full modified AAV9 capsid structural protein that it at least 90% (e.g., at least 92%, at least 95%, at least 98%, at least 98.5%, at least 99%, at least 99.2%, at least 99.5%, or at least 99.8%) identical to SEQ ID NO: 18, wherein the modified AAV9 structural capsid comprises the targeting peptide of SEQ ID NO: 16. The capsid having SEQ ID NO: 18 will also be referred to herein as SAN006. The capsid SAN006 was first described in United States Patent Application Publication No. US 2023/0346981, which is hereby incorporated by reference in its entirety.

In some embodiments, the rAAV vector particle may comprise pAAV-CBA-GBA-WPRE-bGH. In some embodiments, the rAAV vector particle may comprise SAN006-CBA-GBA-WPRE-bGH. In some embodiments, SAN006-CBA-GBA-WPRE-bGH may comprise an ITR-ITR sequence. In some embodiments, the ITR-ITR sequence may comprise annotations. In some embodiments, the ITR-ITR sequence annotations may comprise: bp 1:4473, remaining plasmid sequence 4476:10864 comprise stuffer sequence and bacterial elements for plasmid propagation, with ampicillin resistance for selection.

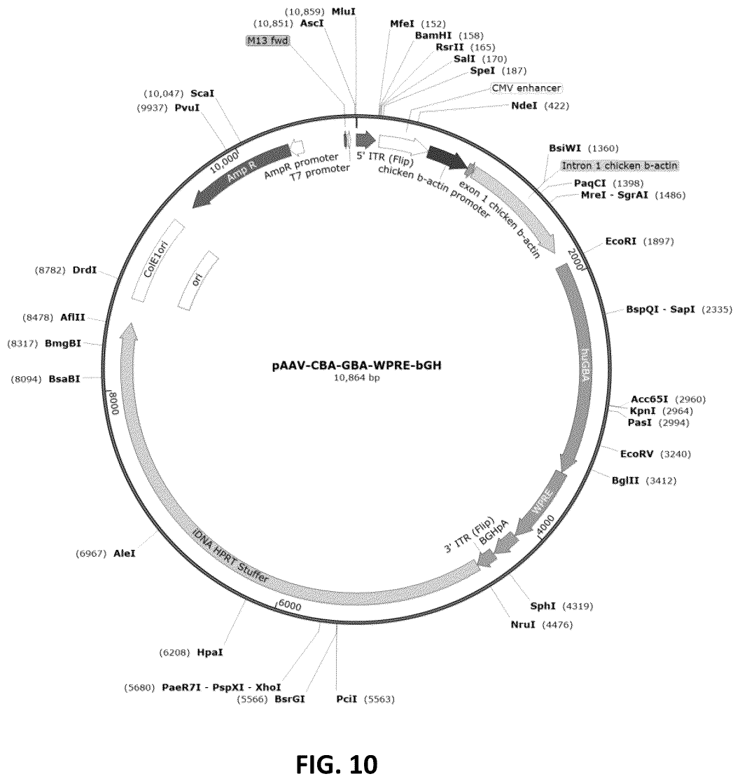

In some embodiments, the plasmid for rAAV packaging (e.g., pAAV-CBA-GBA-WPRE-bGH) may comprise a plurality of elements as depicted in FIG. 10. In some embodiments, pAAV-CBA-GBA-WPRE-bGH may comprise SEQ ID NO: 19.

In some embodiments, SAN006-CBA-GBA-WPRE-bGH may comprise at bases 1:145, a WT AAV2 5′ ITR element in Flip orientation (NCBI Reference Sequence: NC_001401.2). In some embodiments, at bases 173:553, SAN006-CBA-GBA-WPRE-bGH may comprise a CMV Enhancer element (GenBank: K03104.1) (https://www.ncbi.nlm.nih.gov/nucleotide/K03104.1?report=genbank&log$=nuclalign&blast_ra nk=1&RID=G8XBFMKV114). In some embodiments, at bases 554:873, pAAV-CBA-GBA-WPRE-bGH may comprise a chicken β-actin promoter (NCBI Reference Sequence: NC_052545.1). In some embodiments, at bases 874:924 SAN006-CBA-GBA-WPRE-bGH may comprise an exon 1 chicken β-actin (NCBI Reference Sequence: NM_205518.1). In some embodiments, at bases 925:1799, pAAV-CBA-GBA-WPRE-bGH may comprise an Intron 1 chicken b-actin (NCBI Reference Sequence: NC_052545.1). In some embodiments, at bases 1808:1848, pAAV-CBA-GBA-WPRE-bGH may comprise an intron 2 Rabbit beta globin (GenBank: V00882.1). In some embodiments, at bases 1849:1902, pAAV-CBA-GBA-WPRE-bGH may comprise an exon 3 Rabbit beta globin (GenBank: V00882.1). In some embodiments, at bases 1909:3519, pAAV-CBA-GBA-WPRE-bGH may comprise a lysosomal acid glucosylceramidase isoform 1 precursor [Homo sapiens] (NCBI Reference Sequence: NP_000148.2), codon optimized (GeneArt Gene Synthesis). In some embodiments, at bases 3522:4115, pAAV-CBA-GBA-WPRE-bGH may comprise a woodchuck hepatitis virus posttranscriptional regulatory element (e.g., WPRE), (NCBI Reference Sequence: NC_004107.1). In some embodiments, at bases 4119:4321, pAAV-CBA-GBA-WPRE-bGH may comprise a polyA signal sequence; Bovine growth hormone gene polyadenylation sequence (GenBank: M57764.1). In some embodiments, at bases 4329:4473, pAAV-CBA-GBA-WPRE-bGH may comprise a WT AAV2 3′ ITR element in Flip orientation (NCBI Reference Sequence: NC_001401.2).

In some embodiments, the rAAV vector particle may comprise pAAV-CBA-SS1-GBA-WPRE-bGH. In some embodiments, the rAAV vector particle may comprise pAAV-CBA-GBA-WPRE-bGH. In some embodiments, the rAAV vector particle may comprise pAAV-CBA-GBA-WPRE-bGH.

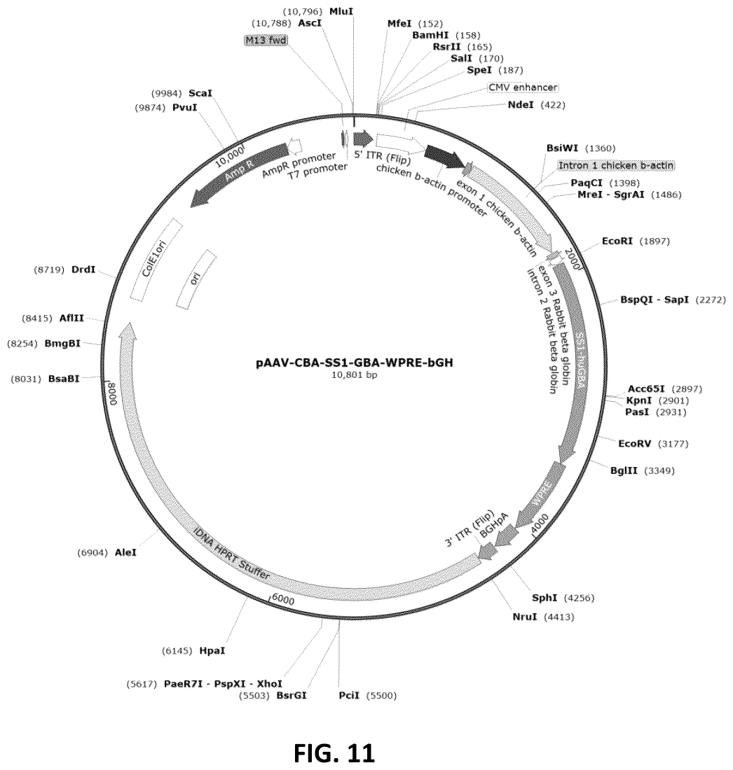

In some embodiments, the plasmid for rAAV packaging, (e.g., pAAV-CBA-SS1-GBA-WPRE-bGH) may comprise a plurality of elements a depicted in FIG. 11. In some embodiments, pAAV-CBA-SS1-GBA-WPRE-bGH may comprise SEQ ID NO. 20.

In some embodiments, at bases 1:145, pAAV-CBA-SS1-GBA-WPRE-bGH may comprise a WT AAV2 5′ ITR element in Flip orientation (NCBI Reference Sequence: NC_001401.2). In some embodiments, at bases 173:553, pAAV-CBA-SS1-GBA-WPRE-bGH may comprise a CMV Enhancer element (GenBank: K03104.1), (https://www.ncbi.nlm.nih.gov/nucleotide/K03104.1?report=genbank&log$=nuclalign&blast_ra nk=1&RID=G8XBFMKV114). In some embodiments, at bases 554:873, pAAV-CBA-SS1-GBA-WPRE-bGH may comprise a chicken β-actin promoter (NCBI Reference Sequence: NC_052545.1). In some embodiments, at bases 874:924, pAAV-CBA-SS1-GBA-WPRE-bGH may comprise an exon 1 chicken R-actin (NCBI Reference Sequence: NM_205518.1). In some embodiments, at bases 925:1799, pAAV-CBA-SS1-GBA-WPRE-bGH may comprise an Intron 1 chicken b-actin (NCBI Reference Sequence: NC_052545.1). In some embodiments, at bases 1808:1848, pAAV-CBA-SS1-GBA-WPRE-bGH may comprise an intron 2 Rabbit beta globin (GenBank: V00882.1). In some embodiments, at bases 1849:1902 pAAV-CBA-SS1-GBA-WPRE-bGH may comprise an exon 3 Rabbit beta globin (GenBank: V00882.1). In some embodiments, at bases 1909:3456, pAAV-CBA-SS1-GBA-WPRE-bGH may comprise a lysosomal acid glucosylceramidase isoform 1 precursor [Homo sapiens] (NCBI Reference Sequence: NP_000148.2), codon optimized (GeneArt Gene Synthesis), endogenous signaling sequence replaced with SS1. In some embodiments, at bases 3459:4052, pAAV-CBA-SS1-GBA-WPRE-bGH may comprise a woodchuck hepatitis virus posttranscriptional regulatory element (WPRE) (NCBI Reference Sequence: NC_004107.1). In some embodiments, at bases 4056:4258, pAAV-CBA-SS1-GBA-WPRE-bGH may comprise a polyA signal sequence; Bovine growth hormone gene polyadenylation sequence (GenBank: M57764.1). In some embodiments, at bases 4266:4410, pAAV-CBA-SS1-GBA-WPRE-bGH may comprise a WT AAV2 3′ ITR element in Flip orientation (NCBI Reference Sequence: NC_001401.2).

In some embodiments, the rAAV vector particle may comprise pAAV-CBA-SS2-GBA-WPRE-bGH. In some embodiments, the rAAV vector particle may comprise SAN006-CBA-SS2-GBA1-WPRE. In some embodiments, the rAAV vector particle may comprise SAN006-CBA-SS2-GBA-WPRE. In some embodiments, pAAV-CBA-SS2-GBA-WPRE-bGH may comprise an ITR-ITR. In some embodiments, the ITR-ITR sequence may comprise sequence annotations, comprising: bp 1:4413, remaining plasmid sequence 4416:10804 comprise stuffer sequence and bacterial elements for plasmid propagation, with ampicillin resistance for selection.

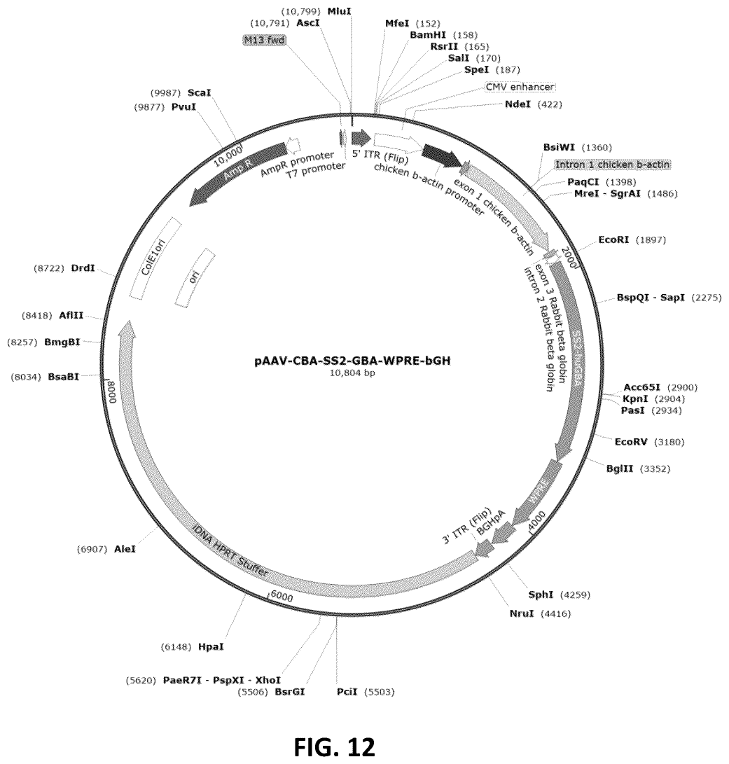

In some embodiments, the plasmid for rAAV packaging (e.g., pAAV-CBA-SS2-GBA-WPRE-bGH), may comprise a plurality of elements as depicted in FIG. 12. In some embodiments, pAAV-CBA-SS2-GBA-WPRE-bGH may comprise SEQ ID NO. 21.

In some embodiments, at bases 1:145, pAAV-CBA-SS2-GBA-WPRE-bGH may comprise a WT AAV2 5′ ITR element in Flip orientation (NCBI Reference Sequence: NC_001401.2); (2) at bases 173:553: CMV Enhancer element (GenBank: K03104.1) (https://www.ncbi.nlm.nih.gov/nucleotide/K03104.1?report=genbank&log$=nuclalign&blast_ra nk=1&RID=G8XBFMKV114). In some embodiments, at bases 554:873, pAAV-CBA-SS2-GBA-WPRE-bGH may comprise a chicken j-actin promoter (NCBI Reference Sequence: NC_052545.1). In some embodiments, at bases 874:924, pAAV-CBA-SS2-GBA-WPRE-bGH may comprise a exon 1 chicken j-actin (NCBI Reference Sequence: NM_205518.1). In some embodiments, at bases 925:1799, pAAV-CBA-SS2-GBA-WPRE-bGH an Intron 1 chicken b-actin (NCBI Reference Sequence: NC_052545.1). In some embodiments, at bases 1808:1848, pAAV-CBA-SS2-GBA-WPRE-bGH may comprise an intron 2 Rabbit beta globin (GenBank: V00882.1). In some embodiments, at bases 1849:1902, pAAV-CBA-SS2-GBA-WPRE-bGH may comprise an exon 3 Rabbit beta globin (GenBank: V00882.1). In some embodiments, at bases 1909:3459, pAAV-CBA-SS2-GBA-WPRE-bGH may comprise a lysosomal acid glucosylceramidase isoform 1 precursor [Homo sapiens] (NCBI Reference Sequence: NP_000148.2), codon optimized (GeneArt Gene Synthesis), endogenous signaling sequence replaced with SS2. In some embodiments, at bases 3462:4055, pAAV-CBA-SS2-GBA-WPRE-bGH may comprise woodchuck hepatitis virus posttranscriptional regulatory element (WPRE), (NCBI Reference Sequence: NC_004107.1). In some embodiments, at bases 4059:4261, pAAV-CBA-SS2-GBA-WPRE-bGH may comprise a polyA signal sequence; Bovine growth hormone gene polyadenylation sequence (GenBank: M57764.1). In some embodiments, at bases 4269:4413, pAAV-CBA-SS2-GBA-WPRE-bGH may comprise a WT AAV2 3′ ITR element in Flip orientation (NCBI Reference Sequence: NC_001401.2).

In some embodiments, the rAAV vector particle may comprise pAAV-CBA-SS3-GBA-WPRE-bGH. In some embodiments, the rAAV vector particle may comprise SAN006-CBA-SS3-GBA1-WPRE. In some embodiments, the rAAV vector particle may comprise SAN006-CBA-SS3-GBA-WPRE.

In some embodiments, pAAV-CBA-SS3-GBA-WPRE-bGH may comprise an ITR-ITR sequence. In some embodiments, pAAV-CBA-SS3-GBA-WPRE-bGH may comprise an ITR-ITR sequence, may comprise sequence annotations comprising at bp 1:4407, remaining plasmid sequence 4410:10798 comprise stuffer sequence and bacterial elements for plasmid propagation, with ampicillin resistance for selection.

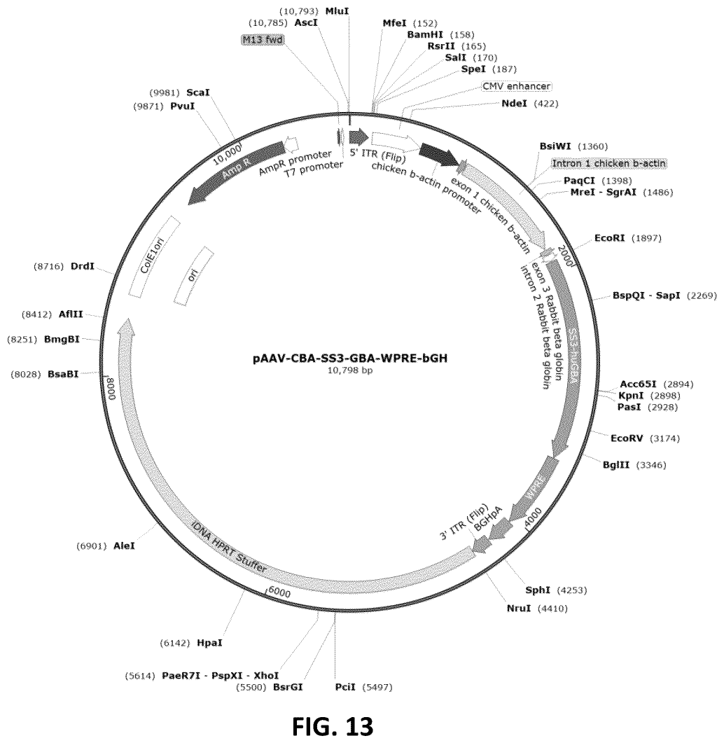

In some embodiments, the plasmid for rAAV packaging (e.g., pAAV-CBA-SS3-GBA-WPRE-bGH), may comprise a plurality of elements as depicted in FIG. 13. In some embodiments, pAAV-CBA-SS3-GBA-WPRE-bGH may comprise SEQ ID NO. 22.

In some embodiments, at bases 1:145, pAAV-CBA-SS3-GBA-WPRE-bGH may comprise the WT AAV2 5′ ITR element in Flip orientation (NCBI Reference Sequence: NC_001401.2). In some embodiments, at bases 173:553, pAAV-CBA-SS3-GBA-WPRE-bGH may comprise a CMV Enhancer element (GenBank: K03104.1) (https://www.ncbi.nlm.nih.gov/nucleotide/K03104.1?report=genbank&log$=nuclalign&blast_ra nk=1&RID=G8XBFMKV114). In some embodiments, at bases 554:873 may comprise a chicken β-actin promoter (NCBI Reference Sequence: NC_052545.1). In some embodiments, bases 874:924, pAAV-CBA-SS3-GBA-WPRE-bGH may comprise an exon 1 chicken R-actin (NCBI Reference Sequence: NM_205518.1). In some embodiments, at bases 925:1799, pAAV-CBA-SS3-GBA-WPRE-bGH may comprise an Intron 1 chicken b-actin (NCBI Reference Sequence: NC_052545.1). In some embodiments, at bases 1808:1848, pAAV-CBA-SS3-GBA-WPRE-bGH may comprise an intron 2 Rabbit beta globin (GenBank: V00882.1). In some embodiments, at bases 1849:1902 pAAV-CBA-SS3-GBA-WPRE-bGH may comprise an exon 3 Rabbit beta globin (GenBank: V00882.1). In some embodiments, at bases 1909:3453, pAAV-CBA-SS3-GBA-WPRE-bGH may comprise a lysosomal acid glucosylceramidase isoform 1 precursor [Homo sapiens] (NCBI Reference Sequence: NP_000148.2), codon optimized (GeneArt Gene Synthesis), endogenous signaling sequence replaced with SS3. In some embodiments, at bases 3456:4049, pAAV-CBA-SS3-GBA-WPRE-bGH may comprise a WPRE; woodchuck hepatitis virus posttranscriptional regulatory element, (NCBI Reference Sequence: NC_004107.1). In some embodiments, at bases 4053:4255, pAAV-CBA-SS3-GBA-WPRE-bGH may comprise a polyA signal sequence; Bovine growth hormone gene polyadenylation sequence (GenBank: M57764.1). In some embodiments, at bases 4263:4407, pAAV-CBA-SS3-GBA-WPRE-bGH may comprise a WT AAV2 3′ ITR element in Flip orientation (NCBI Reference Sequence: NC_001401.2).

In some embodiments, the rAAV vector particle may comprise pAAV-CBA-SS4-GBA-WPRE-bGH. In some embodiments, the rAAV vector particle may comprise SAN006-CBA-SS4-GBA1-WPRE. In some embodiments, the rAAV vector particle may comprise SAN006-CBA-SS4-GBA-WPRE. In some embodiments, pAAV-CBA-SS4-GBA-WPRE-bGH. In some embodiments, the rAAV vector particle may comprise SAN006-CBA-SS4-GBA1-WPRE may comprise an ITR-ITR sequence. In some embodiments, the rAAV vector particle may comprise SAN006-CBA-SS4-GBA1-WPRE may comprise an ITR-ITR sequence may comprise ITR-ITR sequence annotations comprising bp 1:4410 and/or remaining plasmid sequence 4413:10801 comprising stuffer sequence and bacterial elements for plasmid propagation, with ampicillin resistance for selection.

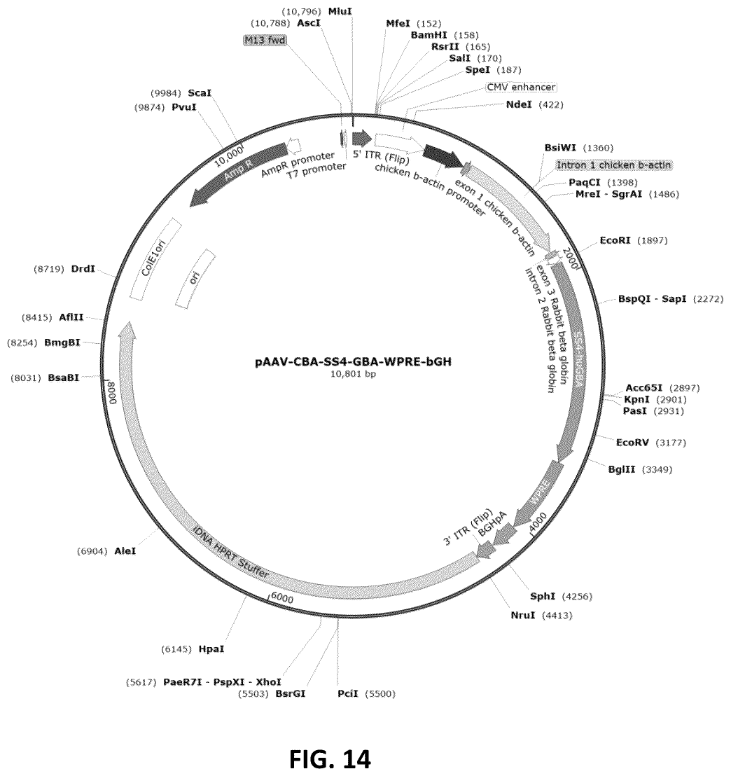

In some embodiments, the plasmid for rAAV packaging (e.g., pAAV-CBA-SS4-GBA-WPRE-bGH, may comprise a plurality of elements as depicted in FIG. 14. In some embodiments, pAAV-CBA-SS4-GBA-WPRE-bGH may comprise SEQ ID NO: 23.

In some embodiments, a bases 1:145, pAAV-CBA-SS4-GBA-WPRE-bGH may comprise a WT AAV2 5′ ITR element in Flip orientation (NCBI Reference Sequence: NC_001401.2). In some embodiments, at bases 173:553, pAAV-CBA-SS4-GBA-WPRE-bGH may comprise a CMV Enhancer element (GenBank: K03104.1) (https://www.ncbi.nlm.nih.gov/nucleotide/K03104.1?report=genbank&log$=nuclalign&blast_ra nk=1&RID=G8XBFMKV114). In some embodiments, at bases 554:873, pAAV-CBA-SS4-GBA-WPRE-bGH may comprise a chicken β-actin promoter (NCBI Reference Sequence: NC_052545.1). In some embodiments, at bases 874:924, pAAV-CBA-SS4-GBA-WPRE-bGH an exon 1 chicken β-actin (NCBI Reference Sequence: NM_205518.1). In some embodiments, at bases 925:1799, pAAV-CBA-SS4-GBA-WPRE-bGH may comprise an Intron 1 chicken b-actin (NCBI Reference Sequence: NC_052545.1). In some embodiments, at bases 1808:1848, pAAV-CBA-SS4-GBA-WPRE-bGH may comprise an intron 2 Rabbit beta globin (GenBank: V00882.1). In some embodiments, at bases 1849:1902, pAAV-CBA-SS4-GBA-WPRE-bGH may comprise an exon 3 Rabbit beta globin (GenBank: V00882.1). In some embodiments, at bases 1909:3456, pAAV-CBA-SS4-GBA-WPRE-bGH may comprise a lysosomal acid glucosylceramidase isoform 1 precursor [Homo sapiens] (NCBI Reference Sequence: NP_000148.2), codon optimized (GeneArt Gene Synthesis), endogenous signaling sequence replaced with SS34. In some embodiments, at bases 3459:4052, pAAV-CBA-SS4-GBA-WPRE-bGH may comprise a woodchuck hepatitis virus posttranscriptional regulatory element (WPRE), (NCBI Reference Sequence: NC_004107.1). In some embodiments, at bases 4056:4258, pAAV-CBA-SS4-GBA-WPRE-bGH may comprise a polyA signal sequence; Bovine growth hormone gene polyadenylation sequence (GenBank: M57764.1). In some embodiments, at bases 4266:4410, pAAV-CBA-SS4-GBA-WPRE-bGH may comprise a WT AAV2 3′ ITR element in Flip orientation (NCBI Reference Sequence: NC_001401.2).

### Production of AAV Particles

Numerous methods are known in the art for production of rAAV vectors, including transfection, stable cell line production, and infectious hybrid virus production systems which include adenovirus-AAV hybrids, herpesvirus-AAV hybrids (Conway, J E et al., (1997) J. Virology 71(11):8780-8789) and baculovirus-AAV hybrids (Urabe, M. et al., (2002) Human Gene Therapy 13(16):1935-1943; Kotin, R. (2011) Hum Mol Genet. 20(R1): R2-R6). rAAV production cultures for the production of rAAV virus particles all require; 1) suitable host cells, 2) suitable helper virus function, 3) AAV rep and cap genes and gene products; 4) a nucleic acid (such as a therapeutic nucleic acid) flanked by at least one AAV ITR sequences (e.g., an AAV genome encoding a GCase enzyme); and 5) suitable media and media components to support rAAV production. In some embodiments, the suitable host cell is a primate host cell. In some embodiments, the suitable host cell is a human-derived cell lines such as HeLa, A549, 293, or Perc.6 cells. In some embodiments, the suitable helper virus function is provided by wild-type or mutant adenovirus (such as temperature sensitive adenovirus), herpes virus (HSV), baculovirus, or a plasmid construct providing helper functions. In some embodiments, the AAV rep and cap gene products may be from any AAV serotype. In general, but not obligatory, the AAV rep gene product is of the same serotype as the ITRs of the rAAV vector genome as long as the rep gene products may function to replicated and package the rAAV genome. Suitable media known in the art may be used for the production of rAAV vectors. These media include, without limitation, media produced by Hyclone Laboratories and JRH including Modified Eagle Medium (MEM), Dulbecco's Modified Eagle Medium (DMEM), custom formulations such as those described in U.S. Pat. No. 6,566,118, and Sf-900 II SFM media as described in U.S. Pat. No. 6,723,551, each of which is incorporated herein by reference in its entirety, particularly with respect to custom media formulations for use in production of recombinant AAV vectors. In some embodiments, the AAV helper functions are provided by adenovirus or HSV. In some embodiments, the AAV helper functions are provided by baculovirus and the host cell is an insect cell (e.g., Spodoptera frugiperda (Sf9) cells).

One method for producing rAAV particles is the triple transfection method. Briefly, a plasmid containing a rep gene and a capsid gene, along with a helper adenoviral plasmid, may be transfected (e.g., using the calcium phosphate method) into a cell line (e.g., HEK-293 cells), and virus may be collected and optionally purified. As such, in some embodiments, the rAAV particle was produced by triple transfection of a nucleic acid encoding the rAAV vector, a nucleic acid encoding AAV rep and cap, and a nucleic acid encoding AAV helper virus functions into a host cell, wherein the transfection of the nucleic acids to the host cells generates a host cell capable of producing rAAV particles.

In some embodiments, rAAV particles may be produced by a producer cell line method (see Martin et al., (2013) Human Gene Therapy Methods 24:253-269; U.S. PG Pub. No. US2004/0224411; and Liu, X. L. et al. (1999) Gene Ther. 6:293-299). Briefly, a cell line (e.g., a HeLa, 293, A549, or Perc.6 cell line) may be stably transfected with a plasmid containing a rep gene, a capsid gene, and a vector genome comprising a promoter-heterologous nucleic acid sequence (e.g., a GCase enzyme). Cell lines may be screened to select a lead clone for rAAV production, which may then be expanded to a production bioreactor and infected with a helper virus (e.g., an adenovirus or HSV) to initiate rAAV production. Virus may subsequently be harvested, adenovirus may be inactivated (e.g., by heat) and/or removed, and the rAAV particles may be purified. As such, in some embodiments, the rAAV particle was produced by a producer cell line comprising one or more of nucleic acid encoding the rAAV vector, a nucleic acid encoding AAV rep and cap, and a nucleic acid encoding AAV helper virus functions. As described herein, the producer cell line method may be advantageous for the production of rAAV particles with an oversized genome, as compared to the triple transfection method.

In some embodiments, the nucleic acid encoding AAV rep and cap genes and/or the rAAV genome are stably maintained in the producer cell line. In some embodiments, nucleic acid encoding AAV rep and cap genes and/or the rAAV genome is introduced on one or more plasmids into a cell line to generate a producer cell line. In some embodiments, the AAV rep, AAV cap, and rAAV genome are introduced into a cell on the same plasmid. In other embodiments, the AAV rep, AAV cap, and rAAV genome are introduced into a cell on different plasmids. In some embodiments, a cell line stably transfected with a plasmid maintains the plasmid for multiple passages of the cell line (e.g., 5, 10, 20, 30, 40, 50 or more than 50 passages of the cell). For example, the plasmid(s) may replicate as the cell replicates, or the plasmid(s) may integrate into the cell genome. A variety of sequences that enable a plasmid to replicate autonomously in a cell (e.g., a human cell) have been identified (see, e.g., Krysan, P. J. et al. (1989) Mol. Cell Biol. 9:1026-1033). In some embodiments, the plasmid(s) may contain a selectable marker (e.g., an antibiotic resistance marker) that allows for selection of cells maintaining the plasmid. Selectable markers commonly used in mammalian cells include without limitation blasticidin, G418, hygromycin B, zeocin, puromycin, and derivatives thereof. Methods for introducing nucleic acids into a cell are known in the art and include without limitation viral transduction, cationic transfection (e.g., using a cationic polymer such as DEAE-dextran or a cationic lipid such as lipofectamine), calcium phosphate transfection, microinjection, particle bombardment, electroporation, and nanoparticle transfection (for more details, see e.g., Kim, T. K. and Eberwine, J. H. (2010) Anal. Bioanal. Chem. 397:3173-3178).

In some embodiments, the nucleic acid encoding AAV rep and cap genes and/or the rAAV genome are stably integrated into the genome of the producer cell line. In some embodiments, nucleic acid encoding AAV rep and cap genes and/or the rAAV genome is introduced on one or more plasmids into a cell line to generate a producer cell line. In some embodiments, the AAV rep, AAV cap, and rAAV genome are introduced into a cell on the same plasmid. In other embodiments, the AAV rep, AAV cap, and rAAV genome are introduced into a cell on different plasmids. In some embodiments, the plasmid(s) may contain a selectable marker (e.g., an antibiotic resistance marker) that allows for selection of cells maintaining the plasmid. Methods for stable integration of nucleic acids into a variety of host cell lines are known in the art. For example, repeated selection (e.g., through use of a selectable marker) may be used to select for cells that have integrated a nucleic acid containing a selectable marker (and AAV cap and rep genes and/or a rAAV genome). In other embodiments, nucleic acids may be integrated in a site-specific manner into a cell line to generate a producer cell line. Several site-specific recombination systems are known in the art, such as FLP/FRT (see, e.g., O'Gorman, S. et al. (1991) Science 251:1351-1355), Cre/loxP (see, e.g., Sauer, B. and Henderson, N. (1988) Proc. Natl. Acad. Sci. 85:5166-5170), and phi C31-att (see, e.g., Groth, A. C. et al. (2000) Proc. Natl. Acad. Sci. 97:5995-6000).

In some embodiments, the producer cell line is derived from a primate cell line (e.g., a non-human primate cell line, such as a Vero or FRhL-2 cell line). In some embodiments, the cell line is derived from a human cell line. In some embodiments, the producer cell line is derived from HeLa, 293, A549, or PERC.6® (Crucell) cells. For example, prior to introduction and/or stable maintenance/integration of nucleic acid encoding AAV rep and cap genes and/or the oversized rAAV genome into a cell line to generate a producer cell line, the cell line is a HeLa, 293, A549, or PERC.6® (Crucell) cell line, or a derivative thereof.

In some embodiments, the producer cell line is adapted for growth in suspension. As is known in the art, anchorage-dependent cells are typically not able to grow in suspension without a substrate, such as microcarrier beads. Adapting a cell line to grow in suspension may include, for example, growing the cell line in a spinner culture with a stirring paddle, using a culture medium that lacks calcium and magnesium ions to prevent clumping (and optionally an antifoaming agent), using a culture vessel coated with a siliconizing compound, and selecting cells in the culture (rather than in large clumps or on the sides of the vessel) at each passage. For further description, see, e.g., ATCC frequently asked questions document (available at www.atcc.org/Global/FAQs/9/1/Adapting%20a%20monolayer%20cell%20line%20to%20suspension-40.aspx) and references cited therein.

In some aspects, a method is provided for producing any rAAV particle as disclosed herein comprising (a) culturing a host cell under a condition that rAAV particles are produced, wherein the host cell comprises (i) one or more AAV package genes, wherein each said AAV packaging gene encodes an AAV replication and/or encapsidation protein; (ii) a rAAV pro-vector comprising a nucleic acid encoding a heterologous nucleic acid as described herein flanked by at least one AAV ITR, and (iii) an AAV helper function; and (b) recovering the rAAV particles produced by the host cell. In some embodiments, said at least one AAV ITR is selected from the group consisting of AAV1, AAV2, AAV3, AAV4, AAV5, AAV6, AAV7, AAV8, AAVrh8, AAVrh8R, AAV9, AAV10, AAVrh10, AAV11, AAV12, a goat AAV, bovine AAV, or mouse AAV serotype ITRs or the like. For example, in some embodiments, the AAV serotype is AAV1, AAV2, AAV5, AAV6, AAV7, AAV8, AAVrh8, AAVrh8R, AAV9, AAV10, or AAVrh10. In certain embodiments, the nucleic acid in the AAV comprises an AAV2 ITR. In some embodiments, said encapsidation protein is selected from the group consisting of AAV1, AAV2, AAV3, AAV4, AAV5, AAV6, AAV7, AAV8, AAVrh8, AAVrh8R, AAV9, AAV1999. AAV10, AAVrh10, AAV11, AAV12, AAV2R471A, AAV2/2-7m8, AAV DJ, AAV2 N587A, AAV2 E548A, AAV2 N708A, AAV V708K, goat AAV, AAV1/AAV2 chimeric, bovine AAV, mouse AAV capsid, rAAV2/HBoV1 serotype, AAV-XL32, or AAV-XL32.1 capsid proteins or mutants thereof. In some embodiments, the encapsidation protein is an AAV8 capsid protein. In some embodiments, the rAAV particles comprise an AAV9 capsid and a recombinant genome comprising AAV2 ITRs, and nucleic acid encoding a therapeutic transgene/nucleic acid (e.g., an expression cassette for expressing a GCase enzyme). In some embodiments, the rAAV particles comprise a SAN006 capsid and a recombinant genome comprising AAV2 ITRs, and nucleic acid encoding a therapeutic transgene/nucleic acid (e.g., an expression cassette for expressing a GCase enzyme).

Suitable rAAV production culture media of the present disclosure may be supplemented with serum or serum-derived recombinant proteins at a level of 0.5%-20% (v/v or w/v). Alternatively, as is known in the art, rAAV vectors may be produced in serum-free conditions which may also be referred to as media with no animal-derived products. One of ordinary skill in the art may appreciate that commercial or custom media designed to support production of rAAV vectors may also be supplemented with one or more cell culture components know in the art, including without limitation glucose, vitamins, amino acids, and or growth factors, in order to increase the titer of rAAV in production cultures.

rAAV production cultures can be grown under a variety of conditions (over a wide temperature range, for varying lengths of time, and the like) suitable to the particular host cell being utilized. As is known in the art, rAAV production cultures include attachment-dependent cultures which can be cultured in suitable attachment-dependent vessels such as, for example, roller bottles, hollow fiber filters, microcarriers, and packed-bed or fluidized-bed bioreactors. rAAV vector production cultures may also include suspension-adapted host cells such as HeLa, 293, and SF-9 cells which can be cultured in a variety of ways including, for example, spinner flasks, stirred tank bioreactors, and disposable systems such as the Wave bag system.

rAAV vector particles of the disclosure may be harvested from rAAV production cultures by lysis of the host cells of the production culture or by harvest of the spent media from the production culture, provided the cells are cultured under conditions known in the art to cause release of rAAV particles into the media from intact cells, as described more fully in U.S. Pat. No. 6,566,118). Suitable methods of lysing cells are also known in the art and include for example multiple freeze/thaw cycles, sonication, microfluidization, and treatment with chemicals, such as detergents and/or proteases.

In a further embodiment, the rAAV particles are purified. The term “purified” as used herein includes a preparation of rAAV particles devoid of at least some of the other components that may also be present where the rAAV particles naturally occur or are initially prepared from. Thus, for example, isolated rAAV particles may be prepared using a purification technique to enrich it from a source mixture, such as a culture lysate or production culture supernatant. Enrichment can be measured in a variety of ways, such as, for example, by the proportion of DNase-resistant particles (DRPs) or genome copies (gc) present in a solution, or by infectivity, or it can be measured in relation to a second, potentially interfering substance present in the source mixture, such as contaminants, including production culture contaminants or in-process contaminants, including helper virus, media components, and the like.

In some embodiments, the rAAV production culture harvest is clarified to remove host cell debris. In some embodiments, the production culture harvest is clarified by filtration through a series of depth filters including, for example, a grade DOHC Millipore Millistak+HC Pod Filter, a grade A1HC Millipore Millistak+HC Pod Filter, and a 0.2 μm Filter Opticap XL1O Millipore Express SHC Hydrophilic Membrane filter. Clarification can also be achieved by a variety of other standard techniques known in the art, such as, centrifugation or filtration through any cellulose acetate filter of 0.2 μm or greater pore size known in the art.

In some embodiments, the rAAV production culture harvest is further treated with Benzonase® to digest any high molecular weight DNA present in the production culture. In some embodiments, the Benzonase® digestion is performed under standard conditions known in the art including, for example, a final concentration of 1-2.5 units/ml of Benzonase® at a temperature ranging from ambient to 37° C. for a period of 30 minutes to several hours.

rAAV particles may be isolated or purified using one or more of the following purification steps: equilibrium centrifugation; flow-through anionic exchange filtration; tangential flow filtration (TFF) for concentrating the rAAV particles; rAAV capture by apatite chromatography; heat inactivation of helper virus; rAAV capture by hydrophobic interaction chromatography; buffer exchange by size exclusion chromatography (SEC); nanofiltration; and rAAV capture by anionic exchange chromatography, cationic exchange chromatography, or affinity chromatography. These steps may be used alone, in various combinations, or in different orders. In some embodiments, the method comprises all the steps in the order as described below. Methods to purify rAAV particles are found, for example, in Xiao et al., (1998) Journal of Virology 72:2224-2232; U.S. Pat. Nos. 6,989,264 and 8,137,948; and WO 2010/148143.

### Methods of Treatment

Certain aspects of the present disclosure relate to methods of treating GD and/or GBA-PD and/or increasing levels of a GCase enzyme in an individual in need thereof. In some embodiments, the disclosure provides methods of treating GD and/or GBA-PD by administering an effective amount of an expression cassette (e.g., an expression cassette delivered in a rAAV particle) for expressing a GBA1 polypeptide of the present disclosure. In some embodiments, the GBA1 polypeptide is a wild type GBA1 polypeptide. The expression cassette (e.g., expression cassette delivered in a rAAV particle) for expressing a GCase enzyme may be administered through various routes. In some embodiments, the administration includes direct spinal cord injection and/or intracerebral administration. In some embodiments, the administration is at a site selected from the cerebrum, medulla, pons, cerebellum, intracranial cavity, meninges surrounding the brain, dura mater, arachnoid mater, pia mater, cerebrospinal fluid (CSF) of the subarachnoid space surrounding the brain, deep cerebellar nuclei of the cerebellum, ventricular system of the cerebrum, subarachnoid space, striatum, cortex, septum, thalamus, hypothalamus, and the parenchyma of the brain. In some embodiments, the administration comprises intracerebroventricular injection into at least one cerebral lateral ventricle. In some embodiments, the administration comprises intrathecal injection in the cervical, thoracic, and/or lumbar region. In some embodiments, the administration comprises intrastriatal injection. In some embodiments, the administration comprises intrathalamic injection. In some embodiments, the administration comprises parenteral administration such as intravenous administration, subcutaneous administration or intramuscular administration.

An effective amount of rAAV (in some embodiments in the form of particles) is administered, depending on the objectives of treatment. For example, where a low percentage of transduction can achieve the desired therapeutic effect, then the objective of treatment is generally to meet or exceed this level of transduction. In some instances, this level of transduction can be achieved by transduction of only about 1 to 5% of the target cells of the desired tissue type, in some embodiments at least about 20% of the cells of the desired tissue type, in some embodiments at least about 50%, in some embodiments at least about 80%, in some embodiments at least about 95%, in some embodiments at least about 99% of the cells of the desired tissue type. The rAAV composition may be administered by one or more administrations, either during the same procedure or spaced apart by days, weeks, months, or years. One or more of any of the routes of administration described herein may be used. In some embodiments, multiple vectors may be used to treat the human.

In some embodiments of the above aspects, the rAAV is administered via direct injection into the spinal cord, via intrathecal injection, or via intracisternal injection. In some embodiments, the rAAV is administered to more than one location of the spinal cord or cisterna magna. In some embodiments, the rAAV is administered to more than one location of the spinal cord. In some embodiments, the rAAV is administered to one or more of a lumbar subarachnoid space, thoracic subarachnoid space and a cervical subarachnoid space of the spinal cord. In some embodiments, the rAAV is administered to the cisterna magna.

Methods to identify cells transduced by AAV viral particles are known in the art; for example, immunohistochemistry or the use of a marker such as enhanced green fluorescent protein can be used to detect transduction of viral particles; for example viral particles comprising a rAAV capsid with one or more substitutions of amino acids.

In some embodiments, an effective amount of rAAV particles is administered to more than one location simultaneously or sequentially. In other embodiments, an effective amount of rAAV particles is administered to a single location more than once (e.g., repeated). In some embodiments, multiple injections of rAAV viral particles are no more than one hour, two hours, three hours, four hours, five hours, six hours, nine hours, twelve hours or 24 hours apart.

In some embodiments, the disclosure provides a method for treating a human with GD and/or GBA-PD by administering an effective amount of a pharmaceutical composition comprising a recombinant viral vector encoding a GCase enzyme of the present disclosure. In some embodiments, the pharmaceutical composition comprises one or more pharmaceutically acceptable excipients.

In some embodiments, the methods comprise administering an effective amount of a pharmaceutical composition comprising a recombinant viral vector encoding a GCase enzyme of the present disclosure to treat GD and/or GBA-PD in an individual in need thereof. In some embodiments, the viral titer of the viral particles (e.g., rAAV particles) is at least about any of 5×10¹², 6×10¹², 7×10¹², 8×10¹², 9×10¹², 10×10¹², 11×10¹², 15×10¹², 20×10¹², 25×10¹² 30×10¹², or 50×10¹² genome copies/mL. In some embodiments, the viral titer of the viral particles (e.g., rAAV particles) is about any of 5×10¹² to 6×10¹², 6×10¹² to 7×10¹², 7×10¹² to 8×10¹², 8×10¹² to 9×10¹², 9×10¹² to 10×10¹², 10×10¹² to 11×10¹², 11×10¹² to 15×10¹², 15×10¹² to 20×10¹², 20×10¹² to 25×10¹², 25×10¹² to 30×10¹², 30×10¹² to 50×10¹², or 50×10¹² to 100×10¹² genome copies/mL. In some embodiments, the viral titer of the viral particles (e.g., rAAV particles) is about any of 5×10¹² to 10×10¹², 10×10¹² to 25×10¹², or 25×10¹² to 50×10¹² genome copies/mL. In some embodiments, the viral titer of the viral particles (e.g., rAAV particles) is at least about any of 5×10⁹, 6×10⁹, 7×10⁹, 8×10⁹, 9×10⁹, 10×10⁹, 11×10⁹, 15×10⁹, 20×10⁹, 25×10⁹, 30×10⁹, or 50×10⁹ transducing units/mL. In some embodiments, the viral titer of the viral particles (e.g., rAAV particles) is about any of 5×10⁹ to 6×10⁹, 6×10⁹ to 7×10⁹, 7×10⁹ to 8×10⁹, 8×10⁹ to 9×10⁹, 9×10⁹ to 10×10⁹, 10×10⁹ to 11×10⁹, 11×10⁹ to 15×10⁹, 15×10⁹ to 20×10⁹, 20×10⁹ to 25×10⁹, 25×10⁹ to 30×10⁹, 30×10⁹ to 50×10⁹ or 50×10⁹ to 100×10⁹ transducing units/mL. In some embodiments, the viral titer of the viral particles (e.g., rAAV particles) is about any of 5×10⁹ to 10×10⁹, 10×10⁹ to 15×10⁹, 15×10⁹ to 25×10⁹, or 25×10⁹ to 50×10⁹ transducing units/mL. In some embodiments, the viral titer of the viral particles (e.g., rAAV particles) is at least any of about 5×10¹⁰, 6×10¹⁰, 7×10¹⁰, 8×10¹⁰, 9×10¹⁰, 10×10¹⁰, 11×10¹⁰, 15×10¹⁰, 20×10¹⁰, 25×10¹⁰, 30×10¹⁰, 40×10¹⁰, or 50×10¹⁰ infectious units/mL. In some embodiments, the viral titer of the viral particles (e.g., rAAV particles) is at least any of about 5×10¹⁰ to 6×10¹⁰, 6×10¹⁰ to 7×10¹⁰, 7×10¹⁰ to 8×10¹⁰, 8×10¹⁰ to 9×10¹⁰, 9×10¹⁰ to 10×10¹⁰, 10×10¹⁰ to 11×10¹⁰, 11×10¹⁰ to 15×10¹⁰, 15×10¹⁰ to 20×10¹⁰, 20×10¹⁰ to 25×10¹⁰, 25×10¹⁰ to 30×10¹⁰, 30×10¹⁰ to 40×10¹⁰, 40×10¹⁰ to 50×10¹⁰, or 50×10¹⁰ to 100×10¹⁰ infectious units/mL. In some embodiments, the viral titer of the viral particles (e.g., rAAV particles) is at least any of about 5×10¹⁰ to 10×10¹⁰, 10×10¹⁰ to 15×10¹⁰, 15×10¹⁰ to 25×10¹⁰, or 25×10¹⁰ to 50×10¹⁰ infectious units/mL. In some embodiments, the viral particles are rAAV particles. In some embodiments, the rAAV particles comprise a SAN006 capsid protein.

In some embodiments, the viral titer of the viral particles (e.g., rAAV particles) is at least about any of 5×10¹², 6×10¹², 7×10¹², 8×10¹², 9×10¹², 10×10¹², 11×10¹², 15×10¹², 20×10¹², 25×10¹², 30×10¹², 50×10¹², 80×10¹², 90×10¹², 100×10¹², 110×10¹², 120×10¹² 130×10¹², 140×10¹², 150×10¹², 175×10¹², 200×10¹², 300×10¹², 400×10¹², 500×10¹², 600×10¹², 700×10¹², 800×10¹², 900×10¹², or at least 100×10¹² genome copies/mL. In some embodiments, the viral titer of the viral particles (e.g., rAAV particles) is at least about any of 100×10¹², 200×10¹², 300×10¹², or 400×10¹² genome copies/mL. In some embodiments, the viral particles are rAAV particles. In some embodiments, the rAAV particles comprise a SAN006 capsid protein.

In some embodiments, the dose of viral particles administered to the individual is at least about any of 1×10⁸ to about 6×10¹³ genome copies/kg of body weight. In some embodiments, the dose of viral particles administered to the individual is about any of 1×10⁸ to about 6×10¹³ genome copies/kg of body weight. In some embodiments, the dose of viral particles administered to the individual is about any of 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 3×10¹¹, 4×10¹¹, 5×10¹¹, 6×10¹¹, 7×10¹¹, 8×10¹¹, 9×10¹¹, 1×10¹², 2×10¹², 13×10¹², 4×10¹², 5×10¹², 6×10¹², 7×10¹², 8×10¹², 9×10¹², or 1×10¹³ genome copies/kg of body weight.

In some embodiments, the total amount of viral particles administered to the individual is at least about any of 1×10⁹ to about 1×10¹⁴ genome copies. In some embodiments, the total amount of viral particles administered to the individual is about any of 1×10⁹ to about 1×10¹⁴ genome copies. In some embodiments, the total amount of viral particles administered to the individual is about any of 1×10¹¹, 2×10¹¹, 3×10¹¹, 4×10¹¹, 5×10¹¹, 6×10¹¹, 7×10¹¹, 8×10¹¹, 9×10¹¹, 1×10¹², 2×10¹², 3×10¹², 4×10¹², 5×10¹², 6×10¹², 7×10¹², 8×10¹², 9×10¹², 1×10¹³, 2×10¹³, 13×10¹³, 4×10¹³, 5×10¹³, 6×10¹³, 7×10¹³, 8×10¹³, 9×10, or 1×10¹⁴ genome copies.

Compositions of the disclosure (e.g., recombinant viral particles comprising a vector encoding a GCase enzyme of the present disclosure) can be used either alone or in combination with one or more additional therapeutic agents for treating GD and/or GBA-PD. The interval between sequential administration can be in terms of at least (or, alternatively, less than) minutes, hours, or days.

An effective amount of rAAV (in some embodiments in the form of particles) is administered, depending on the objectives of treatment. For example, where a low percentage of transduction can achieve the desired therapeutic effect, then the objective of treatment is generally to meet or exceed this level of transduction. In some instances, this level of transduction can be achieved by transduction of only about 1 to 5% of the target cells, in some embodiments at least about 20% of the cells of the desired tissue type, in some embodiments at least about 50%, in some embodiments at least about 80%, in some embodiments at least about 95%, in some embodiments at least about 99% of the cells of the desired tissue type. The rAAV composition may be administered by one or more administrations, either during the same procedure or spaced apart by days, weeks, months, or years. In some embodiments, multiple vectors may be used to treat the mammal (e.g., a human).

In some embodiments, a rAAV composition of the present disclosure may be used for administration to a human. In some embodiments, a rAAV composition of the present disclosure may be used for pediatric administration. In some embodiments, an effective amount of rAAV (in some embodiments in the form of particles) is administered to a patient that is less than one month, less than two months, less than three months, less than four months, less than five months, less than six months, less than seven months, less than eight months, less than nine months, less than ten months, less than eleven months, less than one year, less than 13 months, less than 14 months, less than 15 months, less than 16 months, less than 17 months, less than 18 months, less than 19 months, less than 20 months, less than 21 months, less than 22 months, less than two years, less than three years old, less than five years old or less than seven years old.

In some embodiments, a rAAV composition of the present disclosure may be used for administration to a young adult. In some embodiments, an effective amount of rAAV (in some embodiments in the form of particles) is administered to a patient that is less than 12 years old, less than 13 years old, less than 14 years old, less than 15 years old, less than 16 years old, less than 17 years old, less than 18 years old, less than 19 years old, less than 20 years old, less than 21 years old, less than 22 years old, less than 23 years old, less than 24 years old, or less than 25 years old.

In some aspects, the rAAV viral particle is used to treat GD type 3 or GD type 2. In some embodiments, the rAAV viral particle can be administered to the cerebrospinal fluid (CSF) of the GD type 3 patient or the GD type 2 patient. In some embodiments, the viral particle is administered directly by intra-CSF administration of the patient with GD type 3 or GD type 2. In some embodiments of the above aspects, the rAAV is administered via direct injection into the spinal cord, via intrathecal injection, or via intracisternal injection of the patient with GD type 3 or GD type 2. In some embodiments, the rAAV is administered to more than one location of the spinal cord or cisterna magna of the patient with GD type 3 or GD type 2. In some embodiments, the rAAV is administered to more than one location of the spinal cord of the patient with GD type 3 or GD type 2. In some embodiments, the rAAV is administered to one or more of a lumbar subarachnoid space, thoracic subarachnoid space and a cervical subarachnoid space of the spinal cord of the patient with GD type 3 or GD type 2. In some embodiments, the rAAV is administered to the cisterna magna of the patient with GD type 3 or GD type 2. In some embodiments, the method may comprise treating GD type 3 in a patient in need thereof. In other embodiments, the method may comprise treating GD type 2 in a patient in need thereof. In particular embodiments, the expression cassette of the viral particle is able to drive transgene expression in the central and peripheral nervous systems to treat GD type 3 or GD type 2. In some embodiments, administration of the rAAV particles ameliorates symptoms associated with GD2 or GD3. For instance, administration of the viral particle may reduce or impede the progression of brainstem and cortical dysfunction, seizures and cognitive defects. In some embodiments, administration of the viral particle reduces or clears accumulated toxic lipid substrates (e.g., Lyso-GL1) in the brain of the GD type 3 or GD type 2 patient.

In other aspects, the rAAV viral particle is used to treat GD type 1. In certain embodiments, the rAAV viral particle can be administered parenterally (e.g., intravenously, subcutaneously, or intramuscularly) to the GD type 1 patient. In some embodiments, the rAAV viral particle can be administered intravenously to the GD type 1 patient. In some embodiments, administration of the rAAV viral particle reduces splenomegaly in the GD type 1 patient. In some embodiments, administration of the rAAV viral particle reduces hepatomegaly in the GD type 1 patient. In some embodiments, administration of the rAAV viral particle reduces anemia in the GD type 1 patient. In some embodiments, administration of the rAAV viral particle reduces thrombocytopenia in the GD type 1 patient. In some embodiments, administration of the viral particle reduces or clears accumulated toxic lipid substrates (e.g., Lyso-GL1) in peripheral organs such as the liver, spleen, kidney and/or lungs. In some embodiments, administration of the viral particle reduces or clears accumulated toxic lipid substrates (e.g., Lyso-GL1) in muscle tissues such as the heart, diaphragm, quadriceps and gastrocnemius.

In other aspects, the rAAV viral particle is used to treat GBA-PD. In some embodiments, the rAAV viral particle can be administered to the cerebrospinal fluid (CSF) of the GBA-PD patient. In some embodiments, the viral particle is administered directly by intra-CSF administration of the patient with GBA-PD. In some embodiments of the above aspects, the rAAV is administered via direct injection into the spinal cord, via intrathecal injection, or via intracisternal injection of the patient with GBA-PD. In some embodiments, the rAAV is administered to more than one location of the spinal cord or cisterna magna of the patient with GBA-PD. In some embodiments, the rAAV is administered to more than one location of the spinal cord of the patient with GBA-PD. In some embodiments, the rAAV is administered to one or more of a lumbar subarachnoid space, thoracic subarachnoid space, and a cervical subarachnoid space of the spinal cord of the patient with GBA-PD. In some embodiments, the rAAV is administered to the cisterna magna of the patient with GBA-PD. In some embodiments, the rAAV viral particle is administered to a patient with early stages of PD. In some such embodiments, administration of the viral particle reduces the cognitive decline and disease progression of the PD. In some embodiments, administration of the viral particle reduces or clears accumulated toxic lipid substrates (e.g., Lyso-GL1) in the brain of the GBA-PD patient. In some embodiments, administration of the viral particle reduces α-synuclein in the brain of the GBA-PD patient.

### Kits or Articles of Manufacture

The expression cassettes (e.g., an expression cassette for expressing a GCase enzyme, such as a wild type human GCase enzyme or a GCase enzyme with a mutated signaling peptide), rAAV vectors, particles, and/or pharmaceutical compositions as described herein may be contained within a kit or article of manufacture, e.g., designed for use in one of the methods of the disclosure as described herein.

Generally, the system comprises a cannula, one or more syringes (e.g., 1, 2, 3, 4 or more), and one or more fluids (e.g., 1, 2, 3, 4 or more) suitable for use in the methods of the disclosure.

The syringe may be any suitable syringe, provided it is capable of being connected to the cannula for delivery of a fluid. In some embodiments, the system has one syringe. In some embodiments, the system has two syringes. In some embodiments, the system has three syringes. In some embodiments, the system has four or more syringes. The fluids suitable for use in the methods of the disclosure include those described herein, for example, one or more fluids each comprising an effective amount of one or more vectors as described herein, and one or more fluids comprising one or more therapeutic agents.

In some embodiments, the kit comprises a single fluid (e.g., a pharmaceutically acceptable fluid comprising an effective amount of the vector). In some embodiments, the kit comprises 2 fluids. In some embodiments, the kit comprises 3 fluids. In some embodiments, the kit comprises 4 or more fluids. A fluid may include a diluent, buffer, excipient, or any other liquid described herein or known in the art suitable for delivering, diluting, stabilizing, buffering, or otherwise transporting an expression cassette for expressing a GCase enzyme or rAAV vector composition of the present disclosure. In some embodiments, the kit comprises one or more buffers, e.g., an aqueous pH buffered solution. Examples of buffers may include without limitation phosphate, citrate, Tris, HEPES, and other organic acid buffers.

In some embodiments, the kit comprises a container. Suitable containers may include, e.g., vials, bags, syringes, and bottles. The container may be made of one or more of a material such as glass, metal, or plastic. In some embodiments, the container is used to hold a rAAV composition of the present disclosure. In some embodiments, the container may also hold a fluid and/or other therapeutic agent.

In some embodiments, the kit comprises an additional therapeutic agent with a rAAV composition of the present disclosure. In some embodiments, the rAAV composition and the additional therapeutic agent may be mixed. In some embodiments, the rAAV composition and the additional therapeutic agent may be kept separate. In some embodiments, the rAAV composition and the additional therapeutic agent may be in the same container. In some embodiments, the rAAV composition and the additional therapeutic agent may be in different containers. In some embodiments, the rAAV composition and the additional therapeutic agent may be administered simultaneously. In some embodiments, the rAAV composition and the additional therapeutic agent may be administered on the same day. In some embodiments, the rAAV composition may be administered within one day, two days, three days, four days, five days, six days, seven days, two weeks, three weeks, four weeks, two months, three months, four months, five months, or six months of administration of the additional therapeutic agent.

In some embodiments, the kit comprises a therapeutic agent to transiently suppress the immune system prior to AAV administration. In some embodiments, patients are transiently immune suppressed shortly before and after injection of the virus to inhibit the T cell response to the AAV particles (e.g., see Ferreira et al., Hum. Gene Ther. 25:180-188, 2014). In some embodiments, the kit further provides cyclosporine, mycophenolate mofetil, and/or methylprednisolone.

The rAAV particles and/or compositions of the disclosure may further be packaged into kits including instructions for use. In some embodiments, the kits further comprise a device for delivery (e.g., any type of parenteral administration described herein) of compositions of rAAV particles. In some embodiments, the instructions for use include instructions according to one of the methods described herein. In some embodiments, the instructions are printed on a label provided with (e.g., affixed to) a container. In some embodiments, the instructions for use include instructions for administering to an individual (e.g., a human) an effective amount of rAAV particles, e.g., for treating GBA-PD AND/OR GD in an individual.

### EXAMPLES

The disclosure will be more fully understood by reference to the following examples. They should not, however be construed as limiting the scope of the disclosure. It is understood that the examples and embodiments described herein are for illustrative purposes only and that various modification or changes in light thereof will be suggested to persons skilled in the art and are to be included within the spirit and purview of this application and scope of the appended embodiments.

### General Methods

### Administration of Conduritol β Epoxide (CBE)

CBE (Sigma-Aldrich, 234599) was reconstituted with sterile saline to a concentration of 10 mg/mL just prior to use. For rodents, mice were injected with 100 mg/kg of CBE at 10 mL/kg by intraperitoneal (IP) injection using a 271/2 gauge needle 24 hours prior to necropsy. For non-human primates (NHPs), single dose of CBE was given to 2-4 years old cynomolgus monkeys by intravenous (bolus) infusion to the saphenous vein at 0.5 mL/minutes rate 24-48 hours prior to necropsy.

### Bilateral Intracerebroventricular (ICV) Injection

4-month-old C57BL/6 or mice were anesthetized with isoflurane and received bilateral injections into the lateral ventricles (A-P: −0.40; M-L: ±1.00; D-V: −2.70) with 4-5 μL per site of formulation buffer or AAV.SAN006-GBA1 and variants using a 10-μL Hamilton syringe at a rate of 1 L/min. Following the procedure, the injected mice were monitored, and supportive care was given for recovery.

### Intra-Cisterna Magna (ICM) Injection

Cynomolgus monkeys were fasted overnight (at least 8 hours) prior to dosing procedure. The animals were anesthetized and positioned in lateral Trendelenburg position with an angle of approximately 15-20 degrees. The dosing setup was primed with formulation buffer prior to dosing and fluoroscopy imaging was used to guide the surgeon during the dosing. As a single step, a volume of 2.5 mL of either formulation buffer or AAV.SAN006-GBA1 at 1.25e13 VGs per virus was administered at a rate of 0.125 mL/minute. A flush volume of 0.250 mL was given at the end of the dosing and the needle was left in place for at least 1-3 minutes before removal. Following the procedure, veterinary care was given for recovery.

### Intravenous (IV) Injection

For rodent IV studies, 3-month-old C57B BL/6J mice were injected with 100 μl of formulation buffer or AAV.SAN006 WT GBA1 or AAV.SAN006-SS3-GBA1 viruses to the lateral tail vein using a 30 gauge needle. For NHPs, 2-3 years old cynomolgus monkeys were injected with AAV.SAN006-GFP at 2.5e13 VG/kg by intravenous (bolus) infusion to the saphenous vein at 0.5 mL/minutes rate.

### Tissue Homogenization

NHP and rodent tissues were homogenized in ice cold TE buffer, pH 7.4 (Fisher scientific, BP2476500) either in 1.4 mm ceramic bead tubes (Fisher scientific, 15-340-153) or 2.8 mm ceramic bead tubes (Fisher scientific, 15-340-154) with 6.5 mm ceramic beads (OMNI International, 19-682) using Omni Bead Ruptor 12 (OMNI International). Following homogenization, tissue homogenate aliquots were made for all the subsequent assays.

### Protein Extraction

TE buffer, pH 7.4 (Fisher scientific, BP2476500) supplemented with Nonidet P-40 (Thermo scientific, 28324) and halt protease inhibitor cocktail (Thermo scientific, 87786) was added to collected tissue homogenates to 0.1% NP-40 final concentration. The homogenates were allowed to mix/solubilize at 4° C. for 15 minutes on a tube revolver rotator (Thermo scientific, 88881001) and followed by centrifugation at 4° C. and 18,000×g for 10 minutes. Clear supernatants were collected and transferred into 1.5 mL eppendorf tubes (Eppendorf, 22363204).

### Protein Quantification

Total protein concentration was determined by BCA (bicinchoninic acid) protein assay kit (Thermo Scientific, 23225). Colorimetric detection was done by measuring absorbance at 562 nm with Molecular Devices SpectraMax 340PC 384 96-well microtiter plate reader and SoftMax Pro version 5.4.4 software.

### Glucosylceramidase Activity Assay

Protein lysate from rodent and NHPs were used to determine GCase activity with glucosylceramidase activity assay kit (abcam, ab273339). Fluorescence excitation at 360 nm and emission at 445 nm was measured via Molecular Devices SpectraMax M2e 96-well microtiter plate reader and SoftMax Pro version 7 software.

### Enzyme-Linked Immunosorbent Assay (ELISA)

Human Glucosylceramidase ELISA: 96 well EIA/RIA Assay microplates (Corning, 9018) were either coated with 2.5 μg/mL GBA recombinant rabbit monoclonal Ab (Invitrogen, MA5-38382) or 1.0 μg/mL mouse GBA monoclonal Ab (Novus biologicals, NBP2-45829) in carbonate buffer (Invitrogen, CB01100) overnight at 4° C. Wells were washed 3× in wash buffer (Invitrogen, WB01) and blocked in assay buffer (Invitrogen, DS98200) overnight at 4° C. Standard curve was generated using recombinant human GBA protein (R&D Systems, 7410-GHB-020) for quantification of human GBA protein. Standards and samples were run in duplicate and incubated for 2 hours at room temperature, washed 3× in wash buffer, and then incubated with 2.5 μg/mL recombinant biotin anti-GBA antibody (abcam, ab201496) for 2 hours at room temperature. Wells were washed 3× in wash buffer, incubated with 0.1 μg/mL streptavidin HRP conjugated (Thermo Scientific, 21126) for 1 hour at room temperature. Subsequently, wells were washed 3× in wash buffer and incubated with TMB substrate (Invitrogen, EB02) for 30 minutes before adding stop solution (Invitrogen, SS04). Human GBA protein was quantified by absorbance at 450 nm with wavelength correction set to 540 nm using Molecular Device SpectraMax and Softmax Pro version 7.1.2 software.

### GFP ELISA

GFP ELISA kit (abcam, ab171581) was used to detect the level of GFP protein. Measurement of GFP protein was completed by reading absorbance at 450 nm with Molecular Devices SpectraMax 340PC 384 and Softmax Pro version 5.4.4 software.

### Lipid Extraction

To quantify GlcSph and/or GlcCer, 20 μl of tissue homogenate (100 mg/mL tissue weight) was aliquoted into a labeled 1.5 ml Eppendorf tube followed by 180 μl of internal standard solution (10 ng/ml d5-GlcSph and 20 ng/mL d35-C18GalCer in 30% methanol, 70% acetonitrile with 5 mM ammonium acetate, and 1% acetic acid).

The samples were vortexed for 10 min and sonicated for 10 min. The tubes were sitting at 4 C for 10 min and centrifuged at 13,000×g for 10 min. The supernatant (150 uL) from each tube was transferred into a pre-labeled total recovery MS vial for MS analysis. Calibration curves for GlcSph and GlcCer were prepared in a pooled matrix, and concentrations were ranged from 0.03 to 1000 ng/ml.

### Lipid Mass Spectrometry

Rodent and NHP tissue homogenates were lipid extracted and the lipids were injected (5 l) into an LC/MS/MS system comprised of an Acquity UPLC (Waters, Milford, MA) and Sciex Triple Quad 5000 mass spectrometer (Sciex, Toronto, Canada).

For GlcSph, the chromatographic separation (from GalSph) was achieved with a Waters Acquity BEH HILIC (2.1×100 mm, 1.7 μm particles, Part #186003461) using mobile phases: (A) 96% ACN, 2% MeOH, 1% Acetic acid, 1% H2O, 5 mM Ammonium acetate and (B) 98% MeOH, 1% Acetic acid, 1% H2O, 5 mM Ammonium acetate.

The column was maintained at 30° C. GlcSph was eluted with the following gradient: from 5% B to 50% B over 3 min, then the mobile phase composition was hold constant for 0.5 min followed by a rapid return (0.1 min) to 5% B maintained for 1 min. All experiments were carried out at a flow rate of 0.5 ml/min. Data were analyzed in Analyst (AB Sciex, Toronto, Canada).

For GlcCer, the chromatographic separation (from GalCer) was achieved with a Waters Cortecs HILIC (2.1×100 mm 2.7 μm particles cat #186007427) using mobile phases: (A) 96% ACN, 2% MeOH, 1% Acetic acid, 1% H2O, 5 mM Ammonium acetate and (B) 80% MeOH, 1% Acetic acid, 20% H2O, 5 mM Ammonium acetate.

The column was maintained at 20° C. GlcCer was eluted at an isobaric flow of 2% B for 4.5 min. All experiments were carried out at a flow rate of 0.5 ml/min. Data were analyzed in Analyst (AB Sciex, Toronto, Canada).

### In Situ Hybridization (ISH)

RNAScope In Situ Assay for WPRE was performed using RNAScope 2.0 Brown detection kit (ACD, 320497). Rodent and NHP brain FFPE slides were pre-treated in EDTA Buffer (pH 9.0) at 90° C. followed by protease treatment at 40° C. The RNASCope WPRE ISH (ACD, 410058) assay was carried out in 40° C. oven for hybridization and amplification from Amp 1 to Amp 4, followed by Amp 5 and Amp 6 at room temperature. The WPRE ISH signal was developed using DAB producing brown color.

### Immunohistochemistry (IHC)

GBA IHC: Rodent and NHP brain FFPE slides were treated in EDTA solution (pH.9.0) for antigen retrieval and subsequently blocked with 3% hydrogen peroxide, followed with 5% horse serum. The slides were then incubated with GBA1 antibodies (abcam, ab125065 and Novus, NBP2-45829) 1:100 and 1:400 dilution, respectively. The color development for GBA signal was achieved by DAB resulting brown color staining and the slides were counter stained with hematoxylin for nuclei staining.

### Vector Genome Assessment

gDNA was isolated from rodent or NHP tissue homogenates using QIAamp 96 DNA QIAcube HT kit (QIAGEN, 51331) according to manufacturer's protocol. Briefly, tissue homogenates were treated with proteinase K at 56° C. for overnight and transferred to S block (QIAGEN, 19585). The samples were placed into QIAGEN QIAcube HT instrument and gDNA isolation was performed by following the steps from QIAcube HT Prep Mange Software. After gDNA isolation, the concentration was measured with NANODROP 8000 (Thermo Fisher Scientific) and vector genome was determined by dPCR with QIAGEN QIAcuty instrument. The gDNA samples from rodent and NHP tissues were analyzed using primer-probe combination specific to the bGH, transgene, and housekeeping gene sequences (Integrated DNA Technology) to determine the vector copy number.

### Transcript Assessment

RNA was isolated from rodent or NHP tissue homogenates using RNeasy 96 QIAcube HT kit (QIAGEN, 74171) according to manufacturer's protocol. Briefly, tissue homogenates were mixed with QIAzol lysis reagent (QIAGEN, 79306) and followed by chloroform (Fisher Scientific, C298-1). The mixture was centrifuged at 4° C. and the aqueous phase was transferred to S block (QIAGEN, 19585) for further RNA isolation. The samples were placed into QIAcube HT instrument and RNA isolation was performed by following the steps from QIAcube HT Prep Mange Software with in-column DNase (QIAGEN, 79256) treatment. After RNA isolation, the concentration was measured with NANODROP 8000 (Thermo Fisher Scientific). RT-dPCR was carried out by producing cDNA via QIAcuity OneStep advanced probe kit (QIAGEN, 250132) and using primer-probe combination specific to the transgene, GFP, and housekeeping gene sequences (Integrated DNA Technology) to determine the total transcripts per RNA input.

### Chromogenic In Situ Hybridization (ISH)

RNAScope In Situ Assay for WPRE was performed using RNAScope 2.0 Brown detection kit (ACD, 320497). Rodent and NHP brain FFPE slides were pre-treated in EDTA Buffer (pH 9.0) at 90° C. followed by protease treatment at 40° C. The RNASCope WPRE ISH (ACD, 410058) assay was carried out in 40° C. oven for hybridization and amplification from Amp 1 to Amp 4, followed by Amp 5 and Amp 6 at room temperature. The WPRE ISH signal was developed using DAB producing brown color.

### Chromogenic Immunohistochemistry (IHC)

Rodent and NHP brain FFPE slides were treated in EDTA solution (pH 9.0) for antigen retrieval and subsequently blocked with 3% hydrogen peroxide, followed with 5% horse serum. The slides were then incubated with human GBA1 antibodies (abcam and Novus) 1:100 and 1:400 dilution, respectively. The color development for GBA signal was achieved by DAB resulting brown color staining and the slides were counter stained with hematoxylin for nuclei staining.

### Immunofluorescent RNA/Protein Integrated Co-Detection Assay

Co-detection of WPRE RNA with payload human GBA protein and different cell type markers from NHP brain FFPE slides were performed using Leica BOND RX automated stainer (Leica Biosystems). Multiplexing protocol was programmed for streamline In Situ Hybridization and a single continuous IHC staining for co-detection of RNA and protein markers. RNAscope 2.5 LS multiplex fluorescent kit (ACD, 322800) and Bond polymer refine detection kit (Leica Biosystems, DS9800) were used in this new programmed protocol. WPRE probe (ACD, 410058) was used in In Situ Hybridization step followed by detection and visualization using TSA Vivid Fluorophore 570 (biotechne). In sequential IHC steps, GBA1 antibodies (Novus) 1:6000 and cell type markers were used for detection of human GBA and cell type markers. DAPI signal was used as nuclear counterstaining.

### Statistics

All statistical analyses were performed with GraphPad Prism 9 software (GraphPad, San Diego, CA, USA) using either one-way or two-way ANOVA with Tukey's multiple comparisons test or with Student's t-test depending on the data set.

### Example 1. Payload Engineering Strategy to Generate GBA1 Variants that are Readily Secretable

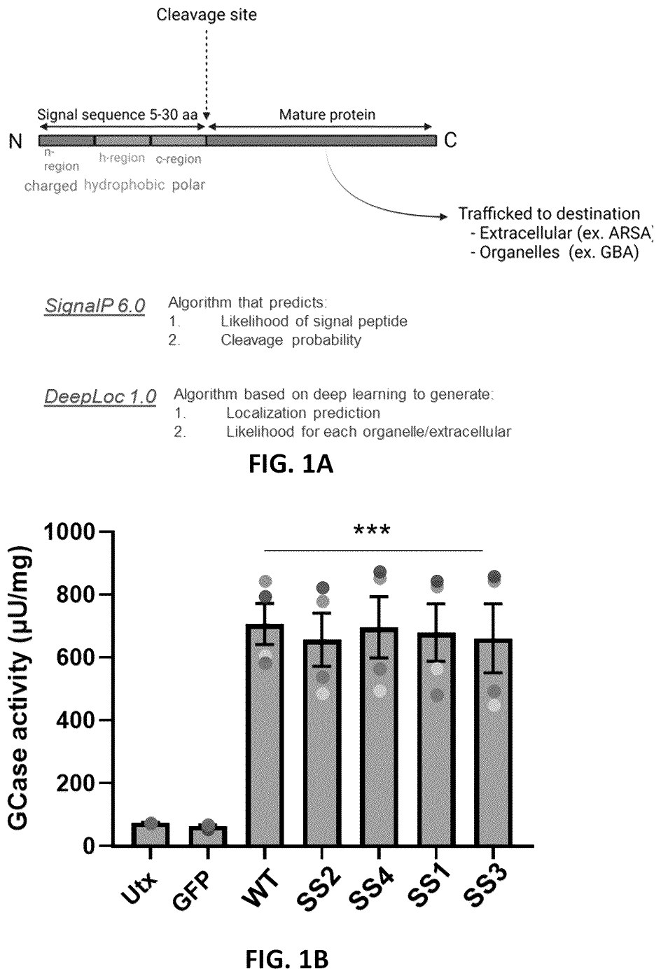

Human GBA1 (NP_000148.2) was engineered to make it readily secretable. To do this, the endogenous signal sequence at the N-terminus of GBA1 protein was replaced with signal sequences of some highly secreted proteins. Using a combination of in silico tools, 4 such signal sequences (SS1 through SS4) were identified that yielded the highest probability of being secreted extracellularly and also had the highest probability (>98%) of the signal sequence being cleaved (FIG. 1A). All sequences were human codon-optimized and driven by the hybrid CMV-chicken β-actin (CBA) constitutive promoter. Transgene expression was further enhanced by the addition of the woodchuck hepatitis virus post-transcriptional regulatory element (WPRE). Detailed vector maps of all constructs are provided.

To ensure GCase activity is not compromised with the swapped signal sequences, HEK293 cells were transfected with the different GBA1 constructs and examined GCase activity in cell lysates 48-hour post transfections. Significantly increased GCase enzyme activity was observed in WT GBA1 transfected HEK cells compared to untransfected cells. Further, all the GBA1 variants that were generated had enzyme activity comparable to that of WT GBA1. No statistical differences were observed within the different GBA1 constructs (FIG. 1B).

### Example 2: Robust Secretion of Some GBA1 Variants In Vivo in Rodent Brain Tissues

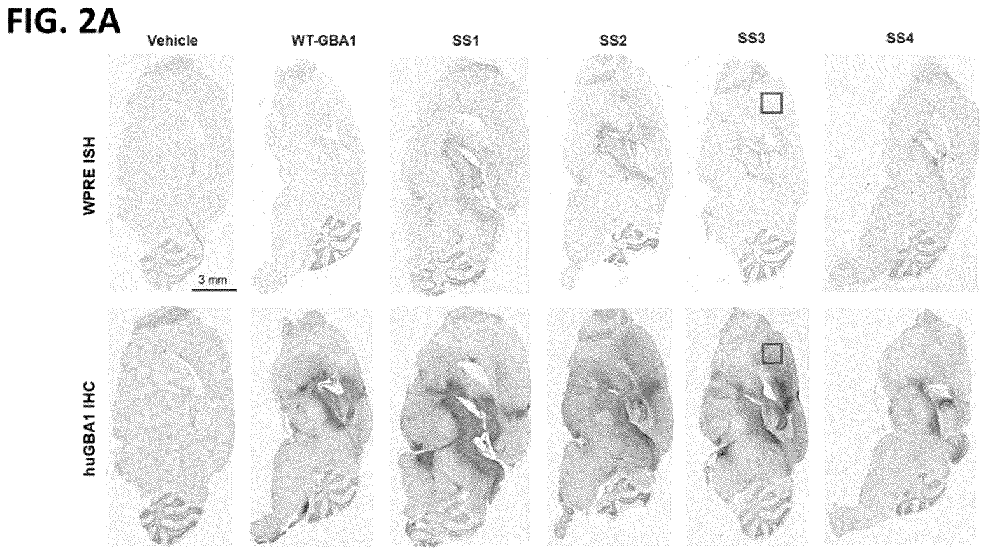

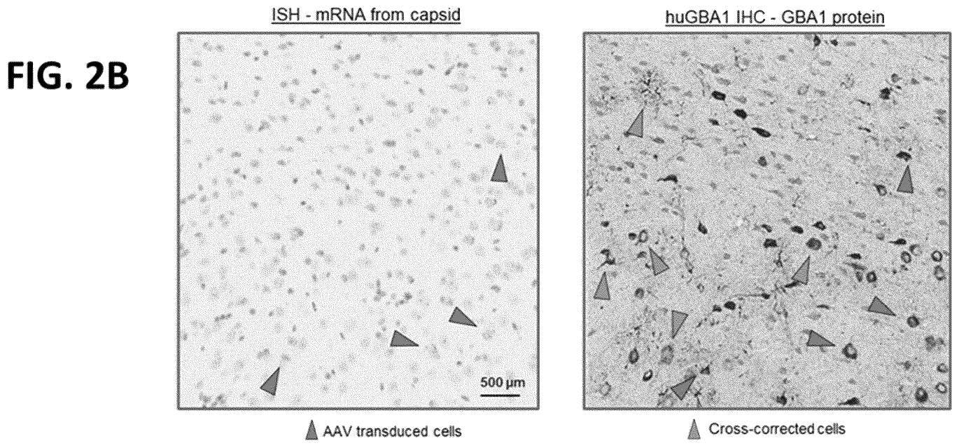

To investigate the ability of engineered constructs to secrete and be taken up by non-AAV transduced cells in vivo, bilateral ICV (intra-cerebroventricular) injections of AAVs (1e11 VG/mouse, 5 ul per hemisphere) were performed in 4-month-old WT mice and analyzed brain sections after 4 weeks of AAV expression. In situ hybridization against the 3′ UTR of the transgene (WPRE) were performed to evaluate distribution of AAV-transduced cells and in adjacent fixed sections, performed immunohistochemistry for human GBA1 protein (FIG. 2A). While the virus was largely localized to regions around the ventricles in the sagittal sections of all AAV treatment groups, a much larger spread of the huGBA1 protein in SS1, SS2, SS3, was observed compared to the WT GBA1. Higher magnification imaging of these sections revealed that not only was there robust secretion of the engineered GBA1, in this case SS3-GBA1 (FIG. 2B), but also efficient uptake of the secreted protein was observed in a variety of cell types in the brain as seen in the morphological differences of cells taking up the secreted protein (marked by green arrows).

### Example 3: Efficient Substrate Clearance by SS3-GBA1 Variant In Vivo in Rodent Brain Tissues

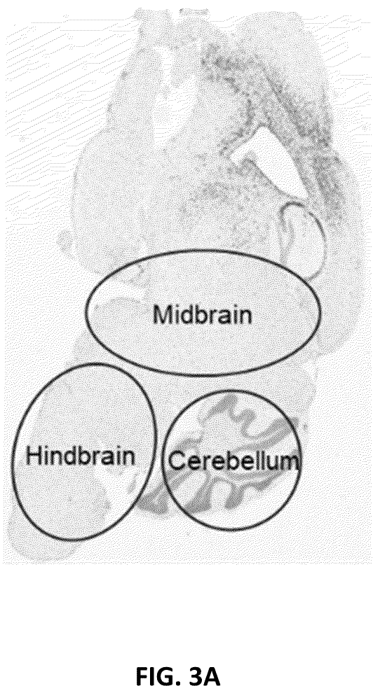

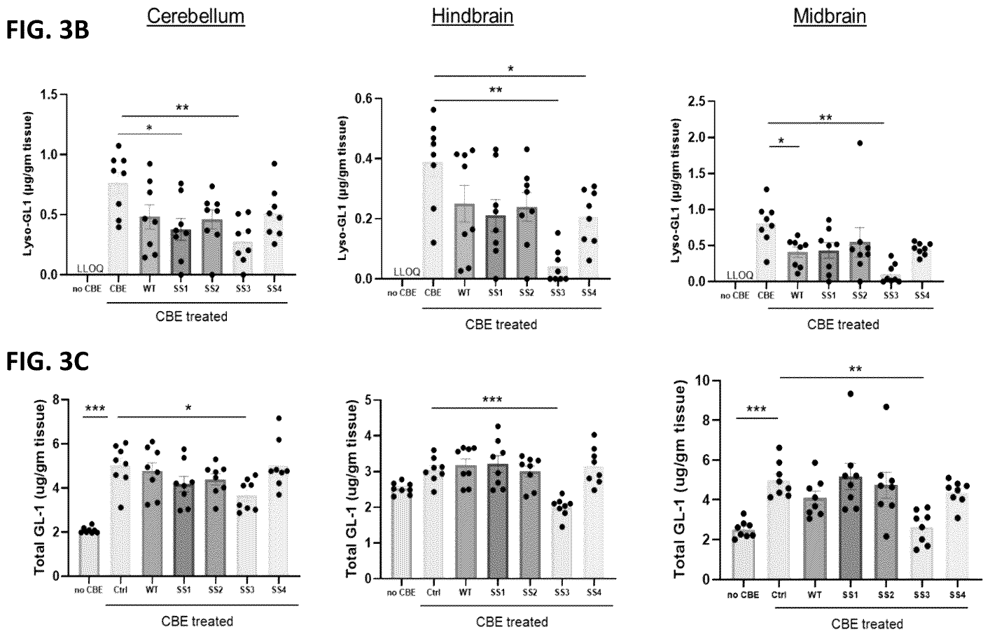

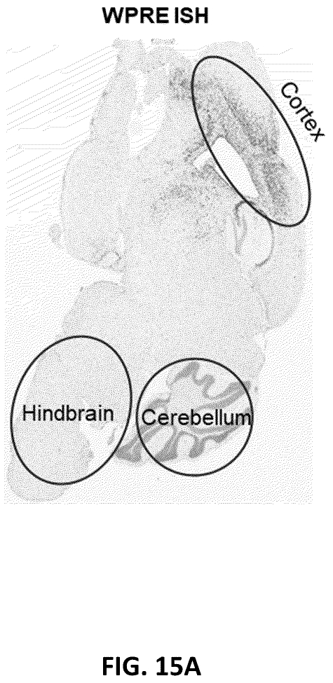

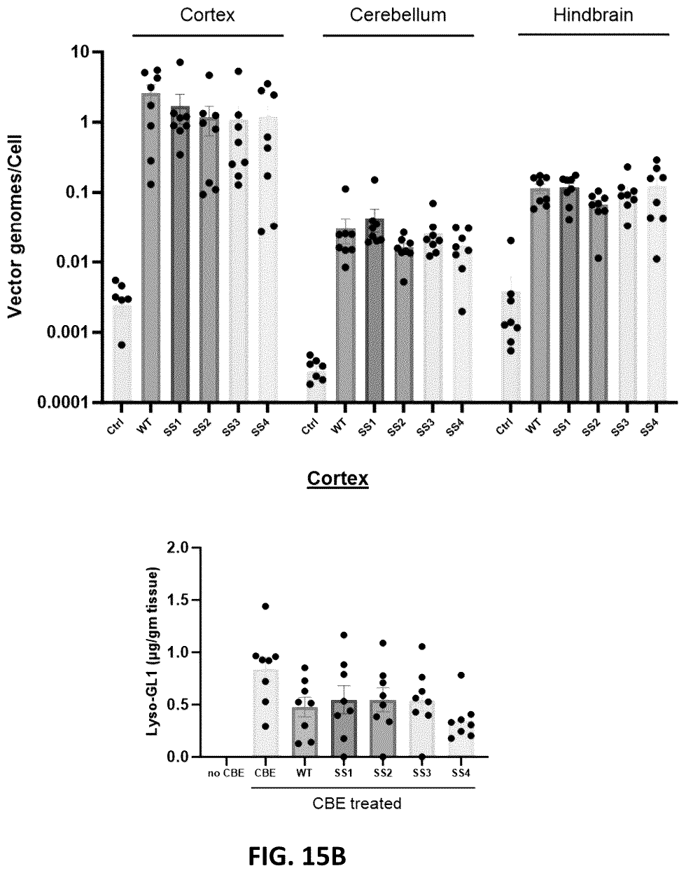

To demonstrate efficacy of GBA1 variants, the AAVs (1e11 VGs) were injected into WT mice for 4 week of expression time and induced lipid accumulation by injecting them with 100 mg/kg CBE (conduritol β-epoxide, IP injection) 24 hours prior to necropsy. The brain was micro-dissected into smaller regions (FIG. 3A and FIG. 15A). Lyso-GL1 and total GL1 levels were assessed via lipidomics mass spectrometry and vector genome distribution was also assessed via dPCR. Under physiological conditions, Lyso-GL1 levels in WT mice (no CBE group) were undetectable or below the LLOQ (lower limit of quantification) and total GL1 levels were 2-3 μg/gm tissue weight, depending on the specific brain region (grey bars in FIG. 3C). Across all regions surveyed, a significant increase in lipid levels (both Lyso-GL1 and GL1) was observed in the CBE treated, vehicle injected group demonstrating the action of CBE to induce lipid accumulation by inhibiting GCase. Surveying both Lyso-GL1 (FIG. 3B) and total GL1 (FIG. 3C) in the cerebellum, hindbrain, and midbrain, SS3-GBA1 effectively reducing lipid accumulation was observed. This construct outperformed all the other constructs generated, and in a few mice, the increased Lyso-GL1 with CBE reverted back to LLOQ as seen with datapoints plotted as 0 in the hindbrain and midbrain tissues.

High levels of AAV genomes proximal to the ventricles and lower levels in distal regions were observed (FIG. 15B, top panel). In the cortex, AAV biodistribution was high as per the measured vector genomes per cell (FIG. 15B, top panel). Further, an equivalent clearance of CBE-induced lyso-GL1 was observed across native and engineered variants (FIG. 15B, bottom panel); however, in more distal regions with lower vector genomes distribution, enhanced clearance of Lyso-GL1 by engineered variants relative to WT was observed.

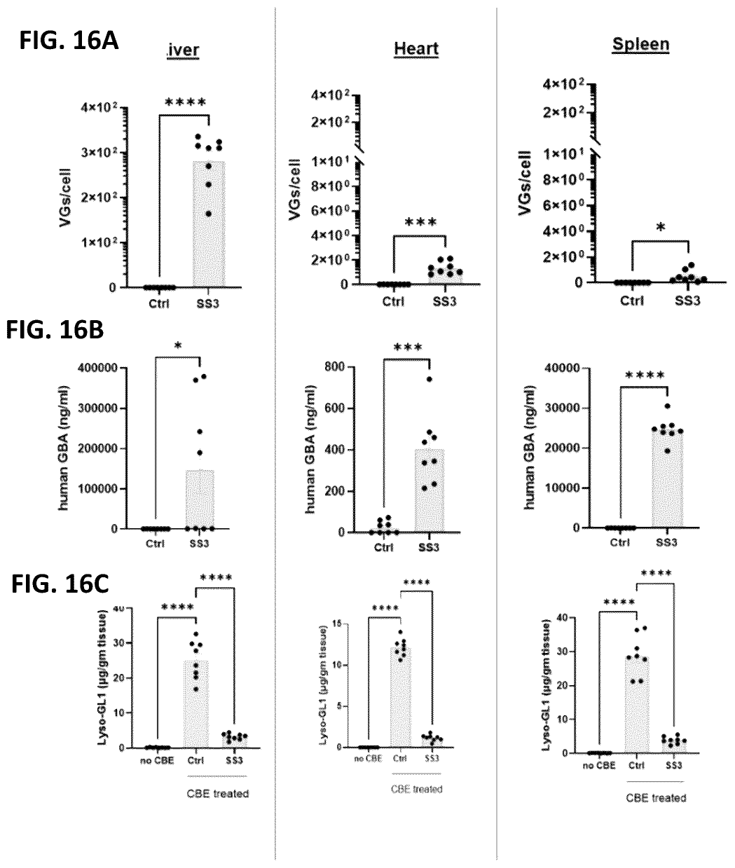

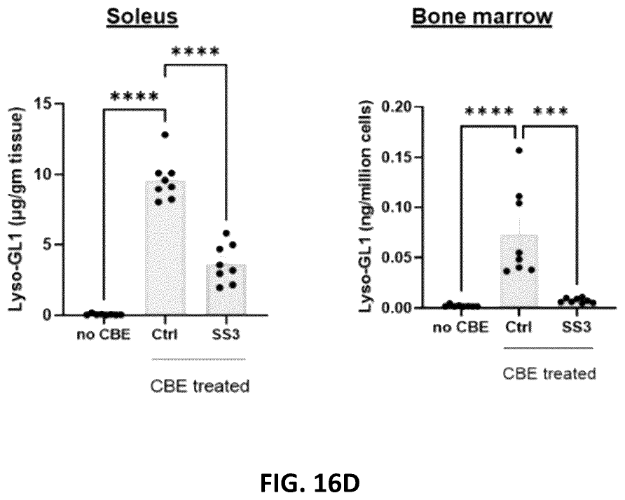

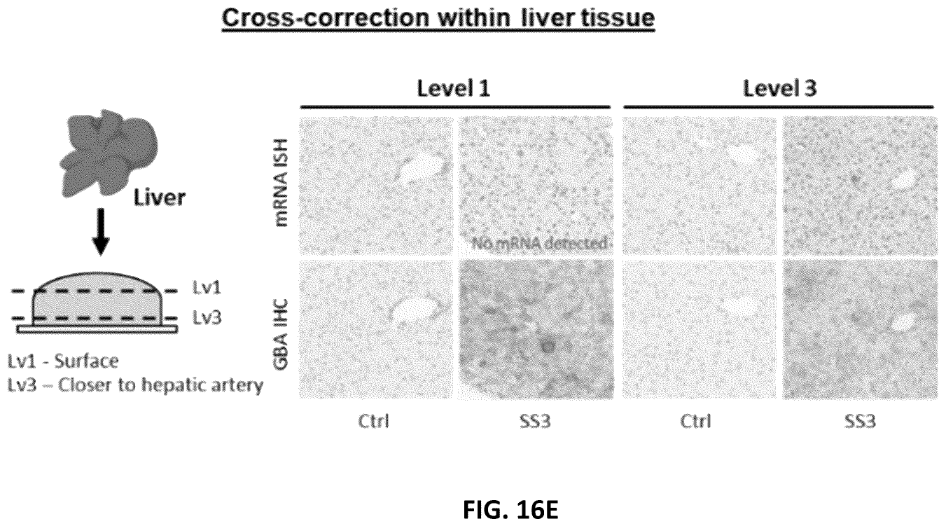

Cross-correction in peripheral tissues of mice following intravenous administration of AAV was also evaluated. AAV (4e13 vg/kg) encoding engineered lysosomal protein (e.g., GBA1) was administered to 3-month-old mice (4e13 VG/kg) and, after 4 weeks, lysosomal dysfunction was induced by CBE (100 mg/kg, IP). Peripheral tissues were collected for analysis one day after CBE. High levels of liver transduction were observed, with significantly less transduction of heart and spleen (FIG. 16A; vector genomes per cell in mice treated with AAV-Ctrl or AAV-SS3-GBA1). Surprisingly, high levels of AAV transgene protein human GBA were observed (FIG. 16B) and robust clearance of CBE-induced lipid substrates (FIG. 16C), indicative of cross-correction from more highly transduced tissues.

In the liver, the distribution of AAV-expressed GBA1 was evaluated. Secretion and uptake in regions with undetectable AAV transgene mRNA were observed (FIG. 16E). Furthermore, some tissues, such as skeletal muscle and bone marrow, are particularly difficult to treat with either standard gene therapy approaches or enzyme replacement therapy. Efficient lipid clearance in soleus and bone marrow samples was observed (FIG. 16D).

These data further demonstrate the ability of cross correcting AAV (e.g., AAV GBA1 with associated signal peptides) to target often-untreated yet relevant tissue-types.

### Example 4: Development of CBE-Induced Lipid Flux Model in Non-Human Primates (NHPs)

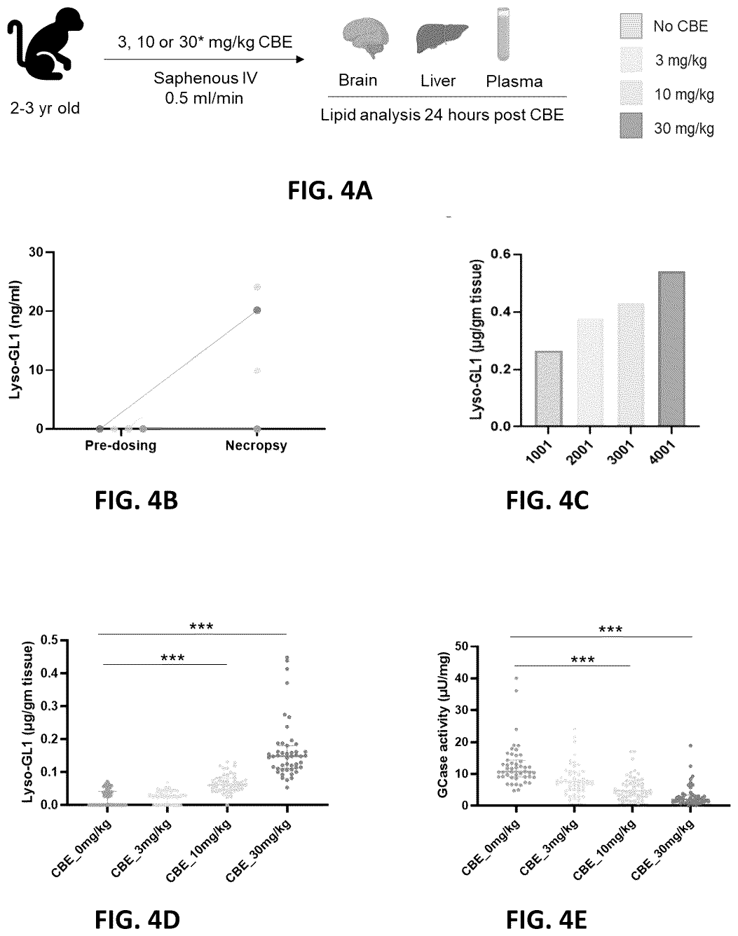

To develop an efficacy model in NHPs, the CBE model developed in rodents and was adapted for NHPs to determine CBE-dosing regimen. Cynomolgus monkeys (Mauritian, 2-3 yr old) were injected with 3, 10 or 30 mg/kg CBE intravenously via the saphenous vein. Brains, liver, and plasma were collected for lipid analyses 24 hours post CBE-dosing (FIG. 4A). Lyso-GL1 levels was detectable in the plasma of all NHPs that got CBE (FIG. 4B) and a dose-dependent increase in Lyso-GL1 was observed in liver from these NHPs (FIG. 4C). For analysis in brains, 47 gray matter brain punches which correspond to 17 different grey mater regions were surveyed. Consistent with the liver data, a dose-dependent increase in Lyso-GL1 was observed across the 4 treatment groups with 10 mg/kg and 30 mg/kg yielding a significant increase in Lyso-GL1 levels compared to the “No CBE” group (FIG. 4D). Furthermore, a concomitant decrease in the GCase activity was observed across all the treatment groups (FIG. 4E) consistent with the mechanism of action of CBE inhibiting GCase enzyme activity.

### Example 5: Effective Reduction of GL1 Lipids in SS3-GBA1 Treated NHPs

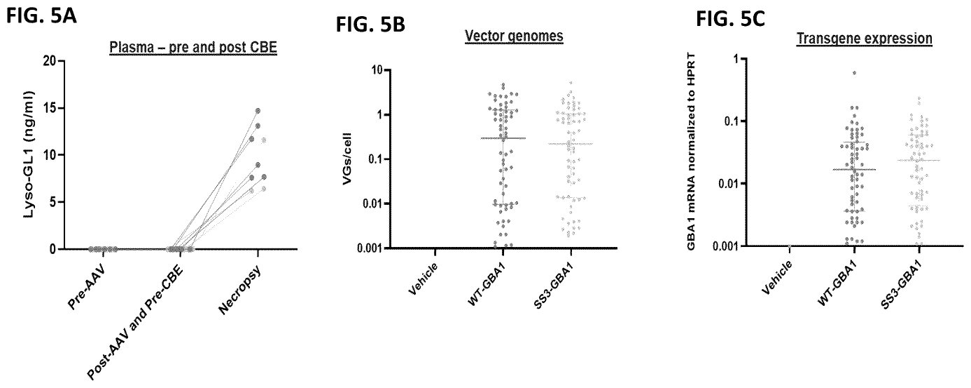

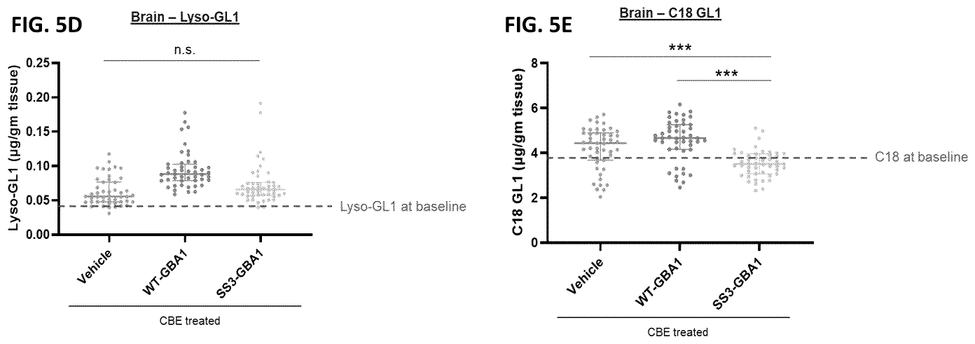

A pharmacology study in Cynomolgus NHPs was performed to determine efficacy of AAVs in CBE-treated NHPs. AAVs (e.g., AAVSAN006 WT GBA1 and AAV.SAN006 SS3-GBA1) were injected in 2-3 yr old NHPs (1.25e¹³ VGs/NHP) via intra-cisterna magna (intra-CSF) dosing. The viruses were allowed to express for 6 weeks and all NHPs were injected with 30 mg/kg CBE intravenously 48 hours prior to necropsy. Plasma was collected at the indicated timepoints such as pre-AAV, pre-AAV and pre-CBE, and post-CBE at necropsy. A spike in Lyso-GL1 in the plasma of all CBE-treated NHPs was observed at necropsy (FIG. 5A).

Vector genome levels (FIG. 5B) and transgene expression of huGBA1 mRNA (FIG. 5C) were comparable across two virus treated groups (WT-GBA1 in red and SS3-GBA1 in light brown). Data is presented as median with inter-quartile range, each data point is average of all NHPs in that group for that punch. Although a significant decrease in Lyso-GL1 levels of AAV treated NHP was not observed when compared to vehicle injected group (FIG. 5D), SS3-GBA1 was able to significantly reduce the accumulated C18 GL1, a predominant isotype of GL1 in the brain (FIG. 5E). Purple line indicates lipid levels under physiological conditions. Importantly, AAV.SAN006-SS3-GBA1 treatment lowers the accumulated C18 GL1 to physiological levels as seen by the median of the data close to the purple line which marks the baseline physiological lipid levels.

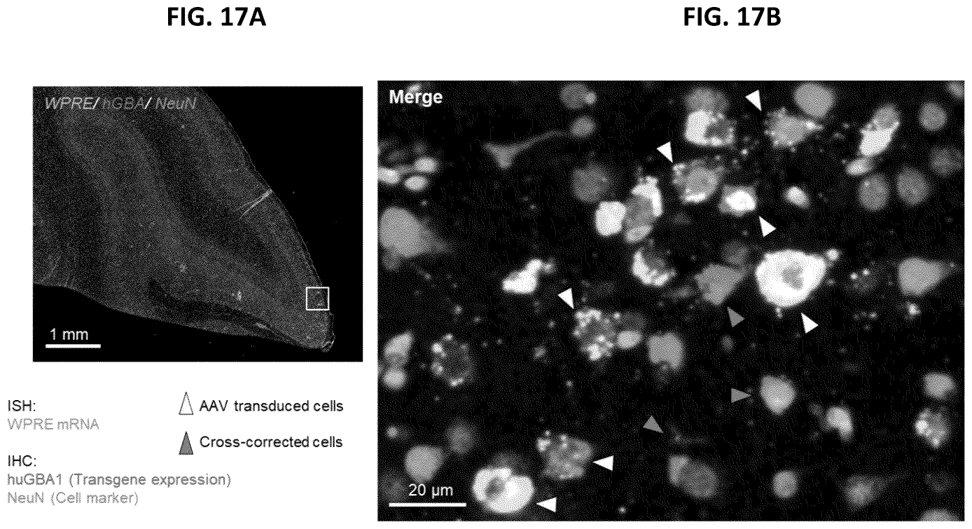

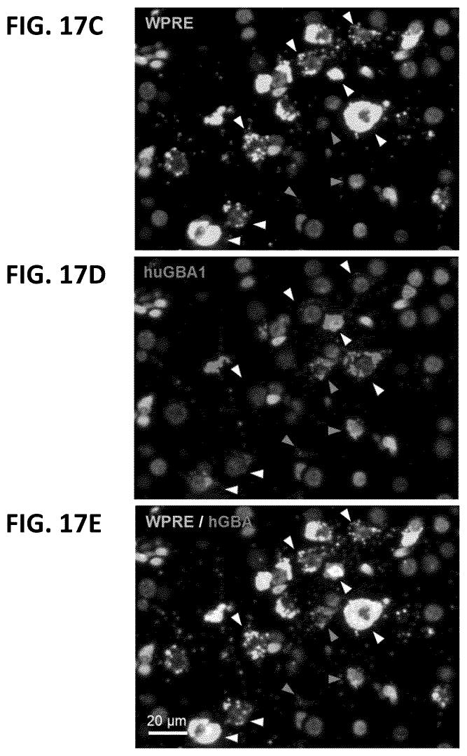

Further, to demonstrate cross-correction of engineered transgene protein human GBA1 in NHP brain, fluorescent multiplex co-detection of AAV transgene mRNA (e.g., WPRE) and therapeutic protein (e.g., huGBA1) was performed. Human GBA was detected in both AAV-transduced cells and cells lacking AAV transgene mRNA (FIGS. 17A-17E). These data indicate that payload engineering enables robust secretion, while preserving endogenous re-uptake capacity.

In summary, this study resulted in: a) comparable vector genomes and transgene expression of WT GBA1 and SS3-GBA1 across NHPs; b) increase in lyso-GL1 in plasma of all NHPs at necropsy, indicative of CBE working effectively; c) statistically significant reduction in C18 GL1 in SS3-GBA1 treated NHPs compared to vehicle and WT-GBA1; and d) histological analyses demonstrate clear secretion and uptake of SS3-GBA1 in non-transduced cells in NHP brain tissues.

### Example 6: Robust Secretion and Diffusion of SS3-GBA1 in Brain Sections of SS3-GBA1 iCM Dosed NHPs

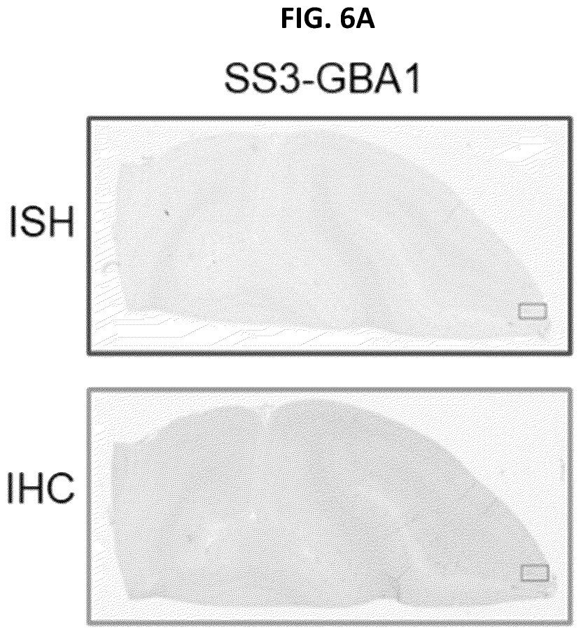

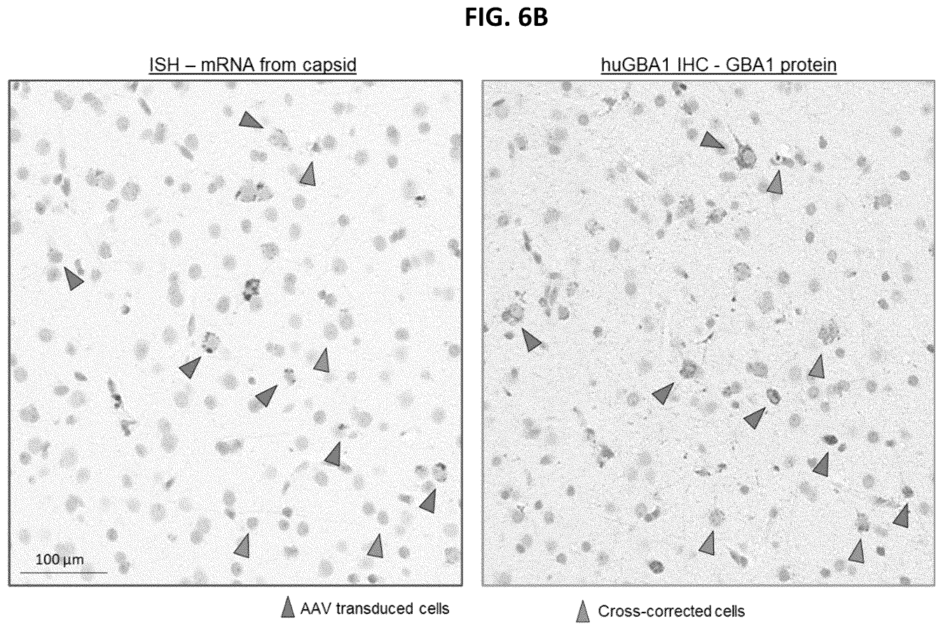

WPRE mRNA was performed in situ hybridization and huGBA1 immunohistochemistry in 5 μm FFPE NHP brain sections from animals were treated with AAV.SAN006-SS3-GBA1 (FIG. 6A). Higher magnification imaging of these images revealed that consistent with previous observations in rodents, SS3-GBA1 was robustly secreted and taken up by non-transduced cells, shown in green arrows (FIG. 6B).

### Example 7: SS3-GBA1 Effectively Promotes Substrate Clearance in Peripheral Tissues in Intravenously Dosed WT Mice

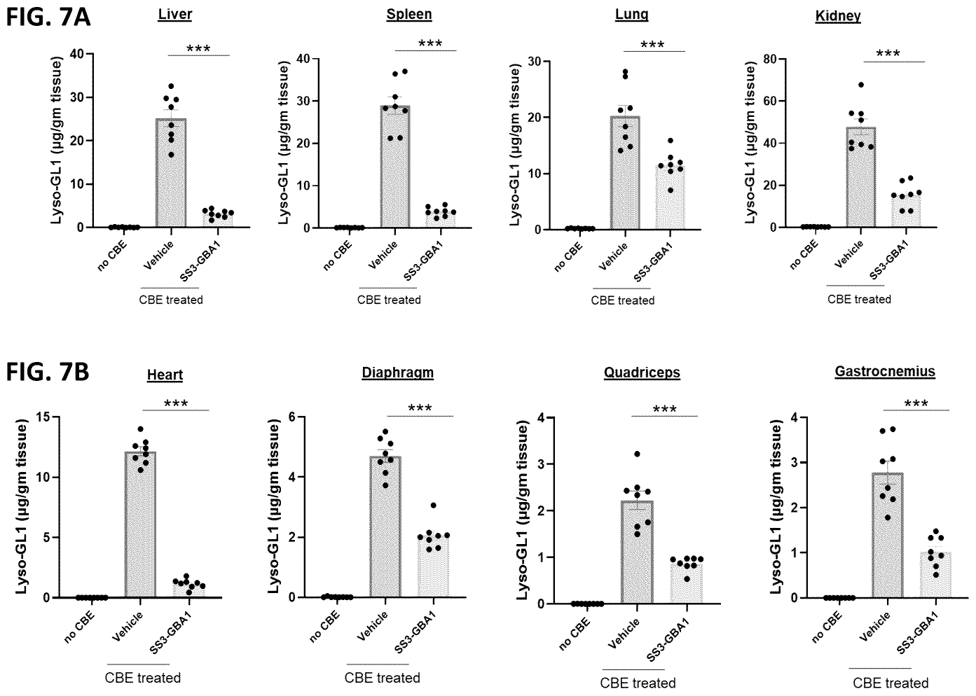

SS3-GBA1 was investigated to determine if it would be able to clear/reduce accumulated lipids even with an IV route of administration as this would facilitate a therapeutic strategy of using a single virus to treat Gaucher patients (Type 1) with IV dosing. Either vehicle or AAV.SAN006 SS3-GBA1 was injected via IV dosing in 3-month-old mice (4e13 VG/kg). AAVs were expressed for 4 weeks followed by 100 mg/kg IP injection of CBE, 24 hours prior to necropsy. Consistent with intra-CSF dosing studies in mice, a robust decrease in Lyso-GL1 was observed across all major peripheral organs (FIG. 7A) as well as all muscles surveyed (FIG. 7B). This establishes the efficacy of AAV.SAN006 SS3-GBA1 across 2 different routes of administration in vivo in mice.

Collectively, across all the rodent and NHP studies shown here with both intra-CSF as well as IV dosing, the data indicate SS3-GBA1 is efficiently secreted, is taken up by non-infected cells and promotes effective clearance of accumulated lipids.

In summary, SS3-GBA1 effectively reduced/cleared accumulated Lyso-GL1 in all major peripheral organs such as liver, spleen, kidney, and lung. Further, SS3-GBA1 also reduced/cleared accumulated Lyso-GL1 in muscle tissues which are implicated in Gaucher Disease. These include the heart, diaphragm, quadriceps, and gastrocnemius.

### Example 8: Broad Transduction and Biodistribution of AAV.SAN006 in IV-Dosed NHPs

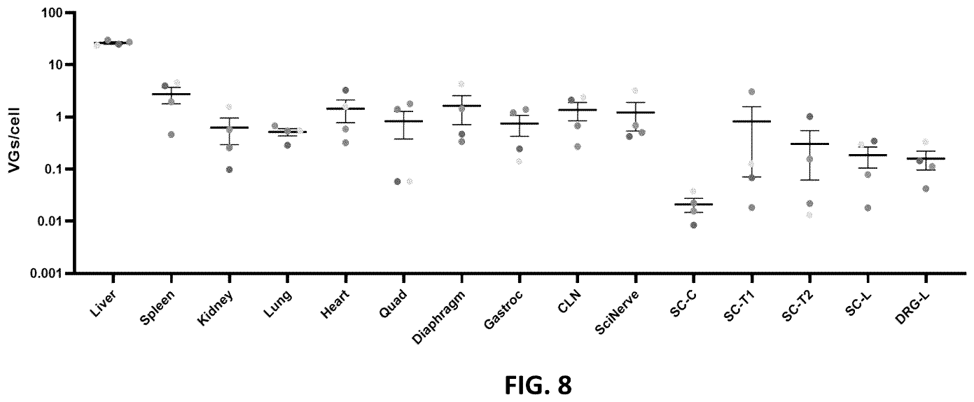

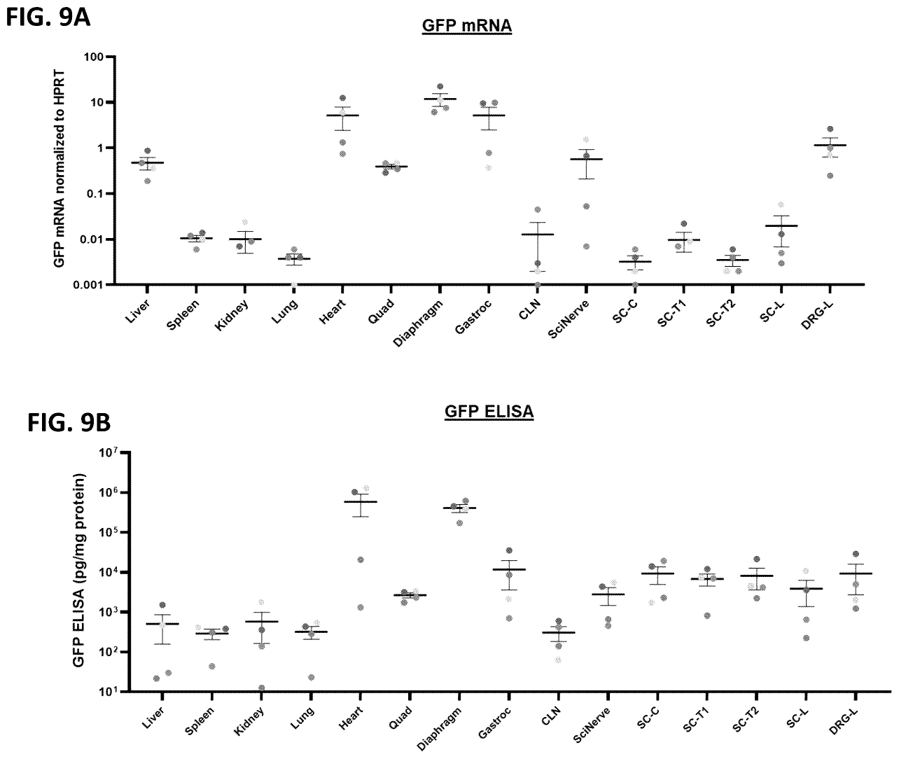

To ensure AAV.SAN006 would also target multiple peripheral tissues in a large animal species such as NHPs, IV injections of AAV.SAN006 expressing GFP (2.5e¹³ VG/kg) were performed in cynomolgus monkeys (female, 2-4 yr old). 3 weeks post-dosing, the NHPs were sacrificed, and multiple tissues were surveyed for vector genomes, mRNA transcript and protein levels. Quantification of vector genome levels across multiple peripheral tissues (FIG. 8) indicates that AAV.SAN006 is able to transduce a wide variety of tissues with IV dosing. GFP mRNA (FIG. 9A) and ELISA protein expression data (FIG. 9B) across tissues surveyed reveal broad transgene expression not only in major peripheral organs such as liver, kidney and lung but also in muscles such as heart, diaphragm, quadriceps and gastrocnemius. GFP ELISA data across these tissues correlate to the mRNA transcript data. Together, these data suggest that AAV.SAN006 is a suitable capsid for not only CNS expression with iCM administration but also for transducing peripheral organs with IV dosing.

### Example 9: Identification of Minimally Efficacious Dose of SS3-GBA1 in NHPs

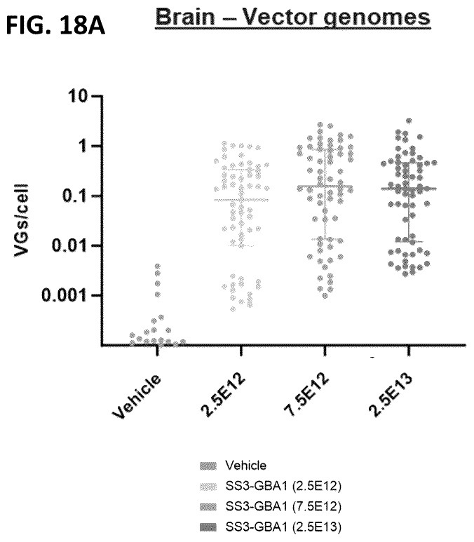

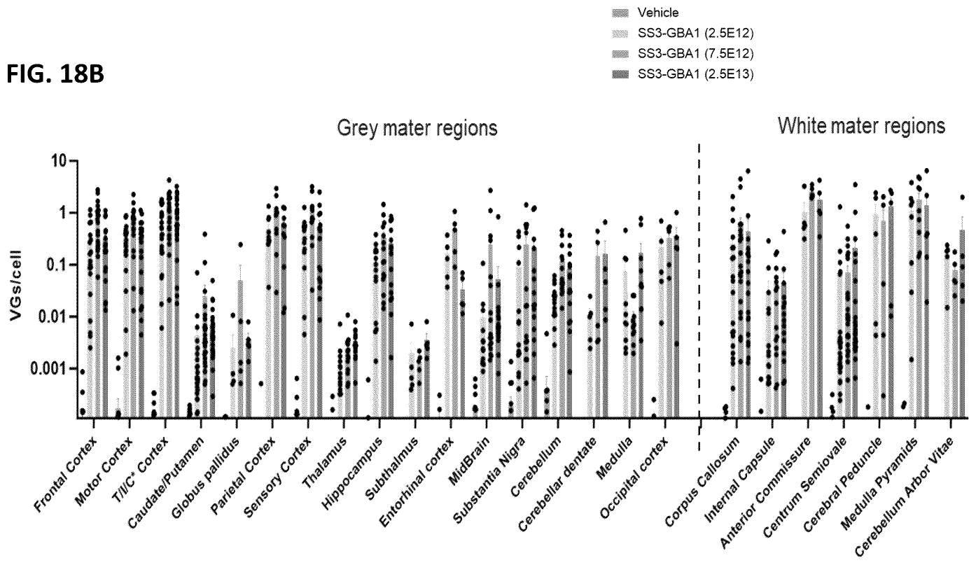

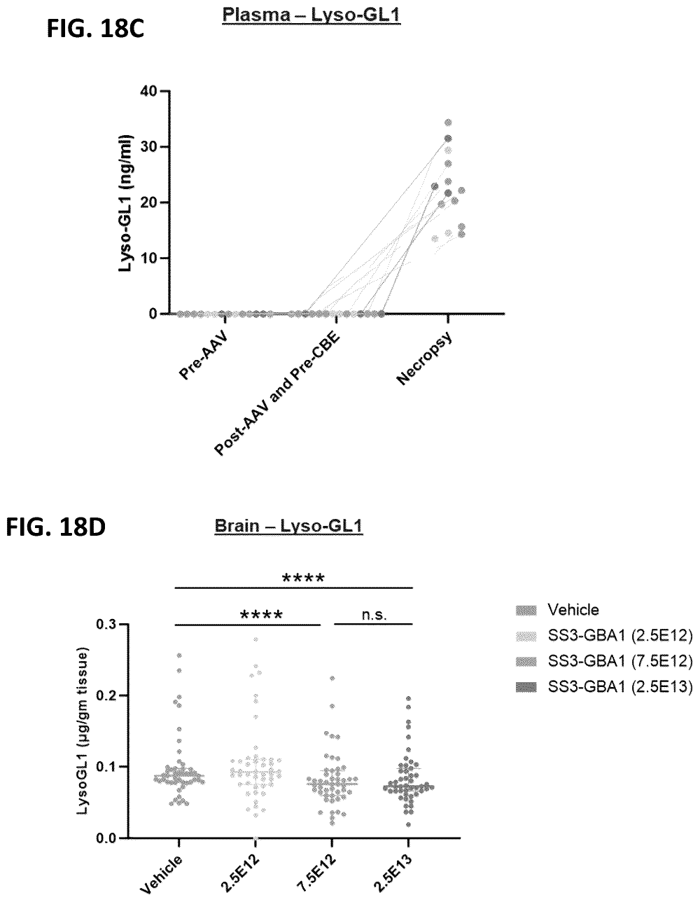

A dose-range finding study was performed in cynomolgus NHPs to determine the minimally efficacious dose (MED) of AAV-SS3-GBA1 in CBE-treated NHPs. Three different doses of AAV.SAN006 SS3-GBA1 were tested (Doses: 1) 2.5e12, 2) 7.5e12, and 3) 2.5e13 total vector genomes). The three doses tested were delivered via intra-cisterna magna (intra-CSF) in 2-3 year old NHPs. The viruses were allowed to express for 8 weeks and all NHPs were injected with 30 mg/kg CBE intravenously, 24 hours prior to necropsy. At all three doses tested, broad brain transduction of SS3-GBA1 in these NHPs was observed (FIGS. 18A-18B).

Administration of CBE causes an accumulation of Lyso-GL1 in plasma and tissues. Consistent with previous work in rodents and NHPs, a robust increase in Lyso-GL1 post CBE injections was observed across both vehicle and AAV-treated groups (FIG. 18C).

Three NHPs were selected from AAV-treated groups for lipid and proteomic mass spectrometry analyses. 47 grey mater punches from the brains of all of the 14 NHPs (5 vehicle treated, 3 AAV-treated for each dose of virus) were assessed. A significant decrease in the accumulated Lyso-GL1 in doses 2) 7.5e12 and 3) 2.5e13 of AAV-SS3-GBA1 was observed (FIG. 18D). Importantly, in doses 2) 7.5e12 and 3) 2.5e13 of AAV-SS3-GBA1 (e.g., the middle and highest dose tested) were not statistically different indicating 7.5e12 VGs (e.g., the mid-dose) to be the MED. These findings are in agreement with previous pharmacology studies where the observed efficacy was at 1.25e13 VGs, which is lower than the highest dose of 2.5e13 VGs tested.

### Example 10: Safety Assessment of AAV-SS3-GBA1 in NHPs

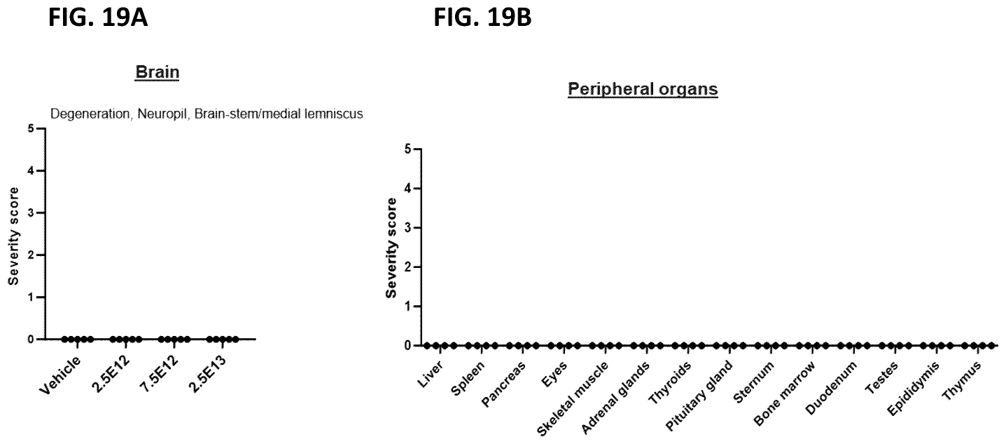

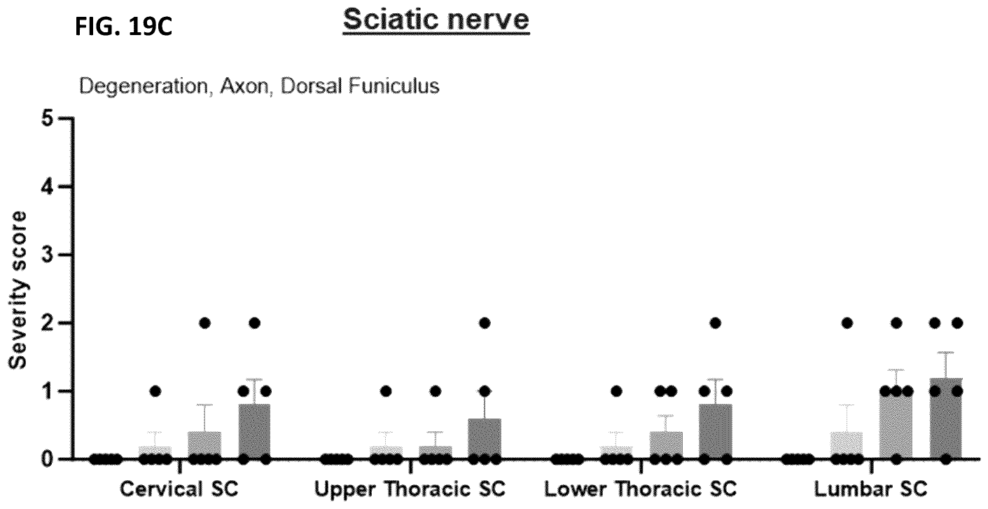

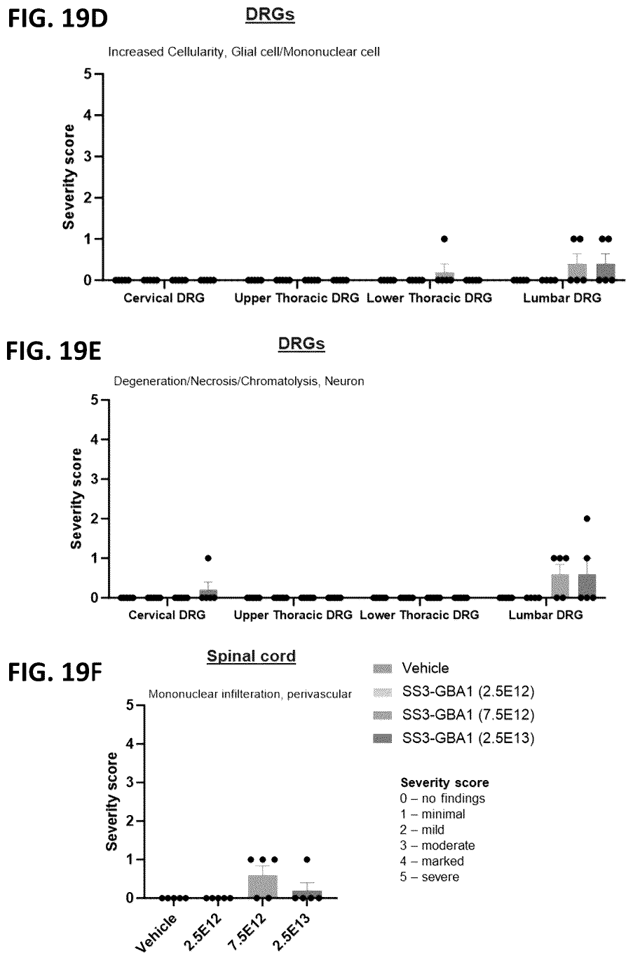

A histopathological analysis across all major organs found little to no pathology across all three doses tested per Example 9. The brain and major peripheral tissues (FIGS. 19A-19B) were completely devoid of any findings. The spinal cord, DRGs, and sciatic nerve (FIGS. 19C-19F) exhibited minimal to mild microscopic findings. These data indicate the safety of SS3-GBA1 when administered into intra-CSF via ICM dosing in NHPs.

### Example 11: Long-Term Efficacy of SS3-GBA1 in Gba^{D409V/+} Mice

In order to demonstrate long-term efficacy and persistence of a one-time administered AAV.SAN006 SS3-GBA1, a long-term study was performed where 3-month-old Gba^{D409V/+} mice were injected with the AAV via bilateral ICV (1.6e11 total VGs) and taken down at 3 months, 6 months and 9 months post dosing. 24 hours prior to necropsy, the mice were treated with 100 mg/kg CBE (conduritol β-epoxide, IP injection) to increase lipid accumulation. At each time-point, lipid clearance in the brains and plasma of the mice was assessed to demonstrate efficacy of SS3-GBA1. The brain was micro-dissected to obtain cortex (e.g., proximal to site of injection) and cerebellum (e.g., distal to site of injection) samples, and lipidomics was performed at all the timepoints to determine efficacy of SS3-GBA1 in lipid clearance on longer time-scales.

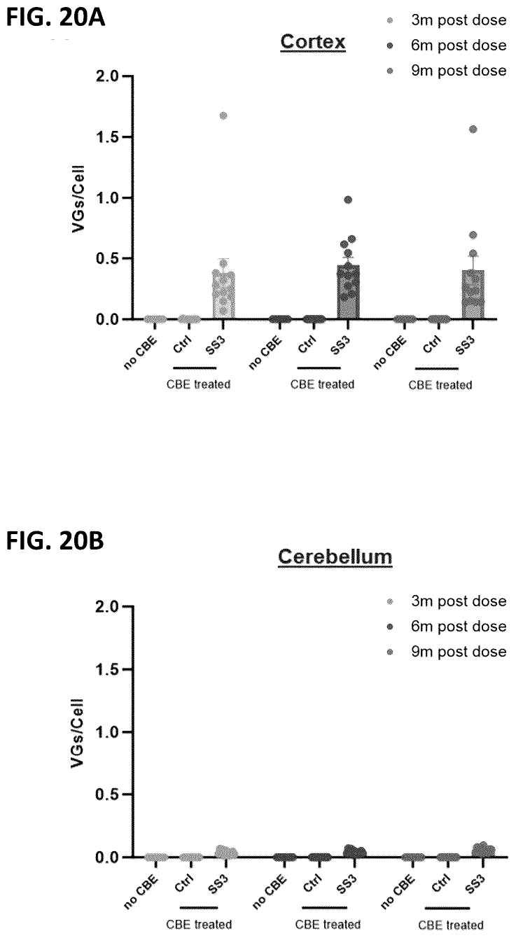

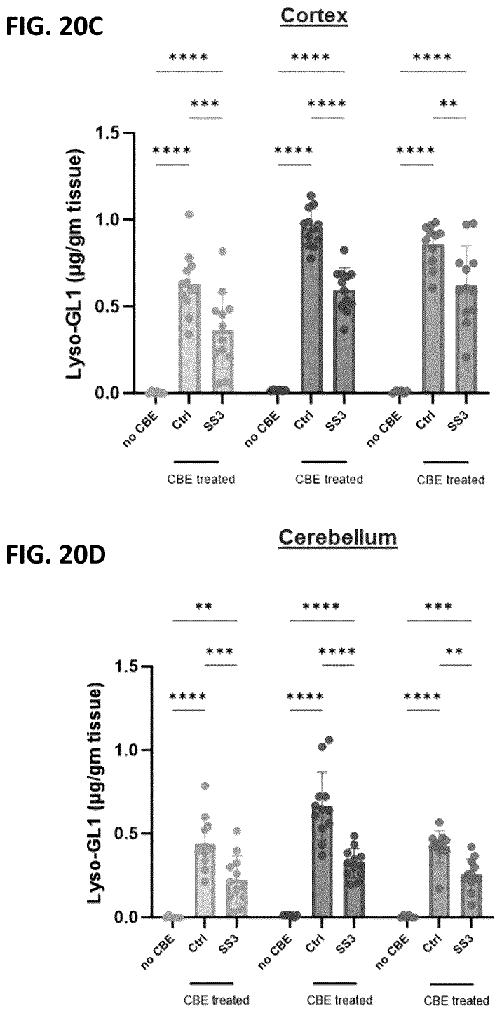

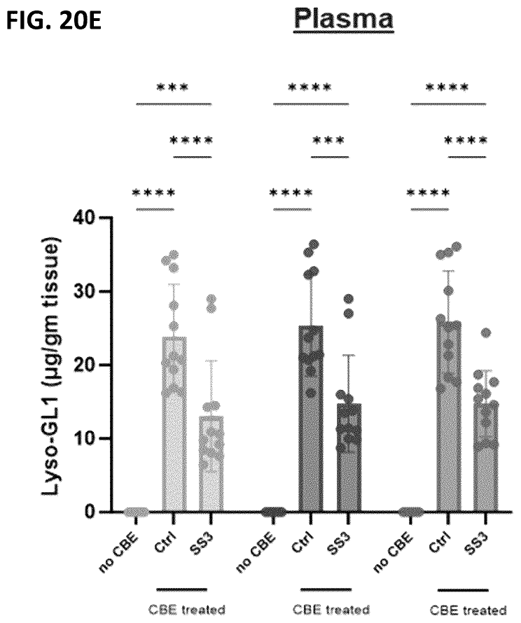

Consistent with bilateral ICV route of administration, viral transduction in the cortex and little to no transduction in the cerebellum was observed (FIGS. 20A-20B). CBE administration caused a spike in Lyso-GL1 across all mice (FIGS. 20C-20E). However, mice treated with SS3-GBA1 had significantly reduced Lyso-GL1 in both cortex and cerebellum, across all time-points (FIGS. 20C-20D).

The importance of these findings is two-fold: 1) long-term efficacy with SS3-GBA1 was observed and 2) this efficacy is due to sustained cross-correction of SS3-GBA1 which causes widespread distribution of SS3-GBA1 in regions beyond those that are transduced with virus, such as the cerebellum in this case. A similar reduction in Lyso-GL1 in the plasma of all the mice when compared to vehicle-treated mice was observed across all time-points (FIG. 20E).

### Sequences

| SEQ | | |
|---|---|---|
| ID NO | Sequence | Notes |
| 1 | MEFSSPSREECPKPLSRVSIMAGSLTGLLLLQAVSWASG | GCase Polypeptide |
| | ARPCIPKSFGYSSVVCVCNATYCDSFDPPTFPALGTFSRY | Sequence (with |
| | ESTRSGRRMELSMGPIQANHTGTGLLLTLQPEQKFQKVK | wild-type signaling |
| | GFGGAMTDAAALNILALSPPAQNLLLKSYFSEEGIGYNII | peptide) |
| | RVPMASCDFSIRTYTYADTPDDFQLHNFSLPEEDTKLKIP | |
| | LIHRALQLAQRPVSLLASPWTSPTWLKTNGAVNGKGSL | |
| | KGQPGDIYHQTWARYFVKFLDAYAEHKLQFWAVTAEN | |
| | EPSAGLLSGYPFQCLGFTPEHQRDFIARDLGPTLANSTHH | |
| | NVRLLMLDDQRLLLPHWAKVVLTDPEAAKYVHGIAVH | |
| | WYLDFLAPAKATLGETHRLFPNTMLFASEACVGSKFWE | |
| | QSVRLGSWDRGMQYSHSIITNLLYHVVGWTDWNLALN | |
| | PEGGPNWVRNFVDSPIIVDITKDTFYKQPMFYHLGHFSK | |
| | FIPEGSQRVGLVASQKNDLDAVALMHPDGSAVVVVLNR | |
| | SSKDVPLTIKDPAVGFLETISPGYSIHTYLWRRQ | |
| 2 | ARPCIPKSFGYSSVVCVCNATYCDSFDPPTFPALGTFSRY | GCase Polypeptide |
| | ESTRSGRRMELSMGPIQANHTGTGLLLTLQPEQKFQKVK | Sequence (following |
| | GFGGAMTDAAALNILALSPPAQNLLLKSYFSEEGIGYNII | cleavage of |
| | RVPMASCDFSIRTYTYADTPDDFQLHNFSLPEEDTKLKIP | signaling peptide) |
| | LIHRALQLAQRPVSLLASPWTSPTWLKTNGAVNGKGSL | |
| | KGQPGDIYHQTWARYFVKFLDAYAEHKLQFWAVTAEN | |
| | EPSAGLLSGYPFQCLGFTPEHQRDFIARDLGPTLANSTHH | |
| | NVRLLMLDDQRLLLPHWAKVVLTDPEAAKYVHGIAVH | |
| | WYLDFLAPAKATLGETHRLFPNTMLFASEACVGSKFWE | |
| | QSVRLGSWDRGMQYSHSIITNLLYHVVGWTDWNLALN | |
| | PEGGPNWVRNFVDSPIIVDITKDTFYKQPMFYHLGHFSK | |
| | FIPEGSQRVGLVASQKNDLDAVALMHPDGSAVVVVLNR | |
| | SSKDVPLTIKDPAVGFLETISPGYSIHTYLWRRQ | |
| 3 | ATGGAGTTCAGCTCTCCGAGCCGGGAAGAGTGCCCCA | Codon-Optimized |
| | AACCCCTGTCGAGAGTGTCCATCATGGCCGGCAGCCT | Human GBA1 |
| | GACCGGCCTGCTGCTGCTGCAAGCTGTTAGCTGGGCC | Sequence with |
| | AGCGGGGCCAGACCTTGTATCCCCAAGAGCTTCGGAT | Endogenous |
| | ATAGCAGCGTGGTCTGCGTGTGCAACGCCACCTACTG | Signaling Peptide |
| | CGATAGCTTCGACCCACCTACCTTTCCAGCTCTGGGCA | |
| | CCTTCTCCAGATACGAGTCTACAAGAAGCGGCAGAAG | |
| | AATGGAACTGTCCATGGGCCCTATCCAGGCCAACCAC | |
| | ACCGGCACAGGCCTCCTGTTGACCCTGCAGCCCGAGC | |
| | AGAAATTTCAGAAAGTGAAGGGATTCGGCGGCGCCAT | |
| | GACCGATGCCGCCGCTCTGAATATCCTGGCGCTGAGC | |
| | CCTCCTGCCCAGAACCTGCTGCTGAAGAGCTACTTTAG | |
| | CGAGGAGGGGATCGGCTACAACATTATCAGAGTGCCC | |
| | ATGGCCAGCTGCGACTTTAGCATCAGAACCTATACAT | |
| | ACGCCGACACCCCAGATGACTTCCAGCTGCACAACTT | |
| | CAGCCTGCCTGAGGAAGATACAAAGCTGAAAATCCCC | |
| | CTGATCCACCGGGCCCTGCAACTGGCTCAGCGGCCAG | |
| | TGTCCCTGCTGGCCTCTCCTTGGACCAGCCCTACCTGG | |
| | CTGAAGACCAATGGCGCCGTGAACGGCAAGGGCTCTC | |
| | TGAAGGGCCAGCCTGGCGACATCTACCATCAGACCTG | |
| | GGCCAGATACTTCGTGAAGTTCCTGGATGCCTACGCT | |
| | GAGCACAAGCTGCAGTTCTGGGCCGTGACCGCCGAGA | |
| | ACGAGCCTTCCGCCGGACTGCTCAGCGGCTATCCTTTC | |
| | CAGTGCCTGGGCTTCACACCTGAGCACCAGCGCGACT | |
| | TCATCGCCAGGGACCTGGGCCCTACACTGGCCAATAG | |
| | CACACACCACAACGTGAGACTGCTGATGCTGGATGAT | |
| | CAGAGACTGCTGCTTCCCCACTGGGCTAAGGTCGTGC | |
| | TGACCGACCCTGAAGCCGCTAAGTACGTGCACGGCAT | |
| | CGCCGTGCACTGGTACCTGGACTTCCTGGCCCCTGCCA | |
| | AGGCCACCCTGGGAGAAACCCACCGGCTGTTCCCTAA | |
| | CACCATGCTGTTTGCCTCAGAGGCCTGTGTGGGCTCCA | |
| | AATTCTGGGAGCAAAGCGTGCGGCTGGGCTCTTGGGA | |
| | CAGAGGAATGCAGTACAGCCACAGCATCATCACCAAC | |
| | CTGCTGTACCACGTGGTGGGCTGGACCGACTGGAACC | |
| | TGGCCCTGAACCCCGAGGGAGGCCCTAACTGGGTCAG | |
| | AAACTTCGTGGACAGCCCCATCATTGTGGATATCACC | |
| | AAGGACACGTTCTACAAGCAGCCAATGTTCTACCACC | |
| | TGGGCCACTTCAGCAAGTTCATCCCTGAAGGCAGTCA | |
| | GAGAGTGGGCCTGGTGGCCTCTCAGAAGAACGACCTG | |
| | GACGCCGTTGCGCTGATGCACCCCGACGGCAGCGCCG | |
| | TGGTGGTGGTGCTGAATAGATCTTCTAAGGATGTGCCT | |
| | CTGACAATCAAGGACCCTGCTGTGGGATTTCTGGAAA | |
| | CAATCAGCCCTGGATACAGCATCCATACATACCTGTG | |
| | GCGGCGGCAGTGA | |
| 4 | ATGGCCTCTCTGTGGCTGCTGAGCTGCTTCAGCCTGGT | Codon-Optimized |
| | GGGCGCCGCTTTCGGCGCCAGACCTTGTATCCCCAAG | Human GBA1 |
| | AGCTTCGGATATAGCAGCGTGGTCTGCGTGTGCAACG | Sequence with |
| | CCACCTACTGCGATAGCTTCGACCCACCTACCTTTCCA | Engineered |
| | GCTCTGGGCACCTTCTCCAGATACGAGTCTACAAGAA | Signaling Peptide |
| | GCGGCAGAAGAATGGAACTGTCCATGGGCCCTATCCA | (SS1) |
| | GGCCAACCACACCGGCACAGGCCTCCTGTTGACCCTG | |
| | CAGCCCGAGCAGAAATTTCAGAAAGTGAAGGGATTCG | |
| | GCGGCGCCATGACCGATGCCGCCGCTCTGAATATCCT | |
| | GGCGCTGAGCCCTCCTGCCCAGAACCTGCTGCTGAAG | |
| | AGCTACTTTAGCGAGGAGGGGATCGGCTACAACATTA | |
| | TCAGAGTGCCCATGGCCAGCTGCGACTTTAGCATCAG | |
| | AACCTATACATACGCCGACACCCCAGATGACTTCCAG | |
| | CTGCACAACTTCAGCCTGCCTGAGGAAGATACAAAGC | |
| | TGAAAATCCCCCTGATCCACCGGGCCCTGCAACTGGC | |
| | TCAGCGGCCAGTGTCCCTGCTGGCCTCTCCTTGGACCA | |
| | GCCCTACCTGGCTGAAGACCAATGGCGCCGTGAACGG | |
| | CAAGGGCTCTCTGAAGGGCCAGCCTGGCGACATCTAC | |
| | CATCAGACCTGGGCCAGATACTTCGTGAAGTTCCTGG | |
| | ATGCCTACGCTGAGCACAAGCTGCAGTTCTGGGCCGT | |
| | GACCGCCGAGAACGAGCCTTCCGCCGGACTGCTCAGC | |
| | GGCTATCCTTTCCAGTGCCTGGGCTTCACACCTGAGCA | |
| | CCAGCGCGACTTCATCGCCAGGGACCTGGGCCCTACA | |
| | CTGGCCAATAGCACACACCACAACGTGAGACTGCTGA | |
| | TGCTGGATGATCAGAGACTGCTGCTTCCCCACTGGGCT | |
| | AAGGTCGTGCTGACCGACCCTGAAGCCGCTAAGTACG | |
| | TGCACGGCATCGCCGTGCACTGGTACCTGGACTTCCTG | |
| | GCCCCTGCCAAGGCCACCCTGGGAGAAACCCACCGGC | |
| | TGTTCCCTAACACCATGCTGTTTGCCTCAGAGGCCTGT | |
| | GTGGGCTCCAAATTCTGGGAGCAAAGCGTGCGGCTGG | |
| | GCTCTTGGGACAGAGGAATGCAGTACAGCCACAGCAT | |
| | CATCACCAACCTGCTGTACCACGTGGTGGGCTGGACC | |
| | GACTGGAACCTGGCCCTGAACCCCGAGGGAGGCCCTA | |
| | ACTGGGTCAGAAACTTCGTGGACAGCCCCATCATTGT | |
| | GGATATCACCAAGGACACGTTCTACAAGCAGCCAATG | |
| | TTCTACCACCTGGGCCACTTCAGCAAGTTCATCCCTGA | |
| | AGGCAGTCAGAGAGTGGGCCTGGTGGCCTCTCAGAAG | |
| | AACGACCTGGACGCCGTTGCGCTGATGCACCCCGACG | |
| | GCAGCGCCGTGGTGGTGGTGCTGAATAGATCTTCTAA | |
| | GGATGTGCCTCTGACAATCAAGGACCCTGCTGTGGGA | |
| | TTTCTGGAAACAATCAGCCCTGGATACAGCATCCATA | |
| | CATACCTGTGGCGGCGGCAGTGA | |
| 5 | ATGTTCCAGACCGGCGGACTGATCGTGTTCTACGGCCT | Codon-Optimized |
| | GCTGGCCCAGACAATGGCCGCCAGACCTTGTATCCCC | Human GBA1 |
| | AAGAGCTTCGGATATAGCAGCGTGGTCTGCGTGTGCA | Sequence with |
| | ACGCCACCTACTGCGATAGCTTCGACCCACCTACCTTT | Engineered |
| | CCAGCTCTGGGCACCTTCTCCAGATACGAGTCTACAA | Signaling Peptide |
| | GAAGCGGCAGAAGAATGGAACTGTCCATGGGCCCTAT | (SS2) |
| | CCAGGCCAACCACACCGGCACAGGCCTCCTGTTGACC | |
| | CTGCAGCCCGAGCAGAAATTTCAGAAAGTGAAGGGAT | |
| | TCGGCGGCGCCATGACCGATGCCGCCGCTCTGAATAT | |
| | CCTGGCGCTGAGCCCTCCTGCCCAGAACCTGCTGCTG | |
| | AAGAGCTACTTTAGCGAGGAGGGGATCGGCTACAACA | |
| | TTATCAGAGTGCCCATGGCCAGCTGCGACTTTAGCATC | |
| | AGAACCTATACATACGCCGACACCCCAGATGACTTCC | |
| | AGCTGCACAACTTCAGCCTGCCTGAGGAAGATACAAA | |
| | GCTGAAAATCCCCCTGATCCACCGGGCCCTGCAACTG | |
| | GCTCAGCGGCCAGTGTCCCTGCTGGCCTCTCCTTGGAC | |
| | CAGCCCTACCTGGCTGAAGACCAATGGCGCCGTGAAC | |
| | GGCAAGGGCTCTCTGAAGGGCCAGCCTGGCGACATCT | |
| | ACCATCAGACCTGGGCCAGATACTTCGTGAAGTTCCT | |
| | GGATGCCTACGCTGAGCACAAGCTGCAGTTCTGGGCC | |
| | GTGACCGCCGAGAACGAGCCTTCCGCCGGACTGCTCA | |
| | GCGGCTATCCTTTCCAGTGCCTGGGCTTCACACCTGAG | |
| | CACCAGCGCGACTTCATCGCCAGGGACCTGGGCCCTA | |
| | CACTGGCCAATAGCACACACCACAACGTGAGACTGCT | |
| | GATGCTGGATGATCAGAGACTGCTGCTTCCCCACTGG | |
| | GCTAAGGTCGTGCTGACCGACCCTGAAGCCGCTAAGT | |
| | ACGTGCACGGCATCGCCGTGCACTGGTACCTGGACTT | |
| | CCTGGCCCCTGCCAAGGCCACCCTGGGAGAAACCCAC | |
| | CGGCTGTTCCCTAACACCATGCTGTTTGCCTCAGAGGC | |
| | CTGTGTGGGCTCCAAATTCTGGGAGCAAAGCGTGCGG | |
| | CTGGGCTCTTGGGACAGAGGAATGCAGTACAGCCACA | |
| | GCATCATCACCAACCTGCTGTACCACGTGGTGGGCTG | |
| | GACCGACTGGAACCTGGCCCTGAACCCCGAGGGAGGC | |
| | CCTAACTGGGTCAGAAACTTCGTGGACAGCCCCATCA | |
| | TTGTGGATATCACCAAGGACACGTTCTACAAGCAGCC | |
| | AATGTTCTACCACCTGGGCCACTTCAGCAAGTTCATCC | |
| | CTGAAGGCAGTCAGAGAGTGGGCCTGGTGGCCTCTCA | |
| | GAAGAACGACCTGGACGCCGTTGCGCTGATGCACCCC | |
| | GACGGCAGCGCCGTGGTGGTGGTGCTGAATAGATCTT | |
| | CTAAGGATGTGCCTCTGACAATCAAGGACCCTGCTGT | |
| | GGGATTTCTGGAAACAATCAGCCCTGGATACAGCATC | |
| | CATACATACCTGTGGCGGCGGCAGTGA | |
| 6 | ATGCTGCTGCTGCTGCTGCTGCTCGGCCTGAGACTGCA | Codon-Optimized |
| | GCTGAGCCTGGGCGCCAGACCTTGTATCCCCAAGAGC | Human GBA1 |
| | TTCGGATATAGCAGCGTGGTCTGCGTGTGCAACGCCA | Sequence with |
| | CCTACTGCGATAGCTTCGACCCACCTACCTTTCCAGCT | Engineered |
| | CTGGGCACCTTCTCCAGATACGAGTCTACAAGAAGCG | Signaling Peptide |
| | GCAGAAGAATGGAACTGTCCATGGGCCCTATCCAGGC | (SS3) |
| | CAACCACACCGGCACAGGCCTCCTGTTGACCCTGCAG | |
| | CCCGAGCAGAAATTTCAGAAAGTGAAGGGATTCGGCG | |
| | GCGCCATGACCGATGCCGCCGCTCTGAATATCCTGGC | |
| | GCTGAGCCCTCCTGCCCAGAACCTGCTGCTGAAGAGC | |
| | TACTTTAGCGAGGAGGGGATCGGCTACAACATTATCA | |
| | GAGTGCCCATGGCCAGCTGCGACTTTAGCATCAGAAC | |
| | CTATACATACGCCGACACCCCAGATGACTTCCAGCTG | |
| | CACAACTTCAGCCTGCCTGAGGAAGATACAAAGCTGA | |
| | AAATCCCCCTGATCCACCGGGCCCTGCAACTGGCTCA | |
| | GCGGCCAGTGTCCCTGCTGGCCTCTCCTTGGACCAGCC | |
| | CTACCTGGCTGAAGACCAATGGCGCCGTGAACGGCAA | |
| | GGGCTCTCTGAAGGGCCAGCCTGGCGACATCTACCAT | |
| | CAGACCTGGGCCAGATACTTCGTGAAGTTCCTGGATG | |
| | CCTACGCTGAGCACAAGCTGCAGTTCTGGGCCGTGAC | |
| | CGCCGAGAACGAGCCTTCCGCCGGACTGCTCAGCGGC | |
| | TATCCTTTCCAGTGCCTGGGCTTCACACCTGAGCACCA | |
| | GCGCGACTTCATCGCCAGGGACCTGGGCCCTACACTG | |
| | GCCAATAGCACACACCACAACGTGAGACTGCTGATGC | |
| | TGGATGATCAGAGACTGCTGCTTCCCCACTGGGCTAA | |
| | GGTCGTGCTGACCGACCCTGAAGCCGCTAAGTACGTG | |
| | CACGGCATCGCCGTGCACTGGTACCTGGACTTCCTGG | |
| | CCCCTGCCAAGGCCACCCTGGGAGAAACCCACCGGCT | |
| | GTTCCCTAACACCATGCTGTTTGCCTCAGAGGCCTGTG | |
| | TGGGCTCCAAATTCTGGGAGCAAAGCGTGCGGCTGGG | |
| | CTCTTGGGACAGAGGAATGCAGTACAGCCACAGCATC | |
| | ATCACCAACCTGCTGTACCACGTGGTGGGCTGGACCG | |
| | ACTGGAACCTGGCCCTGAACCCCGAGGGAGGCCCTAA | |
| | CTGGGTCAGAAACTTCGTGGACAGCCCCATCATTGTG | |
| | GATATCACCAAGGACACGTTCTACAAGCAGCCAATGT | |
| | TCTACCACCTGGGCCACTTCAGCAAGTTCATCCCTGAA | |
| | GGCAGTCAGAGAGTGGGCCTGGTGGCCTCTCAGAAGA | |
| | ACGACCTGGACGCCGTTGCGCTGATGCACCCCGACGG | |
| | CAGCGCCGTGGTGGTGGTGCTGAATAGATCTTCTAAG | |
| | GATGTGCCTCTGACAATCAAGGACCCTGCTGTGGGAT | |
| | TTCTGGAAACAATCAGCCCTGGATACAGCATCCATAC | |
| | ATACCTGTGGCGGCGGCAGTGA | |
| 7 | ATGACCATCCTGTTCCTGACAATGGTGATCAGCTACTT | Codon-Optimized |
| | CGGCTGCATGAAGGCCGCCAGACCTTGTATCCCCAAG | Human GBA1 |
| | AGCTTCGGATATAGCAGCGTGGTCTGCGTGTGCAACG | Sequence with |
| | CCACCTACTGCGATAGCTTCGACCCACCTACCTTTCCA | Engineered |
| | GCTCTGGGCACCTTCTCCAGATACGAGTCTACAAGAA | Signaling Peptide |
| | GCGGCAGAAGAATGGAACTGTCCATGGGCCCTATCCA | (SS4) |
| | GGCCAACCACACCGGCACAGGCCTCCTGTTGACCCTG | |
| | CAGCCCGAGCAGAAATTTCAGAAAGTGAAGGGATTCG | |
| | GCGGCGCCATGACCGATGCCGCCGCTCTGAATATCCT | |
| | GGCGCTGAGCCCTCCTGCCCAGAACCTGCTGCTGAAG | |
| | AGCTACTTTAGCGAGGAGGGGATCGGCTACAACATTA | |
| | TCAGAGTGCCCATGGCCAGCTGCGACTTTAGCATCAG | |
| | AACCTATACATACGCCGACACCCCAGATGACTTCCAG | |
| | CTGCACAACTTCAGCCTGCCTGAGGAAGATACAAAGC | |
| | TGAAAATCCCCCTGATCCACCGGGCCCTGCAACTGGC | |
| | TCAGCGGCCAGTGTCCCTGCTGGCCTCTCCTTGGACCA | |
| | GCCCTACCTGGCTGAAGACCAATGGCGCCGTGAACGG | |
| | CAAGGGCTCTCTGAAGGGCCAGCCTGGCGACATCTAC | |
| | CATCAGACCTGGGCCAGATACTTCGTGAAGTTCCTGG | |
| | ATGCCTACGCTGAGCACAAGCTGCAGTTCTGGGCCGT | |
| | GACCGCCGAGAACGAGCCTTCCGCCGGACTGCTCAGC | |
| | GGCTATCCTTTCCAGTGCCTGGGCTTCACACCTGAGCA | |
| | CCAGCGCGACTTCATCGCCAGGGACCTGGGCCCTACA | |
| | CTGGCCAATAGCACACACCACAACGTGAGACTGCTGA | |
| | TGCTGGATGATCAGAGACTGCTGCTTCCCCACTGGGCT | |
| | AAGGTCGTGCTGACCGACCCTGAAGCCGCTAAGTACG | |
| | TGCACGGCATCGCCGTGCACTGGTACCTGGACTTCCTG | |
| | GCCCCTGCCAAGGCCACCCTGGGAGAAACCCACCGGC | |
| | TGTTCCCTAACACCATGCTGTTTGCCTCAGAGGCCTGT | |
| | GTGGGCTCCAAATTCTGGGAGCAAAGCGTGCGGCTGG | |
| | GCTCTTGGGACAGAGGAATGCAGTACAGCCACAGCAT | |
| | CATCACCAACCTGCTGTACCACGTGGTGGGCTGGACC | |
| | GACTGGAACCTGGCCCTGAACCCCGAGGGAGGCCCTA | |
| | ACTGGGTCAGAAACTTCGTGGACAGCCCCATCATTGT | |
| | GGATATCACCAAGGACACGTTCTACAAGCAGCCAATG | |
| | TTCTACCACCTGGGCCACTTCAGCAAGTTCATCCCTGA | |
| | AGGCAGTCAGAGAGTGGGCCTGGTGGCCTCTCAGAAG | |
| | AACGACCTGGACGCCGTTGCGCTGATGCACCCCGACG | |
| | GCAGCGCCGTGGTGGTGGTGCTGAATAGATCTTCTAA | |
| | GGATGTGCCTCTGACAATCAAGGACCCTGCTGTGGGA | |
| | TTTCTGGAAACAATCAGCCCTGGATACAGCATCCATA | |
| | CATACCTGTGGCGGCGGCAGTGA | |
| 8 | TTGGCCACTCCCTCTCTGCGCGCTCGCTCGCTCACTGA | 5′ AAV2 ITR DNA |
| | GGCCGCCCGGGCAAAGCCCGGGCGTCGGGCGACCTTT | Sequence |
| | GGTCGCCCGGCCTCAGTGAGCGAGCGAGCGCGCAGAG | |
| | AGGGAGTGGCCAACTCCATCACTAGGGGTTCCT | |
| 9 | AGGAACCCCTAGTGATGGAGTTGGCCACTCCCTCTCT | 3′ AAV2 ITR DNA |
| | GCGCGCTCGCTCGCTCACTGAGGCCGCCCGGGCAAAG | Sequence in Flip |
| | CCCGGGCGTCGGGCGACCTTTGGTCGCCCGGCCTCAG | Orientation |
| | TGAGCGAGCGAGCGCGCAGAGAGGGAGTGGCCAA | |
| 10 | GACATTGATTATTGACTAGTTATTAATAGTAATCAATT | CMV Enhancer |
| | ACGGGGTCATTAGTTCATAGCCCATATATGGAGTTCC | Element DNA |
| | GCGTTACATAACTTACGGTAAATGGCCCGCCTGGCTG | Sequence |
| | ACCGCCCAACGACCCCCGCCCATTGACGTCAATAATG | (GenBank: |
| | ACGTATGTTCCCATAGTAACGCCAATAGGGACTTTCC | K03104.1) |
| | ATTGACGTCAATGGGTGGAGTATTTACGGTAAACTGC | |
| | CCACTTGGCAGTACATCAAGTGTATCATATGCCAAGT | |
| | ACGCCCCCTATTGACGTCAATGACGGTAAATGGCCCG | |
| | CCTGGCATTATGCCCAGTACATGACCTTATGGGACTTT | |
| | CCTACTTGGCAGTACATCTACGTATTAGTCATCGCTAT | |
| | TACCATGG | |
| 11 | TCGAGGTGAGCCCCACGTTCTGCTTCACTCTCCCCATC | Chicken-Actin |
| | TCCCCCCCCTCCCCACCCCCAATTTTGTATTTATTTATT | Promoter DNA |
| | TTTTAATTATTTTGTGCAGCGATGGGGGCGGGGGGGG | Sequence |
| | GGGGGGGGCGCGCGCCAGGCGGGGCGGGGCGGGGCG | |
| | AGGGGCGGGGCGGGGCGAGGCGGAGAGGTGCGGCGG | |
| | CAGCCAATCAGAGCGGCGCGCTCCGAAAGTTTCCTTT | |
| | TATGGCGAGGCGGCGGCGGCGGCGGCCCTATAAAAA | |
| | GCGAAGCGCGCGGCGGGCGGGAGTCGCTGCGCGCTGC | |
| | CTTCGCCCCGTGCCCCGCTCCGCCGCC | |
| 12 | TTAATCAACCTCTGGATTACAAAATTTGTGAAAGATTG | WPRE Element |
| | ACTGGTATTCTTAACTATGTTGCTCCTTTTACGCTATGT | DNA Sequence |
| | GGATACGCTGCTTTAATGCCTTTGTATCATGCTATTGC | |
| | TTCCCGTATGGCTTTCATTTTCTCCTCCTTGTATAAATC | |
| | CTGGTTGCTGTCTCTTTATGAGGAGTTGTGGCCCGTTG | |
| | TCAGGCAACGTGGCGTGGTGTGCACTGTGTTTGCTGA | |
| | CGCAACCCCCACTGGTTGGGGCATTGCCACCACCTGT | |
| | CAGCTCCTTTCCGGGACTTTCGCTTTCCCCCTCCCTATT | |
| | GCCACGGCGGAACTCATCGCCGCCTGCCTTGCCCGCT | |
| | GCTGGACAGGGGCTCGGCTGTTGGGCACTGACAATTC | |
| | CGTGGTGTTGTCGGGGAAATCATCGTCCTTTCCTTGGC | |
| | TGCTCGCCTGTGTTGCCACCTGGATTCTGCGCGGGACG | |
| | TCCTTCTGCTACGTCCCTTCGGCCCTCAATCCAGCGGA | |
| | CCTTCCTTCCCGCGGCCTGCTGCCGGCTCTGCGGCCTC | |
| | TTCCGCGTCTTCGCCTTCGCCCTCAGACGAGTCGGATC | |
| | TCCCTTTGGGCCGCCTCCCCGCCTG | |
| 13 | Met Ala Ala Asp Gly Tyr Leu Pro Asp Trp Leu Glu Asp Asn | AAV9 VP1 Capsid |
| | Leu Ser Glu Gly Ile Arg Glu Trp Trp Ala Leu Lys Pro Gly Ala | Amino Acid |
| | Pro Gln Pro Lys Ala Asn Gln Gln His Gln Asp Asn Ala Arg | Sequence (Full |
| | Gly Leu Val Leu Pro Gly Tyr Lys Tyr Leu Gly Pro Gly Asn | Structural Protein) |
| | Gly Leu Asp Lys Gly Glu Pro Val Asn Ala Ala Asp Ala Ala | |
| | Ala Leu Glu His Asp Lys Ala Tyr Asp Gln Gln Leu Lys Ala | |
| | Gly Asp Asn Pro Tyr Leu Lys Tyr Asn His Ala Asp Ala Glu | |
| | Phe Gln Glu Arg Leu Lys Glu Asp Thr Ser Phe Gly Gly Asn | |
| | Leu Gly Arg Ala Val Phe Gln Ala Lys Lys Arg Leu Leu Glu | |
| | Pro Leu Gly Leu Val Glu Glu Ala Ala Lys Thr Ala Pro Gly Lys | |
| | Lys Arg Pro Val Glu Gln Ser Pro Gln Glu Pro Asp Ser Ser Ala | |
| | Gly Ile Gly Lys Ser Gly Ala Gln Pro Ala Lys Lys Arg Leu Asn | |
| | Phe Gly Gln Thr Gly Asp Thr Glu Ser Val Pro Asp Pro Gln Pro | |
| | Ile Gly Glu Pro Pro Ala Ala Pro Ser Gly Val Gly Ser Leu Thr | |
| | Met Ala Ser Gly Gly Gly Ala Pro Val Ala Asp Asn Asn Glu | |
| | Gly Ala Asp Gly Val Gly Ser Ser Ser Gly Asn Trp His Cys Asp | |
| | Ser Gln Trp Leu Gly Asp Arg Val Ile Thr Thr Ser Thr Arg Thr | |
| | Trp Ala Leu Pro Thr Tyr Asn Asn His Leu Tyr Lys Gln Ile Ser | |
| | Asn Ser Thr Ser Gly Gly Ser Ser Asn Asp Asn Ala Tyr Phe Gly | |
| | Tyr Ser Thr Pro Trp Gly Tyr Phe Asp Phe Asn Arg Phe His Cys | |
| | His Phe Ser Pro Arg Asp Trp Gln Arg Leu Ile Asn Asn Asn Trp | |
| | Gly Phe Arg Pro Lys Arg Leu Asn Phe Lys Leu Phe Asn Ile | |
| | Gln Val Lys Glu Val Thr Asp Asn Asn Gly Val Lys Thr Ile Ala | |
| | Asn Asn Leu Thr Ser Thr Val Gln Val Phe Thr Asp Ser Asp | |
| | Tyr Gln Leu Pro Tyr Val Leu Gly Ser Ala His Glu Gly Cys Leu | |
| | Pro Pro Phe Pro Ala Asp Val Phe Met Ile Pro Gln Tyr Gly Tyr | |
| | Leu Thr Leu Asn Asp Gly Ser Gln Ala Val Gly Arg Ser Ser Phe | |
| | Tyr Cys Leu Glu Tyr Phe Pro Ser Gln Met Leu Arg Thr Gly | |
| | Asn Asn Phe Gln Phe Ser Tyr Glu Phe Glu Asn Val Pro Phe | |
| | His Ser Ser Tyr Ala His Ser Gln Ser Leu Asp Arg Leu Met Asn | |
| | Pro Leu Ile Asp Gln Tyr Leu Tyr Tyr Leu Ser Lys Thr Ile Asn | |
| | Gly Ser Gly Gln Asn Gln Gln Thr Leu Lys Phe Ser Val Ala Gly | |
| | Pro Ser Asn Met Ala Val Gln Gly Arg Asn Tyr Ile Pro Gly Pro | |
| | Ser Tyr Arg Gln Gln Arg Val Ser Thr Thr Val Thr Gln Asn Asn | |
| | Asn Ser Glu Phe Ala Trp Pro Gly Ala Ser Ser Trp Ala Leu Asn | |
| | Gly Arg Asn Ser Leu Met Asn Pro Gly Pro Ala Met Ala Ser | |
| | His Lys Glu Gly Glu Asp Arg Phe Phe Pro Leu Ser Gly Ser Leu | |
| | Ile Phe Gly Lys Gln Gly Thr Gly Arg Asp Asn Val Asp Ala | |
| | Asp Lys Val Met Ile Thr Asn Glu Glu Glu Ile Lys Thr Thr Asn | |
| | Pro Val Ala Thr Glu Ser Tyr Gly Gln Val Ala Thr Asn His Gln | |
| | Ser Ala Gln Ala Gln Ala Gln Thr Gly Trp Val Gln Asn Gln Gly | |
| | Ile Leu Pro Gly Met Val Trp Gln Asp Arg Asp Val Tyr Leu Gln | |
| | Gly Pro Ile Trp Ala Lys Ile Pro His Thr Asp Gly Asn Phe His | |
| | Pro Ser Pro Leu Met Gly Gly Phe Gly Met Lys His Pro Pro Pro | |
| | Gln Ile Leu Ile Lys Asn Thr Pro Val Pro Ala Asp Pro Pro Thr | |
| | Ala Phe Asn Lys Asp Lys Leu Asn Ser Phe Ile Thr Gln Tyr Ser | |
| | Thr Gly Gln Val Ser Val Glu Ile Glu Trp Glu Leu Gln Lys Glu | |
| | Asn Ser Lys Arg Trp Asn Pro Glu Ile Gln Tyr Thr Ser Asn Tyr | |
| | Tyr Lys Ser Asn Asn Val Glu Phe Ala Val Asn Thr Glu Gly | |
| | Val Tyr Ser Glu Pro Arg Pro Ile Gly Thr Arg Tyr Leu Thr Arg | |
| | Asn Leu | |
| 14 | Thr Ala Pro Gly Lys Lys Arg Pro Val Glu Gln Ser Pro Gln Glu | AAV9 VP2 Capsid |
| | Pro Asp Ser Ser Ala Gly Ile Gly Lys Ser Gly Ala Gln Pro Ala | Amino Acid |
| | Lys Lys Arg Leu Asn Phe Gly Gln Thr Gly Asp Thr Glu Ser | Sequence |
| | Val Pro Asp Pro Gln Pro Ile Gly Glu Pro Pro Ala Ala Pro Ser | |
| | Gly Val Gly Ser Leu Thr Met Ala Ser Gly Gly Gly Ala Pro Val | |
| | Ala Asp Asn Asn Glu Gly Ala Asp Gly Val Gly Ser Ser Ser | |
| | Gly Asn Trp His Cys Asp Ser Gln Trp Leu Gly Asp Arg Val Ile | |
| | Thr Thr Ser Thr Arg Thr Trp Ala Leu Pro Thr Tyr Asn Asn His | |
| | Leu Tyr Lys Gln Ile Ser Asn Ser Thr Ser Gly Gly Ser Ser Asn | |
| | Asp Asn Ala Tyr Phe Gly Tyr Ser Thr Pro Trp Gly Tyr Phe Asp | |
| | Phe Asn Arg Phe His Cys His Phe Ser Pro Arg Asp Trp Gln | |
| | Arg Leu Ile Asn Asn Asn Trp Gly Phe Arg Pro Lys Arg Leu | |
| | Asn Phe Lys Leu Phe Asn Ile Gln Val Lys Glu Val Thr Asp | |
| | Asn Asn Gly Val Lys Thr Ile Ala Asn Asn Leu Thr Ser Thr Val | |
| | Gln Val Phe Thr Asp Ser Asp Tyr Gln Leu Pro Tyr Val Leu | |
| | Gly Ser Ala His Glu Gly Cys Leu Pro Pro Phe Pro Ala Asp Val | |
| | Phe Met Ile Pro Gln Tyr Gly Tyr Leu Thr Leu Asn Asp Gly Ser | |
| | Gln Ala Val Gly Arg Ser Ser Phe Tyr Cys Leu Glu Tyr Phe Pro | |
| | Ser Gln Met Leu Arg Thr Gly Asn Asn Phe Gln Phe Ser Tyr | |
| | Glu Phe Glu Asn Val Pro Phe His Ser Ser Tyr Ala His Ser Gln | |
| | Ser Leu Asp Arg Leu Met Asn Pro Leu Ile Asp Gln Tyr Leu | |
| | Tyr Tyr Leu Ser Lys Thr Ile Asn Gly Ser Gly Gln Asn Gln Gln | |
| | Thr Leu Lys Phe Ser Val Ala Gly Pro Ser Asn Met Ala Val Gln | |
| | Gly Arg Asn Tyr Ile Pro Gly Pro Ser Tyr Arg Gln Gln Arg Val | |
| | Ser Thr Thr Val Thr Gln Asn Asn Asn Ser Glu Phe Ala Trp Pro | |
| | Gly Ala Ser Ser Trp Ala Leu Asn Gly Arg Asn Ser Leu Met | |
| | Asn Pro Gly Pro Ala Met Ala Ser His Lys Glu Gly Glu Asp | |
| | Arg Phe Phe Pro Leu Ser Gly Ser Leu Ile Phe Gly Lys Gln Gly | |
| | Thr Gly Arg Asp Asn Val Asp Ala Asp Lys Val Met Ile Thr | |
| | Asn Glu Glu Glu Ile Lys Thr Thr Asn Pro Val Ala Thr Glu Ser | |
| | Tyr Gly Gln Val Ala Thr Asn His Gln Ser Ala Gln Ala Ala Ala | |
| | Lys Gly Gly Gly Phe His Gly Ala Ser Ala Gln Ala Gln Thr | |
| | Gly Trp Val Gln Asn Gln Gly Ile Leu Pro Gly Met Val Trp Gln | |
| | Asp Arg Asp Val Tyr Leu Gln Gly Pro Ile Trp Ala Lys Ile Pro | |
| | His Thr Asp Gly Asn Phe His Pro Ser Pro Leu Met Gly Gly Phe | |
| | Gly Met Lys His Pro Pro Pro Gln Ile Leu Ile Lys Asn Thr Pro | |
| | Val Pro Ala Asp Pro Pro Thr Ala Phe Asn Lys Asp Lys Leu | |
| | Asn Ser Phe Ile Thr Gln Tyr Ser Thr Gly Gln Val Ser Val Glu | |
| | Ile Glu Trp Glu Leu Gln Lys Glu Asn Ser Lys Arg Trp Asn Pro | |
| | Glu Ile Gln Tyr Thr Ser Asn Tyr Tyr Lys Ser Asn Asn Val Glu | |
| | Phe Ala Val Asn Thr Glu Gly Val Tyr Ser Glu Pro Arg Pro Ile | |
| | Gly Thr Arg Tyr Leu Thr Arg Asn Leu | |
| 15 | Met Ala Ser Gly Gly Gly Ala Pro Val Ala Asp Asn Asn Glu | AAV9 VP3 Capsid |
| | Gly Ala Asp Gly Val Gly Ser Ser Ser Gly Asn Trp His Cys Asp | Amino Acid |
| | Ser Gln Trp Leu Gly Asp Arg Val Ile Thr Thr Ser Thr Arg Thr | Sequence |
| | Trp Ala Leu Pro Thr Tyr Asn Asn His Leu Tyr Lys Gln Ile Ser | |
| | Asn Ser Thr Ser Gly Gly Ser Ser Asn Asp Asn Ala Tyr Phe Gly | |
| | Tyr Ser Thr Pro Trp Gly Tyr Phe Asp Phe Asn Arg Phe His Cys | |
| | His Phe Ser Pro Arg Asp Trp Gln Arg Leu Ile Asn Asn Asn Trp | |
| | Gly Phe Arg Pro Lys Arg Leu Asn Phe Lys Leu Phe Asn Ile | |
| | Gln Val Lys Glu Val Thr Asp Asn Asn Gly Val Lys Thr Ile Ala | |
| | Asn Asn Leu Thr Ser Thr Val Gln Val Phe Thr Asp Ser Asp | |
| | Tyr Gln Leu Pro Tyr Val Leu Gly Ser Ala His Glu Gly Cys Leu | |
| | Pro Pro Phe Pro Ala Asp Val Phe Met Ile Pro Gln Tyr Gly Tyr | |
| | Leu Thr Leu Asn Asp Gly Ser Gln Ala Val Gly Arg Ser Ser Phe | |
| | Tyr Cys Leu Glu Tyr Phe Pro Ser Gln Met Leu Arg Thr Gly | |
| | Asn Asn Phe Gln Phe Ser Tyr Glu Phe Glu Asn Val Pro Phe | |
| | His Ser Ser Tyr Ala His Ser Gln Ser Leu Asp Arg Leu Met Asn | |
| | Pro Leu Ile Asp Gln Tyr Leu Tyr Tyr Leu Ser Lys Thr Ile Asn | |
| | Gly Ser Gly Gln Asn Gln Gln Thr Leu Lys Phe Ser Val Ala Gly | |
| | Pro Ser Asn Met Ala Val Gln Gly Arg Asn Tyr Ile Pro Gly Pro | |
| | Ser Tyr Arg Gln Gln Arg Val Ser Thr Thr Val Thr Gln Asn Asn | |
| | Asn Ser Glu Phe Ala Trp Pro Gly Ala Ser Ser Trp Ala Leu Asn | |
| | Gly Arg Asn Ser Leu Met Asn Pro Gly Pro Ala Met Ala Ser | |
| | His Lys Glu Gly Glu Asp Arg Phe Phe Pro Leu Ser Gly Ser Leu | |
| | Ile Phe Gly Lys Gln Gly Thr Gly Arg Asp Asn Val Asp Ala | |
| | Asp Lys Val Met Ile Thr Asn Glu Glu Glu Ile Lys Thr Thr Asn | |
| | Pro Val Ala Thr Glu Ser Tyr Gly Gln Val Ala Thr Asn His Gln | |
| | Ser Ala Gln Ala Gln Ala Gln Thr Gly Trp Val Gln Asn Gln Gly | |
| | Ile Leu Pro Gly Met Val Trp Gln Asp Arg Asp Val Tyr Leu Gln | |
| | Gly Pro Ile Trp Ala Lys Ile Pro His Thr Asp Gly Asn Phe His | |
| | Pro Ser Pro Leu Met Gly Gly Phe Gly Met Lys His Pro Pro Pro | |
| | Gln Ile Leu Ile Lys Asn Thr Pro Val Pro Ala Asp Pro Pro Thr | |
| | Ala Phe Asn Lys Asp Lys Leu Asn Ser Phe Ile Thr Gln Tyr Ser | |
| | Thr Gly Gln Val Ser Val Glu Ile Glu Trp Glu Leu Gln Lys Glu | |
| | Asn Ser Lys Arg Trp Asn Pro Glu Ile Gln Tyr Thr Ser Asn Tyr | |
| | Tyr Lys Ser Asn Asn Val Glu Phe Ala Val Asn Thr Glu Gly | |
| | Val Tyr Ser Glu Pro Arg Pro Ile Gly Thr Arg Tyr Leu Thr Arg | |
| | Asn Leu | |
| 16 | KGGGFHG | Targeting Peptide |
| | | Amino Acid |
| | | Sequence |
| 17 | AAAKGGGFHGAS | Targeting Peptide |
| | | Flanked by Linkers- |
| | | Amino Acid |
| | | Sequence |
| 18 | Met Ala Ala Asp Gly Tyr Leu Pro Asp Trp Leu Glu Asp Asn | SAN006 Capsid |
| | Leu Ser Glu Gly Ile Arg Glu Trp Trp Ala Leu Lys Pro Gly Ala | Amino Acid |
| | Pro Gln Pro Lys Ala Asn Gln Gln His Gln Asp Asn Ala Arg | Sequence (Full |
| | Gly Leu Val Leu Pro Gly Tyr Lys Tyr Leu Gly Pro Gly Asn | Structural Protein) |
| | Gly Leu Asp Lys Gly Glu Pro | |
| | Val Asn Ala Ala Asp Ala Ala Ala Leu Glu His Asp Lys Ala | |
| | Tyr Asp Gln Gln Leu Lys Ala Gly Asp Asn Pro Tyr Leu Lys | |
| | Tyr Asn His Ala Asp Ala Glu Phe Gln Glu Arg Leu Lys Glu | |
| | Asp Thr Ser Phe Gly Gly Asn Leu Gly Arg Ala Val Phe Gln | |
| | Ala Lys Lys Arg Leu Leu Glu Pro Leu Gly Leu Val Glu Glu | |
| | Ala Ala Lys Thr Ala Pro Gly Lys Lys Arg Pro Val Glu Gln Ser | |
| | Pro Gln Glu Pro Asp Ser Ser Ala Gly Ile Gly Lys Ser Gly Ala | |
| | Gln Pro Ala Lys Lys Arg Leu Asn Phe Gly Gln Thr Gly Asp | |
| | Thr Glu Ser Val Pro Asp Pro Gln Pro Ile Gly Glu Pro Pro Ala | |
| | Ala Pro Ser Gly Val Gly Ser Leu Thr Met Ala Ser Gly Gly Gly | |
| | Ala Pro Val Ala Asp Asn Asn Glu Gly Ala Asp Gly Val Gly | |
| | Ser Ser Ser Gly Asn Trp His Cys Asp Ser Gln Trp Leu Gly Asp | |
| | Arg Val Ile Thr Thr Ser Thr Arg Thr Trp Ala Leu Pro Thr Tyr | |
| | Asn Asn His Leu Tyr Lys Gln Ile Ser Asn Ser Thr Ser Gly Gly | |
| | Ser Ser Asn Asp Asn | |
| | Ala Tyr Phe Gly Tyr Ser Thr Pro Trp Gly Tyr Phe Asp Phe Asn | |
| | Arg Phe His Cys His Phe Ser Pro Arg Asp Trp Gln Arg Leu Ile | |
| | Asn Asn Asn Trp Gly Phe Arg Pro Lys Arg Leu Asn Phe Lys | |
| | Leu Phe Asn Ile Gln Val Lys Glu Val Thr Asp Asn Asn Gly | |
| | Val Lys Thr Ile Ala Asn Asn Leu Thr Ser Thr Val Gln Val Phe | |
| | Thr Asp Ser Asp Tyr Gln Leu Pro Tyr Val Leu Gly Ser Ala His | |
| | Glu Gly Cys Leu Pro Pro Phe Pro Ala Asp Val Phe Met Ile Pro | |
| | Gln Tyr Gly Tyr Leu Thr Leu Asn Asp Gly Ser Gln Ala Val | |
| | Gly Arg Ser Ser Phe Tyr Cys Leu Glu Tyr Phe Pro Ser Gln Met | |
| | Leu Arg Thr Gly Asn Asn Phe Gln Phe Ser Tyr Glu Phe Glu | |
| | Asn Val Pro Phe His Ser Ser Tyr Ala His Ser Gln Ser Leu Asp | |
| | Arg Leu Met Asn Pro Leu Ile Asp Gln Tyr Leu Tyr Tyr Leu Ser | |
| | Lys Thr Ile Asn Gly Ser Gly Gln Asn Gln Gln Thr Leu Lys Phe | |
| | Ser Val Ala Gly Pro Ser Asn Met Ala Val Gln Gly Arg Asn Tyr | |
| | Ile Pro | |
| | Gly Pro Ser Tyr Arg Gln Gln Arg Val Ser Thr Thr Val Thr Gln | |
| | Asn Asn Asn Ser Glu Phe Ala Trp Pro Gly Ala Ser Ser Trp Ala | |
| | Leu Asn Gly Arg Asn Ser Leu Met Asn Pro Gly Pro Ala Met | |
| | Ala Ser His Lys Glu Gly Glu Asp Arg Phe Phe Pro Leu Ser Gly | |
| | Ser Leu Ile Phe Gly Lys Gln Gly Thr Gly Arg Asp Asn Val Asp | |
| | Ala Asp Lys Val Met Ile Thr Asn Glu Glu Glu Ile Lys Thr Thr | |
| | Asn Pro Val Ala Thr Glu Ser Tyr Gly Gln Val Ala Thr Asn His | |
| | Gln Ser Ala Gln Ala Ala Ala Lys Gly Gly Gly Phe His Gly | |
| | Ala Ser Ala Gln Ala Gln Thr Gly Trp Val Gln Asn Gln Gly Ile | |
| | Leu Pro Gly Met Val Trp Gln Asp Arg Asp Val Tyr Leu Gln | |
| | Gly Pro Ile Trp Ala Lys Ile Pro His Thr Asp Gly Asn Phe His | |
| | Pro Ser Pro Leu Met Gly Gly Phe Gly Met Lys His Pro Pro Pro | |
| | Gln Ile Leu Ile Lys Asn Thr Pro Val Pro Ala Asp Pro Pro Thr | |
| | Ala Phe Asn Lys Asp Lys Leu Asn Ser Phe Ile Thr Gln Tyr Ser | |
| | Thr | |
| | Gly Gln Val Ser Val Glu Ile Glu Trp Glu Leu Gln Lys Glu Asn | |
| | Ser Lys Arg Trp Asn Pro Glu Ile Gln Tyr Thr Ser Asn Tyr Tyr | |
| | Lys Ser Asn Asn Val Glu Phe Ala Val Asn Thr Glu Gly Val | |
| | Tyr Ser Glu Pro Arg Pro Ile Gly Thr Arg Tyr Leu Thr Arg Asn | |
| | Leu | |
| 19 | TTGGCCACTCCCTCTCTGCGCGCTCGCTCGCTCACTGA | Full DNA sequence |
| | GGCCGCCCGGGCAAAGCCCGGGCGTCGGGCGACCTTT | of plasmid for |
| | GGTCGCCCGGCCTCAGTGAGCGAGCGAGCGCGCAGAG | rAAV packaging |
| | AGGGAGTGGCCAACTCCATCACTAGGGGTTCCTTACG | (pAAV-CBA-GBA- |
| | TACAATTGGGATCCCGGACCGTCGACATTGATTATTG | WPRE-bGH); |
| | ACTAGTTATTAATAGTAATCAATTACGGGGTCATTAGT | coding sequence for |
| | TCATAGCCCATATATGGAGTTCCGCGTTACATAACTTA | GBA1 is underlined |
| | CGGTAAATGGCCCGCCTGGCTGACCGCCCAACGACCC | Underlined, codon |
| | CCGCCCATTGACGTCAATAATGACGTATGTTCCCATAG | Optimized GBA1 |
| | TAACGCCAATAGGGACTTTCCATTGACGTCAATGGGT | DNA sequence |
| | GGAGTATTTACGGTAAACTGCCCACTTGGCAGTACAT | (Human) |
| | CAAGTGTATCATATGCCAAGTACGCCCCCTATTGACGT | |
| | CAATGACGGTAAATGGCCCGCCTGGCATTATGCCCAG | |
| | TACATGACCTTATGGGACTTTCCTACTTGGCAGTACAT | |
| | CTACGTATTAGTCATCGCTATTACCATGGTCGAGGTGA | |
| | GCCCCACGTTCTGCTTCACTCTCCCCATCTCCCCCCCC | |
| | TCCCCACCCCCAATTTTGTATTTATTTATTTTTTAATTA | |
| | TTTTGTGCAGCGATGGGGGCGGGGGGGGGGGGGGGG | |
| | CGCGCGCCAGGCGGGGCGGGGCGGGGCGAGGGGCGG | |
| | GGCGGGGCGAGGCGGAGAGGTGCGGCGGCAGCCAAT | |
| | CAGAGCGGCGCGCTCCGAAAGTTTCCTTTTATGGCGA | |
| | GGCGGCGGCGGCGGCGGCCCTATAAAAAGCGAAGCG | |
| | CGCGGCGGGCGGGAGTCGCTGCGCGCTGCCTTCGCCC | |
| | CGTGCCCCGCTCCGCCGCCGCCTCGCGCCGCCCGCCC | |
| | CGGCTCTGACTGACCGCGTTACTCCCACAGGTGAGCG | |
| | GGCGGGACGGCCCTTCTCCTCCGGGCTGTAATTAGCG | |
| | CTTGGTTTAATGACGGCTTGTTTCTTTTCTGTGGCTGC | |
| | GTGAAAGCCTTGAGGGGCTCCGGGAGGGCCCTTTGTG | |
| | CGGGGGGAGCGGCTCGGGGGGTGCGTGCGTGTGTGTG | |
| | TGCGTGGGGAGCGCCGCGTGCGGCTCCGCGCTGCCCG | |
| | GCGGCTGTGAGCGCTGCGGGCGCGGCGCGGGGCTTTG | |
| | TGCGCTCCGCAGTGTGCGCGAGGGGAGCGCGGCCGGG | |
| | GGCGGTGCCCCGCGGTGCGGGGGGGGCTGCGAGGGG | |
| | AACAAAGGCTGCGTGCGGGGTGTGTGCGTGGGGGGGT | |
| | GAGCAGGGGGTGTGGGCGCGTCGGTCGGGCTGCAACC | |
| | CCCCCTGCACCCCCCTCCCCGAGTTGCTGAGCACGGC | |
| | CCGGCTTCGGGTGCGGGGCTCCGTACGGGGCGTGGCG | |
| | CGGGGCTCGCCGTGCCGGGGGGGGGTGGCGGCAGGT | |
| | GGGGGTGCCGGGCGGGGCGGGGCCGCCTCGGGCCGG | |
| | GGAGGGCTCGGGGGAGGGGCGCGGCGGCCCCCGGAG | |
| | CGCCGGCGGCTGTCGAGGCGCGGCGAGCCGCAGCCAT | |
| | TGCCTTTTATGGTAATCGTGCGAGAGGGCGCAGGGAC | |
| | TTCCTTTGTCCCAAATCTGTGCGGAGCCGAAATCTGGG | |
| | AGGCGCCGCCGCACCCCCTCTAGCGGGCGCGGGGCGA | |
| | AGCGGTGCGGCGCCGGCAGGAAGGAAATGGGCGGGG | |
| | AGGGCCTTCGTGCGTCGCCGCGCCGCCGTCCCCTTCTC | |
| | CCTCTCCAGCCTCGGGGCTGTCCGCGGGGGGACGGCT | |
| | GCCTTCGGGGGGGACGGGGCAGGGCGGGGTTCGGCTT | |
| | CTGGCGTGTGACCGGCGGCTCTAGAGCCTCTGCTAAC | |
| | CATGTTCATGCCTTCTTCTTTTTCCTACAGCTCCTGGGC | |
| | AACGTGCTGGTTATTGTGCTGTCTCATCATTTTGGCAA | |
| | AGAATTCGCCACCATGGAGTTCAGCTCTCCGAGCCGG | |
| | GAAGAGTGCCCCAAACCCCTGTCGAGAGTGTCCATCA | |
| | TGGCCGGCAGCCTGACCGGCCTGCTGCTGCTGCAAGC | |
| | TGTTAGCTGGGCCAGCGGGGCCAGACCTTGTATCCCC | |
| | AAGAGCTTCGGATATAGCAGCGTGGTCTGCGTGTGCA | |
| | ACGCCACCTACTGCGATAGCTTCGACCCACCTACCTTT | |
| | CCAGCTCTGGGCACCTTCTCCAGATACGAGTCTACAA | |
| | GAAGCGGCAGAAGAATGGAACTGTCCATGGGCCCTAT | |
| | CCAGGCCAACCACACCGGCACAGGCCTCCTGTTGACC | |
| | CTGCAGCCCGAGCAGAAATTTCAGAAAGTGAAGGGAT | |
| | TCGGCGGCGCCATGACCGATGCCGCCGCTCTGAATAT | |
| | CCTGGCGCTGAGCCCTCCTGCCCAGAACCTGCTGCTG | |
| | AAGAGCTACTTTAGCGAGGAGGGGATCGGCTACAACA | |
| | TTATCAGAGTGCCCATGGCCAGCTGCGACTTTAGCATC | |
| | AGAACCTATACATACGCCGACACCCCAGATGACTTCC | |
| | AGCTGCACAACTTCAGCCTGCCTGAGGAAGATACAAA | |
| | GCTGAAAATCCCCCTGATCCACCGGGCCCTGCAACTG | |
| | GCTCAGCGGCCAGTGTCCCTGCTGGCCTCTCCTTGGAC | |
| | CAGCCCTACCTGGCTGAAGACCAATGGCGCCGTGAAC | |
| | GGCAAGGGCTCTCTGAAGGGCCAGCCTGGCGACATCT | |
| | ACCATCAGACCTGGGCCAGATACTTCGTGAAGTTCCT | |
| | GGATGCCTACGCTGAGCACAAGCTGCAGTTCTGGGCC | |
| | GTGACCGCCGAGAACGAGCCTTCCGCCGGACTGCTCA | |
| | GCGGCTATCCTTTCCAGTGCCTGGGCTTCACACCTGAG | |
| | CACCAGCGCGACTTCATCGCCAGGGACCTGGGCCCTA | |
| | CACTGGCCAATAGCACACACCACAACGTGAGACTGCT | |
| | GATGCTGGATGATCAGAGACTGCTGCTTCCCCACTGG | |
| | GCTAAGGTCGTGCTGACCGACCCTGAAGCCGCTAAGT | |
| | ACGTGCACGGCATCGCCGTGCACTGGTACCTGGACTT | |
| | CCTGGCCCCTGCCAAGGCCACCCTGGGAGAAACCCAC | |
| | CGGCTGTTCCCTAACACCATGCTGTTTGCCTCAGAGGC | |
| | CTGTGTGGGCTCCAAATTCTGGGAGCAAAGCGTGCGG | |
| | CTGGGCTCTTGGGACAGAGGAATGCAGTACAGCCACA | |
| | GCATCATCACCAACCTGCTGTACCACGTGGTGGGCTG | |
| | GACCGACTGGAACCTGGCCCTGAACCCCGAGGGAGGC | |
| | CCTAACTGGGTCAGAAACTTCGTGGACAGCCCCATCA | |
| | TTGTGGATATCACCAAGGACACGTTCTACAAGCAGCC | |
| | AATGTTCTACCACCTGGGCCACTTCAGCAAGTTCATCC | |
| | CTGAAGGCAGTCAGAGAGTGGGCCTGGTGGCCTCTCA | |
| | GAAGAACGACCTGGACGCCGTTGCGCTGATGCACCCC | |
| | GACGGCAGCGCCGTGGTGGTGGTGCTGAATAGATCTT | |
| | CTAAGGATGTGCCTCTGACAATCAAGGACCCTGCTGT | |
| | GGGATTTCTGGAAACAATCAGCCCTGGATACAGCATC | |
| | CATACATACCTGTGGCGGCGGCAGTGAGATTAATCAA | |
| | CCTCTGGATTACAAAATTTGTGAAAGATTGACTGGTAT | |
| | TCTTAACTATGTTGCTCCTTTTACGCTATGTGGATACG | |
| | CTGCTTTAATGCCTTTGTATCATGCTATTGCTTCCCGTA | |
| | TGGCTTTCATTTTCTCCTCCTTGTATAAATCCTGGTTGC | |
| | TGTCTCTTTATGAGGAGTTGTGGCCCGTTGTCAGGCAA | |
| | CGTGGCGTGGTGTGCACTGTGTTTGCTGACGCAACCCC | |
| | CACTGGTTGGGGCATTGCCACCACCTGTCAGCTCCTTT | |
| | CCGGGACTTTCGCTTTCCCCCTCCCTATTGCCACGGCG | |
| | GAACTCATCGCCGCCTGCCTTGCCCGCTGCTGGACAG | |
| | GGGCTCGGCTGTTGGGCACTGACAATTCCGTGGTGTT | |
| | GTCGGGGAAATCATCGTCCTTTCCTTGGCTGCTCGCCT | |
| | GTGTTGCCACCTGGATTCTGCGCGGGACGTCCTTCTGC | |
| | TACGTCCCTTCGGCCCTCAATCCAGCGGACCTTCCTTC | |
| | CCGCGGCCTGCTGCCGGCTCTGCGGCCTCTTCCGCGTC | |
| | TTCGCCTTCGCCCTCAGACGAGTCGGATCTCCCTTTGG | |
| | GCCGCCTCCCCGCCTGATCCTGTGCCTTCTAGTTGCCA | |
| | GCCATCTGTTGTTTGCCCCTCCCCCGTGCCTTCCTTGA | |
| | CCCTGGAAGGTGCCACTCCCACTGTCCTTTCCTAATAA | |
| | AATGAGGAAATTGCATCGCATTGTCTGAGTAGGTGTC | |
| | ATTCTATTCTGGGGGGTGGGGTGGGGCAGGACAGCAA | |
| | GGGGGAGGATTGGGAAGACAATAGCAGGCATGCTCG | |
| | AAATTAGGAACCCCTAGTGATGGAGTTGGCCACTCCC | |
| | TCTCTGCGCGCTCGCTCGCTCACTGAGGCCGCCCGGG | |
| | CAAAGCCCGGGCGTCGGGCGACCTTTGGTCGCCCGGC | |
| | CTCAGTGAGCGAGCGAGCGCGCAGAGAGGGAGTGGC | |
| | CAATCGCGACGGCCGACGTCTTTGTTACAACTTACTAT | |
| | ATATATGCACACATATATATATATTTGGGTATATTGGG | |
| | GGGGTTCTAATTTAAGAAATGCATAATTGGCTATAGA | |
| | CAGACAGTTGTCAGAACTTGGCAATGGGTACGTGCAG | |
| | GTTCATTATACCAAGTCTACTTGTAGTTGTTCAAAATG | |
| | TATCATAATACAAGGCCGGGCGAGGTCGTCACGCCTG | |
| | TAATCCCAGCATTTTGGGAGGCTAAGGCAGGAGGATT | |
| | GCTTGAGGTCAGGAGTTTGTGACCAGCCTGGGCAACA | |
| | GAGCAAGACCCTGTCTCCAAAAAGAAAAAAAATAATT | |
| | TTTTACAAAATAAAAACAAAATGTATCATCAGACGAA | |
| | ATTAAATAAGAGGCAATTCATTGTAATGACAACTTTTC | |
| | CCAGCTTGACATTTAACAAAAAGTCTAAGTCCTCTTAA | |
| | TTCATATTTAATGATCAAATATCAAATACTAATTTTTT | |
| | TTTTTTTTTTTTTTTTGAGACGGAGTCTCGCTCTGTCGC | |
| | CCAGGCTGGAGTGCAGTGGCGCGATCCTGGCTCACTG | |
| | CAAGCTCCGCCTCCCGGGTTCACGCCATTCTCCTGCCT | |
| | CAGCCTCCCGAGTAGCTGGGATTACAGACATGCGCCA | |
| | CCACGCCCGGCTAATTTTGTATTTTTAGTAGAGATGGG | |
| | GTTTCTCCATGTTGGTCAGGCTGGTCTTGAATTTCCCA | |
| | CCTCAGGTGATCTGCCTGCCTCAGCCTCACAAAGCAG | |
| | TAGCTGGGACTACAGGCACCCACCACCACACTTGGTT | |
| | AATTCTTTTGTATTTTTTTTGTAAAGACGGGATTTCAC | |
| | CATGTTAGCCAGGATGGTCTCGATCTCCTGATCTCATG | |
| | ATCCGCCCGCCTCAGCCTCCCAAAGTGCTGGGATTAC | |
| | AGGCGTGAGCCACCCCGCCCGGCCATCAAATACTAAT | |
| | TCTTAAATGGTAAGGACCCACTATTCAGAACCTGTATC | |
| | CTTATCACTAATATGCAAATATTTATTGAATACTTACT | |
| | ATGTCATGCATACTAGAGAGAGTTAGATAAATTTGAT | |
| | ACAGCTACCCTCACAGAACTTACAGTGTAATAGATGG | |
| | CATGACATGTACATGAGTAACTGTGAACAGTGTTAAA | |
| | TTGCTATTTAAAAAAAAAGACGGCTGGGCGCTGTGGC | |
| | TCATGCCTGTAATCCCAGCACTTTGGGAGGCCAAGGC | |
| | AAGTTGATCGCTCGAGGTCAAGAGTTCGAGACCAGCC | |
| | TGGCCAACGTGGTAAAACCCCGTCTCTACTAAAAATA | |
| | CAAAAAAAAAATTAGCCAGGCATGGTGGCACAGGCCT | |
| | GTAATCCCAGCTACTAGGGAGGCTGAGACATGGAGAA | |
| | CTGCTTGAATCCAGGAGGCAGAGGTTACAGTGAGCCG | |
| | AGATCATACCACTACACTCCAGCCTGAGTGACAGAGC | |
| | GAGACTCCTGTCTAAAAAAAAAAAAAAAAAAAAAGA | |
| | TACAGGTTAAGTGTTATGGTAGTTGAAGAGAGAACTC | |
| | AAACTCTGTCTCAGAAGCCTCACTTGCATGTGGACCA | |
| | CTGATATGAAATAATATAAATAGGTATAATTCAATAA | |
| | ATAGGAACTTCAGTTTTAATCATCCCAAACACCAAAA | |
| | CTTCCTATCAAACAGGTCCAATAAACTCAATCTCTATA | |
| | AGAGCTAGACAGAAATCTACTTGGTGGCCTATAATCT | |
| | TATTAGCCCTTACTTGTCCCATCTGATATTAATTAACC | |
| | CCATCTAATATGGATTAGTTAACAATCCAGTGGCTGCT | |
| | TTGACAGGAACAGTTGGAGAGAGTTGGGGATTGCAAC | |
| | ATATTCAATTATACAAAAATGCATTCAGCATCTACCTT | |
| | GATTAAGGCAGTGTGCAACAGAATTTGCAGGAGAGTA | |
| | AAAGAATGATTATAAATTTACAACCCTTAAAGAGCTA | |
| | TAGCTGGGCGTGGTGGCTCATGCCTGTAAATCCCAGC | |
| | ACTTTGGGAGGCTGAGGCGGGTGGATCACCTGAGGCC | |
| | AGAAGTTCAAGACCAGCCTAGCCAACATGGCGAAACC | |
| | CTGTCTCTACAAAAAATACAAAAATTAGCCGGGTGTG | |
| | GTGGCACGTGCCTGTAGTCCCAGTTACTTGGGAGGCC | |
| | GAGGCAGGAGAATCGCTTGAACCTAGGAGGTGGAGG | |
| | CTGCAGTGAGCCGAGATTGTGCCACTGCACTCCACTTC | |
| | AGCCTGGGCGACAAGAGCAAGACTCCGTCACAAAAA | |
| | AAAAAAAAAAAAAAAAGGCTAAAATCTAGTGGGAAA | |
| | GGCATATATACATACAACTAACTGTATAGCATAATAA | |
| | AGCTCATAATCTGTAACAAAATCTAATTCGACAAGCC | |
| | CAGAAACTTGTGATTTACCAAAAACAGTTATATATAC | |
| | ACAAAAAGTAAACCTAGAACCCAAAGTTACCCAGCAC | |
| | CAATGATTCTCTCCCTAAGCAGTATCAAGTTTAAAGCA | |
| | GTGATTACATTCTACTGCCTAGATTGTAAACTGAGTAA | |
| | AGGAGACCAGCACCTTTCTGCTACTGAACTAGCACAG | |
| | CCGTGTAAACCAACAAGGCAATGGCAGTGCCCAACTT | |
| | TCTGTATGAATATAAGTTACATCTGTTTTATTATTTGT | |
| | GACTTGGTGTTGCATGTGGTTATTATCAACACCTTCTG | |
| | AAAGAACAACTACCTGCTCAGGCTGCCATAACAAAAT | |
| | ACCACAGACTGAGTGACTTAACAGAAACTTATTTCTC | |
| | ACAGTTTTGGAGGCTGGGAAGTCCAAAATTAAGCTAA | |
| | CTGCAAGGTAGGTTTCAATCTCAGGCCTCTTCTTTGGC | |
| | TTGAAGGTCTTCTAACTGTGTGCTCACATGACCTCTTC | |
| | TAACAAGCTCTCTGGTGTCTCTTTTTTTTTTTTTTTCTTT | |
| | TTTGAGACAGAGTCTCACTCTGTCACCCAGGCTGGAG | |
| | TACAGTGGCACAATCTGGGCTCACTGCAACCTCCAAC | |
| | TCCCGGGTTCAAGTGATTCTCATGCCTCACCCTCCCGA | |
| | GTAGCTTGGATGACAGGAGCCCGCTACCACACCCAGC | |
| | TAATTTTTGTATTTTTAGTAGAGATGGTGTTTCACTAC | |
| | ATTGGCCAGGCTGGTCTCAAACTCCTGACCTCGTGATC | |
| | CACCCACCTTGGCCTCCCAAAGTGCTGGGATTACAGG | |
| | TGTGAGCCACTGCGCCCGTCCTGGTGTCTTTTCATATA | |
| | AGGGCACTAATCCAATCAGACCTGGGCCCAACCCTCC | |
| | CGACTTCTTCTAACTGTAATTACCTTCCAAAGGCCCTG | |
| | TCTCCAAATACCATCACACTGGGGGTTAGGACTTCAA | |
| | AAAAGGTATGGGGGGGGTGTGGGAGGACATAAATGC | |
| | TCAGTCCATAACAAGCACCCAACATAAAAATGGCTAG | |
| | AACAGATCACAAAAAAAAGGTCCTGTATGGCTTTGGG | |
| | GAAGGGCTCAACCCCAAAATATCTGAAAGCTCTGGAG | |
| | GGGCCTAGAAGTGGTAAATGAATGAAAACGTGGTTAC | |
| | TCTCCCGATCTGCCTTTCCCAAATATGGCCATTCTTGG | |
| | CTGAATCAGAAATCAAAGGACAGGTTATTAATTACTA | |
| | GCTCTAAGTTACTTACCATTTGCTGAGACAGTTCAGAA | |
| | ATCTGACTGCATCTCCTCAGGGATCTAGAACACAGTTC | |
| | TCAAATTCTAACTTACTTGTGATATACTTGTGAATGAT | |
| | AAAAATCGCTACAGGTACTTTTATTAATCTGAAAGAG | |
| | TATTGAGAAATTACCTTTCATTCTGACTTTTGTCTGGA | |
| | ATGAAAATCAATACTTTTGCTATATTCCATTACTGAAA | |
| | TAATTTTACTTTCCAGTAAAACTGGCATTATAATTTTT | |
| | TTTAATTTTTAAAACTTCATAATTTTTTGCCAGACTGA | |
| | CCCATGTAAACATACAAATTACTAATAATTATGCACG | |
| | TCACATCTGTAATAATGGCCTTCATGTAAACATTTTTG | |
| | TGGTTTACACATAAAATCTCTAATTACAAAGCTATATT | |
| | ATCTAAAATTACAGTAAGCAAGAAAATTAATCCAAGC | |
| | TAAGACAATACTTGCAACATCAATTCATCATCTGTGAC | |
| | AAGGACTGCTTAAGTCTCTTTGTGGTTGACGTCATTAA | |
| | TTAACTGGCCTCATGGGCCTTCCGCTCACTGCCCGCTT | |
| | TCCAGTCGGGAAACCTGTCGTGCCAGCTGCATTAACA | |
| | TGGTCATAGCTGTTTCCTTGCGTATTGGGCGCTCTCCG | |
| | CTTCCTCGCTCACTGACTCGCTGCGCTCGGTCGTTCGG | |
| | GTAAAGCCTGGGGTGCCTAATGAGCAAAAGGCCAGCA | |
| | AAAGGCCAGGAACCGTAAAAAGGCCGCGTTGCTGGC | |
| | GTTTTTCCATAGGCTCCGCCCCCCTGACGAGCATCACA | |
| | AAAATCGACGCTCAAGTCAGAGGTGGCGAAACCCGAC | |
| | AGGACTATAAAGATACCAGGCGTTTCCCCCTGGAAGC | |
| | TCCCTCGTGCGCTCTCCTGTTCCGACCCTGCCGCTTAC | |
| | CGGATACCTGTCCGCCTTTCTCCCTTCGGGAAGCGTGG | |
| | CGCTTTCTCATAGCTCACGCTGTAGGTATCTCAGTTCG | |
| | GTGTAGGTCGTTCGCTCCAAGCTGGGCTGTGTGCACG | |
| | AACCCCCCGTTCAGCCCGACCGCTGCGCCTTATCCGGT | |
| | AACTATCGTCTTGAGTCCAACCCGGTAAGACACGACT | |
| | TATCGCCACTGGCAGCAGCCACTGGTAACAGGATTAG | |
| | CAGAGCGAGGTATGTAGGCGGTGCTACAGAGTTCTTG | |
| | AAGTGGTGGCCTAACTACGGCTACACTAGAAGAACAG | |
| | TATTTGGTATCTGCGCTCTGCTGAAGCCAGTTACCTTC | |
| | GGAAAAAGAGTTGGTAGCTCTTGATCCGGCAAACAAA | |
| | CCACCGCTGGTAGCGGTGGTTTTTTTGTTTGCAAGCAG | |
| | CAGATTACGCGCAGAAAAAAAGGATCTCAAGAAGAT | |
| | CCTTTGATCTTTTCTACGGGGTCTGACGCTCAGTGGAA | |
| | CGAAAACTCACGTTAAGGGATTTTGGTCATGAGATTA | |
| | TCAAAAAGGATCTTCACCTAGATCCTTTTAAATTAAAA | |
| | ATGAAGTTTTAAATCAATCTAAAGTATATATGAGTAA | |
| | ACTTGGTCTGACAGTTACCAATGCTTAATCAGTGAGG | |
| | CACCTATCTCAGCGATCTGTCTATTTCGTTCATCCATA | |
| | GTTGCCTGACTCCCCGTCGTGTAGATAACTACGATACG | |
| | GGAGGGCTTACCATCTGGCCCCAGTGCTGCAATGATA | |
| | CCGCGAGAACCACGCTCACCGGCTCCAGATTTATCAG | |
| | CAATAAACCAGCCAGCCGGAAGGGCCGAGCGCAGAA | |
| | GTGGTCCTGCAACTTTATCCGCCTCCATCCAGTCTATT | |
| | AATTGTTGCCGGGAAGCTAGAGTAAGTAGTTCGCCAG | |
| | TTAATAGTTTGCGCAACGTTGTTGCCATTGCTACAGGC | |
| | ATCGTGGTGTCACGCTCGTCGTTTGGTATGGCTTCATT | |
| | CAGCTCCGGTTCCCAACGATCAAGGCGAGTTACATGA | |
| | TCCCCCATGTTGTGCAAAAAAGCGGTTAGCTCCTTCGG | |
| | TCCTCCGATCGTTGTCAGAAGTAAGTTGGCCGCAGTGT | |
| | TATCACTCATGGTTATGGCAGCACTGCATAATTCTCTT | |
| | ACTGTCATGCCATCCGTAAGATGCTTTTCTGTGACTGG | |
| | TGAGTACTCAACCAAGTCATTCTGAGAATAGTGTATG | |
| | CGGCGACCGAGTTGCTCTTGCCCGGCGTCAATACGGG | |
| | ATAATACCGCGCCACATAGCAGAACTTTAAAAGTGCT | |
| | CATCATTGGAAAACGTTCTTCGGGGCGAAAACTCTCA | |
| | AGGATCTTACCGCTGTTGAGATCCAGTTCGATGTAACC | |
| | CACTCGTGCACCCAACTGATCTTCAGCATCTTTTACTT | |
| | TCACCAGCGTTTCTGGGTGAGCAAAAACAGGAAGGCA | |
| | AAATGCCGCAAAAAAGGGAATAAGGGCGACACGGAA | |
| | ATGTTGAATACTCATACTCTTCCTTTTTCAATATTATTG | |
| | AAGCATTTATCAGGGTTATTGTCTCATGAGCGGATAC | |
| | ATATTTGAATGTATTTAGAAAAATAAACAAATAGGGG | |
| | TTCCGCGCACATTTCCCCGAAAAGTGCCACCTAAATTG | |
| | TAAGCGTTAATATTTTGTTAAAATTCGCGTTAAATTTT | |
| | TGTTAAATCAGCTCATTTTTTAACCAATAGGCCGAAAT | |
| | CGGCAAAATCCCTTATAAATCAAAAGAATAGACCGAG | |
| | ATAGGGTTGAGTGGCCGCTACAGGGCGCTCCCATTCG | |
| | CCATTCAGGCTGCGCAACTGTTGGGAAGGGCGTTTCG | |
| | GTGCGGGCCTCTTCGCTATTACGCCAGCTGGCGAAAG | |
| | GGGGATGTGCTGCAAGGCGATTAAGTTGGGTAACGCC | |
| | AGGGTTTTCCCAGTCACGACGTTGTAAAACGACGGCC | |
| | AGTGAGCGCGACGTAATACGACTCACTATAGGGCGAA | |
| | TTGGCGGAAGGCCGTCAAGGCCTAGGCGCGCCAACGC | |
| | GT | |
| 20 | TTGGCCACTCCCTCTCTGCGCGCTCGCTCGCTCACTGA | Full DNA sequence |
| | GGCCGCCCGGGCAAAGCCCGGGCGTCGGGCGACCTTT | of vector (pAAV- |
| | GGTCGCCCGGCCTCAGTGAGCGAGCGAGCGCGCAGAG | CBA-SS1-GBA- |
| | AGGGAGTGGCCAACTCCATCACTAGGGGTTCCTTACG | WPRE-bGH); |
| | TACAATTGGGATCCCGGACCGTCGACATTGATTATTG | coding sequence for |
| | ACTAGTTATTAATAGTAATCAATTACGGGGTCATTAGT | GBA1 is underlined |
| | TCATAGCCCATATATGGAGTTCCGCGTTACATAACTTA | Underlined, codon |
| | CGGTAAATGGCCCGCCTGGCTGACCGCCCAACGACCC | Optimized GBA1 |
| | CCGCCCATTGACGTCAATAATGACGTATGTTCCCATAG | DNA sequence |
| | TAACGCCAATAGGGACTTTCCATTGACGTCAATGGGT | (Human) |
| | GGAGTATTTACGGTAAACTGCCCACTTGGCAGTACAT | |
| | CAAGTGTATCATATGCCAAGTACGCCCCCTATTGACGT | |
| | CAATGACGGTAAATGGCCCGCCTGGCATTATGCCCAG | |
| | TACATGACCTTATGGGACTTTCCTACTTGGCAGTACAT | |
| | CTACGTATTAGTCATCGCTATTACCATGGTCGAGGTGA | |
| | GCCCCACGTTCTGCTTCACTCTCCCCATCTCCCCCCCC | |
| | TCCCCACCCCCAATTTTGTATTTATTTATTTTTTAATTA | |
| | TTTTGTGCAGCGATGGGGGCGGGGGGGGGGGGGGGG | |
| | CGCGCGCCAGGCGGGGCGGGGCGGGGCGAGGGGCGG | |
| | GGCGGGGCGAGGCGGAGAGGTGCGGCGGCAGCCAAT | |
| | CAGAGCGGCGCGCTCCGAAAGTTTCCTTTTATGGCGA | |
| | GGCGGCGGCGGCGGCGGCCCTATAAAAAGCGAAGCG | |
| | CGCGGCGGGCGGGAGTCGCTGCGCGCTGCCTTCGCCC | |
| | CGTGCCCCGCTCCGCCGCCGCCTCGCGCCGCCCGCCC | |
| | CGGCTCTGACTGACCGCGTTACTCCCACAGGTGAGCG | |
| | GGCGGGACGGCCCTTCTCCTCCGGGCTGTAATTAGCG | |
| | CTTGGTTTAATGACGGCTTGTTTCTTTTCTGTGGCTGC | |
| | GTGAAAGCCTTGAGGGGCTCCGGGAGGGCCCTTTGTG | |
| | CGGGGGGAGCGGCTCGGGGGGTGCGTGCGTGTGTGTG | |
| | TGCGTGGGGAGCGCCGCGTGCGGCTCCGCGCTGCCCG | |
| | GCGGCTGTGAGCGCTGCGGGCGCGGCGCGGGGCTTTG | |
| | TGCGCTCCGCAGTGTGCGCGAGGGGAGCGCGGCCGGG | |
| | GGCGGTGCCCCGCGGTGCGGGGGGGGCTGCGAGGGG | |
| | AACAAAGGCTGCGTGCGGGGTGTGTGCGTGGGGGGGT | |
| | GAGCAGGGGGTGTGGGCGCGTCGGTCGGGCTGCAACC | |
| | CCCCCTGCACCCCCCTCCCCGAGTTGCTGAGCACGGC | |
| | CCGGCTTCGGGTGCGGGGCTCCGTACGGGGCGTGGCG | |
| | CGGGGCTCGCCGTGCCGGGCGGGGGGTGGCGGCAGGT | |
| | GGGGGTGCCGGGCGGGGCGGGGCCGCCTCGGGCCGG | |
| | GGAGGGCTCGGGGGAGGGGCGCGGCGGCCCCCGGAG | |
| | CGCCGGCGGCTGTCGAGGCGCGGCGAGCCGCAGCCAT | |
| | TGCCTTTTATGGTAATCGTGCGAGAGGGCGCAGGGAC | |
| | TTCCTTTGTCCCAAATCTGTGCGGAGCCGAAATCTGGG | |
| | AGGCGCCGCCGCACCCCCTCTAGCGGGCGCGGGGCGA | |
| | AGCGGTGCGGCGCCGGCAGGAAGGAAATGGGCGGGG | |
| | AGGGCCTTCGTGCGTCGCCGCGCCGCCGTCCCCTTCTC | |
| | CCTCTCCAGCCTCGGGGCTGTCCGCGGGGGGACGGCT | |
| | GCCTTCGGGGGGGACGGGGCAGGGCGGGGTTCGGCTT | |
| | CTGGCGTGTGACCGGCGGCTCTAGAGCCTCTGCTAAC | |
| | CATGTTCATGCCTTCTTCTTTTTCCTACAGCTCCTGGGC | |
| | AACGTGCTGGTTATTGTGCTGTCTCATCATTTTGGCAA | |
| | AGAATTCGCCACCATGGCCTCTCTGTGGCTGCTGAGCT | |
| | GCTTCAGCCTGGTGGGCGCCGCTTTCGGCGCCAGACC | |
| | TTGTATCCCCAAGAGCTTCGGATATAGCAGCGTGGTCT | |
| | GCGTGTGCAACGCCACCTACTGCGATAGCTTCGACCC | |
| | ACCTACCTTTCCAGCTCTGGGCACCTTCTCCAGATACG | |
| | AGTCTACAAGAAGCGGCAGAAGAATGGAACTGTCCAT | |
| | GGGCCCTATCCAGGCCAACCACACCGGCACAGGCCTC | |
| | CTGTTGACCCTGCAGCCCGAGCAGAAATTTCAGAAAG | |
| | TGAAGGGATTCGGCGGCGCCATGACCGATGCCGCCGC | |
| | TCTGAATATCCTGGCGCTGAGCCCTCCTGCCCAGAACC | |
| | TGCTGCTGAAGAGCTACTTTAGCGAGGAGGGGATCGG | |
| | CTACAACATTATCAGAGTGCCCATGGCCAGCTGCGAC | |
| | TTTAGCATCAGAACCTATACATACGCCGACACCCCAG | |
| | ATGACTTCCAGCTGCACAACTTCAGCCTGCCTGAGGA | |
| | AGATACAAAGCTGAAAATCCCCCTGATCCACCGGGCC | |
| | CTGCAACTGGCTCAGCGGCCAGTGTCCCTGCTGGCCTC | |
| | TCCTTGGACCAGCCCTACCTGGCTGAAGACCAATGGC | |
| | GCCGTGAACGGCAAGGGCTCTCTGAAGGGCCAGCCTG | |
| | GCGACATCTACCATCAGACCTGGGCCAGATACTTCGT | |
| | GAAGTTCCTGGATGCCTACGCTGAGCACAAGCTGCAG | |
| | TTCTGGGCCGTGACCGCCGAGAACGAGCCTTCCGCCG | |
| | GACTGCTCAGCGGCTATCCTTTCCAGTGCCTGGGCTTC | |
| | ACACCTGAGCACCAGCGCGACTTCATCGCCAGGGACC | |
| | TGGGCCCTACACTGGCCAATAGCACACACCACAACGT | |
| | GAGACTGCTGATGCTGGATGATCAGAGACTGCTGCTT | |
| | CCCCACTGGGCTAAGGTCGTGCTGACCGACCCTGAAG | |
| | CCGCTAAGTACGTGCACGGCATCGCCGTGCACTGGTA | |
| | CCTGGACTTCCTGGCCCCTGCCAAGGCCACCCTGGGA | |
| | GAAACCCACCGGCTGTTCCCTAACACCATGCTGTTTGC | |
| | CTCAGAGGCCTGTGTGGGCTCCAAATTCTGGGAGCAA | |
| | AGCGTGCGGCTGGGCTCTTGGGACAGAGGAATGCAGT | |
| | ACAGCCACAGCATCATCACCAACCTGCTGTACCACGT | |
| | GGTGGGCTGGACCGACTGGAACCTGGCCCTGAACCCC | |
| | GAGGGAGGCCCTAACTGGGTCAGAAACTTCGTGGACA | |
| | GCCCCATCATTGTGGATATCACCAAGGACACGTTCTA | |
| | CAAGCAGCCAATGTTCTACCACCTGGGCCACTTCAGC | |
| | AAGTTCATCCCTGAAGGCAGTCAGAGAGTGGGCCTGG | |
| | TGGCCTCTCAGAAGAACGACCTGGACGCCGTTGCGCT | |
| | GATGCACCCCGACGGCAGCGCCGTGGTGGTGGTGCTG | |
| | AATAGATCTTCTAAGGATGTGCCTCTGACAATCAAGG | |
| | ACCCTGCTGTGGGATTTCTGGAAACAATCAGCCCTGG | |
| | ATACAGCATCCATACATACCTGTGGCGGCGGCAGTGA | |
| | GATTAATCAACCTCTGGATTACAAAATTTGTGAAAGA | |
| | TTGACTGGTATTCTTAACTATGTTGCTCCTTTTACGCTA | |
| | TGTGGATACGCTGCTTTAATGCCTTTGTATCATGCTAT | |
| | TGCTTCCCGTATGGCTTTCATTTTCTCCTCCTTGTATAA | |
| | ATCCTGGTTGCTGTCTCTTTATGAGGAGTTGTGGCCCG | |
| | TTGTCAGGCAACGTGGCGTGGTGTGCACTGTGTTTGCT | |
| | GACGCAACCCCCACTGGTTGGGGCATTGCCACCACCT | |
| | GTCAGCTCCTTTCCGGGACTTTCGCTTTCCCCCTCCCT | |
| | ATTGCCACGGCGGAACTCATCGCCGCCTGCCTTGCCC | |
| | GCTGCTGGACAGGGGCTCGGCTGTTGGGCACTGACAA | |
| | TTCCGTGGTGTTGTCGGGGAAATCATCGTCCTTTCCTT | |
| | GGCTGCTCGCCTGTGTTGCCACCTGGATTCTGCGCGGG | |
| | ACGTCCTTCTGCTACGTCCCTTCGGCCCTCAATCCAGC | |
| | GGACCTTCCTTCCCGCGGCCTGCTGCCGGCTCTGCGGC | |
| | CTCTTCCGCGTCTTCGCCTTCGCCCTCAGACGAGTCGG | |
| | ATCTCCCTTTGGGCCGCCTCCCCGCCTGATCCTGTGCC | |
| | TTCTAGTTGCCAGCCATCTGTTGTTTGCCCCTCCCCCG | |
| | TGCCTTCCTTGACCCTGGAAGGTGCCACTCCCACTGTC | |
| | CTTTCCTAATAAAATGAGGAAATTGCATCGCATTGTCT | |
| | GAGTAGGTGTCATTCTATTCTGGGGGGTGGGGTGGGG | |
| | CAGGACAGCAAGGGGGAGGATTGGGAAGACAATAGC | |
| | AGGCATGCTCGAAATTAGGAACCCCTAGTGATGGAGT | |
| | TGGCCACTCCCTCTCTGCGCGCTCGCTCGCTCACTGAG | |
| | GCCGCCCGGGCAAAGCCCGGGCGTCGGGCGACCTTTG | |
| | GTCGCCCGGCCTCAGTGAGCGAGCGAGCGCGCAGAGA | |
| | GGGAGTGGCCAATCGCGACGGCCGACGTCTTTGTTAC | |
| | AACTTACTATATATATGCACACATATATATATATTTGG | |
| | GTATATTGGGGGGGTTCTAATTTAAGAAATGCATAAT | |
| | TGGCTATAGACAGACAGTTGTCAGAACTTGGCAATGG | |
| | GTACGTGCAGGTTCATTATACCAAGTCTACTTGTAGTT | |
| | GTTCAAAATGTATCATAATACAAGGCCGGGCGAGGTC | |
| | GTCACGCCTGTAATCCCAGCATTTTGGGAGGCTAAGG | |
| | CAGGAGGATTGCTTGAGGTCAGGAGTTTGTGACCAGC | |
| | CTGGGCAACAGAGCAAGACCCTGTCTCCAAAAAGAAA | |
| | AAAAATAATTTTTTACAAAATAAAAACAAAATGTATC | |
| | ATCAGACGAAATTAAATAAGAGGCAATTCATTGTAAT | |
| | GACAACTTTTCCCAGCTTGACATTTAACAAAAAGTCTA | |
| | AGTCCTCTTAATTCATATTTAATGATCAAATATCAAAT | |
| | ACTAATTTTTTTTTTTTTTTTTTTTTTGAGACGGAGTCT | |
| | CGCTCTGTCGCCCAGGCTGGAGTGCAGTGGCGCGATC | |
| | CTGGCTCACTGCAAGCTCCGCCTCCCGGGTTCACGCC | |
| | ATTCTCCTGCCTCAGCCTCCCGAGTAGCTGGGATTACA | |
| | GACATGCGCCACCACGCCCGGCTAATTTTGTATTTTTA | |
| | GTAGAGATGGGGTTTCTCCATGTTGGTCAGGCTGGTCT | |
| | TGAATTTCCCACCTCAGGTGATCTGCCTGCCTCAGCCT | |
| | CACAAAGCAGTAGCTGGGACTACAGGCACCCACCACC | |
| | ACACTTGGTTAATTCTTTTGTATTTTTTTTGTAAAGACG | |
| | GGATTTCACCATGTTAGCCAGGATGGTCTCGATCTCCT | |
| | GATCTCATGATCCGCCCGCCTCAGCCTCCCAAAGTGCT | |
| | GGGATTACAGGCGTGAGCCACCCCGCCCGGCCATCAA | |
| | ATACTAATTCTTAAATGGTAAGGACCCACTATTCAGA | |
| | ACCTGTATCCTTATCACTAATATGCAAATATTTATTGA | |
| | ATACTTACTATGTCATGCATACTAGAGAGAGTTAGAT | |
| | AAATTTGATACAGCTACCCTCACAGAACTTACAGTGT | |
| | AATAGATGGCATGACATGTACATGAGTAACTGTGAAC | |
| | AGTGTTAAATTGCTATTTAAAAAAAAAGACGGCTGGG | |
| | CGCTGTGGCTCATGCCTGTAATCCCAGCACTTTGGGAG | |
| | GCCAAGGCAAGTTGATCGCTCGAGGTCAAGAGTTCGA | |
| | GACCAGCCTGGCCAACGTGGTAAAACCCCGTCTCTAC | |
| | TAAAAATACAAAAAAAAAATTAGCCAGGCATGGTGG | |
| | CACAGGCCTGTAATCCCAGCTACTAGGGAGGCTGAGA | |
| | CATGGAGAACTGCTTGAATCCAGGAGGCAGAGGTTAC | |
| | AGTGAGCCGAGATCATACCACTACACTCCAGCCTGAG | |
| | TGACAGAGCGAGACTCCTGTCTAAAAAAAAAAAAAA | |
| | AAAAAAAGATACAGGTTAAGTGTTATGGTAGTTGAAG | |
| | AGAGAACTCAAACTCTGTCTCAGAAGCCTCACTTGCA | |
| | TGTGGACCACTGATATGAAATAATATAAATAGGTATA | |
| | ATTCAATAAATAGGAACTTCAGTTTTAATCATCCCAAA | |
| | CACCAAAACTTCCTATCAAACAGGTCCAATAAACTCA | |
| | ATCTCTATAAGAGCTAGACAGAAATCTACTTGGTGGC | |
| | CTATAATCTTATTAGCCCTTACTTGTCCCATCTGATATT | |
| | AATTAACCCCATCTAATATGGATTAGTTAACAATCCA | |
| | GTGGCTGCTTTGACAGGAACAGTTGGAGAGAGTTGGG | |
| | GATTGCAACATATTCAATTATACAAAAATGCATTCAG | |
| | CATCTACCTTGATTAAGGCAGTGTGCAACAGAATTTG | |
| | CAGGAGAGTAAAAGAATGATTATAAATTTACAACCCT | |
| | TAAAGAGCTATAGCTGGGCGTGGTGGCTCATGCCTGT | |
| | AAATCCCAGCACTTTGGGAGGCTGAGGCGGGTGGATC | |
| | ACCTGAGGCCAGAAGTTCAAGACCAGCCTAGCCAACA | |
| | TGGCGAAACCCTGTCTCTACAAAAAATACAAAAATTA | |
| | GCCGGGTGTGGTGGCACGTGCCTGTAGTCCCAGTTAC | |
| | TTGGGAGGCCGAGGCAGGAGAATCGCTTGAACCTAGG | |
| | AGGTGGAGGCTGCAGTGAGCCGAGATTGTGCCACTGC | |
| | ACTCCACTTCAGCCTGGGCGACAAGAGCAAGACTCCG | |
| | TCACAAAAAAAAAAAAAAAAAAAAAGGCTAAAATCT | |
| | AGTGGGAAAGGCATATATACATACAACTAACTGTATA | |
| | GCATAATAAAGCTCATAATCTGTAACAAAATCTAATT | |
| | CGACAAGCCCAGAAACTTGTGATTTACCAAAAACAGT | |
| | TATATATACACAAAAAGTAAACCTAGAACCCAAAGTT | |
| | ACCCAGCACCAATGATTCTCTCCCTAAGCAGTATCAA | |
| | GTTTAAAGCAGTGATTACATTCTACTGCCTAGATTGTA | |
| | AACTGAGTAAAGGAGACCAGCACCTTTCTGCTACTGA | |
| | ACTAGCACAGCCGTGTAAACCAACAAGGCAATGGCAG | |
| | TGCCCAACTTTCTGTATGAATATAAGTTACATCTGTTT | |
| | TATTATTTGTGACTTGGTGTTGCATGTGGTTATTATCA | |
| | ACACCTTCTGAAAGAACAACTACCTGCTCAGGCTGCC | |
| | ATAACAAAATACCACAGACTGAGTGACTTAACAGAAA | |
| | CTTATTTCTCACAGTTTTGGAGGCTGGGAAGTCCAAAA | |
| | TTAAGCTAACTGCAAGGTAGGTTTCAATCTCAGGCCTC | |
| | TTCTTTGGCTTGAAGGTCTTCTAACTGTGTGCTCACAT | |
| | GACCTCTTCTAACAAGCTCTCTGGTGTCTCTTTTTTTTT | |
| | TTTTTTCTTTTTTGAGACAGAGTCTCACTCTGTCACCCA | |
| | GGCTGGAGTACAGTGGCACAATCTGGGCTCACTGCAA | |
| | CCTCCAACTCCCGGGTTCAAGTGATTCTCATGCCTCAC | |
| | CCTCCCGAGTAGCTTGGATGACAGGAGCCCGCTACCA | |
| | CACCCAGCTAATTTTTGTATTTTTAGTAGAGATGGTGT | |
| | TTCACTACATTGGCCAGGCTGGTCTCAAACTCCTGACC | |
| | TCGTGATCCACCCACCTTGGCCTCCCAAAGTGCTGGG | |
| | ATTACAGGTGTGAGCCACTGCGCCCGTCCTGGTGTCTT | |
| | TTCATATAAGGGCACTAATCCAATCAGACCTGGGCCC | |
| | AACCCTCCCGACTTCTTCTAACTGTAATTACCTTCCAA | |
| | AGGCCCTGTCTCCAAATACCATCACACTGGGGGTTAG | |
| | GACTTCAAAAAAGGTATGGGGGGGGTGTGGGAGGAC | |
| | ATAAATGCTCAGTCCATAACAAGCACCCAACATAAAA | |
| | ATGGCTAGAACAGATCACAAAAAAAAGGTCCTGTATG | |
| | GCTTTGGGGAAGGGCTCAACCCCAAAATATCTGAAAG | |
| | CTCTGGAGGGGCCTAGAAGTGGTAAATGAATGAAAAC | |
| | GTGGTTACTCTCCCGATCTGCCTTTCCCAAATATGGCC | |
| | ATTCTTGGCTGAATCAGAAATCAAAGGACAGGTTATT | |
| | AATTACTAGCTCTAAGTTACTTACCATTTGCTGAGACA | |
| | GTTCAGAAATCTGACTGCATCTCCTCAGGGATCTAGA | |
| | ACACAGTTCTCAAATTCTAACTTACTTGTGATATACTT | |
| | GTGAATGATAAAAATCGCTACAGGTACTTTTATTAATC | |
| | TGAAAGAGTATTGAGAAATTACCTTTCATTCTGACTTT | |
| | TGTCTGGAATGAAAATCAATACTTTTGCTATATTCCAT | |
| | TACTGAAATAATTTTACTTTCCAGTAAAACTGGCATTA | |
| | TAATTTTTTTTAATTTTTAAAACTTCATAATTTTTTGCC | |
| | AGACTGACCCATGTAAACATACAAATTACTAATAATT | |
| | ATGCACGTCACATCTGTAATAATGGCCTTCATGTAAAC | |
| | ATTTTTGTGGTTTACACATAAAATCTCTAATTACAAAG | |
| | CTATATTATCTAAAATTACAGTAAGCAAGAAAATTAA | |
| | TCCAAGCTAAGACAATACTTGCAACATCAATTCATCA | |
| | TCTGTGACAAGGACTGCTTAAGTCTCTTTGTGGTTGAC | |
| | GTCATTAATTAACTGGCCTCATGGGCCTTCCGCTCACT | |
| | GCCCGCTTTCCAGTCGGGAAACCTGTCGTGCCAGCTG | |
| | CATTAACATGGTCATAGCTGTTTCCTTGCGTATTGGGC | |
| | GCTCTCCGCTTCCTCGCTCACTGACTCGCTGCGCTCGG | |
| | TCGTTCGGGTAAAGCCTGGGGTGCCTAATGAGCAAAA | |
| | GGCCAGCAAAAGGCCAGGAACCGTAAAAAGGCCGCG | |
| | TTGCTGGCGTTTTTCCATAGGCTCCGCCCCCCTGACGA | |
| | GCATCACAAAAATCGACGCTCAAGTCAGAGGTGGCGA | |
| | AACCCGACAGGACTATAAAGATACCAGGCGTTTCCCC | |
| | CTGGAAGCTCCCTCGTGCGCTCTCCTGTTCCGACCCTG | |
| | CCGCTTACCGGATACCTGTCCGCCTTTCTCCCTTCGGG | |
| | AAGCGTGGCGCTTTCTCATAGCTCACGCTGTAGGTATC | |
| | TCAGTTCGGTGTAGGTCGTTCGCTCCAAGCTGGGCTGT | |
| | GTGCACGAACCCCCCGTTCAGCCCGACCGCTGCGCCT | |
| | TATCCGGTAACTATCGTCTTGAGTCCAACCCGGTAAG | |
| | ACACGACTTATCGCCACTGGCAGCAGCCACTGGTAAC | |
| | AGGATTAGCAGAGCGAGGTATGTAGGCGGTGCTACAG | |
| | AGTTCTTGAAGTGGTGGCCTAACTACGGCTACACTAG | |
| | AAGAACAGTATTTGGTATCTGCGCTCTGCTGAAGCCA | |
| | GTTACCTTCGGAAAAAGAGTTGGTAGCTCTTGATCCG | |
| | GCAAACAAACCACCGCTGGTAGCGGTGGTTTTTTTGTT | |
| | TGCAAGCAGCAGATTACGCGCAGAAAAAAAGGATCTC | |
| | AAGAAGATCCTTTGATCTTTTCTACGGGGTCTGACGCT | |
| | CAGTGGAACGAAAACTCACGTTAAGGGATTTTGGTCA | |
| | TGAGATTATCAAAAAGGATCTTCACCTAGATCCTTTTA | |
| | AATTAAAAATGAAGTTTTAAATCAATCTAAAGTATAT | |
| | ATGAGTAAACTTGGTCTGACAGTTACCAATGCTTAATC | |
| | AGTGAGGCACCTATCTCAGCGATCTGTCTATTTCGTTC | |
| | ATCCATAGTTGCCTGACTCCCCGTCGTGTAGATAACTA | |
| | CGATACGGGAGGGCTTACCATCTGGCCCCAGTGCTGC | |
| | AATGATACCGCGAGAACCACGCTCACCGGCTCCAGAT | |
| | TTATCAGCAATAAACCAGCCAGCCGGAAGGGCCGAGC | |
| | GCAGAAGTGGTCCTGCAACTTTATCCGCCTCCATCCAG | |
| | TCTATTAATTGTTGCCGGGAAGCTAGAGTAAGTAGTTC | |
| | GCCAGTTAATAGTTTGCGCAACGTTGTTGCCATTGCTA | |
| | CAGGCATCGTGGTGTCACGCTCGTCGTTTGGTATGGCT | |
| | TCATTCAGCTCCGGTTCCCAACGATCAAGGCGAGTTA | |
| | CATGATCCCCCATGTTGTGCAAAAAAGCGGTTAGCTC | |
| | CTTCGGTCCTCCGATCGTTGTCAGAAGTAAGTTGGCCG | |
| | CAGTGTTATCACTCATGGTTATGGCAGCACTGCATAAT | |
| | TCTCTTACTGTCATGCCATCCGTAAGATGCTTTTCTGT | |
| | GACTGGTGAGTACTCAACCAAGTCATTCTGAGAATAG | |
| | TGTATGCGGCGACCGAGTTGCTCTTGCCCGGCGTCAAT | |
| | ACGGGATAATACCGCGCCACATAGCAGAACTTTAAAA | |
| | GTGCTCATCATTGGAAAACGTTCTTCGGGGCGAAAAC | |
| | TCTCAAGGATCTTACCGCTGTTGAGATCCAGTTCGATG | |
| | TAACCCACTCGTGCACCCAACTGATCTTCAGCATCTTT | |
| | TACTTTCACCAGCGTTTCTGGGTGAGCAAAAACAGGA | |
| | AGGCAAAATGCCGCAAAAAAGGGAATAAGGGCGACA | |
| | CGGAAATGTTGAATACTCATACTCTTCCTTTTTCAATA | |
| | TTATTGAAGCATTTATCAGGGTTATTGTCTCATGAGCG | |
| | GATACATATTTGAATGTATTTAGAAAAATAAACAAAT | |
| | AGGGGTTCCGCGCACATTTCCCCGAAAAGTGCCACCT | |
| | AAATTGTAAGCGTTAATATTTTGTTAAAATTCGCGTTA | |
| | AATTTTTGTTAAATCAGCTCATTTTTTAACCAATAGGC | |
| | CGAAATCGGCAAAATCCCTTATAAATCAAAAGAATAG | |
| | ACCGAGATAGGGTTGAGTGGCCGCTACAGGGCGCTCC | |
| | CATTCGCCATTCAGGCTGCGCAACTGTTGGGAAGGGC | |
| | GTTTCGGTGCGGGCCTCTTCGCTATTACGCCAGCTGGC | |
| | GAAAGGGGGATGTGCTGCAAGGCGATTAAGTTGGGTA | |
| | ACGCCAGGGTTTTCCCAGTCACGACGTTGTAAAACGA | |
| | CGGCCAGTGAGCGCGACGTAATACGACTCACTATAGG | |
| | GCGAATTGGCGGAAGGCCGTCAAGGCCTAGGCGCGCC | |
| | AACGCGT | |
| 21 | TTGGCCACTCCCTCTCTGCGCGCTCGCTCGCTCACTGA | Full DNA sequence |
| | GGCCGCCCGGGCAAAGCCCGGGCGTCGGGCGACCTTT | of vector (pAAV- |
| | GGTCGCCCGGCCTCAGTGAGCGAGCGAGCGCGCAGAG | CBA-SS2-GBA- |
| | AGGGAGTGGCCAACTCCATCACTAGGGGTTCCTTACG | WPRE-bGH); |
| | TACAATTGGGATCCCGGACCGTCGACATTGATTATTG | coding sequence for |
| | ACTAGTTATTAATAGTAATCAATTACGGGGTCATTAGT | GBA1 is underlined |
| | TCATAGCCCATATATGGAGTTCCGCGTTACATAACTTA | Underlined, codon |
| | CGGTAAATGGCCCGCCTGGCTGACCGCCCAACGACCC | Optimized GBA1 |
| | CCGCCCATTGACGTCAATAATGACGTATGTTCCCATAG | DNA sequence |
| | TAACGCCAATAGGGACTTTCCATTGACGTCAATGGGT | (Human) |
| | GGAGTATTTACGGTAAACTGCCCACTTGGCAGTACAT | |
| | CAAGTGTATCATATGCCAAGTACGCCCCCTATTGACGT | |
| | CAATGACGGTAAATGGCCCGCCTGGCATTATGCCCAG | |
| | TACATGACCTTATGGGACTTTCCTACTTGGCAGTACAT | |
| | CTACGTATTAGTCATCGCTATTACCATGGTCGAGGTGA | |
| | GCCCCACGTTCTGCTTCACTCTCCCCATCTCCCCCCCC | |
| | TCCCCACCCCCAATTTTGTATTTATTTATTTTTTAATTA | |
| | TTTTGTGCAGCGATGGGGGCGGGGGGGGGGGGGGGG | |
| | CGCGCGCCAGGCGGGGCGGGGCGGGGCGAGGGGCGG | |
| | GGCGGGGCGAGGCGGAGAGGTGCGGCGGCAGCCAAT | |
| | CAGAGCGGCGCGCTCCGAAAGTTTCCTTTTATGGCGA | |
| | GGCGGCGGCGGCGGCGGCCCTATAAAAAGCGAAGCG | |
| | CGCGGCGGGCGGGAGTCGCTGCGCGCTGCCTTCGCCC | |
| | CGTGCCCCGCTCCGCCGCCGCCTCGCGCCGCCCGCCC | |
| | CGGCTCTGACTGACCGCGTTACTCCCACAGGTGAGCG | |
| | GGCGGGACGGCCCTTCTCCTCCGGGCTGTAATTAGCG | |
| | CTTGGTTTAATGACGGCTTGTTTCTTTTCTGTGGCTGC | |
| | GTGAAAGCCTTGAGGGGCTCCGGGAGGGCCCTTTGTG | |
| | CGGGGGGAGCGGCTCGGGGGGTGCGTGCGTGTGTGTG | |
| | TGCGTGGGGAGCGCCGCGTGCGGCTCCGCGCTGCCCG | |
| | GCGGCTGTGAGCGCTGCGGGCGCGGCGCGGGGCTTTG | |
| | TGCGCTCCGCAGTGTGCGCGAGGGGAGCGCGGCCGGG | |
| | GGCGGTGCCCCGCGGTGCGGGGGGGGCTGCGAGGGG | |
| | AACAAAGGCTGCGTGCGGGGTGTGTGCGTGGGGGGGT | |
| | GAGCAGGGGGTGTGGGCGCGTCGGTCGGGCTGCAACC | |
| | CCCCCTGCACCCCCCTCCCCGAGTTGCTGAGCACGGC | |
| | CCGGCTTCGGGTGCGGGGCTCCGTACGGGGCGTGGCG | |
| | CGGGGCTCGCCGTGCCGGGCGGGGGGTGGCGGCAGGT | |
| | GGGGGTGCCGGGCGGGGCGGGGCCGCCTCGGGCCGG | |
| | GGAGGGCTCGGGGGAGGGGCGCGGCGGCCCCCGGAG | |
| | CGCCGGCGGCTGTCGAGGCGCGGCGAGCCGCAGCCAT | |
| | TGCCTTTTATGGTAATCGTGCGAGAGGGCGCAGGGAC | |
| | TTCCTTTGTCCCAAATCTGTGCGGAGCCGAAATCTGGG | |
| | AGGCGCCGCCGCACCCCCTCTAGCGGGCGCGGGGCGA | |
| | AGCGGTGCGGCGCCGGCAGGAAGGAAATGGGCGGGG | |
| | AGGGCCTTCGTGCGTCGCCGCGCCGCCGTCCCCTTCTC | |
| | CCTCTCCAGCCTCGGGGCTGTCCGCGGGGGGACGGCT | |
| | GCCTTCGGGGGGGACGGGGCAGGGCGGGGTTCGGCTT | |
| | CTGGCGTGTGACCGGCGGCTCTAGAGCCTCTGCTAAC | |
| | CATGTTCATGCCTTCTTCTTTTTCCTACAGCTCCTGGGC | |
| | AACGTGCTGGTTATTGTGCTGTCTCATCATTTTGGCAA | |
| | AGAATTCGCCACCATGTTCCAGACCGGCGGACTGATC | |
| | GTGTTCTACGGCCTGCTGGCCCAGACAATGGCCGCCA | |
| | GACCTTGTATCCCCAAGAGCTTCGGATATAGCAGCGT | |
| | GGTCTGCGTGTGCAACGCCACCTACTGCGATAGCTTC | |
| | GACCCACCTACCTTTCCAGCTCTGGGCACCTTCTCCAG | |
| | ATACGAGTCTACAAGAAGCGGCAGAAGAATGGAACT | |
| | GTCCATGGGCCCTATCCAGGCCAACCACACCGGCACA | |
| | GGCCTCCTGTTGACCCTGCAGCCCGAGCAGAAATTTC | |
| | AGAAAGTGAAGGGATTCGGCGGCGCCATGACCGATGC | |
| | CGCCGCTCTGAATATCCTGGCGCTGAGCCCTCCTGCCC | |
| | AGAACCTGCTGCTGAAGAGCTACTTTAGCGAGGAGGG | |
| | GATCGGCTACAACATTATCAGAGTGCCCATGGCCAGC | |
| | TGCGACTTTAGCATCAGAACCTATACATACGCCGACA | |
| | CCCCAGATGACTTCCAGCTGCACAACTTCAGCCTGCCT | |
| | GAGGAAGATACAAAGCTGAAAATCCCCCTGATCCACC | |
| | GGGCCCTGCAACTGGCTCAGCGGCCAGTGTCCCTGCT | |
| | GGCCTCTCCTTGGACCAGCCCTACCTGGCTGAAGACC | |
| | AATGGCGCCGTGAACGGCAAGGGCTCTCTGAAGGGCC | |
| | AGCCTGGCGACATCTACCATCAGACCTGGGCCAGATA | |
| | CTTCGTGAAGTTCCTGGATGCCTACGCTGAGCACAAG | |
| | CTGCAGTTCTGGGCCGTGACCGCCGAGAACGAGCCTT | |
| | CCGCCGGACTGCTCAGCGGCTATCCTTTCCAGTGCCTG | |
| | GGCTTCACACCTGAGCACCAGCGCGACTTCATCGCCA | |
| | GGGACCTGGGCCCTACACTGGCCAATAGCACACACCA | |
| | CAACGTGAGACTGCTGATGCTGGATGATCAGAGACTG | |
| | CTGCTTCCCCACTGGGCTAAGGTCGTGCTGACCGACCC | |
| | TGAAGCCGCTAAGTACGTGCACGGCATCGCCGTGCAC | |
| | TGGTACCTGGACTTCCTGGCCCCTGCCAAGGCCACCCT | |
| | GGGAGAAACCCACCGGCTGTTCCCTAACACCATGCTG | |
| | TTTGCCTCAGAGGCCTGTGTGGGCTCCAAATTCTGGGA | |
| | GCAAAGCGTGCGGCTGGGCTCTTGGGACAGAGGAATG | |
| | CAGTACAGCCACAGCATCATCACCAACCTGCTGTACC | |
| | ACGTGGTGGGCTGGACCGACTGGAACCTGGCCCTGAA | |
| | CCCCGAGGGAGGCCCTAACTGGGTCAGAAACTTCGTG | |
| | GACAGCCCCATCATTGTGGATATCACCAAGGACACGT | |
| | TCTACAAGCAGCCAATGTTCTACCACCTGGGCCACTTC | |
| | AGCAAGTTCATCCCTGAAGGCAGTCAGAGAGTGGGCC | |
| | TGGTGGCCTCTCAGAAGAACGACCTGGACGCCGTTGC | |
| | GCTGATGCACCCCGACGGCAGCGCCGTGGTGGTGGTG | |
| | CTGAATAGATCTTCTAAGGATGTGCCTCTGACAATCA | |
| | AGGACCCTGCTGTGGGATTTCTGGAAACAATCAGCCC | |
| | TGGATACAGCATCCATACATACCTGTGGCGGCGGCAG | |
| | TGAGATTAATCAACCTCTGGATTACAAAATTTGTGAA | |
| | AGATTGACTGGTATTCTTAACTATGTTGCTCCTTTTAC | |
| | GCTATGTGGATACGCTGCTTTAATGCCTTTGTATCATG | |
| | CTATTGCTTCCCGTATGGCTTTCATTTTCTCCTCCTTGT | |
| | ATAAATCCTGGTTGCTGTCTCTTTATGAGGAGTTGTGG | |
| | CCCGTTGTCAGGCAACGTGGCGTGGTGTGCACTGTGTT | |
| | TGCTGACGCAACCCCCACTGGTTGGGGCATTGCCACC | |
| | ACCTGTCAGCTCCTTTCCGGGACTTTCGCTTTCCCCCT | |
| | CCCTATTGCCACGGCGGAACTCATCGCCGCCTGCCTTG | |
| | CCCGCTGCTGGACAGGGGCTCGGCTGTTGGGCACTGA | |
| | CAATTCCGTGGTGTTGTCGGGGAAATCATCGTCCTTTC | |
| | CTTGGCTGCTCGCCTGTGTTGCCACCTGGATTCTGCGC | |
| | GGGACGTCCTTCTGCTACGTCCCTTCGGCCCTCAATCC | |
| | AGCGGACCTTCCTTCCCGCGGCCTGCTGCCGGCTCTGC | |
| | GGCCTCTTCCGCGTCTTCGCCTTCGCCCTCAGACGAGT | |
| | CGGATCTCCCTTTGGGCCGCCTCCCCGCCTGATCCTGT | |
| | GCCTTCTAGTTGCCAGCCATCTGTTGTTTGCCCCTCCC | |
| | CCGTGCCTTCCTTGACCCTGGAAGGTGCCACTCCCACT | |
| | GTCCTTTCCTAATAAAATGAGGAAATTGCATCGCATTG | |
| | TCTGAGTAGGTGTCATTCTATTCTGGGGGGTGGGGTG | |
| | GGGCAGGACAGCAAGGGGGAGGATTGGGAAGACAAT | |
| | AGCAGGCATGCTCGAAATTAGGAACCCCTAGTGATGG | |
| | AGTTGGCCACTCCCTCTCTGCGCGCTCGCTCGCTCACT | |
| | GAGGCCGCCCGGGCAAAGCCCGGGCGTCGGGCGACC | |
| | TTTGGTCGCCCGGCCTCAGTGAGCGAGCGAGCGCGCA | |
| | GAGAGGGAGTGGCCAATCGCGACGGCCGACGTCTTTG | |
| | TTACAACTTACTATATATATGCACACATATATATATAT | |
| | TTGGGTATATTGGGGGGGTTCTAATTTAAGAAATGCA | |
| | TAATTGGCTATAGACAGACAGTTGTCAGAACTTGGCA | |
| | ATGGGTACGTGCAGGTTCATTATACCAAGTCTACTTGT | |
| | AGTTGTTCAAAATGTATCATAATACAAGGCCGGGCGA | |
| | GGTCGTCACGCCTGTAATCCCAGCATTTTGGGAGGCT | |
| | AAGGCAGGAGGATTGCTTGAGGTCAGGAGTTTGTGAC | |
| | CAGCCTGGGCAACAGAGCAAGACCCTGTCTCCAAAAA | |
| | GAAAAAAAATAATTTTTTACAAAATAAAAACAAAATG | |
| | TATCATCAGACGAAATTAAATAAGAGGCAATTCATTG | |
| | TAATGACAACTTTTCCCAGCTTGACATTTAACAAAAA | |
| | GTCTAAGTCCTCTTAATTCATATTTAATGATCAAATAT | |
| | CAAATACTAATTTTTTTTTTTTTTTTTTTTTTGAGACGG | |
| | AGTCTCGCTCTGTCGCCCAGGCTGGAGTGCAGTGGCG | |
| | CGATCCTGGCTCACTGCAAGCTCCGCCTCCCGGGTTCA | |
| | CGCCATTCTCCTGCCTCAGCCTCCCGAGTAGCTGGGAT | |
| | TACAGACATGCGCCACCACGCCCGGCTAATTTTGTATT | |
| | TTTAGTAGAGATGGGGTTTCTCCATGTTGGTCAGGCTG | |
| | GTCTTGAATTTCCCACCTCAGGTGATCTGCCTGCCTCA | |
| | GCCTCACAAAGCAGTAGCTGGGACTACAGGCACCCAC | |
| | CACCACACTTGGTTAATTCTTTTGTATTTTTTTTGTAAA | |
| | GACGGGATTTCACCATGTTAGCCAGGATGGTCTCGAT | |
| | CTCCTGATCTCATGATCCGCCCGCCTCAGCCTCCCAAA | |
| | GTGCTGGGATTACAGGCGTGAGCCACCCCGCCCGGCC | |
| | ATCAAATACTAATTCTTAAATGGTAAGGACCCACTATT | |
| | CAGAACCTGTATCCTTATCACTAATATGCAAATATTTA | |
| | TTGAATACTTACTATGTCATGCATACTAGAGAGAGTTA | |
| | GATAAATTTGATACAGCTACCCTCACAGAACTTACAG | |
| | TGTAATAGATGGCATGACATGTACATGAGTAACTGTG | |
| | AACAGTGTTAAATTGCTATTTAAAAAAAAAGACGGCT | |
| | GGGCGCTGTGGCTCATGCCTGTAATCCCAGCACTTTGG | |
| | GAGGCCAAGGCAAGTTGATCGCTCGAGGTCAAGAGTT | |
| | CGAGACCAGCCTGGCCAACGTGGTAAAACCCCGTCTC | |
| | TACTAAAAATACAAAAAAAAAATTAGCCAGGCATGGT | |
| | GGCACAGGCCTGTAATCCCAGCTACTAGGGAGGCTGA | |
| | GACATGGAGAACTGCTTGAATCCAGGAGGCAGAGGTT | |
| | ACAGTGAGCCGAGATCATACCACTACACTCCAGCCTG | |
| | AGTGACAGAGCGAGACTCCTGTCTAAAAAAAAAAAA | |
| | AAAAAAAAAGATACAGGTTAAGTGTTATGGTAGTTGA | |
| | AGAGAGAACTCAAACTCTGTCTCAGAAGCCTCACTTG | |
| | CATGTGGACCACTGATATGAAATAATATAAATAGGTA | |
| | TAATTCAATAAATAGGAACTTCAGTTTTAATCATCCCA | |
| | AACACCAAAACTTCCTATCAAACAGGTCCAATAAACT | |
| | CAATCTCTATAAGAGCTAGACAGAAATCTACTTGGTG | |
| | GCCTATAATCTTATTAGCCCTTACTTGTCCCATCTGAT | |
| | ATTAATTAACCCCATCTAATATGGATTAGTTAACAATC | |
| | CAGTGGCTGCTTTGACAGGAACAGTTGGAGAGAGTTG | |
| | GGGATTGCAACATATTCAATTATACAAAAATGCATTC | |
| | AGCATCTACCTTGATTAAGGCAGTGTGCAACAGAATT | |
| | TGCAGGAGAGTAAAAGAATGATTATAAATTTACAACC | |
| | CTTAAAGAGCTATAGCTGGGCGTGGTGGCTCATGCCT | |
| | GTAAATCCCAGCACTTTGGGAGGCTGAGGCGGGTGGA | |
| | TCACCTGAGGCCAGAAGTTCAAGACCAGCCTAGCCAA | |
| | CATGGCGAAACCCTGTCTCTACAAAAAATACAAAAAT | |
| | TAGCCGGGTGTGGTGGCACGTGCCTGTAGTCCCAGTT | |
| | ACTTGGGAGGCCGAGGCAGGAGAATCGCTTGAACCTA | |
| | GGAGGTGGAGGCTGCAGTGAGCCGAGATTGTGCCACT | |
| | GCACTCCACTTCAGCCTGGGCGACAAGAGCAAGACTC | |
| | CGTCACAAAAAAAAAAAAAAAAAAAAAGGCTAAAAT | |
| | CTAGTGGGAAAGGCATATATACATACAACTAACTGTA | |
| | TAGCATAATAAAGCTCATAATCTGTAACAAAATCTAA | |
| | TTCGACAAGCCCAGAAACTTGTGATTTACCAAAAACA | |
| | GTTATATATACACAAAAAGTAAACCTAGAACCCAAAG | |
| | TTACCCAGCACCAATGATTCTCTCCCTAAGCAGTATCA | |
| | AGTTTAAAGCAGTGATTACATTCTACTGCCTAGATTGT | |
| | AAACTGAGTAAAGGAGACCAGCACCTTTCTGCTACTG | |
| | AACTAGCACAGCCGTGTAAACCAACAAGGCAATGGCA | |
| | GTGCCCAACTTTCTGTATGAATATAAGTTACATCTGTT | |
| | TTATTATTTGTGACTTGGTGTTGCATGTGGTTATTATC | |
| | AACACCTTCTGAAAGAACAACTACCTGCTCAGGCTGC | |
| | CATAACAAAATACCACAGACTGAGTGACTTAACAGAA | |
| | ACTTATTTCTCACAGTTTTGGAGGCTGGGAAGTCCAAA | |
| | ATTAAGCTAACTGCAAGGTAGGTTTCAATCTCAGGCC | |
| | TCTTCTTTGGCTTGAAGGTCTTCTAACTGTGTGCTCAC | |
| | ATGACCTCTTCTAACAAGCTCTCTGGTGTCTCTTTTTTT | |
| | TTTTTTTTCTTTTTTGAGACAGAGTCTCACTCTGTCACC | |
| | CAGGCTGGAGTACAGTGGCACAATCTGGGCTCACTGC | |
| | AACCTCCAACTCCCGGGTTCAAGTGATTCTCATGCCTC | |
| | ACCCTCCCGAGTAGCTTGGATGACAGGAGCCCGCTAC | |
| | CACACCCAGCTAATTTTTGTATTTTTAGTAGAGATGGT | |
| | GTTTCACTACATTGGCCAGGCTGGTCTCAAACTCCTGA | |
| | CCTCGTGATCCACCCACCTTGGCCTCCCAAAGTGCTGG | |
| | GATTACAGGTGTGAGCCACTGCGCCCGTCCTGGTGTCT | |
| | TTTCATATAAGGGCACTAATCCAATCAGACCTGGGCC | |
| | CAACCCTCCCGACTTCTTCTAACTGTAATTACCTTCCA | |
| | AAGGCCCTGTCTCCAAATACCATCACACTGGGGGTTA | |
| | GGACTTCAAAAAAGGTATGGGGGGGGTGTGGGAGGA | |
| | CATAAATGCTCAGTCCATAACAAGCACCCAACATAAA | |
| | AATGGCTAGAACAGATCACAAAAAAAAGGTCCTGTAT | |
| | GGCTTTGGGGAAGGGCTCAACCCCAAAATATCTGAAA | |
| | GCTCTGGAGGGGCCTAGAAGTGGTAAATGAATGAAAA | |
| | CGTGGTTACTCTCCCGATCTGCCTTTCCCAAATATGGC | |
| | CATTCTTGGCTGAATCAGAAATCAAAGGACAGGTTAT | |
| | TAATTACTAGCTCTAAGTTACTTACCATTTGCTGAGAC | |
| | AGTTCAGAAATCTGACTGCATCTCCTCAGGGATCTAG | |
| | AACACAGTTCTCAAATTCTAACTTACTTGTGATATACT | |
| | TGTGAATGATAAAAATCGCTACAGGTACTTTTATTAAT | |
| | CTGAAAGAGTATTGAGAAATTACCTTTCATTCTGACTT | |
| | TTGTCTGGAATGAAAATCAATACTTTTGCTATATTCCA | |
| | TTACTGAAATAATTTTACTTTCCAGTAAAACTGGCATT | |
| | ATAATTTTTTTTAATTTTTAAAACTTCATAATTTTTTGC | |
| | CAGACTGACCCATGTAAACATACAAATTACTAATAAT | |
| | TATGCACGTCACATCTGTAATAATGGCCTTCATGTAAA | |
| | CATTTTTGTGGTTTACACATAAAATCTCTAATTACAAA | |
| | GCTATATTATCTAAAATTACAGTAAGCAAGAAAATTA | |
| | ATCCAAGCTAAGACAATACTTGCAACATCAATTCATC | |
| | ATCTGTGACAAGGACTGCTTAAGTCTCTTTGTGGTTGA | |
| | CGTCATTAATTAACTGGCCTCATGGGCCTTCCGCTCAC | |
| | TGCCCGCTTTCCAGTCGGGAAACCTGTCGTGCCAGCTG | |
| | CATTAACATGGTCATAGCTGTTTCCTTGCGTATTGGGC | |
| | GCTCTCCGCTTCCTCGCTCACTGACTCGCTGCGCTCGG | |
| | TCGTTCGGGTAAAGCCTGGGGTGCCTAATGAGCAAAA | |
| | GGCCAGCAAAAGGCCAGGAACCGTAAAAAGGCCGCG | |
| | TTGCTGGCGTTTTTCCATAGGCTCCGCCCCCCTGACGA | |
| | GCATCACAAAAATCGACGCTCAAGTCAGAGGTGGCGA | |
| | AACCCGACAGGACTATAAAGATACCAGGCGTTTCCCC | |
| | CTGGAAGCTCCCTCGTGCGCTCTCCTGTTCCGACCCTG | |
| | CCGCTTACCGGATACCTGTCCGCCTTTCTCCCTTCGGG | |
| | AAGCGTGGCGCTTTCTCATAGCTCACGCTGTAGGTATC | |
| | TCAGTTCGGTGTAGGTCGTTCGCTCCAAGCTGGGCTGT | |
| | GTGCACGAACCCCCCGTTCAGCCCGACCGCTGCGCCT | |
| | TATCCGGTAACTATCGTCTTGAGTCCAACCCGGTAAG | |
| | ACACGACTTATCGCCACTGGCAGCAGCCACTGGTAAC | |
| | AGGATTAGCAGAGCGAGGTATGTAGGCGGTGCTACAG | |
| | AGTTCTTGAAGTGGTGGCCTAACTACGGCTACACTAG | |
| | AAGAACAGTATTTGGTATCTGCGCTCTGCTGAAGCCA | |
| | GTTACCTTCGGAAAAAGAGTTGGTAGCTCTTGATCCG | |
| | GCAAACAAACCACCGCTGGTAGCGGTGGTTTTTTTGTT | |
| | TGCAAGCAGCAGATTACGCGCAGAAAAAAAGGATCTC | |
| | AAGAAGATCCTTTGATCTTTTCTACGGGGTCTGACGCT | |
| | CAGTGGAACGAAAACTCACGTTAAGGGATTTTGGTCA | |
| | TGAGATTATCAAAAAGGATCTTCACCTAGATCCTTTTA | |
| | AATTAAAAATGAAGTTTTAAATCAATCTAAAGTATAT | |
| | ATGAGTAAACTTGGTCTGACAGTTACCAATGCTTAATC | |
| | AGTGAGGCACCTATCTCAGCGATCTGTCTATTTCGTTC | |
| | ATCCATAGTTGCCTGACTCCCCGTCGTGTAGATAACTA | |
| | CGATACGGGAGGGCTTACCATCTGGCCCCAGTGCTGC | |
| | AATGATACCGCGAGAACCACGCTCACCGGCTCCAGAT | |
| | TTATCAGCAATAAACCAGCCAGCCGGAAGGGCCGAGC | |
| | GCAGAAGTGGTCCTGCAACTTTATCCGCCTCCATCCAG | |
| | TCTATTAATTGTTGCCGGGAAGCTAGAGTAAGTAGTTC | |
| | GCCAGTTAATAGTTTGCGCAACGTTGTTGCCATTGCTA | |
| | CAGGCATCGTGGTGTCACGCTCGTCGTTTGGTATGGCT | |
| | TCATTCAGCTCCGGTTCCCAACGATCAAGGCGAGTTA | |
| | CATGATCCCCCATGTTGTGCAAAAAAGCGGTTAGCTC | |
| | CTTCGGTCCTCCGATCGTTGTCAGAAGTAAGTTGGCCG | |
| | CAGTGTTATCACTCATGGTTATGGCAGCACTGCATAAT | |
| | TCTCTTACTGTCATGCCATCCGTAAGATGCTTTTCTGT | |
| | GACTGGTGAGTACTCAACCAAGTCATTCTGAGAATAG | |
| | TGTATGCGGCGACCGAGTTGCTCTTGCCCGGCGTCAAT | |
| | ACGGGATAATACCGCGCCACATAGCAGAACTTTAAAA | |
| | GTGCTCATCATTGGAAAACGTTCTTCGGGGCGAAAAC | |
| | TCTCAAGGATCTTACCGCTGTTGAGATCCAGTTCGATG | |
| | TAACCCACTCGTGCACCCAACTGATCTTCAGCATCTTT | |
| | TACTTTCACCAGCGTTTCTGGGTGAGCAAAAACAGGA | |
| | AGGCAAAATGCCGCAAAAAAGGGAATAAGGGCGACA | |
| | CGGAAATGTTGAATACTCATACTCTTCCTTTTTCAATA | |
| | TTATTGAAGCATTTATCAGGGTTATTGTCTCATGAGCG | |
| | GATACATATTTGAATGTATTTAGAAAAATAAACAAAT | |
| | AGGGGTTCCGCGCACATTTCCCCGAAAAGTGCCACCT | |
| | AAATTGTAAGCGTTAATATTTTGTTAAAATTCGCGTTA | |
| | AATTTTTGTTAAATCAGCTCATTTTTTAACCAATAGGC | |
| | CGAAATCGGCAAAATCCCTTATAAATCAAAAGAATAG | |
| | ACCGAGATAGGGTTGAGTGGCCGCTACAGGGCGCTCC | |
| | CATTCGCCATTCAGGCTGCGCAACTGTTGGGAAGGGC | |
| | GTTTCGGTGCGGGCCTCTTCGCTATTACGCCAGCTGGC | |
| | GAAAGGGGGATGTGCTGCAAGGCGATTAAGTTGGGTA | |
| | ACGCCAGGGTTTTCCCAGTCACGACGTTGTAAAACGA | |
| | CGGCCAGTGAGCGCGACGTAATACGACTCACTATAGG | |
| | GCGAATTGGCGGAAGGCCGTCAAGGCCTAGGCGCGCC | |
| | AACGCGT | |
| 22 | TTGGCCACTCCCTCTCTGCGCGCTCGCTCGCTCACTGA | Full DNA sequence |
| | GGCCGCCCGGGCAAAGCCCGGGCGTCGGGCGACCTTT | of vector (pAAV- |
| | GGTCGCCCGGCCTCAGTGAGCGAGCGAGCGCGCAGAG | CBA-SS3-GBA- |
| | AGGGAGTGGCCAACTCCATCACTAGGGGTTCCTTACG | WPRE-bGH); |
| | TACAATTGGGATCCCGGACCGTCGACATTGATTATTG | coding sequence for |
| | ACTAGTTATTAATAGTAATCAATTACGGGGTCATTAGT | GBA1 is underlined |
| | TCATAGCCCATATATGGAGTTCCGCGTTACATAACTTA | Underlined, codon |
| | CGGTAAATGGCCCGCCTGGCTGACCGCCCAACGACCC | Optimized GBA1 |
| | CCGCCCATTGACGTCAATAATGACGTATGTTCCCATAG | DNA sequence |
| | TAACGCCAATAGGGACTTTCCATTGACGTCAATGGGT | (Human) |
| | GGAGTATTTACGGTAAACTGCCCACTTGGCAGTACAT | |
| | CAAGTGTATCATATGCCAAGTACGCCCCCTATTGACGT | |
| | CAATGACGGTAAATGGCCCGCCTGGCATTATGCCCAG | |
| | TACATGACCTTATGGGACTTTCCTACTTGGCAGTACAT | |
| | CTACGTATTAGTCATCGCTATTACCATGGTCGAGGTGA | |
| | GCCCCACGTTCTGCTTCACTCTCCCCATCTCCCCCCCC | |
| | TCCCCACCCCCAATTTTGTATTTATTTATTTTTTAATTA | |
| | TTTTGTGCAGCGATGGGGGCGGGGGGGGGGGGGGGG | |
| | CGCGCGCCAGGCGGGGCGGGGCGGGGCGAGGGGCGG | |
| | GGCGGGGCGAGGCGGAGAGGTGCGGCGGCAGCCAAT | |
| | CAGAGCGGCGCGCTCCGAAAGTTTCCTTTTATGGCGA | |
| | GGCGGCGGCGGCGGCGGCCCTATAAAAAGCGAAGCG | |
| | CGCGGCGGGCGGGAGTCGCTGCGCGCTGCCTTCGCCC | |
| | CGTGCCCCGCTCCGCCGCCGCCTCGCGCCGCCCGCCC | |
| | CGGCTCTGACTGACCGCGTTACTCCCACAGGTGAGCG | |
| | GGCGGGACGGCCCTTCTCCTCCGGGCTGTAATTAGCG | |
| | CTTGGTTTAATGACGGCTTGTTTCTTTTCTGTGGCTGC | |
| | GTGAAAGCCTTGAGGGGCTCCGGGAGGGCCCTTTGTG | |
| | CGGGGGGAGCGGCTCGGGGGGTGCGTGCGTGTGTGTG | |
| | TGCGTGGGGAGCGCCGCGTGCGGCTCCGCGCTGCCCG | |
| | GCGGCTGTGAGCGCTGCGGGCGCGGCGCGGGGCTTTG | |
| | TGCGCTCCGCAGTGTGCGCGAGGGGAGCGCGGCCGGG | |
| | GGCGGTGCCCCGCGGTGCGGGGGGGGCTGCGAGGGG | |
| | AACAAAGGCTGCGTGCGGGGTGTGTGCGTGGGGGGGT | |
| | GAGCAGGGGGTGTGGGCGCGTCGGTCGGGCTGCAACC | |
| | CCCCCTGCACCCCCCTCCCCGAGTTGCTGAGCACGGC | |
| | CCGGCTTCGGGTGCGGGGCTCCGTACGGGGCGTGGCG | |
| | CGGGGCTCGCCGTGCCGGGCGGGGGGGGCGGCAGGT | |
| | GGGGGTGCCGGGCGGGGCGGGGCCGCCTCGGGCCGG | |
| | GGAGGGCTCGGGGGAGGGGCGCGGCGGCCCCCGGAG | |
| | CGCCGGCGGCTGTCGAGGCGCGGCGAGCCGCAGCCAT | |
| | TGCCTTTTATGGTAATCGTGCGAGAGGGCGCAGGGAC | |
| | TTCCTTTGTCCCAAATCTGTGCGGAGCCGAAATCTGGG | |
| | AGGCGCCGCCGCACCCCCTCTAGCGGGCGCGGGGCGA | |
| | AGCGGTGCGGCGCCGGCAGGAAGGAAATGGGCGGGG | |
| | AGGGCCTTCGTGCGTCGCCGCGCCGCCGTCCCCTTCTC | |
| | CCTCTCCAGCCTCGGGGCTGTCCGCGGGGGGACGGCT | |
| | GCCTTCGGGGGGGACGGGGCAGGGCGGGGTTCGGCTT | |
| | CTGGCGTGTGACCGGCGGCTCTAGAGCCTCTGCTAAC | |
| | CATGTTCATGCCTTCTTCTTTTTCCTACAGCTCCTGGGC | |
| | AACGTGCTGGTTATTGTGCTGTCTCATCATTTTGGCAA | |
| | AGAATTCGCCACCATGCTGCTGCTGCTGCTGCTGCTCG | |
| | GCCTGAGACTGCAGCTGAGCCTGGGCGCCAGACCTTG | |
| | TATCCCCAAGAGCTTCGGATATAGCAGCGTGGTCTGC | |
| | GTGTGCAACGCCACCTACTGCGATAGCTTCGACCCAC | |
| | CTACCTTTCCAGCTCTGGGCACCTTCTCCAGATACGAG | |
| | TCTACAAGAAGCGGCAGAAGAATGGAACTGTCCATGG | |
| | GCCCTATCCAGGCCAACCACACCGGCACAGGCCTCCT | |
| | GTTGACCCTGCAGCCCGAGCAGAAATTTCAGAAAGTG | |
| | AAGGGATTCGGCGGCGCCATGACCGATGCCGCCGCTC | |
| | TGAATATCCTGGCGCTGAGCCCTCCTGCCCAGAACCT | |
| | GCTGCTGAAGAGCTACTTTAGCGAGGAGGGGATCGGC | |
| | TACAACATTATCAGAGTGCCCATGGCCAGCTGCGACT | |
| | TTAGCATCAGAACCTATACATACGCCGACACCCCAGA | |
| | TGACTTCCAGCTGCACAACTTCAGCCTGCCTGAGGAA | |
| | GATACAAAGCTGAAAATCCCCCTGATCCACCGGGCCC | |
| | TGCAACTGGCTCAGCGGCCAGTGTCCCTGCTGGCCTCT | |
| | CCTTGGACCAGCCCTACCTGGCTGAAGACCAATGGCG | |
| | CCGTGAACGGCAAGGGCTCTCTGAAGGGCCAGCCTGG | |
| | CGACATCTACCATCAGACCTGGGCCAGATACTTCGTG | |
| | AAGTTCCTGGATGCCTACGCTGAGCACAAGCTGCAGT | |
| | TCTGGGCCGTGACCGCCGAGAACGAGCCTTCCGCCGG | |
| | ACTGCTCAGCGGCTATCCTTTCCAGTGCCTGGGCTTCA | |
| | CACCTGAGCACCAGCGCGACTTCATCGCCAGGGACCT | |
| | GGGCCCTACACTGGCCAATAGCACACACCACAACGTG | |
| | AGACTGCTGATGCTGGATGATCAGAGACTGCTGCTTC | |
| | CCCACTGGGCTAAGGTCGTGCTGACCGACCCTGAAGC | |
| | CGCTAAGTACGTGCACGGCATCGCCGTGCACTGGTAC | |
| | CTGGACTTCCTGGCCCCTGCCAAGGCCACCCTGGGAG | |
| | AAACCCACCGGCTGTTCCCTAACACCATGCTGTTTGCC | |
| | TCAGAGGCCTGTGTGGGCTCCAAATTCTGGGAGCAAA | |
| | GCGTGCGGCTGGGCTCTTGGGACAGAGGAATGCAGTA | |
| | CAGCCACAGCATCATCACCAACCTGCTGTACCACGTG | |
| | GTGGGCTGGACCGACTGGAACCTGGCCCTGAACCCCG | |
| | AGGGAGGCCCTAACTGGGTCAGAAACTTCGTGGACAG | |
| | CCCCATCATTGTGGATATCACCAAGGACACGTTCTAC | |
| | AAGCAGCCAATGTTCTACCACCTGGGCCACTTCAGCA | |
| | AGTTCATCCCTGAAGGCAGTCAGAGAGTGGGCCTGGT | |
| | GGCCTCTCAGAAGAACGACCTGGACGCCGTTGCGCTG | |
| | ATGCACCCCGACGGCAGCGCCGTGGTGGTGGTGCTGA | |
| | ATAGATCTTCTAAGGATGTGCCTCTGACAATCAAGGA | |
| | CCCTGCTGTGGGATTTCTGGAAACAATCAGCCCTGGA | |
| | TACAGCATCCATACATACCTGTGGCGGCGGCAGTGAG | |
| | ATTAATCAACCTCTGGATTACAAAATTTGTGAAAGATT | |
| | GACTGGTATTCTTAACTATGTTGCTCCTTTTACGCTAT | |
| | GTGGATACGCTGCTTTAATGCCTTTGTATCATGCTATT | |
| | GCTTCCCGTATGGCTTTCATTTTCTCCTCCTTGTATAAA | |
| | TCCTGGTTGCTGTCTCTTTATGAGGAGTTGTGGCCCGT | |
| | TGTCAGGCAACGTGGCGTGGTGTGCACTGTGTTTGCTG | |
| | ACGCAACCCCCACTGGTTGGGGCATTGCCACCACCTG | |
| | TCAGCTCCTTTCCGGGACTTTCGCTTTCCCCCTCCCTAT | |
| | TGCCACGGCGGAACTCATCGCCGCCTGCCTTGCCCGC | |
| | TGCTGGACAGGGGCTCGGCTGTTGGGCACTGACAATT | |
| | CCGTGGTGTTGTCGGGGAAATCATCGTCCTTTCCTTGG | |
| | CTGCTCGCCTGTGTTGCCACCTGGATTCTGCGCGGGAC | |
| | GTCCTTCTGCTACGTCCCTTCGGCCCTCAATCCAGCGG | |
| | ACCTTCCTTCCCGCGGCCTGCTGCCGGCTCTGCGGCCT | |
| | CTTCCGCGTCTTCGCCTTCGCCCTCAGACGAGTCGGAT | |
| | CTCCCTTTGGGCCGCCTCCCCGCCTGATCCTGTGCCTT | |
| | CTAGTTGCCAGCCATCTGTTGTTTGCCCCTCCCCCGTG | |
| | CCTTCCTTGACCCTGGAAGGTGCCACTCCCACTGTCCT | |
| | TTCCTAATAAAATGAGGAAATTGCATCGCATTGTCTG | |
| | AGTAGGTGTCATTCTATTCTGGGGGGGGGGTGGGGC | |
| | AGGACAGCAAGGGGGAGGATTGGGAAGACAATAGCA | |
| | GGCATGCTCGAAATTAGGAACCCCTAGTGATGGAGTT | |
| | GGCCACTCCCTCTCTGCGCGCTCGCTCGCTCACTGAGG | |
| | CCGCCCGGGCAAAGCCCGGGCGTCGGGCGACCTTTGG | |
| | TCGCCCGGCCTCAGTGAGCGAGCGAGCGCGCAGAGAG | |
| | GGAGTGGCCAATCGCGACGGCCGACGTCTTTGTTACA | |
| | ACTTACTATATATATGCACACATATATATATATTTGGG | |
| | TATATTGGGGGGGTTCTAATTTAAGAAATGCATAATT | |
| | GGCTATAGACAGACAGTTGTCAGAACTTGGCAATGGG | |
| | TACGTGCAGGTTCATTATACCAAGTCTACTTGTAGTTG | |
| | TTCAAAATGTATCATAATACAAGGCCGGGCGAGGTCG | |
| | TCACGCCTGTAATCCCAGCATTTTGGGAGGCTAAGGC | |
| | AGGAGGATTGCTTGAGGTCAGGAGTTTGTGACCAGCC | |
| | TGGGCAACAGAGCAAGACCCTGTCTCCAAAAAGAAA | |
| | AAAAATAATTTTTTACAAAATAAAAACAAAATGTATC | |
| | ATCAGACGAAATTAAATAAGAGGCAATTCATTGTAAT | |
| | GACAACTTTTCCCAGCTTGACATTTAACAAAAAGTCTA | |
| | AGTCCTCTTAATTCATATTTAATGATCAAATATCAAAT | |
| | ACTAATTTTTTTTTTTTTTTTTTTTTTGAGACGGAGTCT | |
| | CGCTCTGTCGCCCAGGCTGGAGTGCAGTGGCGCGATC | |
| | CTGGCTCACTGCAAGCTCCGCCTCCCGGGTTCACGCC | |
| | ATTCTCCTGCCTCAGCCTCCCGAGTAGCTGGGATTACA | |
| | GACATGCGCCACCACGCCCGGCTAATTTTGTATTTTTA | |
| | GTAGAGATGGGGTTTCTCCATGTTGGTCAGGCTGGTCT | |
| | TGAATTTCCCACCTCAGGTGATCTGCCTGCCTCAGCCT | |
| | CACAAAGCAGTAGCTGGGACTACAGGCACCCACCACC | |
| | ACACTTGGTTAATTCTTTTGTATTTTTTTTGTAAAGACG | |
| | GGATTTCACCATGTTAGCCAGGATGGTCTCGATCTCCT | |
| | GATCTCATGATCCGCCCGCCTCAGCCTCCCAAAGTGCT | |
| | GGGATTACAGGCGTGAGCCACCCCGCCCGGCCATCAA | |
| | ATACTAATTCTTAAATGGTAAGGACCCACTATTCAGA | |
| | ACCTGTATCCTTATCACTAATATGCAAATATTTATTGA | |
| | ATACTTACTATGTCATGCATACTAGAGAGAGTTAGAT | |
| | AAATTTGATACAGCTACCCTCACAGAACTTACAGTGT | |
| | AATAGATGGCATGACATGTACATGAGTAACTGTGAAC | |
| | AGTGTTAAATTGCTATTTAAAAAAAAAGACGGCTGGG | |
| | CGCTGTGGCTCATGCCTGTAATCCCAGCACTTTGGGAG | |
| | GCCAAGGCAAGTTGATCGCTCGAGGTCAAGAGTTCGA | |
| | GACCAGCCTGGCCAACGTGGTAAAACCCCGTCTCTAC | |
| | TAAAAATACAAAAAAAAAATTAGCCAGGCATGGTGG | |
| | CACAGGCCTGTAATCCCAGCTACTAGGGAGGCTGAGA | |
| | CATGGAGAACTGCTTGAATCCAGGAGGCAGAGGTTAC | |
| | AGTGAGCCGAGATCATACCACTACACTCCAGCCTGAG | |
| | TGACAGAGCGAGACTCCTGTCTAAAAAAAAAAAAAA | |
| | AAAAAAAGATACAGGTTAAGTGTTATGGTAGTTGAAG | |
| | AGAGAACTCAAACTCTGTCTCAGAAGCCTCACTTGCA | |
| | TGTGGACCACTGATATGAAATAATATAAATAGGTATA | |
| | ATTCAATAAATAGGAACTTCAGTTTTAATCATCCCAAA | |
| | CACCAAAACTTCCTATCAAACAGGTCCAATAAACTCA | |
| | ATCTCTATAAGAGCTAGACAGAAATCTACTTGGTGGC | |
| | CTATAATCTTATTAGCCCTTACTTGTCCCATCTGATATT | |
| | AATTAACCCCATCTAATATGGATTAGTTAACAATCCA | |
| | GTGGCTGCTTTGACAGGAACAGTTGGAGAGAGTTGGG | |
| | GATTGCAACATATTCAATTATACAAAAATGCATTCAG | |
| | CATCTACCTTGATTAAGGCAGTGTGCAACAGAATTTG | |
| | CAGGAGAGTAAAAGAATGATTATAAATTTACAACCCT | |
| | TAAAGAGCTATAGCTGGGCGTGGTGGCTCATGCCTGT | |
| | AAATCCCAGCACTTTGGGAGGCTGAGGCGGGTGGATC | |
| | ACCTGAGGCCAGAAGTTCAAGACCAGCCTAGCCAACA | |
| | TGGCGAAACCCTGTCTCTACAAAAAATACAAAAATTA | |
| | GCCGGGTGTGGTGGCACGTGCCTGTAGTCCCAGTTAC | |
| | TTGGGAGGCCGAGGCAGGAGAATCGCTTGAACCTAGG | |
| | AGGTGGAGGCTGCAGTGAGCCGAGATTGTGCCACTGC | |
| | ACTCCACTTCAGCCTGGGCGACAAGAGCAAGACTCCG | |
| | TCACAAAAAAAAAAAAAAAAAAAAAGGCTAAAATCT | |
| | AGTGGGAAAGGCATATATACATACAACTAACTGTATA | |
| | GCATAATAAAGCTCATAATCTGTAACAAAATCTAATT | |
| | CGACAAGCCCAGAAACTTGTGATTTACCAAAAACAGT | |
| | TATATATACACAAAAAGTAAACCTAGAACCCAAAGTT | |
| | ACCCAGCACCAATGATTCTCTCCCTAAGCAGTATCAA | |
| | GTTTAAAGCAGTGATTACATTCTACTGCCTAGATTGTA | |
| | AACTGAGTAAAGGAGACCAGCACCTTTCTGCTACTGA | |
| | ACTAGCACAGCCGTGTAAACCAACAAGGCAATGGCAG | |
| | TGCCCAACTTTCTGTATGAATATAAGTTACATCTGTTT | |
| | TATTATTTGTGACTTGGTGTTGCATGTGGTTATTATCA | |
| | ACACCTTCTGAAAGAACAACTACCTGCTCAGGCTGCC | |
| | ATAACAAAATACCACAGACTGAGTGACTTAACAGAAA | |
| | CTTATTTCTCACAGTTTTGGAGGCTGGGAAGTCCAAAA | |
| | TTAAGCTAACTGCAAGGTAGGTTTCAATCTCAGGCCTC | |
| | TTCTTTGGCTTGAAGGTCTTCTAACTGTGTGCTCACAT | |
| | GACCTCTTCTAACAAGCTCTCTGGTGTCTCTTTTTTTTT | |
| | TTTTTTCTTTTTTGAGACAGAGTCTCACTCTGTCACCCA | |
| | GGCTGGAGTACAGTGGCACAATCTGGGCTCACTGCAA | |
| | CCTCCAACTCCCGGGTTCAAGTGATTCTCATGCCTCAC | |
| | CCTCCCGAGTAGCTTGGATGACAGGAGCCCGCTACCA | |
| | CACCCAGCTAATTTTTGTATTTTTAGTAGAGATGGTGT | |
| | TTCACTACATTGGCCAGGCTGGTCTCAAACTCCTGACC | |
| | TCGTGATCCACCCACCTTGGCCTCCCAAAGTGCTGGG | |
| | ATTACAGGTGTGAGCCACTGCGCCCGTCCTGGTGTCTT | |
| | TTCATATAAGGGCACTAATCCAATCAGACCTGGGCCC | |
| | AACCCTCCCGACTTCTTCTAACTGTAATTACCTTCCAA | |
| | AGGCCCTGTCTCCAAATACCATCACACTGGGGGTTAG | |
| | GACTTCAAAAAAGGTATGGGGGGGGTGTGGGAGGAC | |
| | ATAAATGCTCAGTCCATAACAAGCACCCAACATAAAA | |
| | ATGGCTAGAACAGATCACAAAAAAAAGGTCCTGTATG | |
| | GCTTTGGGGAAGGGCTCAACCCCAAAATATCTGAAAG | |
| | CTCTGGAGGGGCCTAGAAGTGGTAAATGAATGAAAAC | |
| | GTGGTTACTCTCCCGATCTGCCTTTCCCAAATATGGCC | |
| | ATTCTTGGCTGAATCAGAAATCAAAGGACAGGTTATT | |
| | AATTACTAGCTCTAAGTTACTTACCATTTGCTGAGACA | |
| | GTTCAGAAATCTGACTGCATCTCCTCAGGGATCTAGA | |
| | ACACAGTTCTCAAATTCTAACTTACTTGTGATATACTT | |
| | GTGAATGATAAAAATCGCTACAGGTACTTTTATTAATC | |
| | TGAAAGAGTATTGAGAAATTACCTTTCATTCTGACTTT | |
| | TGTCTGGAATGAAAATCAATACTTTTGCTATATTCCAT | |
| | TACTGAAATAATTTTACTTTCCAGTAAAACTGGCATTA | |
| | TAATTTTTTTTAATTTTTAAAACTTCATAATTTTTTGCC | |
| | AGACTGACCCATGTAAACATACAAATTACTAATAATT | |
| | ATGCACGTCACATCTGTAATAATGGCCTTCATGTAAAC | |
| | ATTTTTGTGGTTTACACATAAAATCTCTAATTACAAAG | |
| | CTATATTATCTAAAATTACAGTAAGCAAGAAAATTAA | |
| | TCCAAGCTAAGACAATACTTGCAACATCAATTCATCA | |
| | TCTGTGACAAGGACTGCTTAAGTCTCTTTGTGGTTGAC | |
| | GTCATTAATTAACTGGCCTCATGGGCCTTCCGCTCACT | |
| | GCCCGCTTTCCAGTCGGGAAACCTGTCGTGCCAGCTG | |
| | CATTAACATGGTCATAGCTGTTTCCTTGCGTATTGGGC | |
| | GCTCTCCGCTTCCTCGCTCACTGACTCGCTGCGCTCGG | |
| | TCGTTCGGGTAAAGCCTGGGGTGCCTAATGAGCAAAA | |
| | GGCCAGCAAAAGGCCAGGAACCGTAAAAAGGCCGCG | |
| | TTGCTGGCGTTTTTCCATAGGCTCCGCCCCCCTGACGA | |
| | GCATCACAAAAATCGACGCTCAAGTCAGAGGTGGCGA | |
| | AACCCGACAGGACTATAAAGATACCAGGCGTTTCCCC | |
| | CTGGAAGCTCCCTCGTGCGCTCTCCTGTTCCGACCCTG | |
| | CCGCTTACCGGATACCTGTCCGCCTTTCTCCCTTCGGG | |
| | AAGCGTGGCGCTTTCTCATAGCTCACGCTGTAGGTATC | |
| | TCAGTTCGGTGTAGGTCGTTCGCTCCAAGCTGGGCTGT | |
| | GTGCACGAACCCCCCGTTCAGCCCGACCGCTGCGCCT | |
| | TATCCGGTAACTATCGTCTTGAGTCCAACCCGGTAAG | |
| | ACACGACTTATCGCCACTGGCAGCAGCCACTGGTAAC | |
| | AGGATTAGCAGAGCGAGGTATGTAGGCGGTGCTACAG | |
| | AGTTCTTGAAGTGGTGGCCTAACTACGGCTACACTAG | |
| | AAGAACAGTATTTGGTATCTGCGCTCTGCTGAAGCCA | |
| | GTTACCTTCGGAAAAAGAGTTGGTAGCTCTTGATCCG | |
| | GCAAACAAACCACCGCTGGTAGCGGTGGTTTTTTTGTT | |
| | TGCAAGCAGCAGATTACGCGCAGAAAAAAAGGATCTC | |
| | AAGAAGATCCTTTGATCTTTTCTACGGGGTCTGACGCT | |
| | CAGTGGAACGAAAACTCACGTTAAGGGATTTTGGTCA | |
| | TGAGATTATCAAAAAGGATCTTCACCTAGATCCTTTTA | |
| | AATTAAAAATGAAGTTTTAAATCAATCTAAAGTATAT | |
| | ATGAGTAAACTTGGTCTGACAGTTACCAATGCTTAATC | |
| | AGTGAGGCACCTATCTCAGCGATCTGTCTATTTCGTTC | |
| | ATCCATAGTTGCCTGACTCCCCGTCGTGTAGATAACTA | |
| | CGATACGGGAGGGCTTACCATCTGGCCCCAGTGCTGC | |
| | AATGATACCGCGAGAACCACGCTCACCGGCTCCAGAT | |
| | TTATCAGCAATAAACCAGCCAGCCGGAAGGGCCGAGC | |
| | GCAGAAGTGGTCCTGCAACTTTATCCGCCTCCATCCAG | |
| | TCTATTAATTGTTGCCGGGAAGCTAGAGTAAGTAGTTC | |
| | GCCAGTTAATAGTTTGCGCAACGTTGTTGCCATTGCTA | |
| | CAGGCATCGTGGTGTCACGCTCGTCGTTTGGTATGGCT | |
| | TCATTCAGCTCCGGTTCCCAACGATCAAGGCGAGTTA | |
| | CATGATCCCCCATGTTGTGCAAAAAAGCGGTTAGCTC | |
| | CTTCGGTCCTCCGATCGTTGTCAGAAGTAAGTTGGCCG | |
| | CAGTGTTATCACTCATGGTTATGGCAGCACTGCATAAT | |
| | TCTCTTACTGTCATGCCATCCGTAAGATGCTTTTCTGT | |
| | GACTGGTGAGTACTCAACCAAGTCATTCTGAGAATAG | |
| | TGTATGCGGCGACCGAGTTGCTCTTGCCCGGCGTCAAT | |
| | ACGGGATAATACCGCGCCACATAGCAGAACTTTAAAA | |
| | GTGCTCATCATTGGAAAACGTTCTTCGGGGCGAAAAC | |
| | TCTCAAGGATCTTACCGCTGTTGAGATCCAGTTCGATG | |
| | TAACCCACTCGTGCACCCAACTGATCTTCAGCATCTTT | |
| | TACTTTCACCAGCGTTTCTGGGTGAGCAAAAACAGGA | |
| | AGGCAAAATGCCGCAAAAAAGGGAATAAGGGCGACA | |
| | CGGAAATGTTGAATACTCATACTCTTCCTTTTTCAATA | |
| | TTATTGAAGCATTTATCAGGGTTATTGTCTCATGAGCG | |
| | GATACATATTTGAATGTATTTAGAAAAATAAACAAAT | |
| | AGGGGTTCCGCGCACATTTCCCCGAAAAGTGCCACCT | |
| | AAATTGTAAGCGTTAATATTTTGTTAAAATTCGCGTTA | |
| | AATTTTTGTTAAATCAGCTCATTTTTTAACCAATAGGC | |
| | CGAAATCGGCAAAATCCCTTATAAATCAAAAGAATAG | |
| | ACCGAGATAGGGTTGAGTGGCCGCTACAGGGCGCTCC | |
| | CATTCGCCATTCAGGCTGCGCAACTGTTGGGAAGGGC | |
| | GTTTCGGTGCGGGCCTCTTCGCTATTACGCCAGCTGGC | |
| | GAAAGGGGGATGTGCTGCAAGGCGATTAAGTTGGGTA | |
| | ACGCCAGGGTTTTCCCAGTCACGACGTTGTAAAACGA | |
| | CGGCCAGTGAGCGCGACGTAATACGACTCACTATAGG | |
| | GCGAATTGGCGGAAGGCCGTCAAGGCCTAGGCGCGCC | |
| | AACGCGT | |
| 23 | TTGGCCACTCCCTCTCTGCGCGCTCGCTCGCTCACTGA | Full DNA sequence |
| | GGCCGCCCGGGCAAAGCCCGGGCGTCGGGCGACCTTT | of vector (pAAV- |
| | GGTCGCCCGGCCTCAGTGAGCGAGCGAGCGCGCAGAG | CBA-SS4-GBA- |
| | AGGGAGTGGCCAACTCCATCACTAGGGGTTCCTTACG | WPRE-bGH); |
| | TACAATTGGGATCCCGGACCGTCGACATTGATTATTG | coding sequence for |
| | ACTAGTTATTAATAGTAATCAATTACGGGGTCATTAGT | GBA1 is underlined |
| | TCATAGCCCATATATGGAGTTCCGCGTTACATAACTTA | Underlined, codon |
| | CGGTAAATGGCCCGCCTGGCTGACCGCCCAACGACCC | Optimized GBA1 |
| | CCGCCCATTGACGTCAATAATGACGTATGTTCCCATAG | DNA sequence |
| | TAACGCCAATAGGGACTTTCCATTGACGTCAATGGGT | (Human) |
| | GGAGTATTTACGGTAAACTGCCCACTTGGCAGTACAT | |
| | CAAGTGTATCATATGCCAAGTACGCCCCCTATTGACGT | |
| | CAATGACGGTAAATGGCCCGCCTGGCATTATGCCCAG | |
| | TACATGACCTTATGGGACTTTCCTACTTGGCAGTACAT | |
| | CTACGTATTAGTCATCGCTATTACCATGGTCGAGGTGA | |
| | GCCCCACGTTCTGCTTCACTCTCCCCATCTCCCCCCCC | |
| | TCCCCACCCCCAATTTTGTATTTATTTATTTTTTAATTA | |
| | TTTTGTGCAGCGATGGGGGCGGGGGGGGGGGGGGGG | |
| | CGCGCGCCAGGCGGGGGGGGCGGGGCGAGGGGCGG | |
| | GGCGGGGCGAGGCGGAGAGGTGCGGCGGCAGCCAAT | |
| | CAGAGCGGCGCGCTCCGAAAGTTTCCTTTTATGGCGA | |
| | GGCGGCGGCGGCGGCGGCCCTATAAAAAGCGAAGCG | |
| | CGCGGCGGGCGGGAGTCGCTGCGCGCTGCCTTCGCCC | |
| | CGTGCCCCGCTCCGCCGCCGCCTCGCGCCGCCCGCCC | |
| | CGGCTCTGACTGACCGCGTTACTCCCACAGGTGAGCG | |
| | GGCGGGACGGCCCTTCTCCTCCGGGCTGTAATTAGCG | |
| | CTTGGTTTAATGACGGCTTGTTTCTTTTCTGTGGCTGC | |
| | GTGAAAGCCTTGAGGGGCTCCGGGAGGGCCCTTTGTG | |
| | CGGGGGGAGCGGCTCGGGGGGTGCGTGCGTGTGTGTG | |
| | TGCGTGGGGAGCGCCGCGTGCGGCTCCGCGCTGCCCG | |
| | GCGGCTGTGAGCGCTGCGGGCGCGGCGCGGGGCTTTG | |
| | TGCGCTCCGCAGTGTGCGCGAGGGGAGCGCGGCCGGG | |
| | GGCGGTGCCCCGCGGTGCGGGGGGGGCTGCGAGGGG | |
| | AACAAAGGCTGCGTGCGGGGTGTGTGCGTGGGGGGGT | |
| | GAGCAGGGGGTGTGGGCGCGTCGGTCGGGCTGCAACC | |
| | CCCCCTGCACCCCCCTCCCCGAGTTGCTGAGCACGGC | |
| | CCGGCTTCGGGTGCGGGGCTCCGTACGGGGCGTGGCG | |
| | CGGGGCTCGCCGTGCCGGGCGGGGGGTGGCGGCAGGT | |
| | GGGGGTGCCGGGCGGGGCGGGGCCGCCTCGGGCCGG | |
| | GGAGGGCTCGGGGGAGGGGCGCGGCGGCCCCCGGAG | |
| | CGCCGGCGGCTGTCGAGGCGCGGCGAGCCGCAGCCAT | |
| | TGCCTTTTATGGTAATCGTGCGAGAGGGCGCAGGGAC | |
| | TTCCTTTGTCCCAAATCTGTGCGGAGCCGAAATCTGGG | |
| | AGGCGCCGCCGCACCCCCTCTAGCGGGCGCGGGGCGA | |
| | AGCGGTGCGGCGCCGGCAGGAAGGAAATGGGCGGGG | |
| | AGGGCCTTCGTGCGTCGCCGCGCCGCCGTCCCCTTCTC | |
| | CCTCTCCAGCCTCGGGGCTGTCCGCGGGGGGACGGCT | |
| | GCCTTCGGGGGGGACGGGGCAGGGCGGGGTTCGGCTT | |
| | CTGGCGTGTGACCGGCGGCTCTAGAGCCTCTGCTAAC | |
| | CATGTTCATGCCTTCTTCTTTTTCCTACAGCTCCTGGGC | |
| | AACGTGCTGGTTATTGTGCTGTCTCATCATTTTGGCAA | |
| | AGAATTCGCCACCATGACCATCCTGTTCCTGACAATG | |
| | GTGATCAGCTACTTCGGCTGCATGAAGGCCGCCAGAC | |
| | CTTGTATCCCCAAGAGCTTCGGATATAGCAGCGTGGT | |
| | CTGCGTGTGCAACGCCACCTACTGCGATAGCTTCGAC | |
| | CCACCTACCTTTCCAGCTCTGGGCACCTTCTCCAGATA | |
| | CGAGTCTACAAGAAGCGGCAGAAGAATGGAACTGTCC | |
| | ATGGGCCCTATCCAGGCCAACCACACCGGCACAGGCC | |
| | TCCTGTTGACCCTGCAGCCCGAGCAGAAATTTCAGAA | |
| | AGTGAAGGGATTCGGCGGCGCCATGACCGATGCCGCC | |
| | GCTCTGAATATCCTGGCGCTGAGCCCTCCTGCCCAGA | |
| | ACCTGCTGCTGAAGAGCTACTTTAGCGAGGAGGGGAT | |
| | CGGCTACAACATTATCAGAGTGCCCATGGCCAGCTGC | |
| | GACTTTAGCATCAGAACCTATACATACGCCGACACCC | |
| | CAGATGACTTCCAGCTGCACAACTTCAGCCTGCCTGA | |
| | GGAAGATACAAAGCTGAAAATCCCCCTGATCCACCGG | |
| | GCCCTGCAACTGGCTCAGCGGCCAGTGTCCCTGCTGG | |
| | CCTCTCCTTGGACCAGCCCTACCTGGCTGAAGACCAAT | |
| | GGCGCCGTGAACGGCAAGGGCTCTCTGAAGGGCCAGC | |
| | CTGGCGACATCTACCATCAGACCTGGGCCAGATACTT | |
| | CGTGAAGTTCCTGGATGCCTACGCTGAGCACAAGCTG | |
| | CAGTTCTGGGCCGTGACCGCCGAGAACGAGCCTTCCG | |
| | CCGGACTGCTCAGCGGCTATCCTTTCCAGTGCCTGGGC | |
| | TTCACACCTGAGCACCAGCGCGACTTCATCGCCAGGG | |
| | ACCTGGGCCCTACACTGGCCAATAGCACACACCACAA | |
| | CGTGAGACTGCTGATGCTGGATGATCAGAGACTGCTG | |
| | CTTCCCCACTGGGCTAAGGTCGTGCTGACCGACCCTG | |
| | AAGCCGCTAAGTACGTGCACGGCATCGCCGTGCACTG | |
| | GTACCTGGACTTCCTGGCCCCTGCCAAGGCCACCCTG | |
| | GGAGAAACCCACCGGCTGTTCCCTAACACCATGCTGT | |
| | TTGCCTCAGAGGCCTGTGTGGGCTCCAAATTCTGGGA | |
| | GCAAAGCGTGCGGCTGGGCTCTTGGGACAGAGGAATG | |
| | CAGTACAGCCACAGCATCATCACCAACCTGCTGTACC | |
| | ACGTGGTGGGCTGGACCGACTGGAACCTGGCCCTGAA | |
| | CCCCGAGGGAGGCCCTAACTGGGTCAGAAACTTCGTG | |
| | GACAGCCCCATCATTGTGGATATCACCAAGGACACGT | |
| | TCTACAAGCAGCCAATGTTCTACCACCTGGGCCACTTC | |
| | AGCAAGTTCATCCCTGAAGGCAGTCAGAGAGTGGGCC | |
| | TGGTGGCCTCTCAGAAGAACGACCTGGACGCCGTTGC | |
| | GCTGATGCACCCCGACGGCAGCGCCGTGGTGGTGGTG | |
| | CTGAATAGATCTTCTAAGGATGTGCCTCTGACAATCA | |
| | AGGACCCTGCTGTGGGATTTCTGGAAACAATCAGCCC | |
| | TGGATACAGCATCCATACATACCTGTGGCGGCGGCAG | |
| | TGAGATTAATCAACCTCTGGATTACAAAATTTGTGAA | |
| | AGATTGACTGGTATTCTTAACTATGTTGCTCCTTTTAC | |
| | GCTATGTGGATACGCTGCTTTAATGCCTTTGTATCATG | |
| | CTATTGCTTCCCGTATGGCTTTCATTTTCTCCTCCTTGT | |
| | ATAAATCCTGGTTGCTGTCTCTTTATGAGGAGTTGTGG | |
| | CCCGTTGTCAGGCAACGTGGCGTGGTGTGCACTGTGTT | |
| | TGCTGACGCAACCCCCACTGGTTGGGGCATTGCCACC | |
| | ACCTGTCAGCTCCTTTCCGGGACTTTCGCTTTCCCCCT | |
| | CCCTATTGCCACGGCGGAACTCATCGCCGCCTGCCTTG | |
| | CCCGCTGCTGGACAGGGGCTCGGCTGTTGGGCACTGA | |
| | CAATTCCGTGGTGTTGTCGGGGAAATCATCGTCCTTTC | |
| | CTTGGCTGCTCGCCTGTGTTGCCACCTGGATTCTGCGC | |
| | GGGACGTCCTTCTGCTACGTCCCTTCGGCCCTCAATCC | |
| | AGCGGACCTTCCTTCCCGCGGCCTGCTGCCGGCTCTGC | |
| | GGCCTCTTCCGCGTCTTCGCCTTCGCCCTCAGACGAGT | |
| | CGGATCTCCCTTTGGGCCGCCTCCCCGCCTGATCCTGT | |
| | GCCTTCTAGTTGCCAGCCATCTGTTGTTTGCCCCTCCC | |
| | CCGTGCCTTCCTTGACCCTGGAAGGTGCCACTCCCACT | |
| | GTCCTTTCCTAATAAAATGAGGAAATTGCATCGCATTG | |
| | TCTGAGTAGGTGTCATTCTATTCTGGGGGGTGGGGTG | |
| | GGGCAGGACAGCAAGGGGGAGGATTGGGAAGACAAT | |
| | AGCAGGCATGCTCGAAATTAGGAACCCCTAGTGATGG | |
| | AGTTGGCCACTCCCTCTCTGCGCGCTCGCTCGCTCACT | |
| | GAGGCCGCCCGGGCAAAGCCCGGGCGTCGGGCGACC | |
| | TTTGGTCGCCCGGCCTCAGTGAGCGAGCGAGCGCGCA | |
| | GAGAGGGAGTGGCCAATCGCGACGGCCGACGTCTTTG | |
| | TTACAACTTACTATATATATGCACACATATATATATAT | |
| | TTGGGTATATTGGGGGGGTTCTAATTTAAGAAATGCA | |
| | TAATTGGCTATAGACAGACAGTTGTCAGAACTTGGCA | |
| | ATGGGTACGTGCAGGTTCATTATACCAAGTCTACTTGT | |
| | AGTTGTTCAAAATGTATCATAATACAAGGCCGGGCGA | |
| | GGTCGTCACGCCTGTAATCCCAGCATTTTGGGAGGCT | |
| | AAGGCAGGAGGATTGCTTGAGGTCAGGAGTTTGTGAC | |
| | CAGCCTGGGCAACAGAGCAAGACCCTGTCTCCAAAAA | |
| | GAAAAAAAATAATTTTTTACAAAATAAAAACAAAATG | |
| | TATCATCAGACGAAATTAAATAAGAGGCAATTCATTG | |
| | TAATGACAACTTTTCCCAGCTTGACATTTAACAAAAA | |
| | GTCTAAGTCCTCTTAATTCATATTTAATGATCAAATAT | |
| | CAAATACTAATTTTTTTTTTTTTTTTTTTTTTGAGACGG | |
| | AGTCTCGCTCTGTCGCCCAGGCTGGAGTGCAGTGGCG | |
| | CGATCCTGGCTCACTGCAAGCTCCGCCTCCCGGGTTCA | |
| | CGCCATTCTCCTGCCTCAGCCTCCCGAGTAGCTGGGAT | |
| | TACAGACATGCGCCACCACGCCCGGCTAATTTTGTATT | |
| | TTTAGTAGAGATGGGGTTTCTCCATGTTGGTCAGGCTG | |
| | GTCTTGAATTTCCCACCTCAGGTGATCTGCCTGCCTCA | |
| | GCCTCACAAAGCAGTAGCTGGGACTACAGGCACCCAC | |
| | CACCACACTTGGTTAATTCTTTTGTATTTTTTTTGTAAA | |
| | GACGGGATTTCACCATGTTAGCCAGGATGGTCTCGAT | |
| | CTCCTGATCTCATGATCCGCCCGCCTCAGCCTCCCAAA | |
| | GTGCTGGGATTACAGGCGTGAGCCACCCCGCCCGGCC | |
| | ATCAAATACTAATTCTTAAATGGTAAGGACCCACTATT | |
| | CAGAACCTGTATCCTTATCACTAATATGCAAATATTTA | |
| | TTGAATACTTACTATGTCATGCATACTAGAGAGAGTTA | |
| | GATAAATTTGATACAGCTACCCTCACAGAACTTACAG | |
| | TGTAATAGATGGCATGACATGTACATGAGTAACTGTG | |
| | AACAGTGTTAAATTGCTATTTAAAAAAAAAGACGGCT | |
| | GGGCGCTGTGGCTCATGCCTGTAATCCCAGCACTTTGG | |
| | GAGGCCAAGGCAAGTTGATCGCTCGAGGTCAAGAGTT | |
| | CGAGACCAGCCTGGCCAACGTGGTAAAACCCCGTCTC | |
| | TACTAAAAATACAAAAAAAAAATTAGCCAGGCATGGT | |
| | GGCACAGGCCTGTAATCCCAGCTACTAGGGAGGCTGA | |
| | GACATGGAGAACTGCTTGAATCCAGGAGGCAGAGGTT | |
| | ACAGTGAGCCGAGATCATACCACTACACTCCAGCCTG | |
| | AGTGACAGAGCGAGACTCCTGTCTAAAAAAAAAAAA | |
| | AAAAAAAAAGATACAGGTTAAGTGTTATGGTAGTTGA | |
| | AGAGAGAACTCAAACTCTGTCTCAGAAGCCTCACTTG | |
| | CATGTGGACCACTGATATGAAATAATATAAATAGGTA | |
| | TAATTCAATAAATAGGAACTTCAGTTTTAATCATCCCA | |
| | AACACCAAAACTTCCTATCAAACAGGTCCAATAAACT | |
| | CAATCTCTATAAGAGCTAGACAGAAATCTACTTGGTG | |
| | GCCTATAATCTTATTAGCCCTTACTTGTCCCATCTGAT | |
| | ATTAATTAACCCCATCTAATATGGATTAGTTAACAATC | |
| | CAGTGGCTGCTTTGACAGGAACAGTTGGAGAGAGTTG | |
| | GGGATTGCAACATATTCAATTATACAAAAATGCATTC | |
| | AGCATCTACCTTGATTAAGGCAGTGTGCAACAGAATT | |
| | TGCAGGAGAGTAAAAGAATGATTATAAATTTACAACC | |
| | CTTAAAGAGCTATAGCTGGGCGTGGTGGCTCATGCCT | |
| | GTAAATCCCAGCACTTTGGGAGGCTGAGGCGGGTGGA | |
| | TCACCTGAGGCCAGAAGTTCAAGACCAGCCTAGCCAA | |
| | CATGGCGAAACCCTGTCTCTACAAAAAATACAAAAAT | |
| | TAGCCGGGTGTGGTGGCACGTGCCTGTAGTCCCAGTT | |
| | ACTTGGGAGGCCGAGGCAGGAGAATCGCTTGAACCTA | |
| | GGAGGTGGAGGCTGCAGTGAGCCGAGATTGTGCCACT | |
| | GCACTCCACTTCAGCCTGGGCGACAAGAGCAAGACTC | |
| | CGTCACAAAAAAAAAAAAAAAAAAAAAGGCTAAAAT | |
| | CTAGTGGGAAAGGCATATATACATACAACTAACTGTA | |
| | TAGCATAATAAAGCTCATAATCTGTAACAAAATCTAA | |
| | TTCGACAAGCCCAGAAACTTGTGATTTACCAAAAACA | |
| | GTTATATATACACAAAAAGTAAACCTAGAACCCAAAG | |
| | TTACCCAGCACCAATGATTCTCTCCCTAAGCAGTATCA | |
| | AGTTTAAAGCAGTGATTACATTCTACTGCCTAGATTGT | |
| | AAACTGAGTAAAGGAGACCAGCACCTTTCTGCTACTG | |
| | AACTAGCACAGCCGTGTAAACCAACAAGGCAATGGCA | |
| | GTGCCCAACTTTCTGTATGAATATAAGTTACATCTGTT | |
| | TTATTATTTGTGACTTGGTGTTGCATGTGGTTATTATC | |
| | AACACCTTCTGAAAGAACAACTACCTGCTCAGGCTGC | |
| | CATAACAAAATACCACAGACTGAGTGACTTAACAGAA | |
| | ACTTATTTCTCACAGTTTTGGAGGCTGGGAAGTCCAAA | |
| | ATTAAGCTAACTGCAAGGTAGGTTTCAATCTCAGGCC | |
| | TCTTCTTTGGCTTGAAGGTCTTCTAACTGTGTGCTCAC | |
| | ATGACCTCTTCTAACAAGCTCTCTGGTGTCTCTTTTTTT | |
| | TTTTTTTTCTTTTTTGAGACAGAGTCTCACTCTGTCACC | |
| | CAGGCTGGAGTACAGTGGCACAATCTGGGCTCACTGC | |
| | AACCTCCAACTCCCGGGTTCAAGTGATTCTCATGCCTC | |
| | ACCCTCCCGAGTAGCTTGGATGACAGGAGCCCGCTAC | |
| | CACACCCAGCTAATTTTTGTATTTTTAGTAGAGATGGT | |
| | GTTTCACTACATTGGCCAGGCTGGTCTCAAACTCCTGA | |
| | CCTCGTGATCCACCCACCTTGGCCTCCCAAAGTGCTGG | |
| | GATTACAGGTGTGAGCCACTGCGCCCGTCCTGGTGTCT | |
| | TTTCATATAAGGGCACTAATCCAATCAGACCTGGGCC | |
| | CAACCCTCCCGACTTCTTCTAACTGTAATTACCTTCCA | |
| | AAGGCCCTGTCTCCAAATACCATCACACTGGGGGTTA | |
| | GGACTTCAAAAAAGGTATGGGGGGGGTGTGGGAGGA | |
| | CATAAATGCTCAGTCCATAACAAGCACCCAACATAAA | |
| | AATGGCTAGAACAGATCACAAAAAAAAGGTCCTGTAT | |
| | GGCTTTGGGGAAGGGCTCAACCCCAAAATATCTGAAA | |
| | GCTCTGGAGGGGCCTAGAAGTGGTAAATGAATGAAAA | |
| | CGTGGTTACTCTCCCGATCTGCCTTTCCCAAATATGGC | |
| | CATTCTTGGCTGAATCAGAAATCAAAGGACAGGTTAT | |
| | TAATTACTAGCTCTAAGTTACTTACCATTTGCTGAGAC | |
| | AGTTCAGAAATCTGACTGCATCTCCTCAGGGATCTAG | |
| | AACACAGTTCTCAAATTCTAACTTACTTGTGATATACT | |
| | TGTGAATGATAAAAATCGCTACAGGTACTTTTATTAAT | |
| | CTGAAAGAGTATTGAGAAATTACCTTTCATTCTGACTT | |
| | TTGTCTGGAATGAAAATCAATACTTTTGCTATATTCCA | |
| | TTACTGAAATAATTTTACTTTCCAGTAAAACTGGCATT | |
| | ATAATTTTTTTTAATTTTTAAAACTTCATAATTTTTTGC | |
| | CAGACTGACCCATGTAAACATACAAATTACTAATAAT | |
| | TATGCACGTCACATCTGTAATAATGGCCTTCATGTAAA | |
| | CATTTTTGTGGTTTACACATAAAATCTCTAATTACAAA | |
| | GCTATATTATCTAAAATTACAGTAAGCAAGAAAATTA | |
| | ATCCAAGCTAAGACAATACTTGCAACATCAATTCATC | |
| | ATCTGTGACAAGGACTGCTTAAGTCTCTTTGTGGTTGA | |
| | CGTCATTAATTAACTGGCCTCATGGGCCTTCCGCTCAC | |
| | TGCCCGCTTTCCAGTCGGGAAACCTGTCGTGCCAGCTG | |
| | CATTAACATGGTCATAGCTGTTTCCTTGCGTATTGGGC | |
| | GCTCTCCGCTTCCTCGCTCACTGACTCGCTGCGCTCGG | |
| | TCGTTCGGGTAAAGCCTGGGGTGCCTAATGAGCAAAA | |
| | GGCCAGCAAAAGGCCAGGAACCGTAAAAAGGCCGCG | |
| | TTGCTGGCGTTTTTCCATAGGCTCCGCCCCCCTGACGA | |
| | GCATCACAAAAATCGACGCTCAAGTCAGAGGTGGCGA | |
| | AACCCGACAGGACTATAAAGATACCAGGCGTTTCCCC | |
| | CTGGAAGCTCCCTCGTGCGCTCTCCTGTTCCGACCCTG | |
| | CCGCTTACCGGATACCTGTCCGCCTTTCTCCCTTCGGG | |
| | AAGCGTGGCGCTTTCTCATAGCTCACGCTGTAGGTATC | |
| | TCAGTTCGGTGTAGGTCGTTCGCTCCAAGCTGGGCTGT | |
| | GTGCACGAACCCCCCGTTCAGCCCGACCGCTGCGCCT | |
| | TATCCGGTAACTATCGTCTTGAGTCCAACCCGGTAAG | |
| | ACACGACTTATCGCCACTGGCAGCAGCCACTGGTAAC | |
| | AGGATTAGCAGAGCGAGGTATGTAGGCGGTGCTACAG | |
| | AGTTCTTGAAGTGGTGGCCTAACTACGGCTACACTAG | |
| | AAGAACAGTATTTGGTATCTGCGCTCTGCTGAAGCCA | |
| | GTTACCTTCGGAAAAAGAGTTGGTAGCTCTTGATCCG | |
| | GCAAACAAACCACCGCTGGTAGCGGTGGTTTTTTTGTT | |
| | TGCAAGCAGCAGATTACGCGCAGAAAAAAAGGATCTC | |
| | AAGAAGATCCTTTGATCTTTTCTACGGGGTCTGACGCT | |
| | CAGTGGAACGAAAACTCACGTTAAGGGATTTTGGTCA | |
| | TGAGATTATCAAAAAGGATCTTCACCTAGATCCTTTTA | |
| | AATTAAAAATGAAGTTTTAAATCAATCTAAAGTATAT | |
| | ATGAGTAAACTTGGTCTGACAGTTACCAATGCTTAATC | |
| | AGTGAGGCACCTATCTCAGCGATCTGTCTATTTCGTTC | |
| | ATCCATAGTTGCCTGACTCCCCGTCGTGTAGATAACTA | |
| | CGATACGGGAGGGCTTACCATCTGGCCCCAGTGCTGC | |
| | AATGATACCGCGAGAACCACGCTCACCGGCTCCAGAT | |
| | TTATCAGCAATAAACCAGCCAGCCGGAAGGGCCGAGC | |
| | GCAGAAGTGGTCCTGCAACTTTATCCGCCTCCATCCAG | |
| | TCTATTAATTGTTGCCGGGAAGCTAGAGTAAGTAGTTC | |
| | GCCAGTTAATAGTTTGCGCAACGTTGTTGCCATTGCTA | |
| | CAGGCATCGTGGTGTCACGCTCGTCGTTTGGTATGGCT | |
| | TCATTCAGCTCCGGTTCCCAACGATCAAGGCGAGTTA | |
| | CATGATCCCCCATGTTGTGCAAAAAAGCGGTTAGCTC | |
| | CTTCGGTCCTCCGATCGTTGTCAGAAGTAAGTTGGCCG | |
| | CAGTGTTATCACTCATGGTTATGGCAGCACTGCATAAT | |
| | TCTCTTACTGTCATGCCATCCGTAAGATGCTTTTCTGT | |
| | GACTGGTGAGTACTCAACCAAGTCATTCTGAGAATAG | |
| | TGTATGCGGCGACCGAGTTGCTCTTGCCCGGCGTCAAT | |
| | ACGGGATAATACCGCGCCACATAGCAGAACTTTAAAA | |
| | GTGCTCATCATTGGAAAACGTTCTTCGGGGCGAAAAC | |
| | TCTCAAGGATCTTACCGCTGTTGAGATCCAGTTCGATG | |
| | TAACCCACTCGTGCACCCAACTGATCTTCAGCATCTTT | |
| | TACTTTCACCAGCGTTTCTGGGTGAGCAAAAACAGGA | |
| | AGGCAAAATGCCGCAAAAAAGGGAATAAGGGCGACA | |
| | CGGAAATGTTGAATACTCATACTCTTCCTTTTTCAATA | |
| | TTATTGAAGCATTTATCAGGGTTATTGTCTCATGAGCG | |
| | GATACATATTTGAATGTATTTAGAAAAATAAACAAAT | |
| | AGGGGTTCCGCGCACATTTCCCCGAAAAGTGCCACCT | |
| | AAATTGTAAGCGTTAATATTTTGTTAAAATTCGCGTTA | |
| | AATTTTTGTTAAATCAGCTCATTTTTTAACCAATAGGC | |
| | CGAAATCGGCAAAATCCCTTATAAATCAAAAGAATAG | |
| | ACCGAGATAGGGTTGAGTGGCCGCTACAGGGCGCTCC | |
| | CATTCGCCATTCAGGCTGCGCAACTGTTGGGAAGGGC | |
| | GTTTCGGTGCGGGCCTCTTCGCTATTACGCCAGCTGGC | |
| | GAAAGGGGGATGTGCTGCAAGGCGATTAAGTTGGGTA | |
| | ACGCCAGGGTTTTCCCAGTCACGACGTTGTAAAACGA | |
| | CGGCCAGTGAGCGCGACGTAATACGACTCACTATAGG | |
| | GCGAATTGGCGGAAGGCCGTCAAGGCCTAGGCGCGCC | |
| | AACGCGT | |
| 24 | CACTCCCTCTCTGCGCGCTCGCTCGCTCACTGAGGCCG | ITR |
| | GGCGACCAAAGGTCGCCCACGCCCGGGCTTTGCCCGG | |
| | GCG | |
| 25 | MEFSSPSREECPKPLSRVSIMAGSLTGLLLLQAVSWASGa | WT GBA1 Amino |
| | rpcipksfgyssvvcvcnatycdsfdpptfpalgtfsryestrsgrrmelsmgpiqanht | Acid |
| | gtgllltlqpeqkfqkvkgfggamtdaaalnilalsppaqnlllksyfseegigyniirvp | |
| | mascdfsirtytyadtpddfqlhnfslpeedtklkiplihralqlaqrpvsllaspwtspt | |
| | wlktngavngkgslkgqpgdiyhqtwaryfvkfldayaehklqfwavtaenepsagl | |
| | lsgypfqclgftpehqrdfiardlgptlansthhnvrllmlddqrlllphwakvvltdpea | |
| | akyvhgiavhwyldflapakatlgethrlfpntmlfaseacvgskfweqsvrlgswdr | |
| | gmqyshsiitnllyhvvgwtdwnlalnpeggpnwvrnfvdspiivditkdtfykqp | |
| | mfyhlghfskfipegsqrvglvasqkndldavalmhpdgsavvvvlnrsskdvplti | |
| | kdpavgfletispgysihtylwrrq | |
| 26 | MASLWLLSCFSLVGAAFGarpcipksfgyssvvcvcnatycdsfdpptf | SS1-mature GBA1 |
| | palgtfsryestrsgrrmelsmgpiqanhtgtgllltlqpeqkfqkvkgfggamtdaaal | Amino Acid |
| | nilalsppaqnlllksyfseegigyniirvpmascdfsirtytyadtpddfqlhnfslpee | |
| | dtklkiplihralqlaqrpvsllaspwtsptwlktngavngkgslkgqpgdiyhqtwar | |
| | yfvkfldayaehklqfwavtaenepsagllsgypfqclgftpehqrdfiardlgptlanst | |
| | hhnvrllmlddqrlllphwakvvltdpeaakyvhgiavhwyldflapakatlgethrlf | |
| | pntmlfaseacvgskfweqsvrlgswdrgmqyshsiitnllyhvvgwtdwnlalnp | |
| | eggpnwvrnfvdspiivditkdtfykqpmfyhlghfskfipegsqrvglvasqkndld | |
| | avalmhpdgsavvvvlnrsskdvpltikdpavgfletispgysihtylwrrq | |
| 27 | MFQTGGLIVFYGLLAQTMAarpcipksfgyssvvcvcnatycdsfdpp | SS2-mature GBA1 |
| | tfpalgtfsryestrsgrrmelsmgpiqanhtgtgllltlqpeqkfqkvkgfggamtdaa | Amino Acid |
| | alnilalsppaqnlllksyfseegigyniirvpmascdfsirtytyadtpddfqlhnfslpe | |
| | edtklkiplihralqlaqrpvsllaspwtsptwlktngavngkgslkgqpgdiyhqtwa | |
| | ryfvkfldayaehklqfwavtaenepsagllsgypfqclgftpehqrdfiardlgptlan | |
| | sthhnvrllmlddqrlllphwakvvltdpeaakyvhgiavhwyldflapakatlgethr | |
| | lfpntmlfaseacvgskfweqsvrlgswdrgmqyshsiitnllyhvvgwtdwnlaln | |
| | peggpnwvrnfvdspiivditkdtfykqpmfyhlghfskfipegsqrvglvasqkndl | |
| | davalmhpdgsavvvvlnrsskdvpltikdpavgfletispgysihtylwrrq | |
| 28 | MLLLLLLLGLRLQLSLGarpcipksfgyssvvcvcnatycdsfdpptfpal | SS3-mature GBA1 |
| | gtfsryestrsgrrmelsmgpiqanhtgtgllltlqpeqkfqkvkgfggamtdaaalnil | Amino Acid |
| | alsppaqnlllksyfseegigyniirvpmascdfsirtytyadtpddfqlhnfslpeedtk | |
| | lkiplihralqlaqrpvsllaspwtsptwlktngavngkgslkgqpgdiyhqtwaryfv | |
| | kfldayaehklqfwavtaenepsagllsgypfqclgftpehqrdfiardlgptlansthh | |
| | nvrllmlddqrlllphwakvvltdpeaakyvhgiavhwyldflapakatlgethrlfpn | |
| | tmlfaseacvgskfweqsvrlgswdrgmqyshsiitnllyhvvgwtdwnlalnpeg | |
| | gpnwvrnfvdspiivditkdtfykqpmfyhlghfskfipegsqrvglvasqkndldav | |
| | almhpdgsavvvvlnrsskdvpltikdpavgfletispgysihtylwrrq | |
| 29 | MLLLLLLLGLRLQLSLGarpcipksfgyssvvcvcnatycdsfdpptfpal | SS4-mature GBA1 |
| | gtfsryestrsgrrmelsmgpiqanhtgtgllltlqpeqkfqkvkgfggamtdaaalnil | Amino Acid |
| | alsppaqnlllksyfseegigyniirvpmascdfsirtytyadtpddfqlhnfslpeedtk | |
| | lkiplihralqlaqrpvsllaspwtsptwlktngavngkgslkgqpgdiyhqtwaryfv | |
| | kfldayaehklqfwavtaenepsagllsgypfqclgftpehqrdfiardlgptlansthh | |
| | nvrllmlddqrlllphwakvvltdpeaakyvhgiavhwyldflapakatlgethrlfpn | |
| | tmlfaseacvgskfweqsvrlgswdrgmqyshsiitnllyhvvgwtdwnlalnpeg | |
| | gpnwvrnfvdspiivditkdtfykqpmfyhlghfskfipegsqrvglvasqkndldav | |
| | almhpdgsavvvvlnrsskdvpltikdpavgfletispgysihtylwrrq | |
| 30 | MEFSSPSREECPKPLSRVSIMAGSLTGLLLLQAVSWASG | WT SS amino acid |
| 31 | MASLWLLSCFSLVGAAFG | SS1 amino acid |
| 32 | MFQTGGLIVFYGLLAQTMA | SS2 amino acid |
| 33 | MLLLLLLLGLRLQLSLG | SS3 amino acid |
| 34 | MTILFLTMVISYFGCMKA | SS4 amino acid |

1. A recombinant adeno-associated virus (rAAV) particle comprising (1) a rAAV vector comprising an expression cassette for expressing a glucocerebrosidase (GCase) enzyme, wherein the expression cassette comprises a gene encoding the GCase enzyme operably linked to a promoter and optionally an enhancer, and (2) a modified AAV9 capsid protein comprising a targeting peptide that comprises SEQ ID NO: 16.

2. The rAAV particle of claim 1, wherein the GCase enzyme comprises an amino acid sequence of SEQ ID NO: 1.

3. The rAAV particle of claim 1, wherein the GCase enzyme comprises an amino acid sequence of SEQ ID NO: 2.

4. The rAAV particle of claim 1, wherein the GCase enzyme comprises an amino acid sequence of SEQ ID NO: 2 and a mutated (i.e., not endogenous) signaling peptide.

5. The rAAV particle of claim 1, wherein the gene encoding the GCase enzyme is a codon-optimized gene.

6. The rAAV particle of claim 5, wherein the codon-optimized gene has a nucleic acid sequence selected from the group consisting of SEQ ID NO: 3, SEQ ID NO: 4, SEQ ID NO: 5, SEQ ID NO: 6, and SEQ ID NO: 7.

7. The rAAV particle of claim 1, wherein the expression cassette comprises a nucleic acid sequence selected from the group consisting of SEQ ID NO: 19, SEQ ID NO: 20, SEQ ID NO: 21, SEQ ID NO: 22, and SEQ ID NO: 23.

8. The rAAV particle of claim 1, wherein the targeting peptide is flanked by linker sequences on its N-terminal end and its C-terminal end.

9. The rAAV particle of claim 8, wherein the combined targeting peptide and linker sequences comprise SEQ ID NO: 17.

10. The rAAV particle of claim 1, wherein modified capsid protein has a sequence that is at least 98.5% identical to SEQ ID NO: 18.

11. The rAAV particle of claim 10, wherein the modified capsid protein comprises a sequence comprising SEQ ID NO: 18.

12. The rAAV particle of claim 1, wherein the rAAV vector comprises a 5′ AAV2 ITR of SEQ ID NO: 8 and a 3′ AAV2 ITR of SEQ ID NO: 9.

13. The rAAV particle of claim 1, wherein the expression cassette comprises a CMV enhancer element comprising SEQ ID NO: 10.

14. The rAAV particle of claim 1, wherein the expression cassette comprises a chicken b-actin promoter comprising SEQ ID NO: 11.

15. The rAAV particle of claim 1, wherein the rAAV vector further comprises a WPRE element.

16. The rAAV particle of claim 11, wherein the WPRE element comprises a sequence of SEQ ID NO: 12.

17. A recombinant adeno-associated virus (rAAV) particle comprising (1) a rAAV vector comprising an expression cassette for expressing a glucocerebrosidase (GCase) enzyme, wherein the expression cassette comprises a gene encoding the GCase enzyme operably linked to a promoter and optionally an enhancer, and (2) a capsid protein, wherein the expression cassette comprises a nucleic acid sequence selected from the group consisting of SEQ ID NO: 19, SEQ ID NO: 20, SEQ ID NO: 21, SEQ ID NO: 22, and SEQ ID NO: 23.

18. A method of treating Gaucher Disease (GD) type 3 in a human patient in need thereof, comprising administering to the cerebrospinal fluid (CSF) of the patient a composition comprising an effective amount of recombinant adeno-associated virus (rAAV) viral particle of claim 1.

19. The method of claim 18, wherein the composition is administered directly to the CSF of the patient via intracerebroventricular (ICV) administration.

20. The method of claim 18, wherein the composition is administered directly to the CSF of the patient via direct cisterna magna (dCM) administration.

21. The method of claim 18, wherein the composition is administered directly to the CSF of the patient with an intrathecal microcatheter (IT-CM).

22. The method of claim 18, wherein the composition is administered only once over the lifetime of the patient.

23. The method of claim 18, wherein the composition is administered only once yearly to the patient.

24-64. (canceled)
